(19) United States
(12) Patent Application Publication
(10) Pub. No.: US 2013/0129677 A1
(43) Pub. Date: May 23, 2013

(54) Thienopyridine Derivatives for the Treatment and Prevention of Dengue Virus Infections

(71) Applicant: Siga Technologies, Inc., Corvallis, OR (US)

(72) Inventors: Dongcheng Dai, Corvallis, OR (US); James R. Burgeson, (US); Shanthakumar R. Tyavanagimatt, Corvallis, OR (US); Chelsea M. Byrd, Corvallis, OR (US); Dennis E. Hruby, Albany, OR (US)

(73) Assignee: Siga Technologies, Inc., Corvallis, OR (US)

(21) Appl. No.: 13/708,224

(22) Filed: Dec. 7, 2012

Related U.S. Application Data
(63) Continuation-in-part of application No. 13/203,351, filed on Oct. 13, 2011.
(60) Provisional application No. 61/156,132, filed on Feb. 27, 2009.

(51) Int. Cl. C07D 495/04; A61K 39/00; A61K 31/55; A61K 31/5513; A61K 31/5377; A61K 45/00; A61K 31/519; A61K 31/4365; A61K 31/381; C07D 495/14; C07D 409/10; A61K 38/21; A61K 31/496

(52) U.S. Cl. CPC C07D 495/04; A61K 38/21; A61K 39/00; A61K 31/55; A61K 31/5513; A61K 31/5377; A61K 31/496; A61K 31/519; A61K 31/4365; A61K 31/381; C07D 495/14; C07D 409/10; A61K 45/00
USPC 424/85.4; 424/184.1; 514/215; 514/220; 514/233.8; 514/252.11; 514/253.04; 514/267; 514/291; 514/293; 514/301; 514/443; 540/495; 540/578; 544/127; 544/250; 544/357; 544/362; 546/80; 546/83; 546/114; 549/57

(57) ABSTRACT

Methods and pharmaceutical compositions for treating viral infections, by administering certain thienopyridine derivative compounds in therapeutically effective amounts are disclosed. Methods of using the compounds and pharmaceutical compositions thereof are also disclosed. In particular, the treatment of viral infections such as caused by flavivirus is disclosed, i.e., including but not limited to, Dengue virus, West Nile virus, yellow fever virus, Japanese encephalitis virus, and tick-borne encephalitis virus.

### CROSS REFERENCE TO RELATED APPLICATIONS

This application is a continuation-in-part and claims priority to U.S. patent application Ser. No. 13/203,351, filed Oct. 13, 2011, which is a national stage entry under U.S.C. 371(c), and claims priority to International Patent Application Number PCT/US10/25183, filed Feb. 24, 2010, which in turn claims priority to and benefit of U.S. Provisional Application No. 61/156,132, filed Feb. 27, 2009. All the applications are incorporated herein by reference in the entirety and for all purposes.

### STATEMENT REGARDING FEDERALLY SPONSORED RESEARCH OR DEVELOPMENT

This invention was made with U.S. Government support under Grants No. R43AI079937 and R01AI093356 awarded by the National Institute of Health (NIH). The U.S. Government has certain rights in the invention.

### FIELD OF THE INVENTION

This invention relates to the use of thienopyridine derivatives and analogs, as well as compositions containing the same, for the treatment of viral diseases associated with the flavivirus family such as Dengue fever, Yellow fever, West Nile, St. Louis encephalitis, Hepatitis C, Murray Valley encephalitis, and Japanese encephalitis.

### BACKGROUND OF THE INVENTION

Dengue fever (DF) is an acute febrile disease caused by one of four closely related virus serotypes (DEN-1, DEN-2, DEN-3, and DEN-4). Dengue fever is classified based on its clinical characteristics into classical dengue fever, or the more severe forms, dengue hemorrhagic fever syndrome (DHF), and dengue shock syndrome (DSS). Recovery from infection from one serotype produces life-long immunity to that particular serotype, but provides only short-lived and limited protection against any of the other serotypes (32). Dengue is a member of the Flaviviridae family which are enveloped, positive-sense RNA viruses whose human pathogens also include West Nile virus (WNV), yellow fever virus (YFV), Japanese encephalitis virus (JEV), and tick-borne encephalitis virus (TBEV) among others. Dengue transmission is via the bite of an infected Aedes aegypti mosquito which is found in tropical and sub-tropical regions around the world.

Each year regional epidemics of dengue cause significant morbidity and mortality, social disruption and substantial economic burden on the societies affected both in terms of hospitalization and mosquito control. Dengue is considered by the World Health Organization (WHO) to be the most important arthropod-borne viral disease with an estimated 50 million cases of dengue infection, including 500,000 DHF cases and 24,000 deaths worldwide each year (32, 33). WHO estimates that forty percent of the world's population (2.5 billion people) are at risk for DF, DHF, and DSS (32). Dengue is also a NIAID Category A pathogen and in terms of bio-defense, represents a significant threat to United States troops overseas. Dengue is an emerging threat to North America with a dramatic increase in severe disease in the past 25 years including major epidemics in Cuba and Venezuela, and outbreaks in Texas and Hawaii (4). Failure to control the mosquito vector and increases in long-distance travel have contributed to the increase and spread of dengue disease. The characteristics of dengue as a viral hemorrhagic fever virus (arthropod-borne, widely spread, and capable of inducing a great amount of cellular damage and eliciting an immune response that can result in severe hemorrhage, shock, and death) makes this virus a unique threat to deployed military personnel around the world as well as to travelers to tropical regions. Preparedness for both biodefense and for the public health challenges posed by dengue will require the development of new vaccines and antiviral therapeutics.

Dengue causes several illnesses with increasing severity being determined in part by prior infection with a different serotype of the virus. Classic dengue fever (DF) begins 3-8 days after the bite of an infected mosquito and is characterized by sudden onset of fever, headache, back pain, joint pain, a measles-like rash, and nausea and vomiting (20). DF is frequently referred to as “breakbone” fever due to these symptoms. The disease usually resolves after two weeks but a prolonged recovery with weakness and depression is common. The more severe form of the disease, dengue hemorrhagic fever (DHF) has a similar onset and early phase of illness as dengue fever. However, shortly after onset the disease is characterized by high fever, enlargement of the liver, and hemorrhagic phenomena such as bleeding from the nose, mouth, and internal organs due to vascular permeability (33). In dengue shock syndrome (DSS) circulatory failure and hypovolaemic shock resulting from plasma leakage occur and can lead to death in 12-24 hours without plasma replacement (33). The case fatality rate of DHF/DSS can be as high as 20% without treatment. DHF has become a leading cause of hospitalization and death among children in many countries with an estimated 500,000 cases requiring hospitalization each year and a case fatality rate of about 5% (32).

The pathogenesis of DHF/DSS is still being studied but is thought to be due in part to an enhancement of virus replication in macrophages by heterotypic antibodies, termed antibody-dependent enhancement (ADE) (8). During a secondary infection, with a different serotype of dengue virus, cross-reactive antibodies that are not neutralizing form virus-antibody complexes that are taken into monocytes and Langerhans cells (dendritic cells) and increase the number of infected cells (7). This leads to the activation of cytotoxic lymphocytes which can result in plasma leakage and the hemorrhagic features characteristic of DHF and DSS (20). This antibody-dependent enhancement of infection is one reason why the development of a successful vaccine has proven to be so difficult. Although less frequent, DHF/DSS can occur after primary infection (29), so virus virulence (15) and immune activation are also believed to contribute to the pathogenesis of the disease (25).

Dengue is endemic in more than 100 countries in Africa, the Americas, the Eastern Mediterranean, South-east Asia and the Western Pacific. During epidemics, attack rates can be as high as 80-90% of the susceptible population. All four serotypes of the virus are emerging worldwide, increasing the number of cases of the disease as well as the number of explosive outbreaks. In 2002 for example, there were 1,015,420 reported cases of dengue in the Americas alone with 14,374 cases of DHF, which is more than three times the number of dengue cases reported in the Americas in 1995 (23).

The dengue genome, approximately 11 kb in length, consists of a linear, single stranded, infectious, positive sense RNA that is translated as a single long polyprotein (reviewed in (27)). The genome is composed of seven nonstructural (NS) protein genes and three structural protein genes which encode the nucleocapsid protein (C), a membrane-associated protein (M), and an envelope protein (E). The nonstructural proteins are involved in viral RNA replication (31), viral assembly, and the inflammatory components of the disease (18). The structural proteins are involved mainly in viral particle formation (21). The precursor polyprotein is cleaved by cellular proteinases to separate the structural proteins (17), while a virus-encoded proteinase cleaves the nonstructural region of the polyprotein (6). The genome is capped and does not have a poly(A) tail at the 3′ end but instead has a stable stem-loop structure necessary for stability and replication of the genomic RNA (3). The virus binds to cellular receptors via the E protein and undergoes receptor-mediated endocytosis followed by low-pH fusion in lysosomes (19). The viral genome is then uncoated and translated into the viral precursor polyprotein. Co- and posttranslational proteolytic processing separates the structural and nonstructural proteins. The RNA-dependent RNA polymerase along with cofactors synthesizes the minus-strand RNA which serves as a template for the synthesis of the progeny plus-strand RNA (24). Viral replication is membrane associated (1, 30). Following replication, the genome is encapsidated, and the immature virus, surrounded by a lipid envelope buds into the lumen (9). The envelope proteins become glycosylated and mature viruses are released outside the cell. Essential stages or process during the virus life cycle would be possible targets for inhibition from an antiviral drug and include binding of the virus to the cell through the E protein, uptake of the virus into the cell, the capping mechanism, the viral proteinase, the viral RNA-dependent RNA polymerase, and the viral helicase.

Current management of dengue virus-related disease relies solely on vector control. There are no approved antivirals or vaccines for the treatment or prevention of dengue. Ribavirin, a guanosine analogue, has been shown to be effective against a range of RNA virus infections and works against dengue in tissue culture by inhibiting the dengue 2′-O-methyltransferase NS5 domain (2, 10). However, ribavirin did not show protection against dengue in a mouse model (14) or a rhesus monkey model (16), instead it induced anemia and thrombocytosis. While there are no currently available approved vaccines, multivalent dengue vaccines have shown some limited potential in humans (5, 11, 12, 26). However, vaccine development is difficult due to the presence of four distinct serotypes of the virus which each cause disease. Vaccine development also faces the challenge of ADE where unequal protection against the different strains of the virus could actually increase the risk of more serious disease. Therefore there is a need for antiviral drugs that target all of the serotypes of dengue. An antiviral drug administered early during dengue infection that inhibits viral replication would prevent the high viral load associated with DHF and be an attractive strategy in the treatment and prevention of disease. An antiviral drug that inhibits viral replication could be administered prior to travel to a dengue endemic region to prevent acquisition of disease, or for those that have previously been exposed to dengue, could prevent infection by another serotype of virus and decrease the chance of life-threatening DHF and DSS. Having an antiviral drug would also aid vaccine development by having a tool at hand to treat complications that may arise due to unequal immune protection against the different serotypes. Although a successful vaccine could be a critical component of an effective biodefense, the typical delay to onset of immunity, potential side-effects, cost, and logistics associated with large-scale civilian vaccinations against a low-threat risk agent suggest that a comprehensive biodefense include a separate rapid-response element. Thus, there remains an urgent need to develop a safe and effective product to protect against flavivirus infection.

### SUMMARY OF THE INVENTION

The present invention provides a pharmaceutical composition comprising a pharmaceutically acceptable carrier and a compound having the following general Formula I or a pharmaceutically acceptable salt thereof:

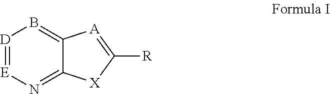

wherein X is selected from the groups consisting of O, S and N—R′, wherein R′ is selected from the group consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl, or R and R¹ together with the carbons they are attached to may form a substituted or unsubstituted ring; and

A, B, D, and E are independently N or C—R¹, C—R², C—R³ and C—R⁴, respectively, wherein R¹, R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R¹ and R together with the carbons they are attached to may form a substituted or unsubstituted ring, or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring. The pharmaceutical composition must be suitable for human or animal administration.

The present invention also provides a pharmaceutical composition comprising a pharmaceutically acceptable carrier and a compound having the following general Formula II or a pharmaceutically acceptable salt thereof:

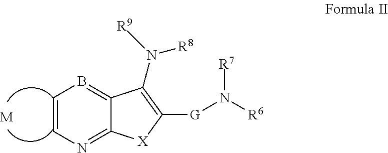

wherein X is selected from the groups consisting of O, S or N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

B is N or C—R², wherein R² is selected from the groups consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro;

G is selected from the group consisting of —C(═O)—, —C(═S)—, —S(═O)₂—, and —C(═NR⁵)—, wherein R⁵ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁵ and R⁶ or R⁷, together with the nitrogen atoms they are attached to, along with the carbon of G, or R⁵ and R⁸ or R⁹, together with the nitrogen atoms they are attached to, along with the carbon of G and two carbons of the X-containing 5-membered ring, may form a substituted or unsubstituted ring, which may be fused with an aromatic or aliphatic ring;

R⁶, R⁷, R⁸, and R⁹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁶ or R⁷ and R⁵, together with the nitrogen atoms they are attached to, along with the carbon of G, or R⁸ or R⁹ and R⁵, together with the nitrogen atoms they are attached to, along with the carbon of G and two carbons of the X-containing 5-membered ring, or R⁶ or R⁷ and R⁸ or R⁹, together with the nitrogen atoms they are attached to, along with the carbon or sulfur of G and two carbons of the X-containing 5-membered ring, or R⁶ and R⁷, together with the nitrogen atom they are attached to, or R⁸ and R⁹, together with the nitrogen atom they are attached to, may form a substituted or unsubstituted ring, which may be fused with an aromatic or aliphatic ring; and

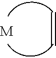

is a 7 or 8-membered ring which contains one or more heteroatoms selected from N, O and S, or a 4-membered ring which may optionally contain one or more heteroatoms selected from N, O and S. The ring may be substituted or unsubstituted, or fused with another ring, which may be aromatic or non-aromatic and may include one or more heteroatoms in the ring and may be fused with an aromatic or aliphatic ring. The pharmaceutical composition must be suitable for human or animal administration.

The present invention further provides a pharmaceutical composition comprising a pharmaceutically acceptable carrier and a compound having the following general Formula III or a pharmaceutically acceptable salt thereof:

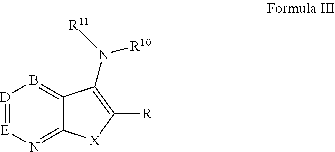

wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;

B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and

R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen,

wherein said pharmaceutical composition is suitable for human or animal administration.

The present invention further provides a pharmaceutical composition comprising a pharmaceutically acceptable carrier and a compound selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide, wherein said pharmaceutical composition is suitable for human or animal administration.

The present invention further provides a pharmaceutical composition comprising a pharmaceutically acceptable carrier and a compound or a pharmaceutically acceptable salt thereof, wherein said compound is selected from the group consisting of: 3-amino-N-(4-bromophenyl)-6-(4-chlorophenyl)thieno[2,3-b]pyridine-2-carboxamide; 3-amino-6-(3-methoxyphenyl)-N-[4-(trifluoromethoxy)phenyl]thieno[2,3-b]pyridine-2-carboxamide; 3-amino-N-(2,5-dichlorophenyl)-6-(2-thienyl)thieno[2,3-b]pyridine-2-carboxamide; 3-amino-N-(2,3-dichlorophenyl)-6-(2-thienyl)thieno[2,3-b]pyridine-2-carboxamide; 3-amino-N-(4-bromophenyl)-6-(3-methoxyphenyl)thieno[2,3-b]pyridine-2-carboxamide; 3-amino-6-(1,3-benzodioxol-5-yl)-N-(2-bromo-4-methyl-phenyl)thieno[2,3-b]pyridine-2-carboxamide; and 3-amino-6-(3-methoxyphenyl)-N-(2-phenoxyphenyl)thieno[2,3-b]pyridine-2-carboxamide, wherein said pharmaceutical composition is suitable for human or animal administration.

The present invention also provides a compound having the following general Formula II or a pharmaceutically acceptable salt thereof:

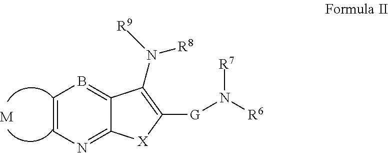

wherein X is selected from the groups consisting of O, S or N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

B is N or C—R², wherein R² is selected from the groups consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro;

G is selected from the group consisting of —C(═O)—, —C(═S)—, —S(═O)₂—, and —C(═NR⁵)—, wherein R⁵ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁵ and R⁶ or R⁷, together with the nitrogen atoms they are attached to, along with the carbon of G, or R⁵ and R⁸ or R⁹, together with the nitrogen atoms they are attached to, along with the carbon of G and two carbons of the X-containing 5-membered ring, may form a substituted or unsubstituted ring, which may be fused with an aromatic or aliphatic ring;

R⁶, R⁷, R⁸, and R⁹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁶ or R⁷ and R⁵, together with the nitrogen atoms they are attached to, along with the carbon of G, or R⁸ or R⁹ and R⁵, together with the nitrogen atoms they are attached to, along with the carbon of G and two carbons of the X-containing 5-membered ring, or R⁶ or R⁷ and R⁸ or R⁹, together with the nitrogen atoms they are attached to, along with the carbon or sulfur of G and two carbons of the X-containing 5-membered ring, or R⁶ and R⁷, together with the nitrogen atom they are attached to, or R⁸ and R⁹, together with the nitrogen atom they are attached to, may form a substituted or unsubstituted ring, which may be fused with an aromatic or aliphatic ring; and

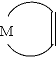

is a 7 or 8-membered ring which contains one or more heteroatoms selected from N, O and S, or a 4-membered ring which may optionally contain one or more heteroatoms selected from N, O and S. The ring may be substituted or unsubstituted, or fused with another ring, which may be aromatic or non-aromatic and may include one or more heteroatoms in the ring and may be fused with an aromatic or aliphatic ring.

The present invention also provides a compound having the following general Formula III or a pharmaceutically acceptable salt thereof:

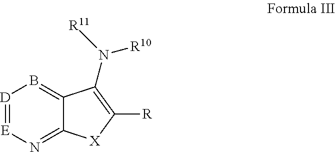

wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;

B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and

R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen.

The present invention also provides a compound selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide.

The present invention further provides a method for the treatment of at least one type of a Dengue virus infection or disease associated therewith, comprising administering in a therapeutically effective amount to a mammal in need thereof, a compound of Formula I, II or III as indicated above or a pharmaceutically acceptable salt thereof.

The present invention also provides a method for the treatment of at least one type of a Dengue infection or disease associated therewith, comprising administering in a therapeutically effective amount to a mammal in need thereof, a compound or a pharmaceutically acceptable salt thereof, wherein said compound is selected from the group consisting of: 3-amino-N-(4-bromophenyl)-6-(4-chlorophenyl)thieno[2,3-b]pyridine-2-carboxamide; 3-amino-6-(3-methoxyphenyl)-N-[4-(trifluoromethoxy)phenyl]thieno[2,3-b]pyridine-2-carboxamide; 3-amino-N-(2,5-dichlorophenyl)-6-(2-thienyl)thieno[2,3-b]pyridine-2-carboxamide; 3-amino-N-(2,3-dichlorophenyl)-6-(2-thienyl)thieno[2,3-b]pyridine-2-carboxamide; 3-amino-N-(4-bromophenyl)-6-(3-methoxyphenyl)thieno[2,3-b]pyridine-2-carboxamide; 3-amino-6-(1,3-benzodioxol-5-yl)-N-(2-bromo-4-methyl-phenyl)thieno[2,3-b]pyridine-2-carboxamide; and 3-amino-6-(3-methoxyphenyl)-N-(2-phenoxyphenyl)thieno[2,3-b]pyridine-2-carboxamide.

The present invention further provides novel intermediate compounds used in the synthesis of the compounds of the present invention. These intermediate compounds are selected from the group consisting of: tert-butyl (4E)-4-(hydroxymethylene)-5-oxoazepane-1-carboxylate; tert-butyl (3E)-3-(hydroxymethylene)-4-oxoazepane-1-carboxylate; tert-butyl 3-cyano-2-thioxo-1,2,5,6,8,9-hexahydro-7H-pyrido[2,3-d]azepine-7-carboxylate; tert-butyl 3-cyano-2-thioxo-1,2,5,7,8,9-hexahydro-6H-pyrido[3,2-c]azepine-6-carboxylate; and 3-amino-7-tert-butyloxycarbonyl-6,7,8,9-tetrahydro-5H-1-thia-7,10-diaza-cyclohepta[f]indene-2-carboxylic acid (5-phenyl-[1,3,4]thiadiazol-2-yl)-amide; and 3-amino-6-tert-butyloxycarbonyl-6,7,8,9-tetrahydro-5H-1-thia-6,10-diaza-cyclohepta[f]indene-2-carboxylic acid (5-phenyl-[1,3,4]thiadiazol-2-yl)-amide.

The present invention further provides a method for the preparation of a mixture of tert-butyl (4E)-4-(hydroxymethylene)-5-oxoazepane-1-carboxylate and tert-butyl (3E)-3-(hydroxymethylene)-4-oxoazepane-1-carboxylate, said method comprising reacting tert-butyl 4-oxoazepane-1-carboxylate with N-[tert-butoxy(dimethylamino)methyl]-N,N-dimethylamine.

The present invention also provides a method for the preparation of a mixture of tert-butyl 3-cyano-2-thioxo-1,2,5,6,8,9-hexahydro-7H-pyrido[2,3-d]azepine-7-carboxylate and tert-butyl 3-cyano-2-thioxo-1,2,5,7,8,9-hexahydro-6H-pyrido[3,2-c]azepine-6-carboxylate said method comprising reacting a mixture of tert-butyl (4E)-4-(hydroxymethylene)-5-oxoazepane-1-carboxylate and tert-butyl (3E)-3-(hydroxymethylene)-4-oxoazepane-1-carboxylate in the presence of 2-cyanoethanethioamide and piperidine acetate.

The present invention further provides a method for the preparation of 3-amino-7-tert-butyloxycarbonyl-6,7,8,9-tetrahydro-5H-1-thia-7,10-diaza-cyclohepta[f]indene-2-carboxylic acid (5-phenyl-[1,3,4]thiadiazol-2-yl)-amide comprising reacting tert-butyl 3-cyano-2-thioxo-1,2,5,6,8,9-hexahydro-7H-pyrido[2,3-d]azepine-7-carboxylate with 2-chloro-N-(5-phenyl-1,3,4-thiadiazol-2-yl)acetamide.

The present invention also provides a method for the preparation of 3-amino-6,7,8,9-tetrahydro-5H-1-thia-7,10-diaza-cyclohepta[f]indene-2-carboxylic acid (5-phenyl-[1,3,4]thiadiazol-2-yl)-amide comprising reacting 3-amino-7-tert-butyloxycarbonyl-6,7,8,9-tetrahydro-5H-1-thia-7,10-diaza-cyclohepta[f]indene-2-carboxylic acid (5-phenyl-[1,3,4]thiadiazol-2-yl)-amide with HCl.

The present invention further provides a method for the preparation of 3-amino-6-tert-butyloxycarbonyl-6,7,8,9-tetrahydro-5H-1-thia-6,10-diaza-cyclohepta[f]indene-2-carboxylic acid (5-phenyl-[1,3,4]thiadiazol-2-yl)-amide comprising reacting tert-butyl 3-cyano-2-thioxo-1,2,5,7,8,9-hexahydro-6H-pyrido[3,2-c]azepine-6-carboxylate with 2-chloro-N-(5-phenyl-1,3,4-thiadiazol-2-yl)acetamide.

The present invention also provides a method for the preparation of 3-amino-6,7,8,9-tetrahydro-5H-1-thia-6,10-diaza-cyclohepta[f]indene-2-carboxylic acid (5-phenyl-[1,3,4]thiadiazol-2-yl)-amide comprising reacting 3-amino-6-tert-butyloxycarbonyl-6,7,8,9-tetrahydro-5H-1-thia-6,10-diaza-cyclohepta[f]indene-2-carboxylic acid (5-phenyl-[1,3,4]thiadiazol-2-yl)-amide with HCl.

Other objects and advantages of the present invention will become apparent from the following description and appended claims.

### DETAILED DESCRIPTION OF THE INVENTION

The compounds of the invention are of the following general Formula I:

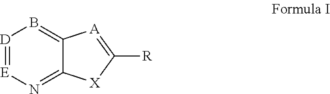

wherein X is selected from the groups consisting of O, S and N—R′, wherein R′ is selected from the group consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl, or R and R¹ together with the carbons they are attached to may form a substituted or unsubstituted ring; and

A, B, D, and E are independently N or C—R¹, C—R², C—R³ and C—R⁴, respectively, wherein R¹, R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R¹ and R together with the carbons they are attached to may form a substituted or unsubstituted ring, or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring.

Preferably, for the compound of Formula I, X is S; A is C—NH₂, B is C—R² and R² is fluoro substituted phenyl or B is C—H; D is a C—H; E is C—R⁴ and R⁴ is a thienyl or D is C—R³ and E is C—R⁴, and R³ and R⁴ form a ring; and/or R is a substituted aminocarbonyl.

Preferably the compound of Formula I of the present invention is selected from the group consisting of: 3-amino-6,7,8,9-tetrahydro-5H-1-thia-10-aza-cyclohepta[f]indene-2-carboxylic acid (5-phenyl-[1,3,4]thiadiazol-2-yl)-amide; 1-amino-5-methyl-6,7,8,9-tetrahydro-thieno[2,3-c]isoquinoline-2-carboxylic acid (4-methyl-thiazol-2-yl)-amide; 3,6-diamino-5-cyano-4-furan-2-yl-thieno[2,3-b]pyridine-2-carboxylic acid (4-bromo-phenyl)-amide; 3-amino-6-ethyl-5,6,7,8-tetrahydro-thieno[2,3-b][1,6]naphthyridine-2-carboxylic acid (4-trifluoromethyl-phenyl)-amide; 4-[(3-amino-6-isopropyl-5,6,7,8-tetrahydro-thieno[2,3-b][1,6]naphthyridine-2-carbonyl)-amino]-benzoic acid ethyl ester; and 3-amino-6-methyl-5,6,7,8-tetrahydro-thieno[2,3-b][1,6]naphthyridine-2-carboxylic acid (4-trifluoromethoxy-phenyl)-amide.

More preferably, the compound of Formula I of the present invention is 3-amino-6,7,8,9-tetrahydro-5H-1-thia-10-aza-cyclohepta[f]indene-2-carboxylic acid (5-phenyl-[1,3,4]thiadiazol-2-yl)-amide.

The compounds of the invention are also of the following general Formula II:

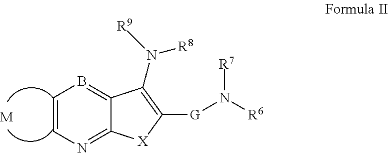

wherein X is selected from the groups consisting of O, S or N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

B is N or C—R², wherein R² is selected from the groups consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro;

G is selected from the group consisting of —C(═O)—, —C(═S)—, —S(═O)₂—, and —C(═NR⁵)—, wherein R⁵ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁵ and R⁶ or R⁷, together with the nitrogen atoms they are attached to, along with the carbon of G, or R⁵ and R⁸ or R⁹, together with the nitrogen atoms they are attached to, along with the carbon of G and two carbons of the X-containing 5-membered ring, may form a substituted or unsubstituted ring, which may be fused with an aromatic or aliphatic ring;

R⁶, R⁷, R⁸, and R⁹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl; or R⁶ or R⁷ and R⁵, together with the nitrogen atoms they are attached to, along with the carbon of G, or R⁸ or R⁹ and R⁵, together with the nitrogen atoms they are attached to, along with the carbon of G and two carbons of the X-containing 5-membered ring, or R⁶ or R⁷ and R⁸ or R⁹, together with the nitrogen atoms they are attached to, along with the carbon or sulfur of G and two carbons of the X-containing 5-membered ring, or R⁶ and R⁷, together with the nitrogen atom they are attached to, or R⁸ and R⁹, together with the nitrogen atom they are attached to, may form a substituted or unsubstituted ring, which may be fused with an aromatic or aliphatic ring; and

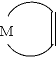

is a 7 or 8-membered ring which contains one or more heteroatoms selected from N, O and S, or a 4-membered ring which may optionally contain one or more heteroatoms selected from N, O and S. The ring may be substituted or unsubstituted, or fused with another ring, which may be aromatic or non-aromatic and may include one or more heteroatoms in the ring and may be fused with an aromatic or aliphatic ring.

Preferably, for the compound of Formula II, X is S; B is CH; each of R⁸ and R⁹ is H; G is —C(═O)—; R⁶ is a hydrogen; R⁷ is a heteroaryl; and

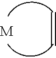

is a 7-membered ring which contains N as a heteroatom.

Preferably, the compound of Formula II of the present invention is 3-amino-6,7,8,9-tetrahydro-5H-1-thia-6,10-diaza-cyclohepta[f]indene-2-carboxylic acid (5-phenyl-[1,3,4]thiadiazol-2-yl)-amide.

Also preferably, the compound of Formula II of the present invention is 3-amino-6,7,8,9-tetrahydro-5H-1-thia-7,10-diaza-cyclohepta[f]indene-2-carboxylic acid (5-phenyl-[1,3,4]thiadiazol-2-yl)-amide.

The compounds of the present invention are also of the following general Formula III:

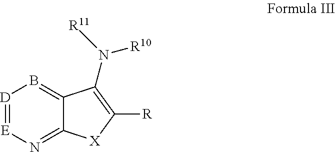

wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;

R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;

B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and

R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen.

Preferably, for the compound of Formula III, X is S; B is C—H; D is C—H; and E is C—R⁴ and R⁴ is a heteroaryl. Also preferably, D is C—R³ and E is C—R⁴, and R³ and R⁴ form a ring. Again preferably, R is a substituted aminocarbonyl.

Preferably, the compound of Formula III of the present invention is 3-[N-[3-amino-6-(2-thienyl)thieno[2,3-b]pyridine-2-carbonyl]-3-(trifluoromethyl)anilino]propanoic acid.

The compounds of the present invention also include compounds or a pharmaceutically acceptable salt thereof selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide. Preferred among said compounds are 3-amino-N,6-bis(4-chlorophenyl)thieno[2,3-b]pyridine-2-carboxamide and 3-amino-6-[3-(difluoromethoxy)phenyl]-N-[4-(difluoromethoxy)phenyl]thieno[2,3-b]pyridine-2-carboxamide.

The compounds of the present invention also include a compound or a pharmaceutically acceptable salt thereof, wherein said compound is selected from the group consisting of: 3-amino-N-(4-bromophenyl)-6-(4-chlorophenyl)thieno[2,3-b]pyridine-2-carboxamide; 3-amino-6-(3-methoxyphenyl)-N-[4-(trifluoromethoxy)phenyl]thieno[2,3-b]pyridine-2-carboxamide; 3-amino-N-(2,5-dichlorophenyl)-6-(2-thienyl)thieno[2,3-b]pyridine-2-carboxamide; 3-amino-N-(2,3-dichlorophenyl)-6-(2-thienyl)thieno[2,3-b]pyridine-2-carboxamide; 3-amino-N-(4-bromophenyl)-6-(3-methoxyphenyl)thieno[2,3-b]pyridine-2-carboxamide; 3-amino-6-(1,3-benzodioxol-5-yl)-N-(2-bromo-4-methyl-phenyl)thieno[2,3-b]pyridine-2-carboxamide; and 3-amino-6-(3-methoxyphenyl)-N-(2-phenoxyphenyl)thieno[2,3-b]pyridine-2-carboxamide. Preferably said compound is 3-amino-N-(4-bromophenyl)-6-(4-chlorophenyl)thieno[2,3-b]pyridine-2-carboxamide or 3-amino-6-(3-methoxyphenyl)-N-[4-(trifluoromethoxy)phenyl]thieno[2,3-b]pyridine-2-carboxamide.

The method of the present invention is for the treatment of at least one type of a Dengue virus infection or disease associated therewith (each type of Dengue virus infection being caused by a Dengue virus serotype), comprising administering in a therapeutically effective amount to a mammal in need thereof, a compound of Formula I, Formula II, Formula III or other compounds of the present invention as described above.

Preferably, the mammal is a human and the viral infection is a flavivirus infection. More preferably, the flavivirus is selected from the group consisting of Dengue virus, West Nile virus, yellow fever virus, Japanese encephalitis virus, and tick-borne encephalitis virus. Most preferably, the flavivirus is a Dengue virus selected from the group consisting of DEN-1, DEN-2, DEN-3, and DEN-4.

Preferably, the viral infection is associated with a condition selected from the group consisting of Dengue fever, Yellow fever, West Nile, St. Louis encephalitis, Hepatitis C, Murray Valley encephalitis, and Japanese encephalitis. Most preferably, the viral infection is associated with Dengue fever wherein said Dengue fever is selected from the group consisting of classical dengue fever and dengue hemorrhagic fever.

The method of the present invention may also comprise co-administration of: a) other antivirals; b) vaccines; and/or c) interferons or pegylated interferons.

The present invention also provides for methods of synthesis of compounds of the present invention, in particular 3-amino-6,7,8,9-tetrahydro-5H-1-thia-7,10-diaza-cyclohepta[f]indene-2-carboxylic acid (5-phenyl-[1,3,4]thiadiazol-2-yl)-amide and 3-amino-6,7,8,9-tetrahydro-5H-1-thia-6,10-diaza-cyclohepta[f]indene-2-carboxylic acid (5-phenyl-[1,3,4]thiadiazol-2-yl)-amide. These methods of synthesis are provided below in Examples 14 and 15.

Novel Intermediates in the synthesis of the compounds of the present invention include but are not limited to each of tert-butyl (4E)-4-(hydroxymethylene)-5-oxoazepane-1-carboxylate; tert-butyl (3E)-3-(hydroxymethylene)-4-oxoazepane-1-carboxylate; tert-butyl 3-cyano-2-thioxo-1,2,5,6,8,9-hexahydro-7H-pyrido[2,3-d]azepine-7-carboxylate; tert-butyl 3-cyano-2-thioxo-1,2,5,7,8,9-hexahydro-6H-pyrido[3,2-c]azepine-6-carboxylate; and 3-amino-7-tert-butyloxycarbonyl-6,7,8,9-tetrahydro-5H-1-thia-7,10-diaza-cyclohepta[f]indene-2-carboxylic acid (5-phenyl-[1,3,4]thiadiazol-2-yl)-amide; and 3-amino-6-tert-butyloxycarbonyl-6,7,8,9-tetrahydro-5H-1-thia-6,10-diaza-cyclohepta[f]indene-2-carboxylic acid (5-phenyl-[1,3,4]thiadiazol-2-yl)-amide.

### DEFINITIONS

In accordance with this detailed description, the following abbreviations and definitions apply. It must be noted that as used herein, the singular forms “a”, “an”, and “the” include plural referents unless the context clearly dictates otherwise.

The publications discussed herein are provided solely for their disclosure. Nothing herein is to be construed as an admission regarding antedating the publications. Further, the dates of publication provided may be different from the actual publications dates, which may need to be independently confirmed.

Where a range of values is provided, it is understood that each intervening value is encompassed. The upper and lower limits of these smaller ranges may independently be included in the smaller, subject to any specifically-excluded limit in the stated range. Where the stated range includes one or both of the limits, ranges excluding either both of those included limits are also included in the invention. Also contemplated are any values that fall within the cited ranges.

Unless defined otherwise, all technical and scientific terms used herein have the same meaning as commonly understood by one of ordinary skill in the art. Any methods and materials similar or equivalent to those described herein can also be used in practice or testing. All publications mentioned herein are incorporated herein by reference to disclose and describe the methods and/or materials in connection with which the publications are cited.

By “patient” or “subject” is meant to include any mammal. A “mammal”, for purposes of treatment, refers to any animal classified as a mammal, including but not limited to, humans, experimental animals including rats, mice, and guinea pigs, domestic and farm animals, and zoo, sports, or pet animals, such as dogs, horses, cats, cows, and the like.

The term “efficacy” as used herein refers to the effectiveness of a particular treatment regime. Efficacy can be measured based on change of the course of the disease in response to an agent.

The term “success” as used herein in the context of a chronic treatment regime refers to the effectiveness of a particular treatment regime. This includes a balance of efficacy, toxicity (e.g., side effects and patient tolerance of a formulation or dosage unit), patient compliance, and the like. For a chronic administration regime to be considered “successful” it must balance different aspects of patient care and efficacy to produce a favorable patient outcome.

The terms “treating”, “treatment”, and the like are used herein to refer to obtaining a desired pharmacological and physiological effect. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment”, as used herein, covers any treatment of a disease in a mammal, such as a human, and includes: (a) preventing the disease from occurring in a subject which may be predisposed to the disease but has not yet been diagnosed as having it, i.e., causing the clinical symptoms of the disease not to develop in a subject that may be predisposed to the disease but does not yet experience or display symptoms of the disease; (b) inhibiting the disease, i.e., arresting or reducing the development of the disease or its clinical symptoms; and (c) relieving the disease, i.e., causing regression of the disease and/or its symptoms or condition. Treating a patient's suffering from disease related to a pathological inflammation is contemplated. Preventing, inhibiting, or relieving adverse effects attributed to pathological inflammation over long periods of time and/or are such caused by the physiological responses to inappropriate inflammation present in a biological system over long periods of time are also contemplated.

As used herein, “acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkylamino” refers to the group —NRR where each R is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R is joined to form together with the nitrogen atom a heterocyclic or substituted heterocyclic ring wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” refers to alkenyl group preferably having from 2 to 10 carbon atoms and more preferably 2 to 6 carbon atoms and having at least 1 and preferably from 1-2 sites of alkenyl unsaturation.

“Alkoxy” refers to the group “alkyl-O—” which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkyl” refers to linear or branched alkyl groups having from 1 to 10 carbon atoms, alternatively 1 to 6 carbon atoms. This term is exemplified by groups such as methyl, t-butyl, n-heptyl, octyl and the like.

“Amino” refers to the group —NH₂.

“Aryl” or “Ar” refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one, and the like) provided that the point of attachment is through an aromatic ring atom.

“Substituted aryl” refers to aryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OS(O)₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Cycloalkyl” refers to cyclic alkyl groups of from 3 to 8 carbon atoms having a single cyclic ring including, by way of example, cyclopropyl, cyclobutyl, cyclopentyl, cyclohexyl, cyclooctyl and the like. Excluded from this definition are multi-ring alkyl groups such as adamantanyl, etc.

“Halo” or “halogen” refers to fluoro, chloro, bromo and iodo.

“Heteroaryl” refers to an aromatic carbocyclic group of from 2 to 10 carbon atoms and 1 to 4 heteroatoms selected from the group consisting of oxygen, nitrogen and sulfur within the ring or oxides thereof. Such heteroaryl groups can have a single ring (e.g., pyridyl or furyl) or multiple condensed rings (e.g., indolizinyl or benzothienyl) wherein one or more of the condensed rings may or may not be aromatic provided that the point of attachment is through an aromatic ring atom. Additionally, the heteroatoms of the heteroaryl group may be oxidized, i.e., to form pyridine N-oxides or 1,1-dioxo-1,2,5-thiadiazoles and the like. Additionally, the carbon atoms of the ring may be substituted with an oxo (═O). The term “heteroaryl having two nitrogen atoms in the heteroaryl, ring” refers to a heteroaryl group having two, and only two, nitrogen atoms in the heteroaryl ring and optionally containing 1 or 2 other heteroatoms in the heteroaryl ring, such as oxygen or sulfur.

“Substituted heteroaryl” refers to heteroaryl groups which are substituted with from 1 to 3 substituents selected from the group consisting of hydroxy, acyl, acylamino, thiocarbonylamino, acyloxy, alkyl, substituted alkyl, alkoxy, substituted alkoxy, alkenyl, substituted alkenyl, alkynyl, substituted alkynyl, amidino, alkylamidino, thioamidino, amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, carboxylamido, cyano, thiol, thioalkyl, substituted thioalkyl, thioaryl, substituted thioaryl, thioheteroaryl, substituted thioheteroaryl, thiocycloalkyl, substituted thiocycloalkyl, thioheterocyclic, substituted thioheterocyclic, cycloalkyl, substituted cycloalkyl, guanidino, guanidinosulfone, halo, nitro, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NRR where R is hydrogen or alkyl, —NRS(O)₂-alkyl, —NRS(O)₂-substituted alkyl, —NRS(O)₂-aryl, —NRS(O)₂-substituted aryl, —NRS(O)₂-heteroaryl, —NRS(O)₂-substituted heteroaryl, —NRS(O)₂-heterocyclic, —NRS(O)₂-substituted heterocyclic, —NRS(O)₂—NR-alkyl, —NRS(O)₂—NR-substituted alkyl, —NRS(O)₂—NR-aryl, —NRS(O)₂—NR-substituted aryl, —NRS(O)₂—NR-heteroaryl, —NRS(O)₂—NR-substituted heteroaryl, —NRS(O)₂—NR-heterocyclic, —NRS(O)₂—NR-substituted heterocyclic where R is hydrogen or alkyl, mono- and di-alkylamino, mono- and di-(substituted alkyl)amino, mono- and di-arylamino, mono- and di-substituted arylamino, mono- and di-heteroarylamino, mono- and di-substituted heteroarylamino, mono- and di-heterocyclic amino, mono- and di-substituted heterocyclic amino, unsymmetric di-substituted amines having different substituents independently selected from the group consisting of alkyl, substituted alkyl, aryl, substituted aryl, heteroaryl, substituted heteroaryl, heterocyclic and substituted heterocyclic and amino groups on the substituted aryl blocked by conventional blocking groups such as Boc, Cbz, formyl, and the like or substituted with —SO₂NRR where R is hydrogen or alkyl.

“Sulfonyl” refers to the group —S(O)₂R where R is selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Optionally substituted” means that the recited group may be unsubstituted or the recited group may be substituted.

“Pharmaceutically-acceptable carrier” means a carrier that is useful in preparing a pharmaceutical composition or formulation that is generally safe, non-toxic, and neither biologically nor otherwise undesirable, and includes a carrier that is acceptable for veterinary use as well as human pharmaceutical use.

“Pharmaceutically-acceptable cation” refers to the cation of a pharmaceutically-acceptable salt.

“Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

Pharmaceutically-acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful, for example, carboxylic acid amides, including carboxamides, lower alkyl carboxamides, dialkyl carboxamides, and the like.

Pharmaceutically-acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

A compound may act as a pro-drug. Pro-drug means any compound which releases an active parent drug in vivo when such pro-drug is administered to a mammalian subject. Pro-drugs are prepared by modifying functional groups present in such a way that the modifications may be cleaved in vivo to release the parent compound. Pro-drugs include compounds wherein a hydroxy, amino, or sulfhydryl group is bonded to any group that may be cleaved in vivo to regenerate the free hydroxyl, amino, or sulfhydryl group, respectively. Examples of pro-drugs include, but are not limited to esters (e.g., acetate, formate, and benzoate derivatives), carbamates (e.g., N,N-dimethylamino-carbonyl) of hydroxy functional groups, and the like.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically-effective amount” means the amount of a compound that, when administered to a mammal for treating a disease, is sufficient to effect such treatment for the disease. The “therapeutically-effective amount” will vary depending on the compound, the disease, and its severity and the age, weight, etc., of the mammal to be treated.

### Pharmaceutical Formulations of the Compounds

“Pharmaceutical composition” refers to a composition intended and suitable for human or animal administration. A composition containing a compound of the present invention dissolved in a solvent such as water, organic solvent, alcohol or DMSO for the intended purpose of in-vitro testing or for any type of testing outside of an animal or human body is not considered a pharmaceutical composition as defined herein.

In general, compounds will be administered in a therapeutically-effective amount by any of the accepted modes of administration for these compounds. The compounds can be administered by a variety of routes, including, but not limited to, oral, parenteral (e.g., subcutaneous, subdural, intravenous, intramuscular, intrathecal, intraperitoneal, intracerebral, intraarterial, or intralesional routes of administration), topical, intranasal, localized (e.g., surgical application or surgical suppository), rectal, and pulmonary (e.g., aerosols, inhalation, or powder). Accordingly, these compounds are effective as both injectable and oral compositions. The compounds can be administered continuously by infusion or by bolus injection.

The actual amount of the compound, i.e., the active ingredient, will depend on a number of factors, such as the severity of the disease, i.e., the condition or disease to be treated, age, and relative health of the subject, the potency of the compound used, the route and form of administration, and other factors.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compounds lies within a range of circulating concentrations that include the ED₅₀ with little or no toxicity. The dosage may vary within this range depending upon the dosage form employed and the route of administration utilized. For any compound used, the therapeutically-effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The amount of the pharmaceutical composition administered to the patient will vary depending upon what is being administered, the purpose of the administration, such as prophylaxis or therapy, the state of the patient, the manner of administration, and the like. In therapeutic applications, compositions are administered to a patient already suffering from a disease in an amount sufficient to cure or at least partially arrest the symptoms of the disease and its complications. An amount adequate to accomplish this is defined as “therapeutically-effective dose.” Amounts effective for this use will depend on the disease condition being treated as well as by the judgment of the attending clinician depending upon factors such as the severity of the inflammation, the age, weight, and general condition of the patient, and the like.

The compositions administered to a patient are in the form of pharmaceutical compositions described supra. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. It will be understood that use of certain of the foregoing excipients, carriers, or stabilizers will result in the formation of pharmaceutical salts.

The active compound is effective over a wide dosage range and is generally administered in a pharmaceutically- or therapeutically-effective amount. The therapeutic dosage of the compounds will vary according to, for example, the particular use for which the treatment is made, the manner of administration of the compound, the health and condition of the patient, and the judgment of the prescribing physician. For example, for intravenous administration, the dose will typically be in the range of about 0.5 mg to about 100 mg per kilogram body weight. Effective doses can be extrapolated from dose-response curves derived from in vitro or animal model test systems. Typically, the clinician will administer the compound until a dosage is reached that achieves the desired effect.

When employed as pharmaceuticals, the compounds are usually administered in the form of pharmaceutical compositions. Pharmaceutical compositions contain as the active ingredient one or more of the compounds above, associated with one or more pharmaceutically-acceptable carriers or excipients. The excipient employed is typically one suitable for administration to human subjects or other mammals. In making the compositions, the active ingredient is usually mixed with an excipient, diluted by an excipient, or enclosed within a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained, or delayed-release of the active ingredient after administration to the patient by employing procedures known in the art.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically-discrete units suitable as unitary dosages for human subjects and other mammals, each unit containing a predetermined quantity of active material calculated to produce the desired therapeutic effect, in association with a suitable pharmaceutical excipient.

The compound can be formulated for parenteral administration in a suitable inert carrier, such as a sterile physiological saline solution. The dose administered will be determined by route of administration.

Administration of therapeutic agents by intravenous formulation is well known in the pharmaceutical industry. An intravenous formulation should possess certain qualities aside from being just a composition in which the therapeutic agent is soluble. For example, the formulation should promote the overall stability of the active ingredient(s), also, the manufacture of the formulation should be cost-effective. All of these factors ultimately determine the overall success and usefulness of an intravenous formulation.

Other accessory additives that may be included in pharmaceutical formulations and compounds as follow: solvents: ethanol, glycerol, propylene glycol; stabilizers: EDTA (ethylene diamine tetraacetic acid), citric acid; antimicrobial preservatives: benzyl alcohol, methyl paraben, propyl paraben; buffering agents: citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate, phosphoric acid/disodium hydrogen phosphate; and tonicity modifiers: sodium chloride, mannitol, dextrose.

The presence of a buffer is necessary to maintain the aqueous pH in the range of from about 4 to about 8. The buffer system is generally a mixture of a weak acid and a soluble salt thereof, e.g., sodium citrate/citric acid; or the monocation or dication salt of a dibasic acid, e.g., potassium hydrogen tartrate; sodium hydrogen tartrate, phosphoric acid/potassium dihydrogen phosphate, and phosphoric acid/disodium hydrogen phosphate.

The amount of buffer system used is dependent on (1) the desired pH; and (2) the amount of drug. Generally, the amount of buffer used is able to maintain a formulation pH in the range of 4 to 8. Generally, a 1:1 to 10:1 mole ratio of buffer (where the moles of buffer are taken as the combined moles of the buffer ingredients, e.g., sodium citrate and citric acid) to drug is used.

A useful buffer is sodium citrate/citric acid in the range of 5 to 50 mg per ml. sodium citrate to 1 to 15 mg per ml. citric acid, sufficient to maintain an aqueous pH of 4-6 of the composition.

The buffer agent may also be present to prevent the precipitation of the drug through soluble metal complex formation with dissolved metal ions, e.g., Ca, Mg, Fe, Al, Ba, which may leach out of glass containers or rubber stoppers or be present in ordinary tap water. The agent may act as a competitive complexing agent with the drug and produce a soluble metal complex leading to the presence of undesirable particulates.

In addition, the presence of an agent, e.g., sodium chloride in an amount of about of 1-8 mg/ml, to adjust the tonicity to the same value of human blood may be required to avoid the swelling or shrinkage of erythrocytes upon administration of the intravenous formulation leading to undesirable side effects such as nausea or diarrhea and possibly to associated blood disorders. In general, the tonicity of the formulation matches that of human blood which is in the range of 282 to 288 mOsm/kg, and in general is 285 mOsm/kg, which is equivalent to the osmotic pressure corresponding to a 0.9% solution of sodium chloride.

An intravenous formulation can be administered by direct intravenous injection, i.v. bolus, or can be administered by infusion by addition to an appropriate infusion solution such as 0.9% sodium chloride injection or other compatible infusion solution.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 2000 mg of the active ingredient.

The tablets or pills may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions may be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically-acceptable, aqueous or organic solvents, or mixtures thereof, and powders. The liquid or solid compositions may contain suitable pharmaceutically-acceptable excipients as described supra. Compositions in pharmaceutically-acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered from devices which deliver the formulation in an appropriate manner.

The compounds can be administered in a sustained release form. Suitable examples of sustained-release preparations include semipermeable matrices of solid hydrophobic polymers containing the compounds, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res. 15: 167-277 (1981) and Langer, Chem. Tech. 12: 98-105 (1982) or poly(vinyl alcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid (EP 133,988).

The compounds can be administered in a sustained-release form, for example a depot injection, implant preparation, or osmotic pump, which can be formulated in such a manner as to permit a sustained-release of the active ingredient. Implants for sustained-release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host.

Transdermal delivery devices (“patches”) may also be employed. Such transdermal patches may be used to provide continuous or discontinuous infusion of the compounds in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on-demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference.

Indirect techniques usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid-soluble and amenable to transportation across the blood-brain barrier. Alternatively, the delivery of hydrophilic drugs may be enhanced by intra-arterial infusion of hypertonic solutions which can transiently open the blood-brain barrier.

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka et al., U.S. Pat. Nos. 4,235,871, 4,501,728 and 4,837,028 each of which is incorporated herein by reference.

Pharmaceutical compositions are suitable for use in a variety of drug delivery systems. Suitable formulations for use in the present invention are found in Remington's Pharmaceutical Sciences, Mace Publishing Company, Philadelphia, Pa., 17th ed. (1985).

In the examples below, if an abbreviation is not defined above, it has its generally accepted meaning. Further, all temperatures are in degrees Celsius (unless otherwise indicated). The following Methods were used to prepare the compounds set forth below as indicated.

### Example 1

### Formulation 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Example 2

### Formulation 2

A tablet formula is prepared using the ingredients below:

| | Quantity |
|---|---|
| Ingredient | (mg/capsule) |
| Active ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Example 3

### Formulation 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### Example 4

### Formulation 4

Tablets, each containing 30 mg of active ingredient, are prepared as follows:

| | Quantity | |
|---|---|---|
| Ingredient | (mg/capsule) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in water) | | |
| Sodium Carboxymethyl starch | 4.5 | mg |
| Magnesium stearate | 0.5 | mg |
| Talc | 1.0 | mg |
| Total | 120 | mg |

The active ingredient, starch, and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules, which after mixing, are compressed on a tablet machine to yield tablets each weighing 150 mg.

### Example 5

### Formulation 5

Capsules, each containing 40 mg of medicament, are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, cellulose, starch, an magnesium stearate are blended, passed through a No. 20 mesh U.S. sieve, and filled into hard gelatin capsules in 150 mg quantities.

### Example 6

### Formulation 6

Suppositories, each containing 25 mg of active ingredient, are made as follows:

| Ingredient | Amount | |
|---|---|---|
| Active Ingredient | 25 | mg |
| Saturated fatty acids glycerides | to 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Example 7

### Formulation 7

Suspensions, each containing 50 mg of medicament per 5.0 ml dose, are made as follows:

| Ingredient | Amount | |
|---|---|---|
| Active Ingredient | 50.0 | mg |
| Xanthan gum | 4.0 | mg |
| Sodium carboxymethyl cellose (11%) | 500 | mg |
| Microcrystalline cellulose (89%) | | |
| Sucrose | 1.75 | g |
| Sodium benzoate | 10.0 | mg |
| Flavor and color | q.v. | |
| Purified water | to 5.0 | ml |

The medicament, sucrose, and xanthan gum are blended, passed through a NO. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Example 8

### Formulation 8

Hard gelatin tablets, each containing 15 mg of active ingredient, are made as follows:

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, cellulose, starch, and magnesium stearate are blended, passed through a No. 20 mesh U.S. sieve, and filled into hard gelatin capsules in 560 mg quantities.

### Example 9

### Formulation 9

An intravenous formulation may be prepared as follows:

| Ingredient | (mg/capsule) |
|---|---|
| Active Ingredient | 250.0 mg |
| Isotonic saline | 1000 ml |

Therapeutic compound compositions generally are placed into a container having a sterile access port, for example, an intravenous solution bag or vial having a stopper pierceable by a hypodermic injection needle or similar sharp instrument.

### Example 10

### Formulation 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### Example 11

### Formulation 11

An aerosol formulation may be prepared as follows: A solution of the candidate compound in 0.5% sodium bicarbonate/saline (w/v) at a concentration of 30.0 mg/mL is prepared using the following procedure:

Preparation of 0.5% Sodium Bicarbonate/Saline Stock Solution: 100.0 mL

| Ingredient | Gram/100.0 mL | | Final Concentration |
|---|---|---|---|
| Sodium Bicarbonate | 0.5 | g | 0.5% |
| Saline | q.s. ad 100.0 | mL | q.s. ad 100% |

Procedure:

1. Add 0.5 g sodium bicarbonate into a 100 mL volumetric flask.
2. Add approximately 90.0 mL saline and sonicate until dissolved.
3. Q.S. to 100.0 mL with saline and mix thoroughly.

Preparation of 30.0 mg/mL Candidate Compound: 10.0 mL

| Ingredient | Gram/100.0 mL | | Final Concentration |
|---|---|---|---|
| Candidate Compound | 0.300 | g | 30.0 mg/mL |
| .05% Sodium | q.s. ad 10.0 | mL | q.s. ad 100% |
| Bicarbonate/Saline Stock | | | |
| Solution | | | |

Procedure:

1. Add 0.300 g of the candidate compound into a 10.0 mL volumetric flask.
2. Add approximately 9.7 mL of 0.5% sodium bicarbonate/saline stock solution.
3. Sonicate until the candidate compound is completely dissolved.
4. Q.S. to 10.0 mL with 0.5% sodium bicarbonate/saline stock solution and mix.

### Example 12

### Development of a High-Throughput Screening Assay for Measurement of Dengue Virus-Induced Cytopathic Effect

A sensitive and reproducible high-throughput screening (HTS) assay has been established to measure dengue virus-induced cytopathic effect (CPE). To determine the amount of dengue virus stock required to produce complete CPE in 5 days, Vero cell monolayers were seeded on 96-well plates and infected with 10-fold serial dilutions of the dengue virus stock representing a multiplicity of infection (MOI) of approximately 0.001 PFU/cell to 0.1 PFU/cell. At 5 days post-infection, the cultures were fixed with 5% glutaraldehyde and stained with 0.1% crystal violet. Virus-induced CPE was quantified spectrophotometrically at OD₅₇₀. From this analysis, an MOI of 0.1 PFU/cell of dengue virus stock was chosen for use in the HTS assay. To establish the signal-to-noise ratio (S/N) of the 96-well assay and evaluate the well-to-well and assay-to-assay variability, five independent experiments were performed. Vero cell monolayers were infected with 0.1 PFU/cell of dengue virus stock. Each plate contained the following controls: quadruplicate virus-infected wells, quadruplicate uninfected cell wells and a dose response curve in duplicate for ribavirin at 500, 250, 125 and 62 μM, as reference standards. At day 5 post-infection, the plates were processed as described above.

The dengue virus CPE assay was used to evaluate compounds from the SIGA chemical library for those that inhibit dengue virus-induced CPE. Each evaluation run consisted of 48 96-well plates with 80 compounds per plate to generate 4,608 data points per run. At this throughput we are capable of evaluating 200,000 compounds in about 52 weeks. Compounds were dissolved in DMSO and diluted in medium such that the final concentration in each well was 5 μM compound and 0.5% DMSO. The compounds were added robotically to the culture medium using the PerkinElmer MultiPROBE® II HT PLUS robotic system. Following compound addition, cultures were infected with dengue virus (DEN-2 strain New Guinea C). After 5 days incubation, plates were processed and CPE quantified on a PerkinElmer EnVision II plate reader system.

The results of these experiments indicated that the 96-well assay format is robust and reproducible. The S/N ratio (ratio of signal of cell control wells (signal) to virus control wells (noise)) was 5.0±1.2. The well-to-well variability was determined for each individual plate and found to have a coefficient of variance of less than 10% for both positive control and negative control wells, and overall assay-to-assay variability was less than 15%. Using this assay, the EC₅₀ values for ribavirin were determined to be 125±25 μM, respectively. The effectiveness of ribavirin against dengue varies with the cell type used, but the values we obtained were within the range of published values for this compound (2, 13, 28). Taken together, these results show that a sensitive and reproducible HTS assay has been successfully developed to evaluate our compound library for inhibitors of dengue virus replication.

### Example 13

### Determining Anti-Dengue-2 Activity of Compounds of the Invention

The assay described in Example 12 was the basis of a high-throughput screen for dengue virus inhibitors, against which a library of 210,000 compounds was tested. Compounds that inhibited dengue virus induced CPE by at least 50% were further investigated for chemical tractability, potency, and selectivity.

Initially, the chemical structures of the hit compounds were examined for chemical tractability. A chemically tractable compound is defined as one that is synthetically accessible using reasonable chemical methodology, and which possesses chemically stable functionalities and potential drug-like qualities. Hits that passed this medicinal chemistry filter were evaluated for their potency. Compound potency was determined by evaluating inhibitory activity across a broad range of concentrations. Nonlinear regression was used to generate best-fit inhibition curves and to calculate the 50% effective concentration (EC₅₀). The selectivity or specificity of a given compound is typically expressed as a ratio of its cytotoxicity to its biological effect. A cell proliferation assay is used to calculate a 50% cytotoxicity concentration (CC₅₀); the ratio of this value to the EC₅₀ is referred to as the therapeutic index (T.I.=CC₅₀/EC₅₀). Two types of assays have been used to determine cytotoxicity, both of which are standard methods for quantitating the reductase activity produced in metabolically active cells (22). One is a colorimetric method that measures the reduction of 3-(4,5-dimethylthiazol-2-yl)-2,5-diphenyl-tetrazolium bromide (MTT), and the other uses fluorimetry to measure the reduction of resazurin (Alamar Blue). Selectivity could be further characterized by assessing the inhibitory action against viruses from unrelated virus families. Sixteen quality dengue hits were discovered in the pool of initial hits from the HTS screening, all with EC₅₀ values below 25 μM. Verification that these compounds act against each of the four serotypes of dengue was done with yield assays carried out at several drug concentrations, and the titer determined for each.

Compounds that were active in the primary screen were tested for activity in viral yield assays. Table 1 shows some of the compounds that were tested for activity against Dengue-2 (Strain New Guinea C) in a viral yield assay at a range of concentrations. Vero cells in 12-well plates were infected with dengue-2 virus at a multiplicity of infection (MOI) of 0.1, treated with compound (or DMSO as a control), incubated at 37° C., harvested 48 hours post infection and titered on Vero cells as described above. The EC₅₀ was calculated through ExcelFit. Activities against other serotypes of dengue virus were determined in a similar way.

Compound 1 was identified as one of the most potent and selective compounds from within the pool of the initial quality hits, with activity against all four serotypes of dengue. Chemical analogs of this compound were obtained, and these analogs were tested as described in order to define the relationship between chemical structure and biological activity (see Table 1). All of the compounds in Table 1, labeled A or B, are active against dengue with EC₅₀ values at or below 25 μM.

TABLE 1
Compounds active against Dengue-2 Virus in Vero cells

| | | | | Activity |
|---|---|---|---|---|
| | | | | A: EC_{₅₀}≦ 5 uM; |
| | | | | B: |
| Com- | | | | 5 < EC_{₅₀}≦ 25 uM; |
| pound | Chemical Structure | Molecular Formula | Chemical Name | C: EC_{₅₀}> 25 uM |
| 1 | 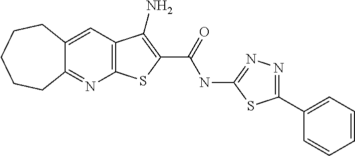 [SMILES: Nc1c(C(=O)Nc2nnc(-c3ccccc3)s2)sc2nc3c(cc12)CCCCC3] | C21H19N5OS2 | 3-Amino-6,7,8,9-tetrahydro- 5H-1-thia-10-aza- cyclohepta[f]indene-2- carboxylic acid (5-phenyl- [1,3,4]thiadiazol-2-yl)-amide | A |
| 2 | 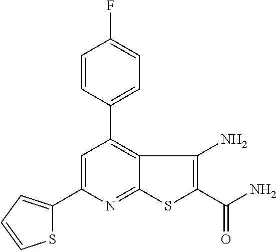 [SMILES: NC(=O)c1sc2nc(-c3cccs3)cc(-c3ccc(F)cc3)c2c1N] | C18H12FN3OS2 | 3-Amino-4-(4-fluoro-phenyl)-6- thiophen-2-yl-thieno[2,3- b]pyridine-2-carboxylic acid amide | A |
| 3 | 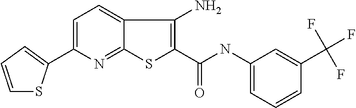 [SMILES: Nc1c(C(=O)Nc2cccc(C(F)(F)F)c2)sc2nc(-c3cccs3)ccc12] | C19H12F3N3OS2 | 3-Amino-6-thiophen-2-yl- thieno[2,3-b]pyridine-2- carboxylic acid (3- trifluoromethyl-phenyl)-amide | A |
| 4 | 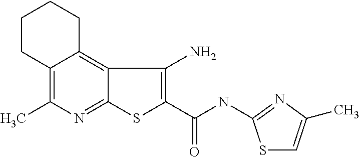 [SMILES: Cc1csc(NC(=O)c2sc3nc(C)c4c(c3c2N)CCCC4)n1] | C17H18N4OS2 | 1-Amino-5-methyl-6,7,8,9- tetrahydro-thieno[2,3- c]isoquinoline-2-carboxylic acid (4-methyl-thiazol-2-yl)- amide | A |
| 5 | 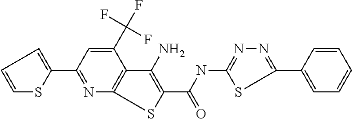 [SMILES: Nc1c(C(=O)Nc2nnc(-c3ccccc3)s2)sc2nc(-c3cccs3)cc(C(F)(F)F)c12] | C21H12F3N5OS3 | 3-Amino-6-thiophen-2-yl-4- trifluoromethyl-thieno[2,3- b]pyridine-2-carboxylic acid (5-phenyl-[1,3,4]thiadiazol-2- yl)-amide | A |
| 6 | 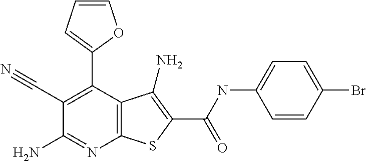 [SMILES: N#Cc1c(N)nc2sc(C(=O)Nc3ccc(Br)cc3)c(N)c2c1-c1ccco1] | C19H12BrN5O2S | 3,6-Diamino-5-cyano-4-furan- 2-yl-thieno[2,3-b]pyridine-2- carboxylic acid (4-bromo- phenyl)-amide | A |
| 7 | 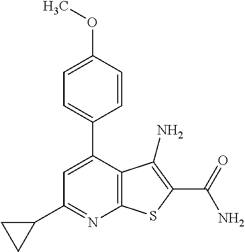 [SMILES: COc1ccc(-c2cc(C3CC3)nc3sc(C(N)=O)c(N)c23)cc1] | C18H17N3O2S | 3-Amino-6-cyclopropyl-4-(4- methoxy-phenyl)-thieno[2,3- b]pyridine-2-carboxylic acid amide | A |
| 8 | 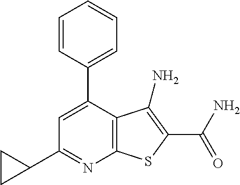 [SMILES: NC(=O)c1sc2nc(C3CC3)cc(-c3ccccc3)c2c1N] | C17H15N3OS | 3-Amino-6-cyclopropyl-4- phenyl-thieno[2,3-b]pyridine-2- carboxylic acid amide | A |
| 9 | 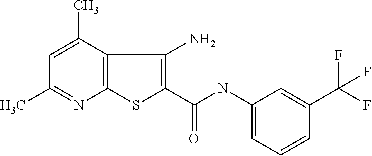 [SMILES: Cc1cc(C)c2c(N)c(C(=O)Nc3cccc(C(F)(F)F)c3)sc2n1] | C17H14F3N3OS | 3-Amino-4,6-dimethyl- thieno[2,3-b]pyridine-2- carboxylic acid (3- trifluoromethyl-phenyl)-amide | A |
| 10 | 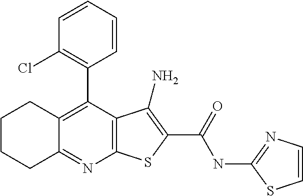 [SMILES: Nc1c(C(=O)Nc2nccs2)sc2nc3c(c(-c4ccccc4Cl)c12)CCCC3] | C21H17ClN4OS2 | 3-Amino-4-(2-chloro-phenyl)- 5,6,7,8-tetrahydro-thieno[2,3- b]quinoline-2-carboxylic acid thiazol-2-ylamide | A |
| 11 | 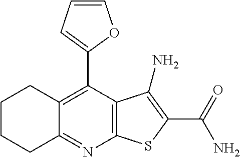 [SMILES: NC(=O)c1sc2nc3c(c(-c4ccco4)c2c1N)CCCC3] | C16H15N3O2S | 3-Amino-4-furan-2-yl-5,6,7,8- tetrahydro-thieno[2,3- b]quinoline-2-carboxylic acid amide | A |
| 12 | 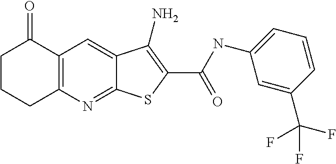 [SMILES: Nc1c(C(=O)Nc2cccc(C(F)(F)F)c2)sc2nc3c(cc12)C(=O)CCC3] | C19H14F3N3O2S | 3-Amino-5-oxo-5,6,7,8- tetrahydro-thieno[2,3- b]quinoline-2-carboxylic acid (3-trifluoromethyl-phenyl)- amide | A |
| 13 | 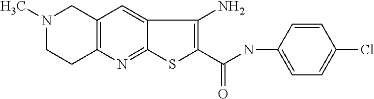 [SMILES: CN1CCc2nc3sc(C(=O)Nc4ccc(Cl)cc4)c(N)c3cc2C1] | C18H17ClN4OS | 3-Amino-6-methyl-5,6,7,8- tetrahydro-thieno[2,3- b][1,6]naphthyridine-2- carboxylic acid (4-chloro- phenyl)-amide | A |
| 14 | 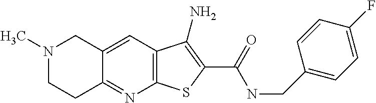 [SMILES: CN1CCc2nc3sc(C(=O)NCc4ccc(F)cc4)c(N)c3cc2C1] | C19H19FN4OS | 3-Amino-6-methyl-5,6,7,8- tetrahydro-thieno[2,3- b][1,6]naphthyridine-2- carboxylic acid 4-fluoro- benzylamide | A |
| 15 | 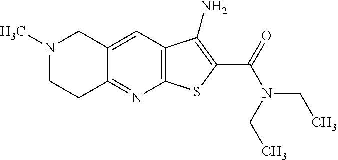 [SMILES: CCN(CC)C(=O)c1sc2nc3c(cc2c1N)CN(C)CC3] | C16H22N4OS | 3-Amino-6-methyl-5,6,7,8- tetrahydro-thieno[2,3- b][1,6]naphthyridine-2- carboxylic acid diethylamide | A |
| 16 | 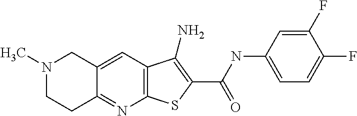 [SMILES: CN1CCc2nc3sc(C(=O)Nc4ccc(F)c(F)c4)c(N)c3cc2C1] | C18H16F2N4OS | 3-Amino-6-methyl-5,6,7,8- tetrahydro-thieno[2,3- b][1,6]naphthyridine-2- carboxylic acid (3,4-difluoro- phenyl)-amide | A |
| 17 | 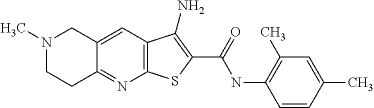 | C20H22N4OS | 3-Amino-6-methyl-5,6,7,8- tetrahydro-thieno[2,3- b][1,6]naphthyridine-2- carboxylic acid (2,4-dimethyl- phenyl)-amide | A |
| 18 | 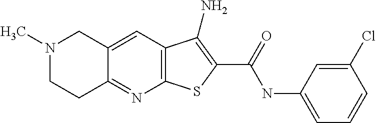 [SMILES: CN1CCc2nc3sc(C(=O)Nc4cccc(Cl)c4)c(N)c3cc2C1] | C18H17ClN4OS | 3-Amino-6-methyl-5,6,7,8- tetrahydro-thieno[2,3- b][1,6]naphthyridine-2- carboxylic acid (3-chloro- phenyl)-amide | A |
| 19 | 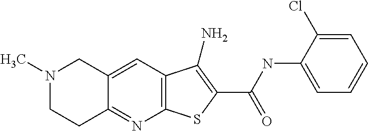 [SMILES: CN1CCc2nc3sc(C(=O)Nc4ccccc4Cl)c(N)c3cc2C1] | C18H17ClN4OS | 3-Amino-6-methyl-5,6,7,8- tetrahydro-thieno[2,3- b][1,6]naphthyridine-2- carboxylic acid (2-chloro- phenyl)-amide | A |
| 20 | 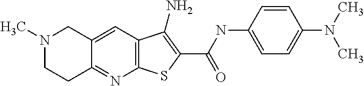 | C20H23N5OS | 3-Amino-6-methyl-5,6,7,8- tetrahydro-thieno[2,3- b][1,6]naphthyridine-2- carboxylic acid (4- dimethylamino-phenyl)-amide | A |
| 21 | 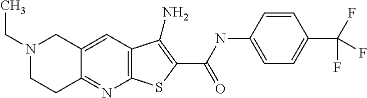 [SMILES: CCN1CCc2nc3sc(C(=O)Nc4ccc(C(F)(F)F)cc4)c(N)c3cc2C1] | C20H19F3N4OS | 3-Amino-6-ethyl-5,6,7,8- tetrahydro-thieno[2,3- b][1,6]naphthyridine-2- carboxylic acid (4- trifluoromethyl-phenyl)-amide | A |
| 22 | 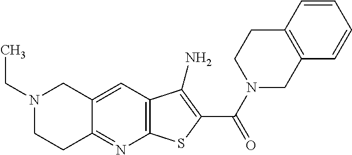 [SMILES: CCN1CCc2nc3sc(C(=O)N4CCc5ccccc5C4)c(N)c3cc2C1] | C22H24N4OS | (3-Amino-6-ethyl-5,6,7,8- tetrahydro-thieno[2,3- b][1,6]naphthyridin-2-yl)-(3,4- dihydro-1H-isoquinolin-2-yl)- methanone | A |
| 23 | 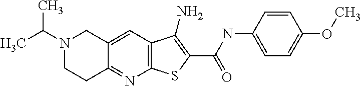 [SMILES: COc1ccc(NC(=O)c2sc3nc4c(cc3c2N)CN(C(C)C)CC4)cc1] | C21H24N4O2S | 3-Amino-6-isopropyl-5,6,7,8- tetrahydro-thieno[2,3- b][1,6]naphthyridine-2- carboxylic acid (4-methoxy- phenyl)-amide | A |
| 24 | 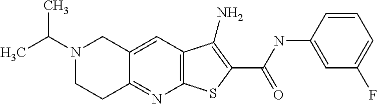 [SMILES: CC(C)N1CCc2nc3sc(C(=O)Nc4cccc(F)c4)c(N)c3cc2C1] | C20H21FN4OS | 3-Amino-6-isopropyl-5,6,7,8- tetrahydro-thieno[2,3- b][1,6]naphthyridine-2- carboxylic acid (3-fluoro- phenyl)-amide | A |
| 25 | 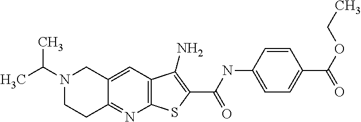 [SMILES: CCOC(=O)c1ccc(NC(=O)c2sc3nc4c(cc3c2N)CN(C(C)C)CC4)cc1] | C23H26N4O3S | 4-[(3-Amino-6-isopropyl- 5,6,7,8-tetrahydro-thieno[2,3- b][1,6]naphthyridine-2- carbonyl)-amino]-benzoic acid ethyl ester | A |
| 26 | 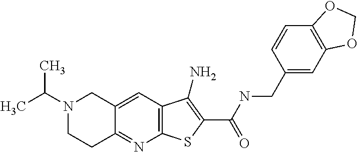 [SMILES: CC(C)N1CCc2nc3sc(C(=O)NCc4ccc5c(c4)OCO5)c(N)c3cc2C1] | C22H24N4O3S | 3-Amino-6-isopropyl-5,6,7,8- tetrahydro-thieno[2,3- b][1,6]naphthyridine-2- carboxylic acid (benzo[1,3]dioxol-5-ylmethyl)- amide | A |
| 27 | 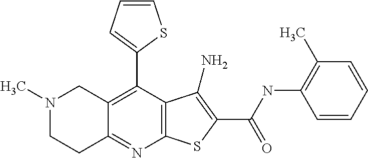 [SMILES: Cc1ccccc1NC(=O)c1sc2nc3c(c(-c4cccs4)c2c1N)CN(C)CC3] | C23H22N4OS2 | 3-Amino-6-methyl-4-thiophen- 2-yl-5,6,7,8-tetrahydro- thieno[2,3- b][1,6]naphthyridine-2- carboxylic acid o-tolylamide | A |
| 28 | 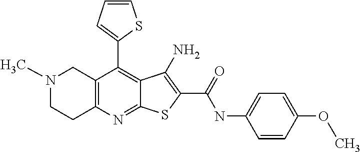 | C23H22N4O2S2 | 3-Amino-6-methyl-4-thiophen- 2-yl-5,6,7,8-tetrahydro- thieno[2,3- b][1,6]naphthyridine-2- carboxylic acid (4-methoxy- phenyl)-amide | A |
| 29 | 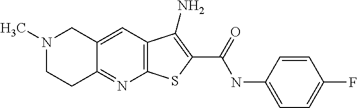 [SMILES: CN1CCc2nc3sc(C(=O)Nc4ccc(F)cc4)c(N)c3cc2C1] | C18H17FN4OS | 3-Amino-6-methyl-5,6,7,8- tetrahydro-thieno[2,3- b][1,6]naphthyridine-2- carboxylic acid (4-fluoro- phenyl)-amide | A |
| 30 | 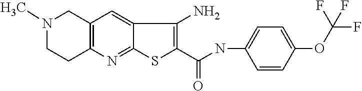 [SMILES: CN1CCc2nc3sc(C(=O)Nc4ccc(OC(F)(F)F)cc4)c(N)c3cc2C1] | C19H17F3N4O2S | 3-Amino-6-methyl-5,6,7,8- tetrahydro-thieno[2,3- b][1,6]naphthyridine-2- carboxylic acid (4- trifluoromethoxy-phenyl)- amide | A |
| 31 | 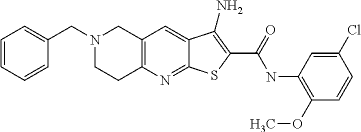 [SMILES: COc1ccc(Cl)cc1NC(=O)c1sc2nc3c(cc2c1N)CN(Cc1ccccc1)CC3] | C25H23ClN4O2S | 3-Amino-6-benzyl-5,6,7,8- tetrahydro-thieno[2,3- b][1,6]naphthyridine-2- carboxylic acid (5-chloro-2- methoxy-phenyl)-amide | A |
| 32 | 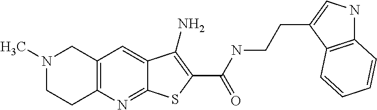 [SMILES: CN1CCc2nc3sc(C(=O)NCCc4c[nH]c5ccccc45)c(N)c3cc2C1] | C22H23N5OS | 3-Amino-6-methyl-5,6,7,8- tetrahydro-thieno[2,3- b][1,6]naphthyridine-2- carboxylic acid [2-(1H-indol-3- yl)-ethyl]-amide | A |
| 33 | 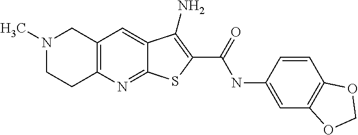 [SMILES: CN1CCc2nc3sc(C(=O)Nc4ccc5c(c4)OCO5)c(N)c3cc2C1] | C19H18N4O3S | 3-Amino-6-methyl-5,6,7,8- tetrahydro-thieno[2,3- b][1,6]naphthyridine-2- carboxylic acid benzo[1,3]dioxol-5-ylamide | A |
| 34 | 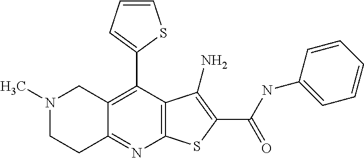 [SMILES: CN1CCc2nc3sc(C(=O)Nc4ccccc4)c(N)c3c(-c3cccs3)c2C1] | C22H20N4OS2 | 3-Amino-6-methyl-4-thiophen- 2-yl-5,6,7,8-tetrahydro- thieno[2,3- b][1,6]naphthyridine-2- carboxylic acid phenylamide | A |
| 35 | 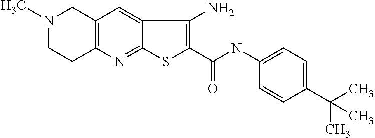 [SMILES: CN1CCc2nc3sc(C(=O)Nc4ccc(C(C)(C)C)cc4)c(N)c3cc2C1] | C22H26N4OS | 3-Amino-6-methyl-5,6,7,8- tetrahydro-thieno[2,3- b][1,6]naphthyridine-2- carboxylic acid (4-tert-butyl- phenyl)-amide | A |
| 36 | 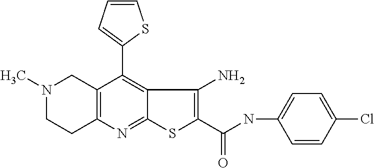 [SMILES: CN1CCc2nc3sc(C(=O)Nc4ccc(Cl)cc4)c(N)c3c(-c3cccs3)c2C1] | C22H19ClN4OS2 | 3-Amino-6-methyl-4-thiophen- 2-yl-5,6,7,8-tetrahydro- thieno[2,3- b][1,6]naphthyridine-2- carboxylic acid (4-chloro- phenyl)-amide | A |
| 37 | 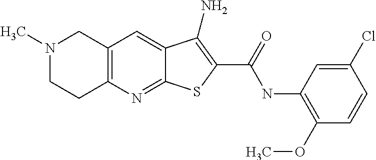 [SMILES: COc1ccc(Cl)cc1NC(=O)c1sc2nc3c(cc2c1N)CN(C)CC3] | C19H19ClN4O2S | 3-Amino-6-methyl-5,6,7,8- tetrahydro-thieno[2,3- b][1,6]naphthyridine-2- carboxylic acid (5-chloro-2- methoxy-phenyl)-amide | A |
| 38 | 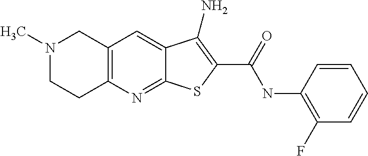 [SMILES: CN1CCc2nc3sc(C(=O)Nc4ccccc4F)c(N)c3cc2C1] | C18H17FN4OS | 3-Amino-6-methyl-5,6,7,8- tetrahydro-thieno[2,3- b][1,6]naphthyridine-2- carboxylic acid (2-fluoro- phenyl)-amide | A |
| 39 | 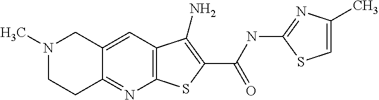 | C16H17N5OS2 | 3-Amino-6-methyl-5,6,7,8- tetrahydro-thieno[2,3- b][1,6]naphthyridine-2- carboxylic acid (4-methyl- thiazol-2-yl)-amide | A |
| 40 | 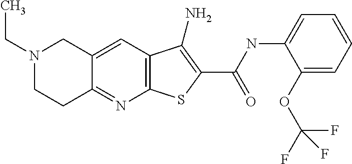 [SMILES: CCN1CCc2nc3sc(C(=O)Nc4ccccc4OC(F)(F)F)c(N)c3cc2C1] | C20H19F3N4O2S | 3-Amino-6-ethyl-5,6,7,8- tetrahydro-thieno[2,3- b][1,6]naphthyridine-2- carboxylic acid (2- trifluoromethoxy-phenyl)- amide | A |
| 41 | 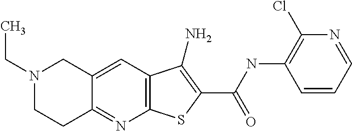 [SMILES: CCN1CCc2nc3sc(C(=O)Nc4cccnc4Cl)c(N)c3cc2C1] | C18H18ClN5OS | 3-Amino-6-ethyl-5,6,7,8- tetrahydro-thieno[2,3- b][1,6]naphthyridine-2- carboxylic acid (2-chloro- pyridin-3-yl)-amide | A |
| 42 | 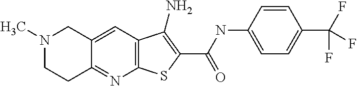 [SMILES: CN1CCc2nc3sc(C(=O)Nc4ccc(C(F)(F)F)cc4)c(N)c3cc2C1] | C19H17F3N4OS | 3-Amino-6-methyl-5,6,7,8- tetrahydro-thieno[2,3- b][1,6]naphthyridine-2- carboxylic acid (4- trifluoromethyl-phenyl)-amide | A |
| 43 | 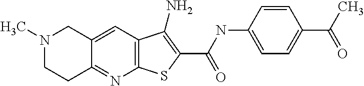 [SMILES: CC(=O)c1ccc(NC(=O)c2sc3nc4c(cc3c2N)CN(C)CC4)cc1] | C20H20N4O2S | 3-Amino-6-methyl-5,6,7,8- tetrahydro-thieno[2,3- b][1,6]naphthyridine-2- carboxylic acid (4-acetyl- phenyl)-amide | A |
| 44 | 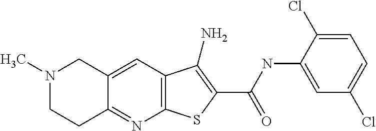 [SMILES: CN1CCc2nc3sc(C(=O)Nc4cc(Cl)ccc4Cl)c(N)c3cc2C1] | C18H16Cl2N4OS | 3-Amino-6-methyl-5,6,7,8- tetrahydro-thieno[2,3- b][1,6]naphthyridine-2- carboxylic acid (2,5-dichloro- phenyl)-amide | A |
| 45 | 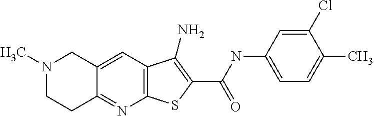 | C19H19ClN4OS | 3-Amino-6-methyl-5,6,7,8- tetrahydro-thieno[2,3- b][1,6]naphthyridine-2- carboxylic acid (3-chloro-4- methyl-phenyl)-amide | A |
| 46 | 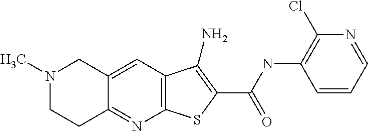 [SMILES: CN1CCc2nc3sc(C(=O)Nc4cccnc4Cl)c(N)c3cc2C1] | C17H16ClN5OS | 3-Amino-6-methyl-5,6,7,8- tetrahydro-thieno[2,3- b][1,6]naphthyridine-2- carboxylic acid (2-chloro- pyridin-3-yl)-amide | A |
| 47 | 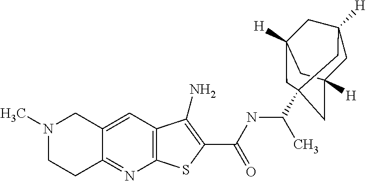 [SMILES: CC(NC(=O)c1sc2nc3c(cc2c1N)CN(C)CC3)[C@]12C[C@H]3C[C@H](C[C@H](C3)C1)C2] | C24H32N4OS | 3-Amino-6-methyl-5,6,7,8- tetrahydro-thieno[2,3- b][1,6]naphthyridine-2- carboxylic acid (1-adamantan- 1-yl-ethyl)-amide | A |
| 48 | 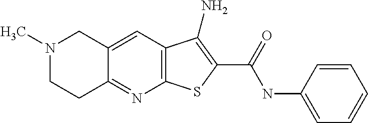 [SMILES: CN1CCc2nc3sc(C(=O)Nc4ccccc4)c(N)c3cc2C1] | C18H18N4OS | 3-Amino-6-methyl-5,6,7,8- tetrahydro-thieno[2,3- b][1,6]naphthyridine-2- carboxylic acid phenylamide | A |
| 49 | 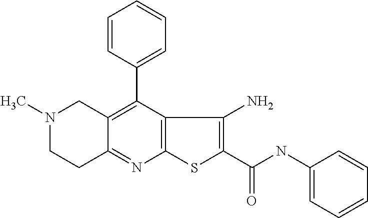 [SMILES: CN1CCc2nc3sc(C(=O)Nc4ccccc4)c(N)c3c(-c3ccccc3)c2C1] | C24H22N4OS | 3-Amino-6-methyl-4-phenyl- 5,6,7,8-tetrahydro-thieno[2,3- b][1,6]naphthyridine-2- carboxylic acid phenylamide | A |
| 50 | 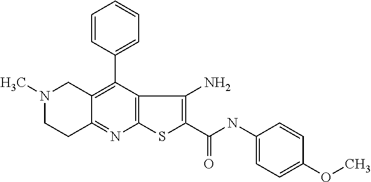 | C25H24N4O2S | 3-Amino-6-methyl-4-phenyl- 5,6,7,8-tetrahydro-thieno[2,3- b][1,6]naphthyridine-2- carboxylic acid (4-methoxy- phenyl)-amide | A |
| 51 | 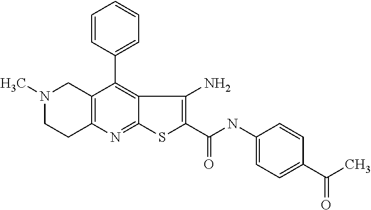 [SMILES: CC(=O)c1ccc(NC(=O)c2sc3nc4c(c(-c5ccccc5)c3c2N)CN(C)CC4)cc1] | C26H24N4O2S | 3-Amino-6-methyl-4-phenyl- 5,6,7,8-tetrahydro-thieno[2,3- b][1,6]naphthyridine-2- carboxylic acid (4-acetyl- phenyl)-amide | A |
| 52 | 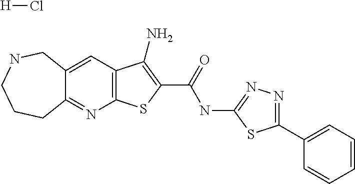 [SMILES: Cl ; Nc1c(C(=O)Nc2nnc(-c3ccccc3)s2)sc2nc3c(cc12)CNCCC3] | C20H18N6OS2•HCl | 3-Amino-6,7,8,9-tetrahydro- 5H-1-thia-6,10-diaza cyclohepta[f]indene-2- carboxylic acid (5-phenyl- [1,3,4]thiadiazol-2-yl)-amide hydrochloride | A |
| 53 | 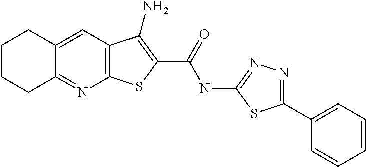 [SMILES: Nc1c(C(=O)Nc2nnc(-c3ccccc3)s2)sc2nc3c(cc12)CCCC3] | C20H17N5OS2 | 3-Amino-5,6,7,8-tetrahydro- thieno[2,3-b]quinoline-2- carboxylic acid (5-phenyl- [1,3,4]thiadiazol-2-yl)-amide | A |
| 54 | 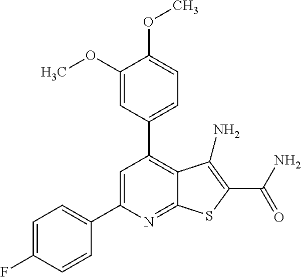 [SMILES: COc1ccc(-c2cc(-c3ccc(F)cc3)nc3sc(C(N)=O)c(N)c23)cc1OC] | C22H18FN3O3S | 3-Amino-4-(3,4-dimethoxy- phenyl)-6-(4-fluoro-phenyl)- thieno[2,3-b]pyridine-2- carboxylic acid amide | A |
| 55 | 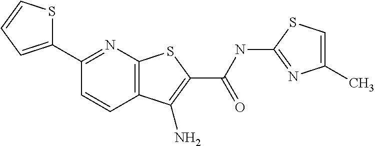 [SMILES: Cc1csc(NC(=O)c2sc3nc(-c4cccs4)ccc3c2N)n1] | C16H12N4OS3 | 3-Amino-6-thiophen-2-yl- thieno[2,3-b]pyridine-2- carboxylic acid (4-methyl- thiazol-2-yl)-amide | A |
| 56 | 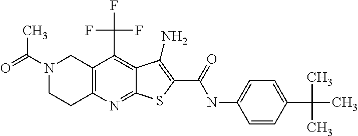 [SMILES: CC(=O)N1CCc2nc3sc(C(=O)Nc4ccc(C(C)(C)C)cc4)c(N)c3c(C(F)(F)F)c2C1] | C24H25F3N4O2S | 6-Acetyl-3-amino-4- trifluoromethyl-5,6,7,8- tetrahydro-thieno[2,3- b][1,6]naphthyridine-2- carboxylic acid (4-tert-butyl- phenyl)-amide | A |
| 57 | 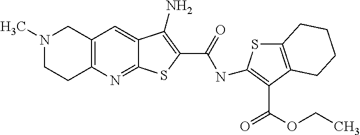 [SMILES: CCOC(=O)c1c(NC(=O)c2sc3nc4c(cc3c2N)CN(C)CC4)sc2c1CCCC2] | C23H26N4O3S2 | 2-[(3-Amino-6-methyl-5,6,7,8- tetrahydro-thieno[2,3- b][1,6]naphthyridine-2- carbonyl)-amino]-4,5,6,7- tetrahydro-benzo[b]thiophene- 3-carboxylic acid ethyl ester | A |
| 58 | 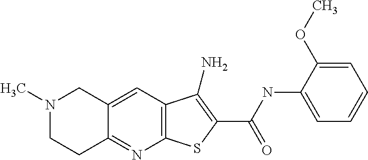 [SMILES: COc1ccccc1NC(=O)c1sc2nc3c(cc2c1N)CN(C)CC3] | C19H20N4O2S | 3-Amino-6-methyl-5,6,7,8- tetrahydro-thieno[2,3- b][1,6]naphthyridine-2- carboxylic acid (2-methoxy- phenyl)-amide | A |
| 59 | 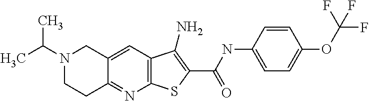 [SMILES: CC(C)N1CCc2nc3sc(C(=O)Nc4ccc(OC(F)(F)F)cc4)c(N)c3cc2C1] | C21H21F3N4O2S | 3-Amino-6-isopropyl-5,6,7,8- tetrahydro-thieno[2,3- b][1,6]naphthyridine-2- carboxylic acid (4- trifluoromethoxy-phenyl)- amide | A |
| 60 | 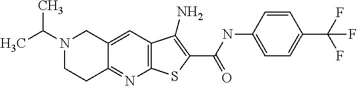 [SMILES: CC(C)N1CCc2nc3sc(C(=O)Nc4ccc(C(F)(F)F)cc4)c(N)c3cc2C1] | C21H21F3N4OS | 3-Amino-6-isopropyl-5,6,7,8- tetrahydro-thieno[2,3- b][1,6]naphthyridine-2- carboxylic acid (4- trifluoromethyl-phenyl)-amide | A |
| 61 | 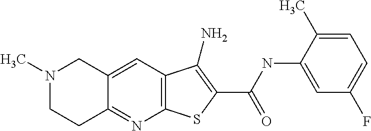 [SMILES: Cc1ccc(F)cc1NC(=O)c1sc2nc3c(cc2c1N)CN(C)CC3] | C19H19FN4OS | 3-Amino-6-methyl-5,6,7,8- tetrahydro-thieno[2,3- b][1,6]naphthyridine-2- carboxylic acid (5-fluoro-2- methyl-phenyl)-amide | A |
| 62 | 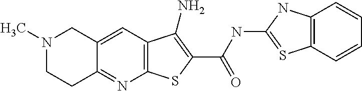 | C19H17N5OS2 | 3-Amino-6-methyl-5,6,7,8- tetrahydro-thieno[2,3- b][1,6]naphthyridine-2- carboxylic acid benzothiazol- 2-ylamide | A |
| 63 | 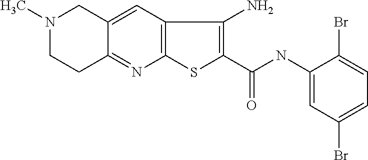 [SMILES: CN1CCc2nc3sc(C(=O)Nc4cc(Br)ccc4Br)c(N)c3cc2C1] | C18H16Br2N4OS | 3-Amino-6-methyl-5,6,7,8- tetrahydro-thieno[2,3- b][1,6]naphthyridine-2- carboxylic acid (2,5-dibromo- phenyl)-amide | A |
| 64 | 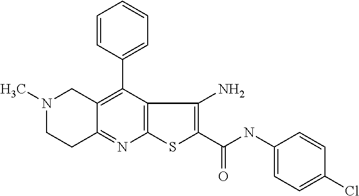 [SMILES: CN1CCc2nc3sc(C(=O)Nc4ccc(Cl)cc4)c(N)c3c(-c3ccccc3)c2C1] | C24H21ClN4OS | 3-Amino-6-methyl-4-phenyl- 5,6,7,8-tetrahydro-thieno[2,3- b][1,6]naphthyridine-2- carboxylic acid (4-chloro- phenyl)-amide | A |
| 65 | 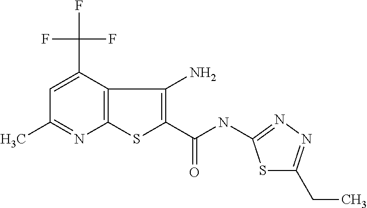 [SMILES: CCc1nnc(NC(=O)c2sc3nc(C)cc(C(F)(F)F)c3c2N)s1] | C14H12F3N5OS2 | 3-Amino-6-methyl-4- trifluoromethyl-thieno[2,3- b]pyridine-2-carboxylic acid (5-ethyl-[1,3,4]thiadiazol-2-yl)- amide | B |
| 66 | 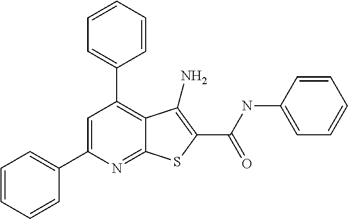 [SMILES: Nc1c(C(=O)Nc2ccccc2)sc2nc(-c3ccccc3)cc(-c3ccccc3)c12] | C26H19N3OS | 3-Amino-4,6-diphenyl- thieno[2,3-b]pyridine-2- carboxylic acid phenylamide | B |
| 67 | 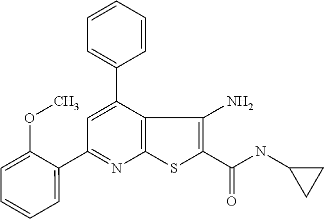 [SMILES: COc1ccccc1-c1cc(-c2ccccc2)c2c(N)c(C(=O)NC3CC3)sc2n1] | C24H21N3O2S | 3-Amino-6-(2-methoxy- phenyl)-4-phenyl-thieno[2,3- b]pyridine-2-carboxylic acid cyclopropylamide | B |
| 68 | 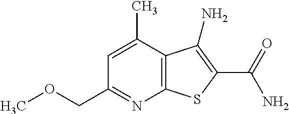 [SMILES: COCc1cc(C)c2c(N)c(C(N)=O)sc2n1] | C11H13N3O2S | 3-Amino-6-methoxymethyl-4- methyl-thieno[2,3-b]pyridine- 2-carboxylic acid amide | B |
| 69 | 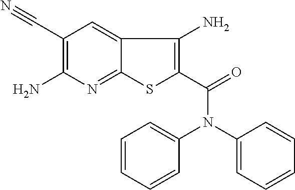 [SMILES: N#Cc1cc2c(N)c(C(=O)N(c3ccccc3)c3ccccc3)sc2nc1N] | C21H15N5OS | 3,6-Diamino-5-cyano- thieno[2,3-b]pyridine-2- carboxylic acid diphenylamide | B |
| 70 | 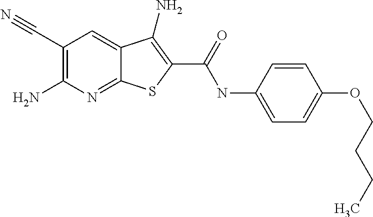 [SMILES: CCCCOc1ccc(NC(=O)c2sc3nc(N)c(C#N)cc3c2N)cc1] | C19H19N5O2S | 3,6-Diamino-5-cyano- thieno[2,3-b]pyridine-2- carboxylic acid (4-butoxy- phenyl)-amide | B |
| 71 | 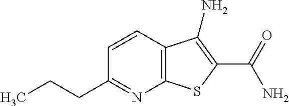 [SMILES: CCCc1ccc2c(N)c(C(N)=O)sc2n1] | C11H13N3OS | 3-Amino-6-propyl-thieno[2,3- b]pyridine-2-carboxylic acid amide | B |
| 72 | 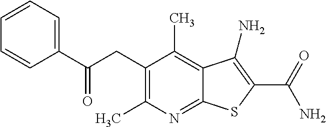 [SMILES: Cc1nc2sc(C(N)=O)c(N)c2c(C)c1CC(=O)c1ccccc1] | C18H17N3O2S | 3-Amino-4,6-dimethyl-5-(2- oxo-2-phenyl-ethyl)-thieno[2,3- b]pyridine-2-carboxylic acid amide | B |
| 73 | 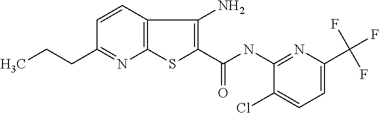 [SMILES: CCCc1ccc2c(N)c(C(=O)Nc3nc(C(F)(F)F)ccc3Cl)sc2n1] | C17H14ClF3N4OS | 3-Amino-6-propyl-thieno[2,3- b]pyridine-2-carboxylic acid (3-chloro-6-trifluoromethyl- pyridin-2-yl)-amide | B |
| 74 | 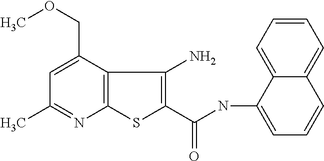 [SMILES: COCc1cc(C)nc2sc(C(=O)Nc3cccc4ccccc34)c(N)c12] | C21H19N3O2S | 3-Amino-4-methoxymethyl-6- methyl-thieno[2,3-b]pyridine-2- carboxylic acid naphthalen-1- ylamide | B |
| 75 | 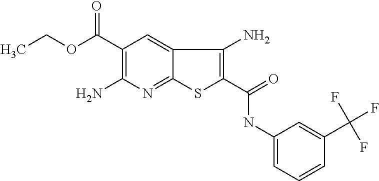 [SMILES: CCOC(=O)c1cc2c(N)c(C(=O)Nc3cccc(C(F)(F)F)c3)sc2nc1N] | C18H15F3N4O3S | 3,6-Diamino-2-(3- trifluoromethyl- phenylcarbamoyl)-thieno[2,3- b]pyridine-5-carboxylic acid ethyl ester | B |
| 76 | 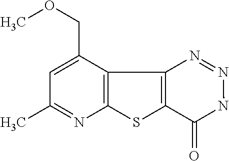 [SMILES: COCc1cc(C)nc2sc3c(=O)[nH]nnc3c12] | C11H10N4O2S | 9-Methoxymethyl-7-methyl- 3H-pyrido[3′,2′:4,5]thieno[3,2- d][1,2,3]triazin-4-one | B |
| 77 | 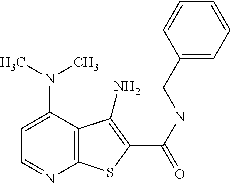 [SMILES: CN(C)c1ccnc2sc(C(=O)NCc3ccccc3)c(N)c12] | C17H18N4OS | 3-Amino-4-dimethylamino- thieno[2,3-b]pyridine-2- carboxylic acid benzylamide | B |
| 78 | 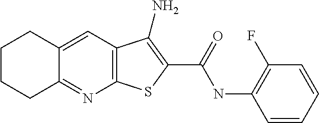 [SMILES: Nc1c(C(=O)Nc2ccccc2F)sc2nc3c(cc12)CCCC3] | C18H16FN3OS | 3-Amino-5,6,7,8-tetrahydro- thieno[2,3-b]quinoline-2- carboxylic acid (2-fluoro- phenyl)-amide | B |
| 79 | 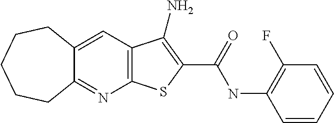 [SMILES: Nc1c(C(=O)Nc2ccccc2F)sc2nc3c(cc12)CCCCC3] | C19H18FN3OS | 3-Amino-6,7,8,9-tetrahydro- 5H-1-thia-10-aza- cyclohepta[f]indene-2- carboxylic acid (2-fluoro- phenyl)-amide | B |
| 80 | 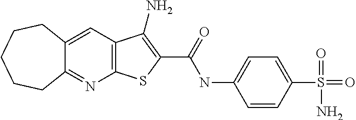 [SMILES: Nc1c(C(=O)Nc2ccc(S(N)(=O)=O)cc2)sc2nc3c(cc12)CCCCC3] | C19H20N4O3S2 | 3-Amino-6,7,8,9-tetrahydro- 5H-1-thia-10-aza- cyclohepta[f]indene-2- carboxylic acid (4-sulfamoyl- phenyl)-amide | B |
| 81 | 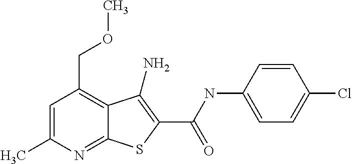 [SMILES: COCc1cc(C)nc2sc(C(=O)Nc3ccc(Cl)cc3)c(N)c12] | C17H16ClN3O2S | 3-Amino-4-methoxymethyl-6- methyl-thieno[2,3-b]pyridine-2- carboxylic acid (4-chloro- phenyl)-amide | B |
| 82 | 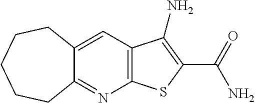 [SMILES: NC(=O)c1sc2nc3c(cc2c1N)CCCCC3] | C13H15N3OS | 3-Amino-6,7,8,9-tetrahydro- 5H-1-thia-10-aza- cyclohepta[f]indene-2- carboxylic acid amide | B |
| 83 | 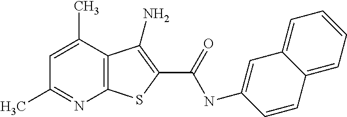 [SMILES: Cc1cc(C)c2c(N)c(C(=O)Nc3ccc4ccccc4c3)sc2n1] | C20H17N3OS | 3-Amino-4,6-dimethyl- thieno[2,3-b]pyridine-2- carboxylic acid naphthalen-2- ylamide | B |
| 84 | 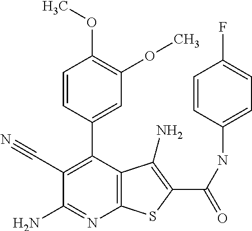 [SMILES: COc1ccc(-c2c(C#N)c(N)nc3sc(C(=O)Nc4ccc(F)cc4)c(N)c23)cc1OC] | C23H18FN5O3S | 3,6-Diamino-5-cyano-4-(3,4- dimethoxy-phenyl)-thieno-[2,3- b]pyridine-2-carboxylic acid (4-fluoro-phenyl)-amide | B |
| 85 | 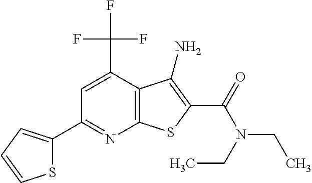 [SMILES: CCN(CC)C(=O)c1sc2nc(-c3cccs3)cc(C(F)(F)F)c2c1N] | C17H16F3N3OS2 | 3-Amino-6-thiophen-2-yl-4- trifluoromethyl-thieno[2,3- b]pyridine-2-carboxylic acid diethylamide | B |
| 86 | 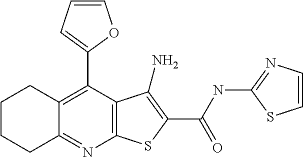 [SMILES: Nc1c(C(=O)Nc2nccs2)sc2nc3c(c(-c4ccco4)c12)CCCC3] | C19H16N4O2S2 | 3-Amino-4-furan-2-yl-5,6,7,8- tetrahydro-thieno[2,3- b]quinoline-2-carboxylic acid thiazol-2-ylamide | B |
| 87 | 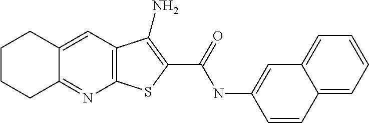 [SMILES: Nc1c(C(=O)Nc2ccc3ccccc3c2)sc2nc3c(cc12)CCCC3] | C22H19N3OS | 3-Amino-5,6,7,8-tetrahydro- thieno[2,3-b]quinoline-2- carboxylic acid naphthalen-2- ylamide | B |
| 88 | 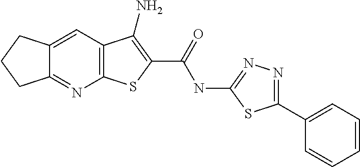 [SMILES: Nc1c(C(=O)Nc2nnc(-c3ccccc3)s2)sc2nc3c(cc12)CCC3] | C19H15N5OS2 | 3-Amino-6,7-dihydro-5H- cyclopenta[b]thieno[3,2- e]pyridine-2-carboxylic acid (5-phenyl-[1,3,4]thiadiazol-2- yl)-amide | B |
| 89 | 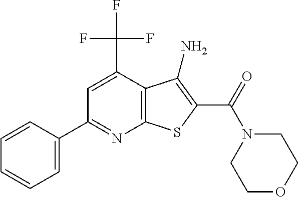 [SMILES: Nc1c(C(=O)N2CCOCC2)sc2nc(-c3ccccc3)cc(C(F)(F)F)c12] | C19H16F3N3O2S | (3-Amino-6-phenyl-4- trifluoromethyl-thieno[2,3- b]pyridin-2-yl)-morpholin-4-yl- methanone | B |
| 90 | 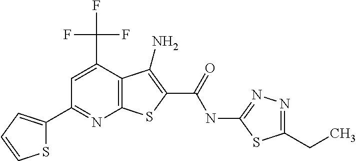 [SMILES: CCc1nnc(NC(=O)c2sc3nc(-c4cccs4)cc(C(F)(F)F)c3c2N)s1] | C17H12F3N5OS3 | 3-Amino-6-thiophen-2-yl-4- trifluoromethyl-thieno[2,3- b]pyridine-2-carboxylic acid (5-ethyl-[1,3,4]thiadiazol-2-yl)- amide | B |
| 91 | 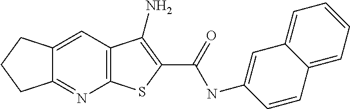 [SMILES: Nc1c(C(=O)Nc2ccc3ccccc3c2)sc2nc3c(cc12)CCC3] | C21H17N3OS | 3-Amino-6,7-dihydro-5H- cyclopenta[b]thieno[3,2- e]pyridine-2-carboxylic acid naphthalen-2-ylamide | B |
| 92 | 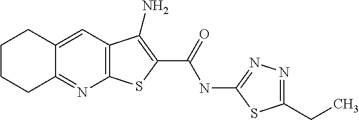 [SMILES: CCc1nnc(NC(=O)c2sc3nc4c(cc3c2N)CCCC4)s1] | C16H17N5OS2 | 3-Amino-5,6,7,8-tetrahydro- thieno[2,3-b]quinoline-2- carboxylic acid (5-ethyl- [1,3,4]thiadiazol-2-yl)-amide | B |
| 93 | 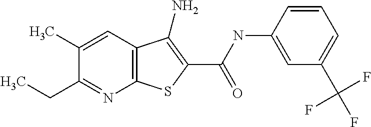 [SMILES: CCc1nc2sc(C(=O)Nc3cccc(C(F)(F)F)c3)c(N)c2cc1C] | C18H16F3N3OS | 3-Amino-6-ethyl-5-methyl- thieno[2,3-b]pyridine-2- carboxylic acid (3- trifluoromethyl-phenyl)-amide | B |
| 94 | 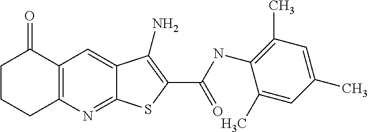 [SMILES: Cc1cc(C)c(NC(=O)c2sc3nc4c(cc3c2N)C(=O)CCC4)c(C)c1] | C21H21N3O2S | 3-Amino-5-oxo-5,6,7,8- tetrahydro-thieno[2,3- b]quinoline-2-carboxylic acid (2,4,6-trimethyl-phenyl)-amide | B |
| 95 | 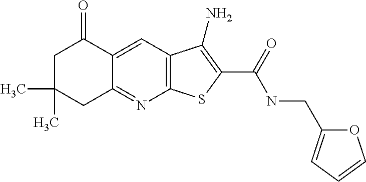 [SMILES: CC1(C)CC(=O)c2cc3c(N)c(C(=O)NCc4ccco4)sc3nc2C1] | C19H19N3O3S | 3-Amino-7,7-dimethyl-5-oxo- 5,6,7,8-tetrahydro-thieno[2,3- b]quinoline-2-carboxylic acid (furan-2-ylmethyl)-amide | B |
| 96 | 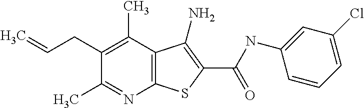 [SMILES: C=CCc1c(C)nc2sc(C(=O)Nc3cccc(Cl)c3)c(N)c2c1C] | C19H18ClN3OS | 5-Allyl-3-amino-4,6-dimethyl- thieno[2,3-b]pyridine-2- carboxylic acid (3-chloro- phenyl)-amide | B |
| 97 | 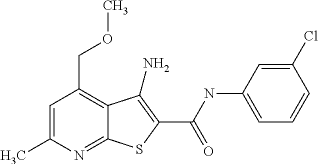 [SMILES: COCc1cc(C)nc2sc(C(=O)Nc3cccc(Cl)c3)c(N)c12] | C17H16ClN3O2S | 3-Amino-4-methoxymethyl-6- methyl-thieno[2,3-b]pyridine-2- carboxylic acid (3-chloro- phenyl)-amide | B |
| 98 | 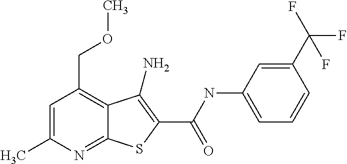 [SMILES: COCc1cc(C)nc2sc(C(=O)Nc3cccc(C(F)(F)F)c3)c(N)c12] | C18H16F3N3O2S | 3-Amino-4-methoxymethyl-6- methyl-thieno[2,3-b]pyridine-2- carboxylic acid (3- trifluoromethyl-phenyl)-amide | B |
| 99 | 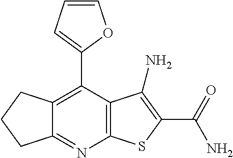 [SMILES: NC(=O)c1sc2nc3c(c(-c4ccco4)c2c1N)CCC3] | C15H13N3O2S | 3-Amino-4-furan-2-yl-6,7- dihydro-5H- cyclopenta[b]thieno[3,2- e]pyridine-2-carboxylic acid amide | B |
| 100 | 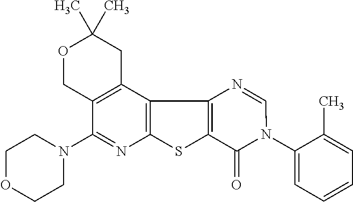 [SMILES: Cc1ccccc1-n1cnc2c(sc3nc(N4CCOCC4)c4c(c32)CC(C)(C)OC4)c1=O] | C25H26N4O3S | 2,2-Dimethyl-5-morpholin-4-yl- 9-o-tolyl-1,4-dihydro-2H,9H-3- oxa-7-thia-6,9,11-triaza- benzo[c]fluoren-8-one | B |
| 101 | 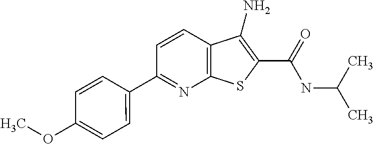 [SMILES: COc1ccc(-c2ccc3c(N)c(C(=O)NC(C)C)sc3n2)cc1] | C18H19N3O2S | 3-Amino-6-(4-methoxy- phenyl)-thieno[2,3-b]pyridine- 2-carboxylic acid isopropylamide | B |
| 102 | 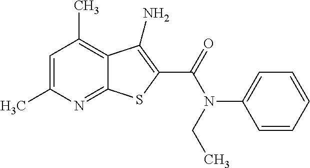 [SMILES: CCN(C(=O)c1sc2nc(C)cc(C)c2c1N)c1ccccc1] | C18H19N3OS | 3-Amino-4,6-dimethyl- thieno[2,3-b]pyridine-2- carboxylic acid ethyl-phenyl- amide | B |
| 103 | 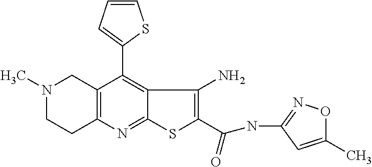 | C20H19N5O2S2 | 3-Amino-6-methyl-4-thiophen- 2-yl-5,6,7,8-tetrahydro- thieno[2,3- b][1,6]naphthyridine-2- carboxylic acid (5-methyl- isoxazol-3-yl)-amide | B |
| 104 | 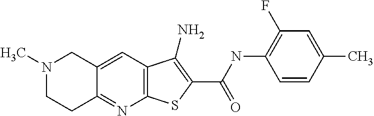 | C19H19FN4OS | 3-Amino-6-methyl-5,6,7,8- tetrahydro-thieno[2,3- b][1,6]naphthyridine-2- carboxylic acid (2-fluoro-4- methyl-phenyl)-amide | B |
| 105 | 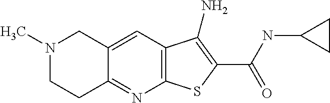 [SMILES: CN1CCc2nc3sc(C(=O)NC4CC4)c(N)c3cc2C1] | C15H18N4OS | 3-Amino-6-methyl-5,6,7,8- tetrahydro-thieno[2,3- b][1,6]naphthyridine-2- carboxylic acid cyclopropylamide | B |
| 106 | 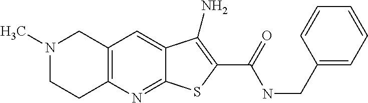 [SMILES: CN1CCc2nc3sc(C(=O)NCc4ccccc4)c(N)c3cc2C1] | C19H20N4OS | 3-Amino-6-methyl-5,6,7,8- tetrahydro-thieno[2,3- b][1,6]naphthyridine-2- cartoxylic acid benzylamide | B |
| 107 | 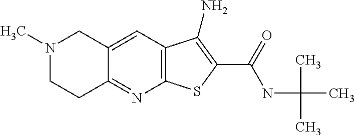 [SMILES: CN1CCc2nc3sc(C(=O)NC(C)(C)C)c(N)c3cc2C1] | C16H22N4OS | 3-Amino-6-methyl-5,6,7,8- tetrahydro-thieno[2,3- b][1,6]naphthyridine-2- carboxylic acid tert- butylamide | B |
| 108 | 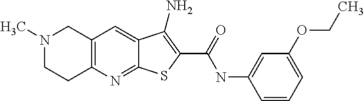 [SMILES: CCOc1cccc(NC(=O)c2sc3nc4c(cc3c2N)CN(C)CC4)c1] | C20H22N4O2S | 3-Amino-6-methyl-5,6,7,8- tetrahydro-thieno[2,3- b][1,6]naphthyridine-2- carboxylic acid (3-ethoxy- phenyl)-amide | B |
| 109 | 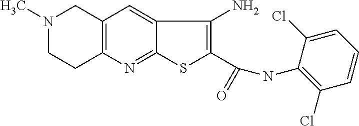 [SMILES: CN1CCc2nc3sc(C(=O)Nc4c(Cl)cccc4Cl)c(N)c3cc2C1] | C18H16Cl2N4OS | 3-Amino-6-methyl-5,6,7,8- tetrahydro-thieno[2,3- b][1,6]naphthyridine-2- carboxylic acid (2,6-dichloro- phenyl)-amide | B |
| 110 | 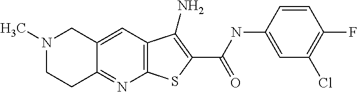 [SMILES: CN1CCc2nc3sc(C(=O)Nc4ccc(F)c(Cl)c4)c(N)c3cc2C1] | C18H16ClFN4OS | 3-Amino-6-methyl-5,6,7,8- tetrahydro-thieno[2,3- b][1,6]naphthyridine-2- carboxylic acid (3-chloro-4- fluoro-phenyl)-amide | B |
| 111 | 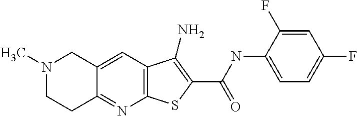 [SMILES: CN1CCc2nc3sc(C(=O)Nc4ccc(F)cc4F)c(N)c3cc2C1] | C18H16F2N4OS | 3-Amino-6-methyl-5,6,7,8- tetrahydro-thieno[2,3- b][1,6]naphthyridine-2- carboxylic acid (2,4-difluoro- phenyl)-amide | B |
| 112 | 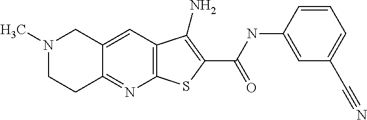 [SMILES: CN1CCc2nc3sc(C(=O)Nc4cccc(C#N)c4)c(N)c3cc2C1] | C19H17N5OS | 3-Amino-6-methyl-5,6,7,8- tetrahydro-thieno[2,3- b][1,6]naphthyridine-2- carboxylic acid (3-cyano- phenyl)-amide | B |
| 113 | 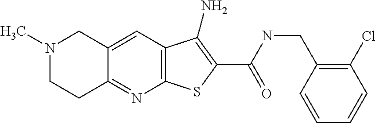 [SMILES: CN1CCc2nc3sc(C(=O)NCc4ccccc4Cl)c(N)c3cc2C1] | C19H19ClN4OS | 3-Amino-6-methyl-5,6,7,8- tetrahydro-thieno[2,3- b][1,6]naphthyridine-2- carboxylic acid 2-chloro- benzylamide | B |
| 114 | 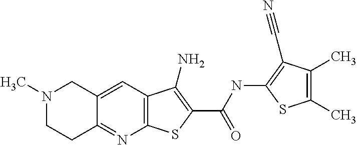 | C19H19N5OS2 | 3-Amino-6-methyl-5,6,7,8- tetrahydro-thieno[2,3- b][1,6]naphthyridine-2- carboxylic acid (3-cyano-4,5- dimethyl-thiophen-2-yl)-amide | B |
| 115 | 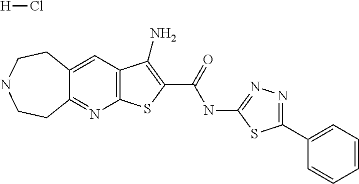 [SMILES: Cl ; Nc1c(C(=O)Nc2nnc(-c3ccccc3)s2)sc2nc3c(cc12)CCNCC3] | C20H18N6OS2•HCl | 3-Amino-6,7,8,9-tetrahydro- 5H-1-thia-7,10-diaza- cyclohepla[f]indene-2- carboxylic acid (5-phenyl- [1,3,4]thiadiazol-2-yl)-amide hydrochloride | B |
| 116 | 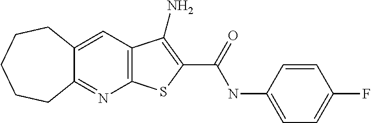 [SMILES: Nc1c(C(=O)Nc2ccc(F)cc2)sc2nc3c(cc12)CCCCC3] | C19H18FN3OS | 3-Amino-6,7,8,9-tetrahydro- 5H-1-thia-10-aza- cyclohepta[f]indene-2- carboxylic acid (4-fluoro- phenyl)-amide | C |
| 117 | 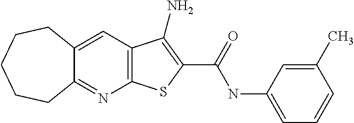 [SMILES: Cc1cccc(NC(=O)c2sc3nc4c(cc3c2N)CCCCC4)c1] | C20H21N3OS | 3-Amino-6,7,8,9-tetrahydro- 5H-1-thia-10-aza- cyclohepta[f]indene-2- carboxylic acid m-tolylamide | C |
| 118 | 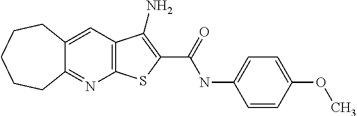 [SMILES: COc1ccc(NC(=O)c2sc3nc4c(cc3c2N)CCCCC4)cc1] | C20H21N3O2S | 3-Amino-6,7,8,9-tetrahydro- 5H-1-thia-10-aza- cyclohepta[f]indene-2- carboxylic acid (4-methoxy- phenyl)-amide | C |
| 119 | 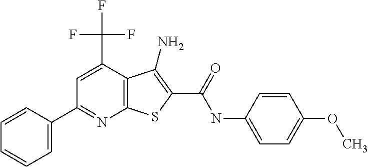 [SMILES: COc1ccc(NC(=O)c2sc3nc(-c4ccccc4)cc(C(F)(F)F)c3c2N)cc1] | C22H16F3N3O2S | 3-Amino-6-phenyl-4- trifluoromethyl-thieno[2,3- b]pyridine-2-carboxylic acid (4-methoxy-phenyl)-amide | C |
| 120 | 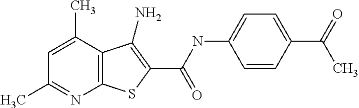 [SMILES: CC(=O)c1ccc(NC(=O)c2sc3nc(C)cc(C)c3c2N)cc1] | C18H17N3O2S | 3-Amino-4,6-dimethyl- thieno[2,3-b]pyridine-2- carboxylic acid (4-acetyl- phenyl)-amide | C |
| 121 | 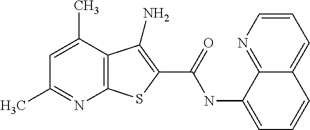 [SMILES: Cc1cc(C)c2c(N)c(C(=O)Nc3cccc4cccnc34)sc2n1] | C19H16N4OS | 3-Amino-4,6-dimethyl- thieno[2,3-b]pyridine-2- carboxylic acid quinolin-8- ylamide | C |
| 122 | 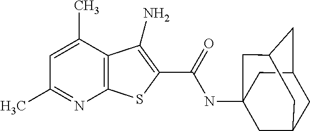 [SMILES: Cc1cc(C)c2c(N)c(C(=O)NC34CC5CC(CC(C5)C3)C4)sc2n1] | C20H25N3OS | 3-Amino-4,6-dimethyl- thieno[2,3-b]pyridine-2- carboxylic acid adamantan-1- ylamide | C |
| 123 | 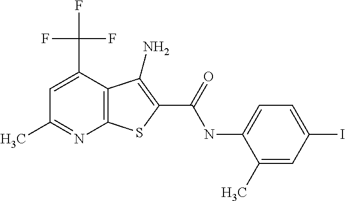 [SMILES: Cc1cc(C(F)(F)F)c2c(N)c(C(=O)Nc3ccc(I)cc3C)sc2n1] | C17H13F3IN3OS | 3-Amino-6-methyl-4- trifluoromethyl-thieno[2,3- b]pyridine-2-carboxylic acid (4-iodo-2-methyl-phenyl)- amide | C |
| 124 | 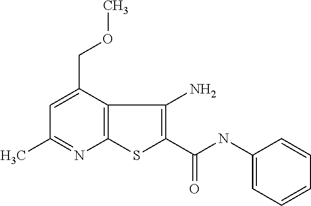 [SMILES: COCc1cc(C)nc2sc(C(=O)Nc3ccccc3)c(N)c12] | C17H17N3O2S | 3-Amino-4-methoxymethyl-6- methyl-thieno[2,3-b]pyridine-2- carboxylic acid phenylamide | C |
| 125 | 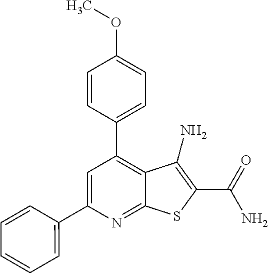 [SMILES: COc1ccc(-c2cc(-c3ccccc3)nc3sc(C(N)=O)c(N)c23)cc1] | C21H17N3O2S | 3-Amino-4-(4-methoxy- phenyl)-6-phenyl-thieno[2,3- b]pyridine-2-carboxylic acid amide | C |
| 126 | 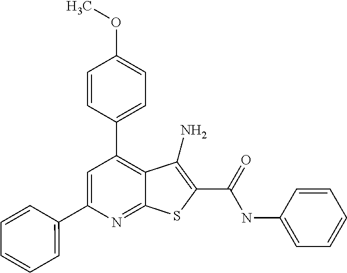 [SMILES: COc1ccc(-c2cc(-c3ccccc3)nc3sc(C(=O)Nc4ccccc4)c(N)c23)cc1] | C27H21N3O2S | 3-Amino-4-(4-methoxy- phenyl)-6-phenyl-thieno[2,3- b]pyridine-2-carboxylic acid phenylamide | C |
| 127 | 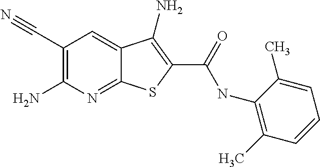 [SMILES: Cc1cccc(C)c1NC(=O)c1sc2nc(N)c(C#N)cc2c1N] | C17H15N5OS | 3,6-Diamino-5-cyano- thieno[2,3-b]pyridine-2- carboxylic acid (2,6-dimethyl- phenyl)-amide | C |
| 128 | 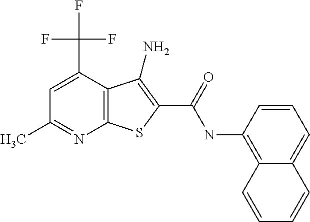 [SMILES: Cc1cc(C(F)(F)F)c2c(N)c(C(=O)Nc3cccc4ccccc34)sc2n1] | C20H14F3N3OS | 3-Amino-6-methyl-4- trifluoromethyl-thieno[2,3- b]pyridine-2-carboxylic acid naphthalen-1-ylamide | C |
| 129 | 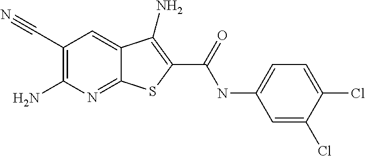 [SMILES: N#Cc1cc2c(N)c(C(=O)Nc3ccc(Cl)c(Cl)c3)sc2nc1N] | C15H9Cl2N5OS | 3,6-Diamino-5-cyano- thieno[2,3-b]pyridine-2- carboxylic acid (3,4-dichloro- phenyl)-amide | C |
| 130 | 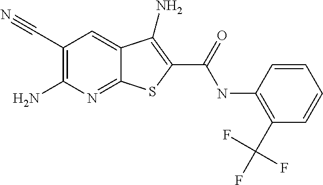 [SMILES: N#Cc1cc2c(N)c(C(=O)Nc3ccccc3C(F)(F)F)sc2nc1N] | C16H10F3N5OS | 3,6-Diamino-5-cyano- thieno[2,3-b]pyridine-2- carboxylic acid (2- trifluoromethyl-phenyl)-amide | C |
| 131 | 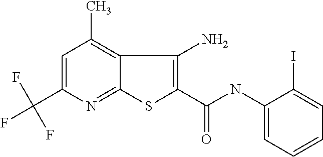 [SMILES: Cc1cc(C(F)(F)F)nc2sc(C(=O)Nc3ccccc3I)c(N)c12] | C16H11F3IN3OS | 3-Amino-4-methyl-6- trifluoromethyl-thieno[2,3- b]pyridine-2-carboxylic acid (2-iodo-phenyl)-amide | C |
| 132 | 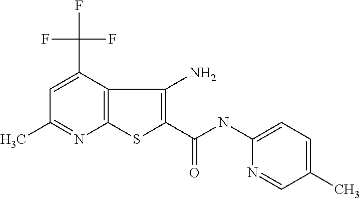 | C16H13F3N4OS | 3-Amino-6-methyl-4- trifluoromethyl-thieno[2,3- b]pyridine-2-carboxylic acid (5-methyl-pyridin-2-yl)-amide | C |
| 133 | 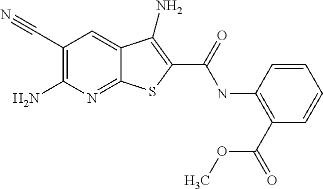 [SMILES: COC(=O)c1ccccc1NC(=O)c1sc2nc(N)c(C#N)cc2c1N] | C17H13N5O3S | 2-[(3,6-Diamino-5-cyano- thieno[2,3-b]pyridine-2- carbonyl)-amino]-benzoic acid methyl ester | C |
| 134 | 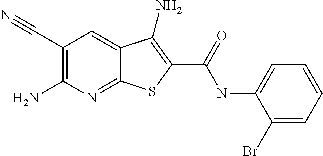 [SMILES: N#Cc1cc2c(N)c(C(=O)Nc3ccccc3Br)sc2nc1N] | C15H10BrN5OS | 3,6-Diamino-5-cyano- thieno[2,3-b]pyridine-2- carboxylic acid (2-bromo- phenyl)-amide | C |
| 135 | 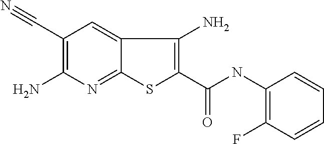 [SMILES: N#Cc1cc2c(N)c(C(=O)Nc3ccccc3F)sc2nc1N] | C15H10FN5OS | 3,6-Diamino-5-cyano- thieno[2,3-b]pyridine-2- carboxylic acid (2-fluoro- phenyl)-amide | C |
| 136 | 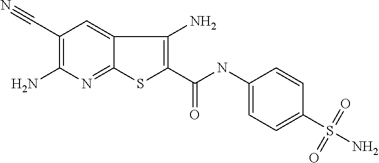 [SMILES: N#Cc1cc2c(N)c(C(=O)Nc3ccc(S(N)(=O)=O)cc3)sc2nc1N] | C15H12N6O3S2 | 3,6-Diamino-5-cyano- thieno[2,3-b]pyridine-2- carboxylic acid (4-sulfamoyl- phenyl)-amide | C |
| 137 | 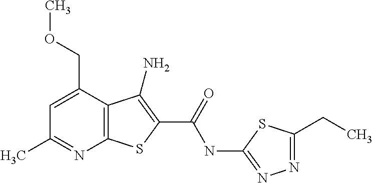 [SMILES: CCc1nnc(NC(=O)c2sc3nc(C)cc(COC)c3c2N)s1] | C15H17N5O2S2 | 3-Amino-4-methoxymethyl-6- methyl-thieno[2,3-b]pyridine-2- carboxylic acid (5-ethyl- [1,3,4]thiadiazol-2-yl)-amide | C |
| 138 | 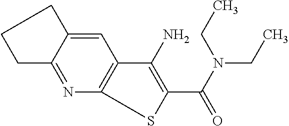 [SMILES: CCN(CC)C(=O)c1sc2nc3c(cc2c1N)CCC3] | C15H19N3OS | 3-Amino-6,7-dihydro-5H- cyclopenta[b]thieno[3,2- e]pyridine-2-carboxylic acid diethylamide | C |
| 139 | 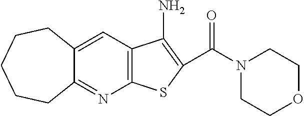 [SMILES: Nc1c(C(=O)N2CCOCC2)sc2nc3c(cc12)CCCCC3] | C17H21N3O2S | (3-Amino-6,7,8,9-tetrahydro- 5H-1-thia-10-aza- cyclohepta[f]inden-2-yl)- morpholin-4-yl-methanone | C |
| 140 | 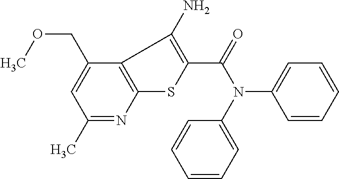 [SMILES: COCc1cc(C)nc2sc(C(=O)N(c3ccccc3)c3ccccc3)c(N)c12] | C23H21N3O2S | 3-Amino-4-methoxymethyl-6- methyl-thieno[2,3-b]pyridine-2- carboxylic acid diphenylamide | C |
| 141 | 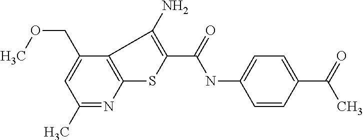 [SMILES: COCc1cc(C)nc2sc(C(=O)Nc3ccc(C(C)=O)cc3)c(N)c12] | C19H19N3O3S | 3-Amino-4-methoxymethyl-6- methyl-thieno[2,3-b]pyridine-2- carboxylic acid (4-acetyl- phenyl)-amide | C |
| 142 | 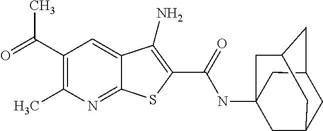 [SMILES: CC(=O)c1cc2c(N)c(C(=O)NC34CC5CC(CC(C5)C3)C4)sc2nc1C] | C21H25N3O2S | 5-Acetyl-3-amino-6-methyl- thieno[2,3-b]pyridine-2- carboxylic acid adamantan-1- ylamide | C |
| 143 | 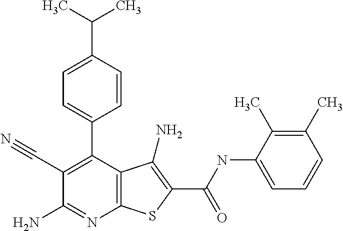 [SMILES: Cc1cccc(NC(=O)c2sc3nc(N)c(C#N)c(-c4ccc(C(C)C)cc4)c3c2N)c1C] | C26H25N5OS | 3,6-Diamino-5-cyano-4-(4- isopropyl-phenyl)-thieno[2,3- b]pyridine-2-carboxylic acid (2,3-dimethyl-phenyl)-amide | C |
| 144 | 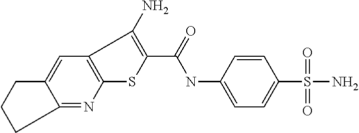 [SMILES: Nc1c(C(=O)Nc2ccc(S(N)(=O)=O)cc2)sc2nc3c(cc12)CCC3] | C17H16N4O3S2 | 3-Amino-6,7-dihydro-5H- cyclopenta[b]thieno[3,2- e]pyridine-2-carboxylic acid (4-sulfamoyl-phenyl)-amide | C |
| 145 | 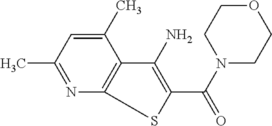 [SMILES: Cc1cc(C)c2c(N)c(C(=O)N3CCOCC3)sc2n1] | C14H17N3O2S | (3-Amino-4,6-dimethyl- thieno[2,3-b]pyridin-2-yl)- morpholin-4-yl-methanone | C |
| 146 | 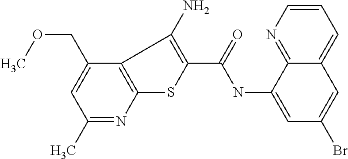 [SMILES: COCc1cc(C)nc2sc(C(=O)Nc3cc(Br)cc4cccnc34)c(N)c12] | C20H17BrN4O2S | 3-Amino-4-methoxymethyl-6- methyl-thieno[2,3-b]pyridine-2- carboxylic acid (6-bromo- quinolin-8-yl)-amide | C |
| 147 | 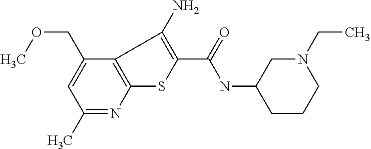 [SMILES: CCN1CCCC(NC(=O)c2sc3nc(C)cc(COC)c3c2N)C1] | C18H26N4O2S | 3-Amino-4-methoxymethyl-6- methyl-thieno[2,3-b]pyridine-2- carboxylic acid (1-ethyl- piperidin-3-yl)-amide | C |
| 148 | 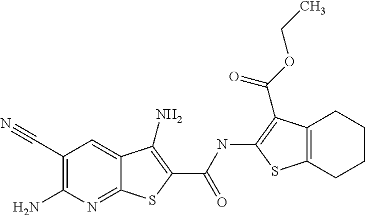 [SMILES: CCOC(=O)c1c(NC(=O)c2sc3nc(N)c(C#N)cc3c2N)sc2c1CCCC2] | C20H19N5O3S2 | 2-[(3,6-Diamino-5-cyano- thieno[2,3-b]pyridine-2- carbonyl)-amino]-4,5,6,7- tetrahydro-benzo[b]thiophene- 3-carboxylic acid ethyl ester | C |
| 149 | 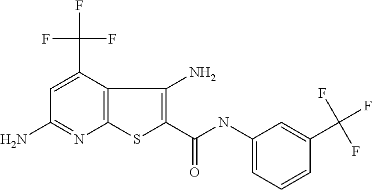 [SMILES: Nc1cc(C(F)(F)F)c2c(N)c(C(=O)Nc3cccc(C(F)(F)F)c3)sc2n1] | C17H11F6N3OS | 3-Amino-6-methyl-4- trifluoromethyl-thieno[2,3- b]pyridine-2-carboxylic acid (3-trifluoromethyl-phenyl)- amide | C |
| 150 | 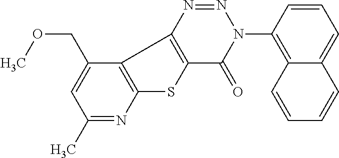 [SMILES: COCc1cc(C)nc2sc3c(=O)n(-c4cccc5ccccc45)nnc3c12] | C21H16N4O2S | 9-Methoxymethyl-7-methyl-3- naphthalen-1-yl-3H- pyrido[3′,2′:4,5]thieno[3,2- d][1,2,3]triazin-4-one | C |
| 151 | 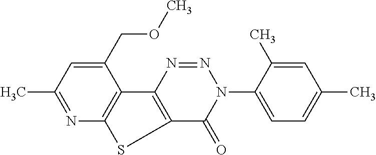 | C19H18N4O2S | 3-(2,4-Dimethyl-phenyl)-9- methoxymethyl-7-methyl-3H- pyrido[3′,2′:4,5]thienol[3,2- d][1,2,3]triazin-4-one | C |
| 152 | 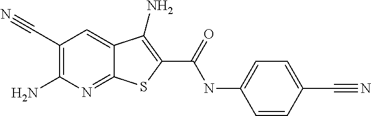 [SMILES: N#Cc1ccc(NC(=O)c2sc3nc(N)c(C#N)cc3c2N)cc1] | C16H10N6OS | 3,6-Diamino-5-cyano- thieno[2,3-b]pyridine-2- carboxylic acid (4-cyano- phenyl)-amide | C |
| 153 | 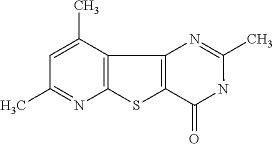 | C12H11N3OS | 2,7,9-Trimethyl-3H- pyrido[3′,2′:4,5]thieno[3,2- d]pyrimidin-4-one | C |
| 154 | 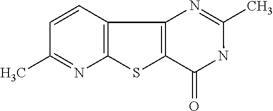 | C11H9N3OS | 2,7-Dimethyl-3H- pyrido[3′,2′:4,5]thieno[3,2- d]pyrimidin-4-one | C |
| 155 | 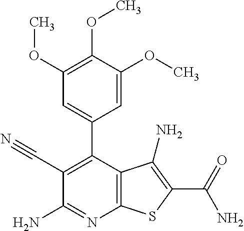 [SMILES: COc1cc(-c2c(C#N)c(N)nc3sc(C(N)=O)c(N)c23)cc(OC)c1OC] | C18H17N5O4S | 3,6-Diamino-5-cyano-4-(3,4,5- trimethoxy-phenyl)-thieno[2,3- b]pyridine-2-carboxylic acid amide | C |
| 156 | 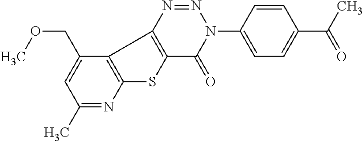 [SMILES: COCc1cc(C)nc2sc3c(=O)n(-c4ccc(C(C)=O)cc4)nnc3c12] | C19H16N4O3S | 3-(4-Acetyl-phenyl)-9- methoxymethyl-7-methyl-3H- pyrido[3′,2′:4,5]thieno[2,3- d][1,2,3]triazin-4-one | C |
| 157 | 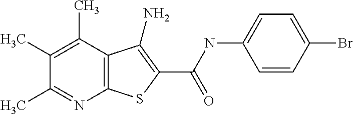 [SMILES: Cc1nc2sc(C(=O)Nc3ccc(Br)cc3)c(N)c2c(C)c1C] | C17H16BrN3OS | 3-Amino-4,5,6-trimethyl- thieno[2,3-b]pyridine-2- carboxylic acid (4-bromo- phenyl)-amide | C |
| 158 | 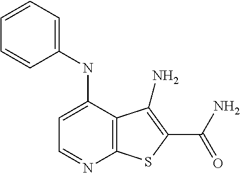 [SMILES: NC(=O)c1sc2nccc(Nc3ccccc3)c2c1N] | C14H12N4OS | 3-Amino-4-phenylamino- thieno[2,3-b]pyridine-2- carboxylic acid amide | C |
| 159 | 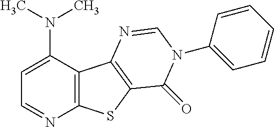 [SMILES: CN(C)c1ccnc2sc3c(=O)n(-c4ccccc4)cnc3c12] | C17H14N4OS | 9-Dimethylamino-3-phenyl-3H- pyrido[3′,2′:4,5]thieno[3,2- d]pyrimidin-4-one | C |
| 160 | 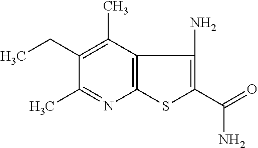 [SMILES: CCc1c(C)nc2sc(C(N)=O)c(N)c2c1C] | C12H15N3OS | 3-A mino-5-ethyl-4,6-dimethyl- thieno[2,3-b]pyridine-2- carboxylic acid amide | C |
| 161 | 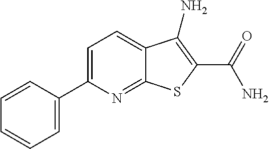 [SMILES: NC(=O)c1sc2nc(-c3ccccc3)ccc2c1N] | C14H11N3OS | 3-Amino-6-phenyl-thieno[2,3- b]pyridine-2-carboxylic acid amide | C |
| 162 | 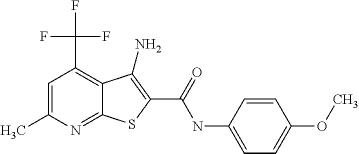 | C17H14F3N3O2S | 3-Amino-6-methyl-4- trifluoromethyl-thieno[2,3- b]pyridine-2-carboxylic acid (4-methoxy-phenyl)-amide | C |
| 163 | 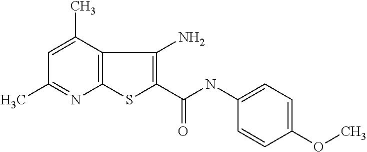 | C17H17N3O2S | 3-Amino-4,6-dimethyl- thieno[2,3-b]pyridine-2- carboxylic acid (4-methoxy- phenyl)-amide | C |
| 164 | 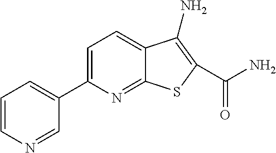 [SMILES: NC(=O)c1sc2nc(-c3cccnc3)ccc2c1N] | C13H10N4OS | 3-Amino-6-pyridin-3-yl- thieno[2,3-b]pyridine-2- carboxylic acid amide | C |
| 165 | 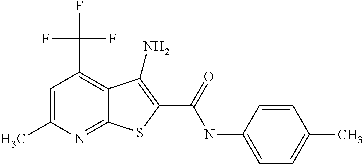 | C17H14F3N3OS | 3-Amino-6-methyl-4- trifluoromethyl-thieno[2,3- b]pyridine-2-carboxylic acid p- tolylamide | C |
| 166 | 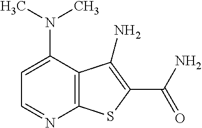 [SMILES: CN(C)c1ccnc2sc(C(N)=O)c(N)c12] | C10H12N4OS | 3-Amino-4-dimethylamino- thieno[2,3-b]pyridine-2- carboxylic acid amide | C |
| 167 | 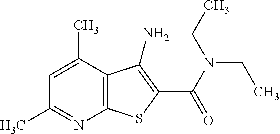 [SMILES: CCN(CC)C(=O)c1sc2nc(C)cc(C)c2c1N] | C14H19N3OS | 3-Amino-4,6-dimethyl- thieno[2,3-b]pyridine-2- carboxylic acid diethylamide | C |
| 168 | 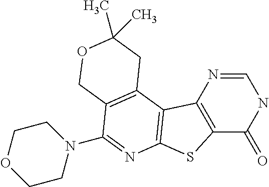 [SMILES: CC1(C)Cc2c(c(N3CCOCC3)nc3sc4c(=O)[nH]cnc4c23)CO1] | C18H20N4O3S | 2,2-Dimethyl-5-morpholin-4-yl- 1,4-dihydro-2H,9H-3-oxa-7- thia-6,9,11-triaza- benzo[c]fluoren-8-one | C |
| 169 | 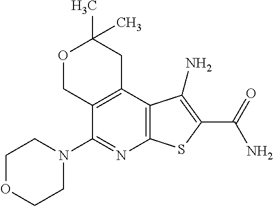 [SMILES: CC1(C)Cc2c(c(N3CCOCC3)nc3sc(C(N)=O)c(N)c23)CO1] | C17H22N4O3S | 1-Amino-8,8-dimethyl-5- morpholin-4-yl-8,9-dihydro-6H- 7-oxa-3-thia-4-aza- cyclopenta[a]naphthalene-2- carboxylic acid amide | C |
| 170 | 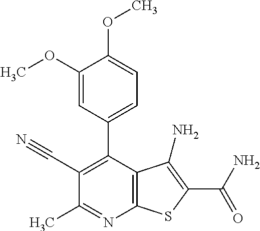 [SMILES: COc1ccc(-c2c(C#N)c(C)nc3sc(C(N)=O)c(N)c23)cc1OC] | C17H15N5O3S | 3,6-Diamino-5-cyano-4-(3,4- dimethoxy-phenyl)-thieno[2,3- b]pyridine-2-carboxylic acid amide | C |
| 171 | 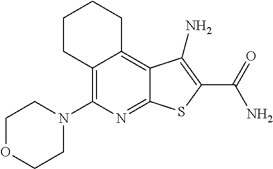 [SMILES: NC(=O)c1sc2nc(N3CCOCC3)c3c(c2c1N)CCCC3] | C16H20N4O2S | 1-Amino-5-morpholin-4-yl- 6,7,8,9-tetrahydro-thieno[2,3- c]isoquinoline-2-carboxylic acid amide | C |
| 172 | 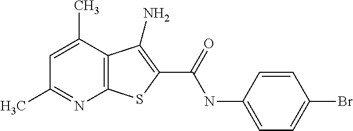 [SMILES: Cc1cc(C)c2c(N)c(C(=O)Nc3ccc(Br)cc3)sc2n1] | C16H14BrN3OS | 3-Amino-4,6-dimethyl- thieno[2,3-b]pyridine-2- carboxylic acid (4-bromo- phenyl)-amide | C |
| 173 | 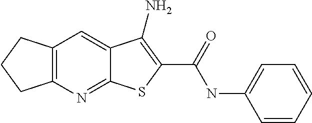 [SMILES: Nc1c(C(=O)Nc2ccccc2)sc2nc3c(cc12)CCC3] | C17H15N3OS | 3-Amino-6,7-dihydro-5H- cyclopenta[b]thieno[3,2- e]pyridine-2-carboxylic acid phenylamide | C |
| 174 | 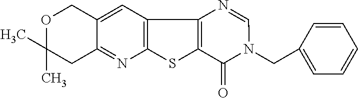 [SMILES: CC1(C)Cc2nc3sc4c(=O)n(Cc5ccccc5)cnc4c3cc2CO1] | C21H19N3O2S | 2-Benzyl-8,8-dimethyl-8,9- dihydro-2H,6H-7-oxa-11-thia- 2,4,10-triaza-benzo[b]fluoren- 1-one | C |
| 175 | 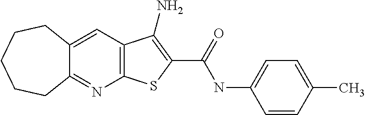 [SMILES: Cc1ccc(NC(=O)c2sc3nc4c(cc3c2N)CCCCC4)cc1] | C20H21N3OS | 3-Amino-6,7,8,9-tetrahydro- 5H-1-thia-10-aza- cyclohepta[f]indene-2- carboxylic acid p-tolylamide | C |
| 176 | 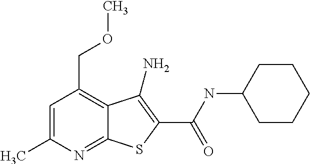 [SMILES: COCc1cc(C)nc2sc(C(=O)NC3CCCCC3)c(N)c12] | C17H23N3O2S | 3-Amino-4-methoxymethyl-6- methyl-thieno[2,3-b]pyridine-2- carboxylic acid cyclohexylamide | C |
| 177 | 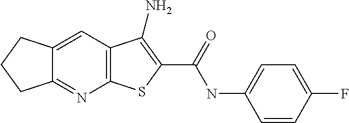 [SMILES: Nc1c(C(=O)Nc2ccc(F)cc2)sc2nc3c(cc12)CCC3] | C17H14FN3OS | 3-Amino-6,7-dihydro-5H- cyclopenta[b]thieno[3,2- e]pyridine-2-carboxylic acid (4-fluoro-phenyl)-amide | C |
| 178 | 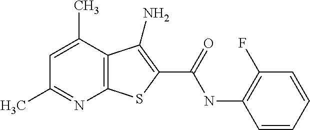 [SMILES: Cc1cc(C)c2c(N)c(C(=O)Nc3ccccc3F)sc2n1] | C16H14FN3OS | 3-Amino-4,6-dimethyl- thieno[2,3-b]pyridine-2- carboxylic acid (2-fluoro- phenyl)-amide | C |
| 179 | 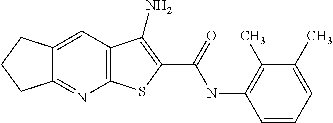 [SMILES: Cc1cccc(NC(=O)c2sc3nc4c(cc3c2N)CCC4)c1C] | C19H19N3OS | 3-Amino-6,7-dihydro-5H- cyclopenta[b]thieno[3,2- e]pyridine-2-carboxylic acid (2,3-dimethyl-phenyl)-amide | C |
| 180 | 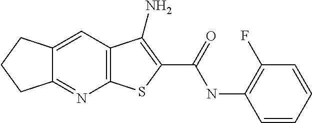 [SMILES: Nc1c(C(=O)Nc2ccccc2F)sc2nc3c(cc12)CCC3] | C17H14FN3OS | 3-Amino-6,7-dihydro-5H- cyclopenta[b]thieno[3,2- e]pyridine-2-carboxylic acid (2-fluoro-phenyl)-amide | C |
| 181 | 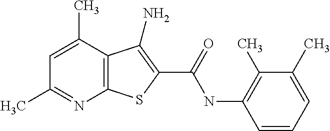 | C18H19N3OS | 3-Amino-4,6-dimethyl- thieno[2,3-b]pyridine-2- carboxylic acid (2,3-dimethyl- phenyl)-amide | C |
| 182 | 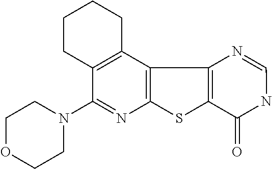 [SMILES: O=c1[nH]cnc2c1sc1nc(N3CCOCC3)c3c(c12)CCCC3] | C17H18N4O2S | 5-Morpholin-4-yl-1,2,3,4- tetrahydro-9H-7-thia-6,9,11- triaza-benzo[c]fluoren-8-one | C |
| 183 | 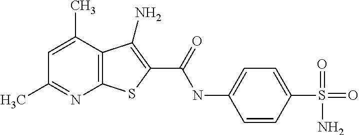 [SMILES: Cc1cc(C)c2c(N)c(C(=O)Nc3ccc(S(N)(=O)=O)cc3)sc2n1] | C16H16N4O3S2 | 3-Amino-4,6-dimethyl- thieno[2,3-b]pyridine-2- carboxylic acid (4-sulfamoyl- phenyl)-amide | C |
| 184 | 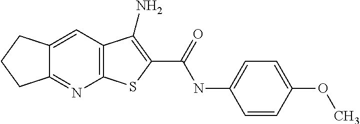 [SMILES: COc1ccc(NC(=O)c2sc3nc4c(cc3c2N)CCC4)cc1] | C18H17N3O2S | 3-Amino-6,7-dihydro-5H- cyclopenta[b]thieno[3,2- e]pyridine-2-carboxylic acid (4-methoxy-phenyl)-amide | C |
| 185 | 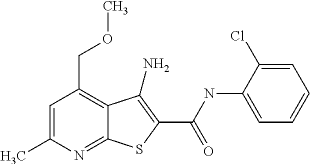 [SMILES: COCc1cc(C)nc2sc(C(=O)Nc3ccccc3Cl)c(N)c12] | C17H16ClN3O2S | 3-Amino-4-methoxymethyl-6- methyl-thieno[2,3-b]pyridine-2- carboxylic acid (2-chloro- phenyl)-amide | C |
| 186 | 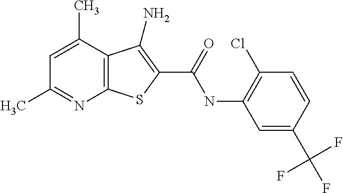 [SMILES: Cc1cc(C)c2c(N)c(C(=O)Nc3cc(C(F)(F)F)ccc3Cl)sc2n1] | C17H13ClF3N3OS | 3-Amino-4,6-dimethyl- thieno[2,3-b]pyridine-2- carboxylic acid (2-chloro-5- trifluoromethyl-phenyl)-amide | C |
| 187 | 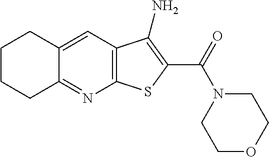 [SMILES: Nc1c(C(=O)N2CCOCC2)sc2nc3c(cc12)CCCC3] | C16H19N3O2S | (3-Amino-5,6,7,8-tetrahydro- thieno[2,3-b]quinolin-2-yl)- morpholin-4-yl-methanone | C |
| 188 | 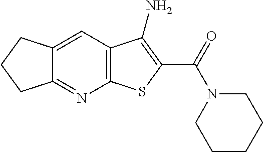 [SMILES: Nc1c(C(=O)N2CCCCC2)sc2nc3c(cc12)CCC3] | C16H19N3OS | (3-Amino-6,7-dihydro-5H- cyclopenta[b]thieno[3,2- e]pyridin-2-yl)-piperidin-1-yl- methanone | C |
| 189 | 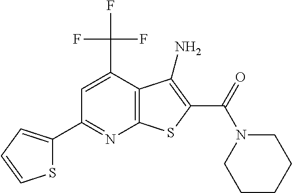 [SMILES: Nc1c(C(=O)N2CCCCC2)sc2nc(-c3cccs3)cc(C(F)(F)F)c12] | C18H16F3N3OS2 | (3-Amino-6-thiophen-2-yl-4- trifluoromethyl-thieno[2,3- b]pyridin-2-yl)-piperidin-1-yl- methanone | C |
| 190 | 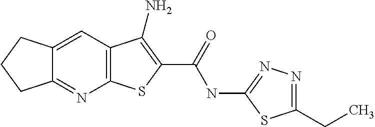 [SMILES: CCc1nnc(NC(=O)c2sc3nc4c(cc3c2N)CCC4)s1] | C15H15N5OS2 | 3-Amino-6,7-dihydro-5H- cyclopenta[b]thieno[3,2- e]pyridine-2-carboxylic acid (5-ethyl-[1,3,4]thiadiazol-2-yl)- amide | C |
| 191 | 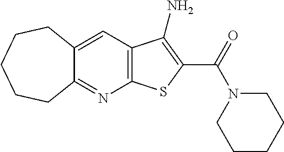 [SMILES: Nc1c(C(=O)N2CCCCC2)sc2nc3c(cc12)CCCCC3] | C18H23N3OS | (3-Amino-6,7,8,9-tetrahydro- 5H-1-thia-10-aza- cyclohepta[f]inden-2-yl)- piperidin-1-yl-methanone | C |
| 192 | 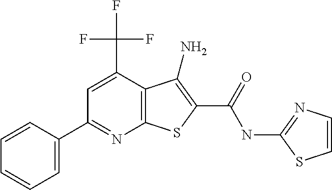 [SMILES: Nc1c(C(=O)Nc2nccs2)sc2nc(-c3ccccc3)cc(C(F)(F)F)c12] | C18H11F3N4OS2 | 3-Amino-6-phenyl-4- trifluoromethyl-thieno[2,3- b]pyridine-2-carboxylic acid thiazol-2-ylamide | C |
| 193 | 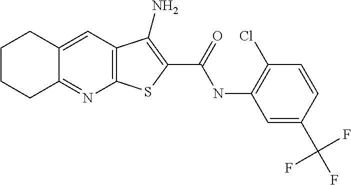 [SMILES: Nc1c(C(=O)Nc2cc(C(F)(F)F)ccc2Cl)sc2nc3c(cc12)CCCC3] | C19H15ClF3N3OS | 3-Amino-5,6,7,8-tetrahydro- thieno[2,3-b]quinoline-2- carboxylic acid (2-chloro-5- trifluoromethyl-phenyl)-amide | C |
| 194 | 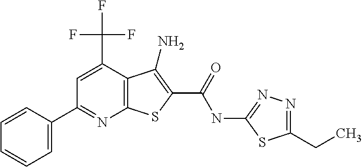 [SMILES: CCc1nnc(NC(=O)c2sc3nc(-c4ccccc4)cc(C(F)(F)F)c3c2N)s1] | C19H14F3N5OS2 | 3-Amino-6-phenyl-4- trifluoromethyl-thieno[2,3- b]pyridine-2-carboxylic acid (5-ethyl-[1,3,4]thiadiazol-2-yl)- amide | C |
| 195 | 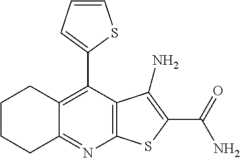 [SMILES: NC(=O)c1sc2nc3c(c(-c4cccs4)c2c1N)CCCC3] | C16H15N3OS2 | 3-Amino-4-thiophen-2-yl- 5,6,7,8-tetrahydro-thieno[2,3- b]quinoline-2-carboxylic acid amide | C |
| 196 | 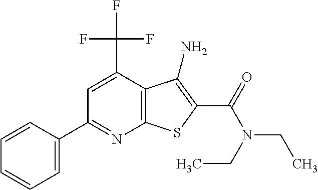 [SMILES: CCN(CC)C(=O)c1sc2nc(-c3ccccc3)cc(C(F)(F)F)c2c1N] | C19H18F3N3OS | 3-Amino-6-phenyl-4- trifluoromethyl-thieno[2,3- b]pyridine-2-carboxylic acid diethylamide | C |
| 197 | 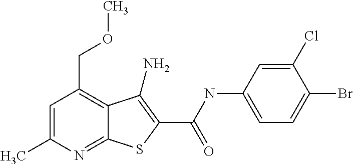 [SMILES: COCc1cc(C)nc2sc(C(=O)Nc3ccc(Br)c(Cl)c3)c(N)c12] | C17H15BrClN3O2S | 3-Amino-4-methoxymethyl-6- methyl-thieno[2,3-b]pyridine-2- carboxylic acid (4-bromo-3- chloro-phenyl)-amide | C |
| 198 | 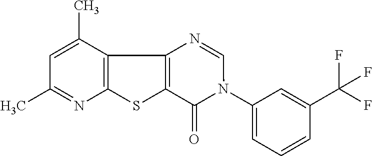 [SMILES: Cc1cc(C)c2c(n1)sc1c(=O)n(-c3cccc(C(F)(F)F)c3)cnc12] | C18H12F3N3OS | 7,9-Dimethyl-3-(3- trifluoromethyl-phenyl)-3H- pyrido[3′,2′:4,5]thieno[3,2- d]pyrimidin-4-one | C |
| 199 | 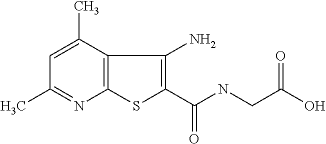 [SMILES: Cc1cc(C)c2c(N)c(C(=O)NCC(=O)O)sc2n1] | C12H13N3O3S | [(3-Amino-4,6-dimethyl- thieno[2,3-b]pyridine-2- carbonyl)-amino]-acetic acid | C |
| 200 | 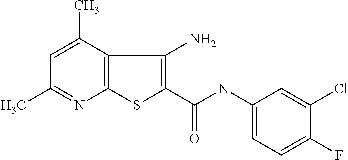 [SMILES: Cc1cc(C)c2c(N)c(C(=O)Nc3ccc(F)c(Cl)c3)sc2n1] | C16H13ClFN3OS | 3-Amino-4,6-dimethyl- thieno[2,3-b]pyridine-2- carboxylic acid (3-chloro-4- fluoro-phenyl)-amide | C |
| 201 | 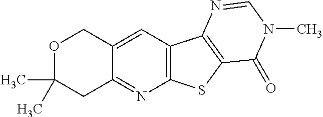 | C15H15N3O2S | 2,8,8-Trimethyl-8,9-dihydro- 2H,6H-7-oxa-11-thia-2,4,10- triaza-benzo[b]fluoren-1-one | C |
| 202 | 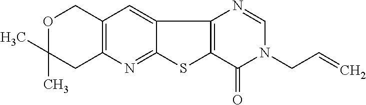 [SMILES: C=CCn1cnc2c(sc3nc4c(cc32)COC(C)(C)C4)c1=O] | C17H17N3O2S | 2-Allyl-8,8-dimethyl-8,9- dihydro-2H,6H-7-oxa-11-thia- 2,4,10-triaza-benzo[b]fluoren- 1-one | C |
| 203 | 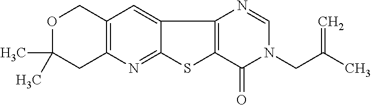 [SMILES: C=C(C)Cn1cnc2c(sc3nc4c(cc32)COC(C)(C)C4)c1=O] | C18H19N3O2S | 8,8-Dimethyl-2-(2-methyl- allyl)-8,9-dihydro-2H,6H-7-oxa- 11-thia-2,4,10-triaza- benzo[b]fluoren-1-one | C |
| 204 | 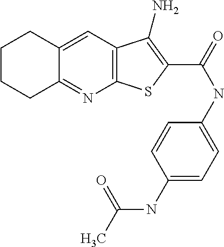 [SMILES: CC(=O)Nc1ccc(NC(=O)c2sc3nc4c(cc3c2N)CCCC4)cc1] | C20H20N4O2S | 3-Amino-5,6,7,8-tetrahydro- thieno[2,3-b]quinoline-2- carboxylic acid (4- acetylamino-phenyl)-amide | C |
| 205 | 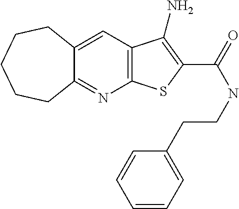 [SMILES: Nc1c(C(=O)NCCc2ccccc2)sc2nc3c(cc12)CCCCC3] | C21H23N3OS | 3-Amino-6,7,8,9-tetrahydro- 5H-1-thia-10-aza- cyclohepta[f]indene-2- carboxylic acid phenethyl- amide | C |
| 206 | 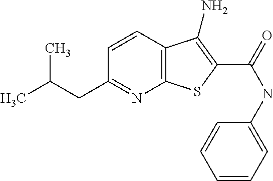 [SMILES: CC(C)Cc1ccc2c(N)c(C(=O)Nc3ccccc3)sc2n1] | C18H19N3OS | 3-Amino-6-isobutyl-thieno[2,3- b]pyridine-2-carboxylic acid phenylamide | C |
| 207 | 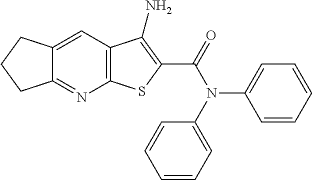 [SMILES: Nc1c(C(=O)N(c2ccccc2)c2ccccc2)sc2nc3c(cc12)CCC3] | C23H19N3OS | 3-Amino-6,7-dihydro-5H- cyclopenta[b]thieno[3,2- e]pyridine-2-carboxylic acid diphenylamide | C |
| 208 | 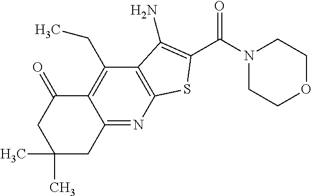 [SMILES: CCc1c2c(nc3sc(C(=O)N4CCOCC4)c(N)c13)CC(C)(C)CC2=O] | C20H25N3O3S | 3-Amino-4-ethyl-7,7-dimethyl- 2-(morpholine-4-carbonyl)-7,8- dihydro-6H-thieno[2,3- b]quinolin-5-one | C |
| 209 | 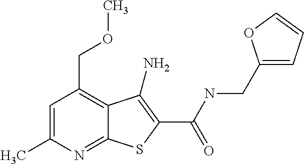 [SMILES: COCc1cc(C)nc2sc(C(=O)NCc3ccco3)c(N)c12] | C16H17N3O3S | 3-Amino-4-methoxymethyl-6- methyl-thieno[2,3-b]pyridine-2- carboxylic acid (furan-2- ylmethyl)-amide | C |
| 210 | 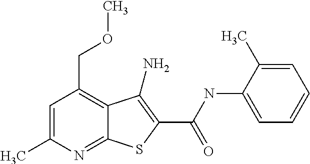 [SMILES: COCc1cc(C)nc2sc(C(=O)Nc3ccccc3C)c(N)c12] | C18H19N3O2S | 3-Amino-4-methoxymethyl-6- methyl-thieno[2,3-b]pyridine-2- carboxylic acid o-tolylamide | C |
| 211 | 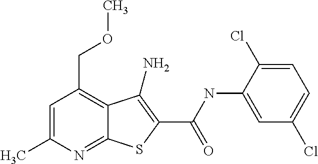 [SMILES: COCc1cc(C)nc2sc(C(=O)Nc3cc(Cl)ccc3Cl)c(N)c12] | C17H15Cl2N3O2S | 3-Amino-4-methoxymethyl-6- methyl-thieno[2,3-b]pyridine-2- carboxylic acid (2,5-dichloro- phenyl)-amide | C |
| 212 | 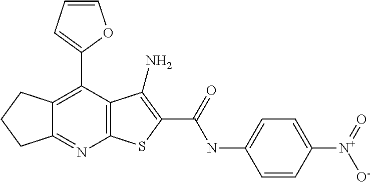 [SMILES: Nc1c(C(=O)Nc2ccc([N+](=O)[O-])cc2)sc2nc3c(c(-c4ccco4)c12)CCC3] | C21H16N4O4S | 3-Amino-4-furan-2-yl-6,7- dihydro-5H- cyclopenta[b]thieno[3,2- e]pyridine-2-carboxylic acid (4-nitro-phenyl)-amide | C |
| 213 | 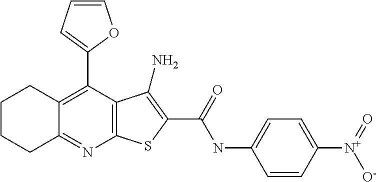 [SMILES: Nc1c(C(=O)Nc2ccc([N+](=O)[O-])cc2)sc2nc3c(c(-c4ccco4)c12)CCCC3] | C22H18N4O4S | 3-Amino-4-furan-2-yl-5,6,7,8- tetrahydro-thieno[2,3- b]quinoline-2-carboxylic acid (4-nitro-phenyl)-amide | C |
| 214 | 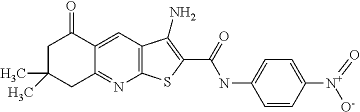 [SMILES: CC1(C)CC(=O)c2cc3c(N)c(C(=O)Nc4ccc([N+](=O)[O-])cc4)sc3nc2C1] | C20H18N4O4S | 3-Amino-7,7-dimethyl-5-oxo- 5,6,7,8-tetrahydro-thieno[2,3- b]quinoline-2-carboxylic acid (4-nitro-phenyl)-amide | C |
| 215 | 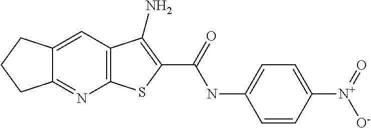 [SMILES: Nc1c(C(=O)Nc2ccc([N+](=O)[O-])cc2)sc2nc3c(cc12)CCC3] | C17H14N4O3S | 3-Amino-6,7-dihydro-5H- cyclopenta[b]thieno[3,2- e]pyridine-2-carboxylic acid (4-nitro-phenyl)-amide | C |
| 216 | 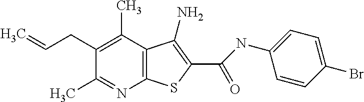 [SMILES: C=CCc1c(C)nc2sc(C(=O)Nc3ccc(Br)cc3)c(N)c2c1C] | C19H18BrN3OS | 5-Allyl-3-amino-4,6-dimethyl- thieno[2,3-b]pyridine-2- carboxylic acid (4-bromo- phenyl)-amide | C |
| 217 | 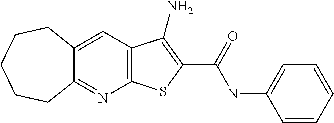 [SMILES: Nc1c(C(=O)Nc2ccccc2)sc2nc3c(cc12)CCCCC3] | C19H19N3OS | 3-Amino-6,7,8,9-tetrahydro- 5H-1-thia-10-aza- cyclohepta[f]indene-2- carboxylic acid phenylamide | C |
| 218 | 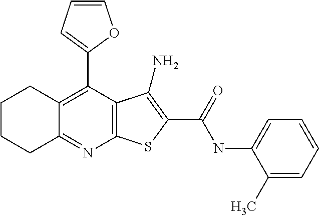 [SMILES: Cc1ccccc1NC(=O)c1sc2nc3c(c(-c4ccco4)c2c1N)CCCC3] | C23H21N3O2S | 3-Amino-4-furan-2-yl-5,6,7,8- tetrahydro-thieno[2,3- b]quinoline-2-carboxylic acid o-tolylamide | C |
| 219 | 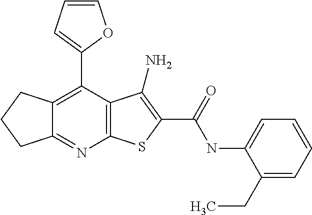 [SMILES: CCc1ccccc1NC(=O)c1sc2nc3c(c(-c4ccco4)c2c1N)CCC3] | C23H21N3O2S | 3-Amino-4-furan-2-yl-6,7- dihydro-5H- cyclopenta[b]thieno[3,2- e]pyridine-2-carboxylic acid (2-ethyl-phenyl)-amide | C |
| 220 | 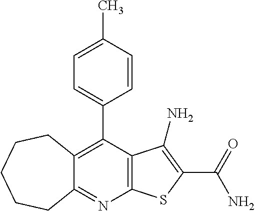 [SMILES: Cc1ccc(-c2c3c(nc4sc(C(N)=O)c(N)c24)CCCCC3)cc1] | C20H21N3OS | 3-Amino-1-p-tolyl-6,7,8,9- tetrahydro-5H-1-thia-10-aza- cyclohepta[f]indene-2- carboxylic acid amide | C |
| 221 | 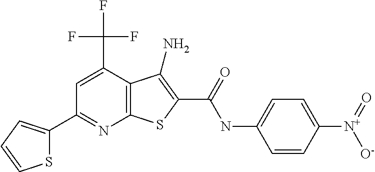 [SMILES: Nc1c(C(=O)Nc2ccc([N+](=O)[O-])cc2)sc2nc(-c3cccs3)cc(C(F)(F)F)c12] | C19H11F3N4O3S2 | 3-Amino-6-thiophen-2-yl-4- trifluoromethyl-thieno[2,3- b]pyridine-2-carboxylic acid (4-nitro-phenyl)-amide | C |
| 222 | 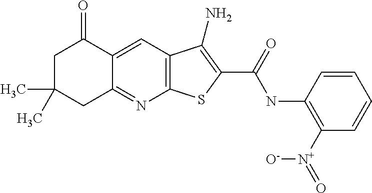 [SMILES: CC1(C)CC(=O)c2cc3c(N)c(C(=O)Nc4ccccc4[N+](=O)[O-])sc3nc2C1] | C20H18N4O4S | 3-Amino-7,7-dimethyl-5-oxo- 5,6,7,8-tetrahydro-thieno[2,3- b]quinoline-2-carboxylic acid (2-nitro-phenyl)-amide | C |
| 223 | 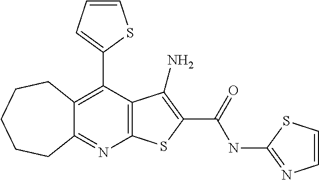 [SMILES: Nc1c(C(=O)Nc2nccs2)sc2nc3c(c(-c4cccs4)c12)CCCCC3] | C20H18N4OS3 | 3-Amino-4-thiophen-2-yl- 6,7,8,9-tetrahydro-5H-1-thia- 10-aza-cyclohepta[f]indene-2- carboxylic acid thiazol-2- ylamide | C |
| 224 | 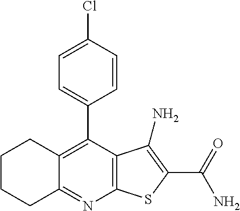 [SMILES: NC(=O)c1sc2nc3c(c(-c4ccc(Cl)cc4)c2c1N)CCCC3] | C18H16ClN3OS | 3-Amino-4-(4-chloro-phenyl)- 5,6,7,8-tetrahydro-thieno[2,3- b]quinoline-2-carboxylic acid amide | C |
| 225 | 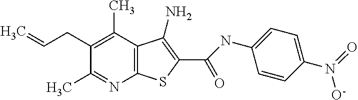 | C19H18N4O3S | 5-Allyl-3-amino-4,6-dimethyl- thieno[2,3-b]pyridine-2- carboxylic acid (4-nitro- phenyl)-amide | C |
| 226 | 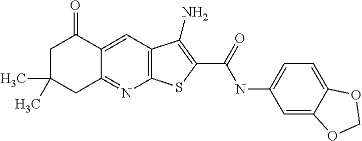 [SMILES: CC1(C)CC(=O)c2cc3c(N)c(C(=O)Nc4ccc5c(c4)OCO5)sc3nc2C1] | C21H19N3O4S | 3-Amino-7,7-dimethyl-5-oxo- 5,6,7,8-tetrahydro-thieno[2,3- b]quinoline-2-carboxylic acid benzo[1,3]dioxol-5-ylamide | C |
| 227 | 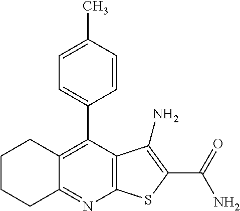 [SMILES: Cc1ccc(-c2c3c(nc4sc(C(N)=O)c(N)c24)CCCC3)cc1] | C19H19N3OS | 3-Amino-4-p-tolyl-5,6,7,8- tetrahydro-thieno[2,3- b]quinoline-2-carboxylic acid amide | C |
| 228 | 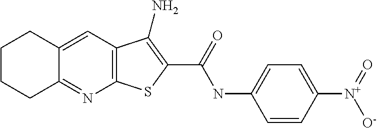 [SMILES: Nc1c(C(=O)Nc2ccc([N+](=O)[O-])cc2)sc2nc3c(cc12)CCCC3] | C18H16N4O3S | 3-Amino-5,6,7,8-tetrahydro- thieno[2,3-b]quinoline-2- carboxylic acid (4-nitro- phenyl)-amide | C |
| 229 | 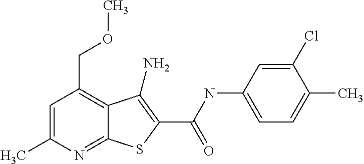 | C18H18ClN3O2S | 3-Amino-4-methoxymethyl-6- methyl-thieno[2,3-b]pyridine-2- carboxylic acid (3-chloro-4- methyl-phenyl)-amide | C |
| 230 | 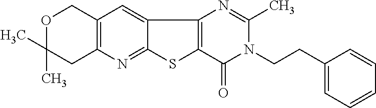 [SMILES: Cc1nc2c(sc3nc4c(cc32)COC(C)(C)C4)c(=O)n1CCc1ccccc1] | C23H23N3O2S | 3,8,8-Trimethyl-2-phenethyl- 8,9-dihydro-2H,6H-7-oxa-11- thia-2,4,10-triaza- benzo[b]fluoren-1-one | C |
| 231 | 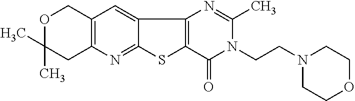 [SMILES: Cc1nc2c(sc3nc4c(cc32)COC(C)(C)C4)c(=O)n1CCN1CCOCC1] | C21H26N4O3S | 3,8,8-Trimethyl-2-(2-morpholin- 4-yl-ethyl)-8,9-dihydro-2H,6H- 7-oxa-11-thia-2,4,10-triaza- benzo[b]fluoren-1-one | C |
| 232 | 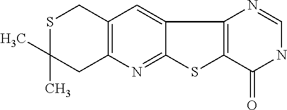 [SMILES: CC1(C)Cc2nc3sc4c(=O)[nH]cnc4c3cc2CS1] | C14H13N3OS2 | 8,8-Dimethyl-8,9-dihydro- 2H,6H-7,11-dithia-2,4,10- triaza-benzo[b]fluoren-1-one | C |
| 233 | 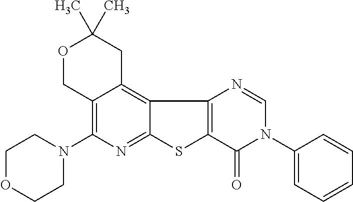 [SMILES: CC1(C)Cc2c(c(N3CCOCC3)nc3sc4c(=O)n(-c5ccccc5)cnc4c23)CO1] | C24H24N4O3S | 2,2-Dimethyl-5-morpholin-4-yl- 9-phenyl-1,4-dihydro-2H,9H-3- oxa-7-thia-6,9,11-triaza- benzo[c]fluoren-8-one | C |
| 234 | 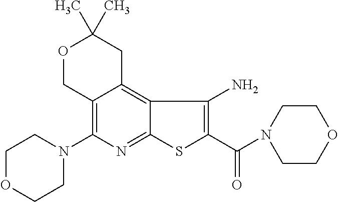 [SMILES: CC1(C)Cc2c(c(N3CCOCC3)nc3sc(C(=O)N4CCOCC4)c(N)c23)CO1] | C21H28N4O4S | (1-Amino-8,8-dimethyl-5- morpholin-4-yl-8,9-dihydro-6H- 7-oxa-3-thia-4-aza- cyclopenta[a]naphthalen-2-yl)- morpholin-4-yl-methanone | C |
| 235 | 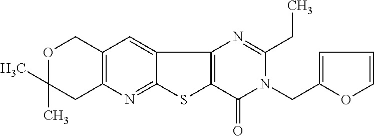 [SMILES: CCc1nc2c(sc3nc4c(cc32)COC(C)(C)C4)c(=O)n1Cc1ccco1] | C21H21N3O3S | 3-Ethyl-2-furan-2-ylmethyl-8,8- dimethyl-8,9-dihydro-2H,6H-7- oxa-11-thia-2,4,10-triaza- benzo[b]fluoren-1-one | C |
| 236 | 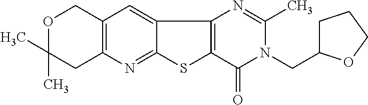 [SMILES: Cc1nc2c(sc3nc4c(cc32)COC(C)(C)C4)c(=O)n1CC1CCCO1] | C20H23N3O3S | 3,8,8-Trimethyl-2-(tetrahydro- furan-2-ylmethyl)-8,9-dihydro- 2H,6H-7-oxa-11-thia-2,4,10- triaza-benzo[b]fluoren-1-one | C |
| 237 | 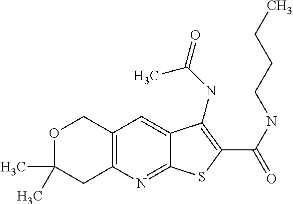 [SMILES: CCCCNC(=O)c1sc2nc3c(cc2c1NC(C)=O)COC(C)(C)C3] | C19H25N3O3S | 3-Acetylamino-7,7-dimethyl- 7,8-dihydro-5H-pyrano[4,3- b]thieno[3,2-e]pyridine-2- carboxylic acid butylamide | C |
| 238 | 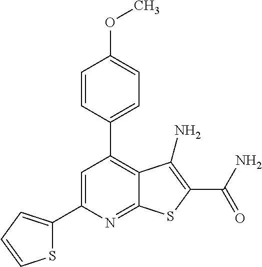 [SMILES: COc1ccc(-c2cc(-c3cccs3)nc3sc(C(N)=O)c(N)c23)cc1] | C19H15N3O2S2 | 3-Amino-4-(4-methoxy- phenyl)-6-thiophen-2-yl- thieno[2,3-b]pyridine-2- carboxylic acid amide | C |
| 239 | 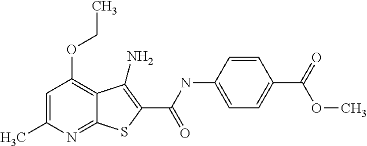 | C19H19N3O4S | 4-[(3-Amino-4-methoxymethyl- 6-methyl-thieno[2,3-b]pyridine- 2-carbonyl)-amino]-benzoic acid methyl ester | C |
| 240 | 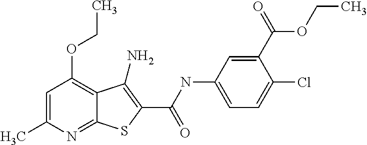 [SMILES: CCOC(=O)c1cc(NC(=O)c2sc3nc(C)cc(OCC)c3c2N)ccc1Cl] | C20H20ClN3O4S | 5-[(3-Amino-4-methoxymethyl- 6-methyl-thieno[2,3-b]pyridine- 2-carbonyl)-amino]-2-chloro- benzoic acid ethyl ester | C |
| 241 | 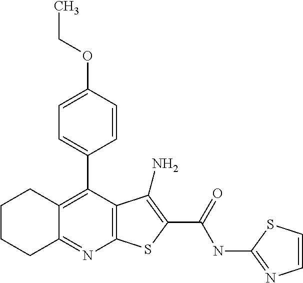 [SMILES: CCOc1ccc(-c2c3c(nc4sc(C(=O)Nc5nccs5)c(N)c24)CCCC3)cc1] | C23H22N4O2S2 | 3-Amino-4-(4-ethoxy-phenyl)- 5,6,7,8-tetrahydro-thieno[2,3- b]quinoline-2-carboxylic acid thiazol-2-ylamide | C |
| 242 | 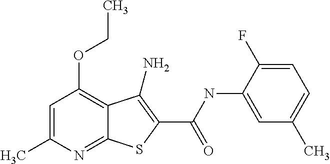 | C18H18FN3O2S | 3-Amino-4-methoxymethyl-6- methyl-thieno[2,3-b]pyridine-2- carboxylic acid (2-fluoro-5- methyl-phenyl)-amide | C |
| 243 | 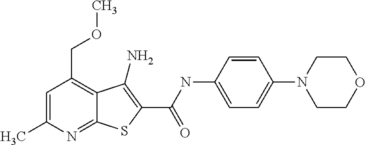 [SMILES: COCc1cc(C)nc2sc(C(=O)Nc3ccc(N4CCOCC4)cc3)c(N)c12] | C21H24N4O3S | 3-Amino-4-methoxymethyl-6- methyl-thieno[2,3-b]pyridine-2- carboxylic acid (4-morpholin- 4-yl-phenyl)-amide | C |
| 244 | 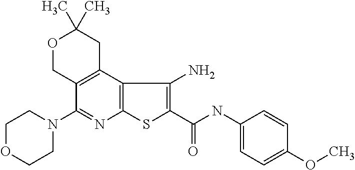 [SMILES: COc1ccc(NC(=O)c2sc3nc(N4CCOCC4)c4c(c3c2N)CC(C)(C)OC4)cc1] | C24H28N4O4S | 1-Amino-8,8-dimethyl-5- morpholin-4-yl-8,9-dihydro-6H- 7-oxa-3-thia-4-aza- cyclopenta[a]naphthalene-2- carboxylic acid (4-methoxy- phenyl)-amide | C |
| 245 | 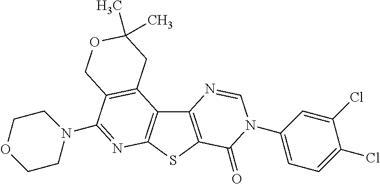 [SMILES: CC1(C)Cc2c(c(N3CCOCC3)nc3sc4c(=O)n(-c5ccc(Cl)c(Cl)c5)cnc4c23)CO1] | C24H22Cl2N4O3S | 9-(3,4-Dichloro-phenyl)-2,2- dimethyl-5-morpholin-4-yl-1,4- dihydro-2H,9H-3-oxa-7-thia- 6,9,11-triaza-benzo[c]fluoren- 8-one | C |
| 246 | 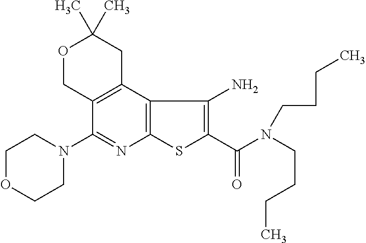 [SMILES: CCCCN(CCCC)C(=O)c1sc2nc(N3CCOCC3)c3c(c2c1N)CC(C)(C)OC3] | C25H38N4O3S | 1-Amino-8,8-dimethyl-5- morpholin-4-yl-8,9-dihydro-6H- 7-oxa-3-thia-4-aza- cyclopenta[a]naphthalene-2- carboxylic acid dibutylamide | C |
| 247 | 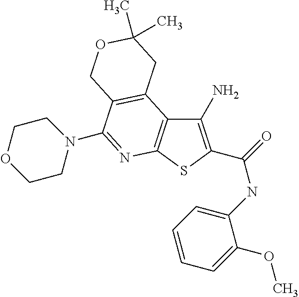 [SMILES: COc1ccccc1NC(=O)c1sc2nc(N3CCOCC3)c3c(c2c1N)CC(C)(C)OC3] | C24H28N4O4S | 1-Amino-8,8-dimethyl-5- morpholin-4-yl-8,9-dihydro-6H- 7-oxa-3-thia-4-aza- cyclopenta[a]naphthalene-2- carboxylic acid (2-methoxy- phenyl)-amide | C |
| 248 | 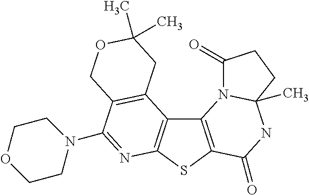 [SMILES: CC1(C)Cc2c(c(N3CCOCC3)nc3sc4c(c23)N2C(=O)CCC2(C)NC4=O)CO1] | C22H26N4O4S | 2,2,9a-Trimethyl-5-(4- morpholinyl)-1,4,9,9a,10,11- hexahydro-2H- pyrano[4″,3″:4′,5′]pyrido[3′,2′:4, 5]thieno[2,3-e]pyrrolo[1,2- a]pyrimidine-8,12-dione | C |
| 249 | 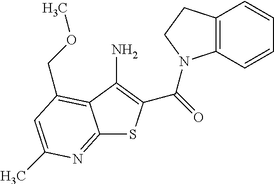 [SMILES: COCc1cc(C)nc2sc(C(=O)N3CCc4ccccc43)c(N)c12] | C19H19N3O2S | (3-Amino-4-methoxymethyl-6- methyl-thieno[2,3-b]pyridin-2- yl)-(2,3-dihydro-indol-1-yl)- methanone | C |
| 250 | 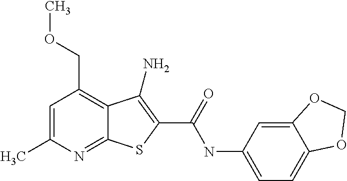 [SMILES: COCc1cc(C)nc2sc(C(=O)Nc3ccc4c(c3)OCO4)c(N)c12] | C18H17N3O4S | 3-Amino-4-methoxymethyl-6- methyl-thieno[2,3-b]pyridine-2- carboxylic acid benzo[1,3]dioxol-5-ylamide | C |
| 251 | 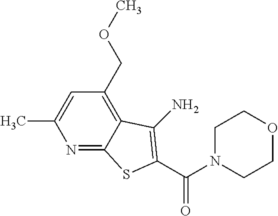 [SMILES: COCc1cc(C)nc2sc(C(=O)N3CCOCC3)c(N)c12] | C15H19N3O3S | (3-Amino-4-methoxymethyl-6- methyl-thieno[2,3-b]pyridin-2- yl)-morpholin-4-yl-methanone | C |
| 252 | 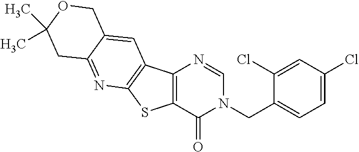 [SMILES: CC1(C)Cc2nc3sc4c(=O)n(Cc5ccc(Cl)cc5Cl)cnc4c3cc2CO1] | C21H17Cl2N3O2S | 2-(2,4-Dichloro-benzyl)-8,8- dimethyl-8,9-dihydro-2H,6H-7- oxa-11-thia-2,4,10-triaza- benzo[b]fluoren-1-one | C |
| 253 | 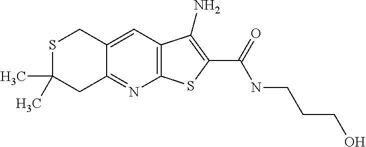 [SMILES: CC1(C)Cc2nc3sc(C(=O)NCCCO)c(N)c3cc2CS1] | C16H21N3O2S2 | 3-Amino-7,7-dimethyl-7,8- dihydro-5H-1,6-dithia-9-aza- cyclopenta[b]naphthalene-2- carboxylic acid (3-hydroxy- propyl)-amide | C |
| 254 | 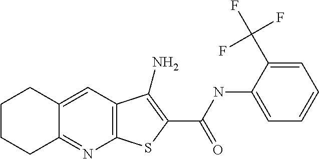 [SMILES: Nc1c(C(=O)Nc2ccccc2C(F)(F)F)sc2nc3c(cc12)CCCC3] | C19H16F3N3OS | 3-Amino-5,6,7,8- tetrahydro- thieno[2,3-b]quinoline-2- carboxylic acid (2- trifluoromethyl-phenyl)-amide | C |
| 255 | 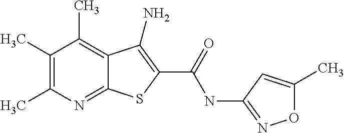 | C15H16N4O2S | 3-Amino-4,5,6-trimethyl- thieno[2,3-b]pyridine-2- carboxylic acid (5-methyl- isoxazol-3-yl)-amide | C |
| 256 | 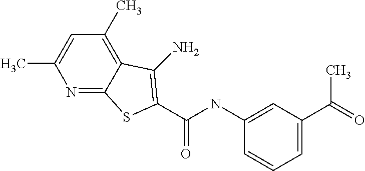 [SMILES: CC(=O)c1cccc(NC(=O)c2sc3nc(C)cc(C)c3c2N)c1] | C18H17N3O2S | 3-Amino-4,6-dimethyl- thieno[2,3-b]pyridine-2- carboxylic acid (3-acetyl- phenyl)-amide | C |
| 257 | 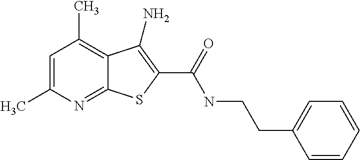 [SMILES: Cc1cc(C)c2c(N)c(C(=O)NCCc3ccccc3)sc2n1] | C18H19N3OS | 3-Amino-4,6-dimethyl- thieno[2,3-b]pyridine-2- carboxylic acid phenethyl- amide | C |
| 258 | 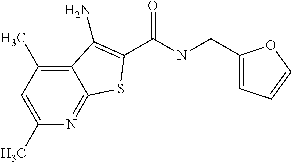 [SMILES: Cc1cc(C)c2c(N)c(C(=O)NCc3ccco3)sc2n1] | C15H15N3O2S | 3-Amino-4,6-dimethyl- thieno[2,3-b]pyridine-2- carboxylic acid (furan-2- ylmethyl)-amide | C |
| 259 | 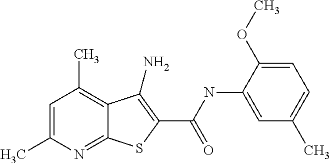 | C18H19N3O2S | 3-Amino-4,6-dimethyl- thieno[2,3-b]pyridine-2- carboxylic acid (2-methoxy-5- methyl-phenyl)-amide | C |
| 260 | 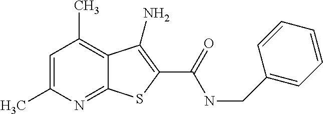 [SMILES: Cc1cc(C)c2c(N)c(C(=O)NCc3ccccc3)sc2n1] | C17H17N3OS | 3-Amino-4,6-dimethyl- thieno[2,3-b]pyridine-2- carboxylic acid benzylamide | C |
| 261 | 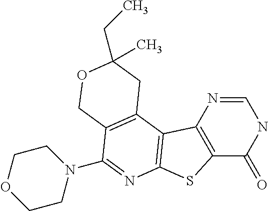 [SMILES: CCC1(C)Cc2c(c(N3CCOCC3)nc3sc4c(=O)[nH]cnc4c23)CO1] | C19H22N4O3S | 2-Ethyl-2-methyl-5-morpholin- 4-yl-1,4-dihydro-2H,9H-3-oxa- 7-thia-6,9,11-triaza- benzo[c]fluoren-8-one | C |
| 262 | 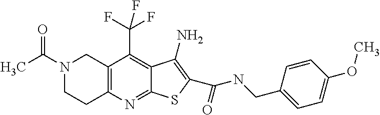 [SMILES: COc1ccc(CNC(=O)c2sc3nc4c(c(C(F)(F)F)c3c2N)CN(C(C)=O)CC4)cc1] | C22H21F3N4O3S | 6-Acetyl-3-amino-4- trifluoromethyl-5,6,7,8- tetrahydro-thieno[2,3- b][1,6]naphthyridine-2- carboxylic acid 4-methoxy- benzylamide | C |
| 263 | 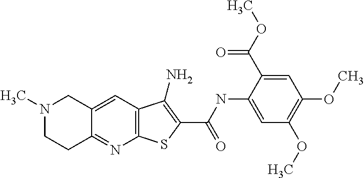 | C22H24N4O5S | 2-[(3-Amino-6-methyl-5,6,7,8- tetrahydro-thieno[2,3- b][1,6]naphthyridine-2- carbonyl)-amino]-4,5- dimethoxy-benzoic acid methyl ester | C |
| 264 | 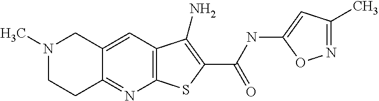 | C16H17N5O2S | 3-Amino-6-methyl-5,6,7,8- tetrahydro-thieno[2,3- b][1,6]naphthyridine-2- carboxylic acid (3-methyl- isoxazol-5-yl)-amide | C |
| 265 | 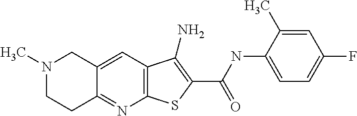 [SMILES: Cc1cc(F)ccc1NC(=O)c1sc2nc3c(cc2c1N)CN(C)CC3] | C19H19FN4OS | 3-Amino-6-methyl-5,6,7,8- tetrahydro-thieno[2,3- b][1,6]naphthyridine-2- carboxylic acid (4-fluoro-2- methyl-phenyl)-amide | C |
| 266 | 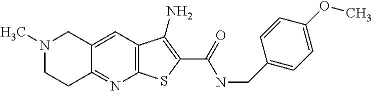 | C20H22N4O2S | 3-Amino-6-methyl-5,6,7,8- tetrahydro-thieno[2,3- b][1,6]naphthyridine-2- carboxylic acid 4-methoxy- benzylamide | C |
| 267 | 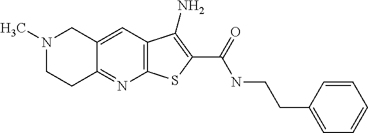 [SMILES: CN1CCc2nc3sc(C(=O)NCCc4ccccc4)c(N)c3cc2C1] | C20H22N4OS | 3-Amino-6-methyl-5,6,7,8- tetrahydro-thieno[2,3- b][1,6]naphthyridine-2- carboxylic acid phenethyl- amide | C |
| 268 | 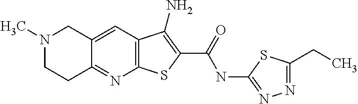 [SMILES: CCc1nnc(NC(=O)c2sc3nc4c(cc3c2N)CN(C)CC4)s1] | C16H18N6OS2 | 3-Amino-6-methyl-5,6,7,8- tetrahydro-thieno[2,3- b][1,6]naphthyridine-2- carboxylic acid (5-ethyl- [1,3,4]thiadiazol-2-yl)-amide | C |
| 269 | 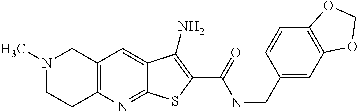 [SMILES: CN1CCc2nc3sc(C(=O)NCc4ccc5c(c4)OCO5)c(N)c3cc2C1] | C20H20N4O3S | 3-Amino-6-methyl-5,6,7,8- tetrahydro-thieno[2,3- b][1,6]naphthyridine-2- carboxylic acid (benzo[1,3]dioxol-5-ylmethyl)- amide | C |
| 270 | 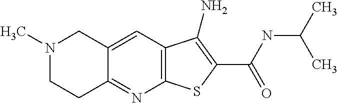 [SMILES: CC(C)NC(=O)c1sc2nc3c(cc2c1N)CN(C)CC3] | C15H20N4OS | 3-Amino-6-methyl-5,6,7,8- tetrahydro-thieno[2,3- b][1,6]naphthyridine-2- carboxylic acid isopropylamide | C |
| 271 | 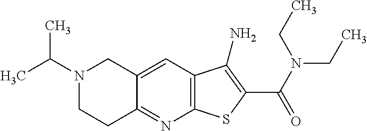 [SMILES: CCN(CC)C(=O)c1sc2nc3c(cc2c1N)CN(C(C)C)CC3] | C18H26N4OS | 3-Amino-6-isopropyl-5,6,7,8- tetrahydro-thieno[2,3- b][1,6]naphthyridine-2- carboxylic acid diethylamide | C |
| 272 | 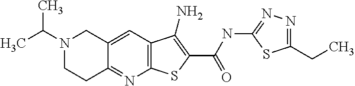 [SMILES: CCc1nnc(NC(=O)c2sc3nc4c(cc3c2N)CN(C(C)C)CC4)s1] | C18H22N6OS2 | 3-Amino-6-isopropyl-5,6,7,8- tetrahydro-thieno[2,3- b][1,6]naphthyridine-2- carboxylic acid (5-ethyl- [1,3,4]thiadiazol-2-yl)-amide | C |
| 273 | 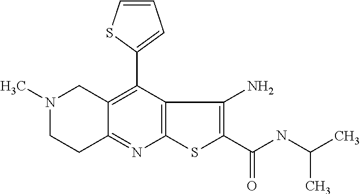 [SMILES: CC(C)NC(=O)c1sc2nc3c(c(-c4cccs4)c2c1N)CN(C)CC3] | C19H22N4OS2 | 3-A mino-6-methyl-4-thiophen- 2-yl-5,6,7,8-tetrahydro- thieno[2,3- b][1,6]naphthyridine-2- carboxylic acid isopropylamide | C |
| 274 | 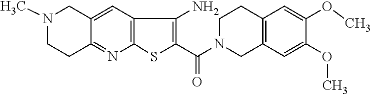 | C23H26N4O3S | (3-Amino-6-methyl-5,6,7,8- tetrahydro-thieno[2,3- b][1,6]naphthyridin-2-yl)-(6,7- dimethoxy-3,4-dihydro-1H- isoquinolin-2-yl)-methanone | C |
| 275 | 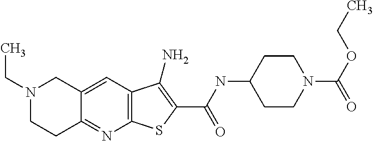 [SMILES: CCOC(=O)N1CCC(NC(=O)c2sc3nc4c(cc3c2N)CN(CC)CC4)CC1] | C21H29N5O3S | 4-[(3-Amino-6-ethyl-5,6,7,8- tetrahydro-thieno[2,3- b][1,6]naphthyridine-2- carbonyl)-amino]-piperidine-1- carboxylic acid ethyl ester | C |
| 276 | 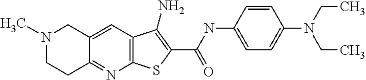 [SMILES: CCN(CC)c1ccc(NC(=O)c2sc3nc4c(cc3c2N)CN(C)CC4)cc1] | C22H27N5OS | 3-Amino-6-methyl-5,6,7,8- tetrahydro-thieno[2,3- b][1,6]naphthyridine-2- carboxylic acid (4- diethylamino-phenyl)-amide | C |
| 277 | 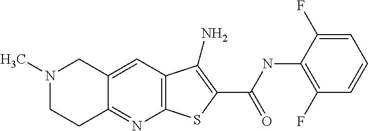 [SMILES: CN1CCc2nc3sc(C(=O)Nc4c(F)cccc4F)c(N)c3cc2C1] | C18H16F2N4OS | 3-Amino-6-methyl-5,6,7,8- tetrahydro-thieno[2,3- b][1,6]naphthyridine-2- carboxylic acid (2,6-difluoro- phenyl)-amide | C |
| 278 | 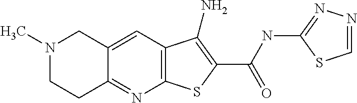 [SMILES: CN1CCc2nc3sc(C(=O)Nc4nncs4)c(N)c3cc2C1] | C14H14N6OS2 | 3-Amino-6-methyl-5,6,7,8- tetrahydro-thieno[2,3- b][1,6]naphthyridine-2- carboxylic acid [1,3,4]thiadiazol-2-ylamide | C |
| 279 | 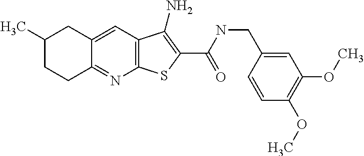 | C21H24N4O3S | 3-Amino-6-methyl-5,6,7,8- tetrahydro-thieno[2,3- b][1,6]naphthyridine-2- carboxylic acid 3,4- dimethoxy-benzylamide | C |
| 280 | 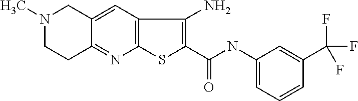 [SMILES: CN1CCc2nc3sc(C(=O)Nc4cccc(C(F)(F)F)c4)c(N)c3cc2C1] | C19H17F3N4OS | 3-Amino-6-methyl-5,6,7,8- tetrahydro-thieno[2,3- b][1,6]naphthyridine-2- carboxylic acid (3- trifluoromethyl-phenyl)-amide | C |

TABLE 2
Novel Compounds of Formula III of the present
invention.

| | | Molecular | | |
|---|---|---|---|---|
| Cmpd | Chemical Structure | Formula | Analytical Data | Chemical Name |
| 285 | 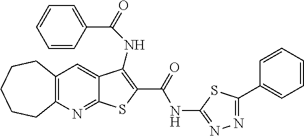 [SMILES: O=C(Nc1c(C(=O)Nc2nnc(-c3ccccc3)s2)sc2nc3c(cc12)CCCCC3)c1ccccc1] | C28 H23 N5 O2 S2 | 1H NMR in THF-d8: δ 8.46 (s, 1H), 8.16- 8.19 (m, 2H), 7.95-7.98 (m, 2H), 7.48-7.62 (m, 6H), 3.15 (d, 2H), 2.93 (d, 2H), 1.90 (s, 2H), 1.72 (s, 4H); Mass Spec: 526.2 (M + H)+ | 3-benzamido-N-(5- phenyl-1,3,4- thiadiazol-2-yl)- 6,7,8,9-tetrahydro-5H- cyclohepta[b]thieno[3, 2-e]pyridine-2- carboxamide |
| 289 | 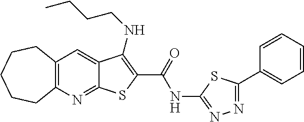 [SMILES: CCCCNc1c(C(=O)Nc2nnc(-c3ccccc3)s2)sc2nc3c(cc12)CCCCC3] | C25 H27 N5 O S2 | 1H NMR in THF-d8: δ 7.83-7.91 (m, 3H), 7.48-7.50 (m, 3H), 6.91 (s, 2H), 4.47-4.52 (m, 2H), 3.11 (d, 2H), 2.89 (d, 2H), 1.88- 2.00 (m, 4H), 1.72 (s, 4H), 1.43-1.50 (m, 2H), 1.03 (t, 3H); Mass Spec: 478.2 (M + H)+ | 3-(butylamino)-N-(5- phenyl-1,3,4- thiadiazol-2-yl)- 6,7,8,9-tetrahydro-5H- cyclohepta[b]thieno[3, 2-e]pyridine-2- carboxamide |
| 293 | 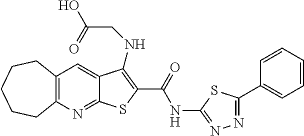 [SMILES: O=C(O)CNc1c(C(=O)Nc2nnc(-c3ccccc3)s2)sc2nc3c(cc12)CCCCC3] | C23 H21 N5 O3 S2 | 1H NMR in DMSO-d6: δ 8.18 (s, 1H), 7.87 (d, 2H), 7.57 (s, 3H), 7.37 (s, 2H), 4.67 (s, 2H), 3.08 (d, 2H), 2.84 (d, 2H), 1.84 (s, 2H), 1.65 (s, 4H); Mass Spec: 480.1 (M + H)+ | 2-((2-((5-phenyl-1,3,4- thiadiazol-2- yl)carbamoyl)-6,7,8,9- tetrahydro-5H- cyclohepta[b]thieno[3, 2-e]pyridin-3- yl)amino)acetic acid |
| 294 | 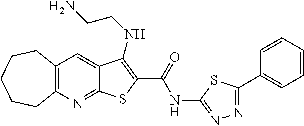 [SMILES: NCCNc1c(C(=O)Nc2nnc(-c3ccccc3)s2)sc2nc3c(cc12)CCCCC3] | C23 H24 N6 O S2 | 1H NMR in DMSO-d6: δ 8.32 (s, 1H), 8.21 (s, 2H), 7.90-7.92 (m, 2H), 7.58-7.60 (m, 3H), 4.69 (t, 2H), 3.46-3.52 (m, 2H), 3.02-3.11 (m, 4H), 2.88 (d, 2H), 1.86 (s, 2H), 1.67 (s, 4H); Mass Spec: 465.2 (M + H)+ | 3-((2- aminoethyl)amino)-N- (5-phenyl-1,3,4- thiadiazol-2-yl)- 6,7,8,9-tetrahydro-5H- cyclohepta[b]thieno[3, 2-e]pyridine-2- carboxamide |
| 295 | 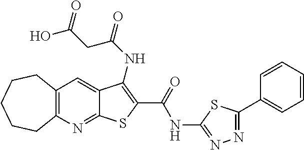 [SMILES: O=C(O)CC(=O)Nc1c(C(=O)Nc2nnc(-c3ccccc3)s2)sc2nc3c(cc12)CCCCC3] | C24 H21 N5 O4 S2 | 1H NMR in DMSO-d6: δ 7.98 (s, 1H), 7.89 (d, 2H), 7.37-7.50 (m, 3H), 3.26 (s, 2H), 3.08 (d, 2H), 2.85 (d, 2H), 1.85 (s, H), 1.66 (s, 4H); Mass Spec: 508.1 (M + H)+ | 3-oxo-3-((2-((5- phenyl-1,3,4- thiadiazol-2- yl)carbamoyl)-6,7,8,9- tetrahydro-5H- cyclohepta[b]thieno[3, 2-e]pyridin-3- yl)amino)propanoic acid |
| 296 | 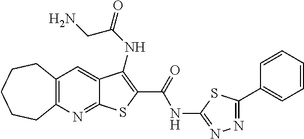 [SMILES: NCC(=O)Nc1c(C(=O)Nc2nnc(-c3ccccc3)s2)sc2nc3c(cc12)CCCCC3] | C23 H22 N6 O2 S2 | 1H NMR in DMSO-d6: δ 11.02 (s, 1H), 8.39 (s, 3H), 8.11 (s, 1H), 7.93-7.96 (m, 2H), 7.57-7.61 (m, 3H), 4.04 (d, 2H), 3.13 (d, 2H), 2.90 (d, 2H), 1.87 (s, 2H), 1.67 (s, 4H); Mass Spec: 479.1 (M + H)+ | 3-(2-aminoacetamido)- N-(5-phenyl-1,3,4- thiadiazol-2-yl)- 6,7,8,9-tetrahydro-5H- cyclohepta[b]thieno[3, 2-e]pyridine-2- carboxamide |
| 297 | 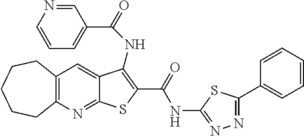 [SMILES: O=C(Nc1c(C(=O)Nc2nnc(-c3ccccc3)s2)sc2nc3c(cc12)CCCCC3)c1cccnc1] | C27 H22 N6 O2 S2 | 1H NMR in DMSO-d6: δ 11.33 (s, 1H), 9.46 (s, 1H), 9.03 (d, 1H), 8.82 (d, 1H), 8.13 (s, 1H), 7.92-8.03 (m, 3H), 7.54-7.56 (m, 3H), 3.15 (d, 2H), 2.93 (d, 2H), 1.86 (s, 2H), 1.68 (s, 4H); Mass Spec: 527.1 (M + H)+ | 3-(nicotinamido)-N- (5-phenyl-1,3,4- thiadiazol-2-yl)- 6,7,8,9-tetrahydro-5H- cyclohepta[b]thieno[3, 2-e]pyridine-2- carboxamide |
| 298 | 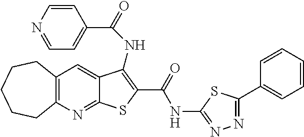 [SMILES: O=C(Nc1c(C(=O)Nc2nnc(-c3ccccc3)s2)sc2nc3c(cc12)CCCCC3)c1ccncc1] | C27 H22 N6 O2 S2 | 1H NMR in DMSO-d6: δ 11.40 (s, 1H), 9.09 (d, 2H), 8.36 (d, 2H), 8.12 (s, 1H), 7.95 (d, 2H), 7.56-7.58 (m, 3H), 3.16 (s, 2H), 2.94 (s, 2H), 1.88 (s, 2H), 1.69 (s, 4H); Mass Spec: 527.1 (M + H)+ | 3-(isonicotinamido)- N-(5-phenyl-1,3,4- thiadiazol-2-yl)- 6,7,8,9-tetrahydro-5H- cyclohepta[b]thieno[3, 2-e]pyridine-2- carboxamide |
| 299 | 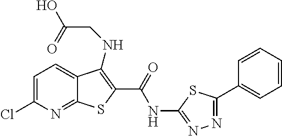 [SMILES: O=C(O)CNc1c(C(=O)Nc2nnc(-c3ccccc3)s2)sc2nc(Cl)ccc12] | C18 H12 Cl N5 O3 S2 | 1H NMR in DMSO-d6: δ 8.53 (d, 1H), 7.88 (s, 2H), 7.50-7.58 (m, 5H), 4.68 (s, 2H); Mass Spec: 446.0 (M + H)+ | 2-[[6-chloro-2-[(5- phenyl-1,3,4- thiadiazol-2- yl)carbamoyl]thieno[2, 3-b]pyridin-3- yl]amino]acetic acid |
| 300 | 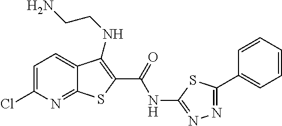 [SMILES: NCCNc1c(C(=O)Nc2nnc(-c3ccccc3)s2)sc2nc(Cl)ccc12] | C18 H15 Cl N6 O S2 | 1H NMR in DMSO-d6: δ 8.61 (d, 1H), 8.30 (s, 2H), 7.89-7.92 (m, 2H), 7.54-7.60 (m, 3H), 4.69-4.73 (m, 2H), 3.46-3.49 (m, 2H); Mass Spec: 431.1 (M + H)+ | 3-(2-aminoethylamino)-6- chloro-N-(5-phenyl- 1,3,4-thiadiazol-2- yl)thieno[2,3- b]pyridine-2- carboxamide |
| 302 | 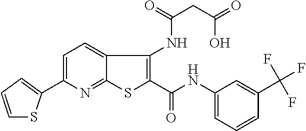 [SMILES: O=C(O)CC(=O)Nc1c(C(=O)Nc2cccc(C(F)(F)F)c2)sc2nc(-c3cccs3)ccc12] | C22 H14 F3 N3 O4 S2 | 1H NMR in DMSO-d6: δ 8.35 (s, 2H), 7.80-7.86 (m, 3H), 7.68 (d, 1H), 7.47 (t, 1H), 7.18-7.25 (m, 2H), 3.28-3.60 (bs, 2H); | 3-oxo-3-[[6-(2- thienyl)-2-[[3- (trifluoromethyl)phenyl]- carbamoyl]thieno[2,3- b]pyridin-3- yl]amino]propanoic acid |
| 303 | 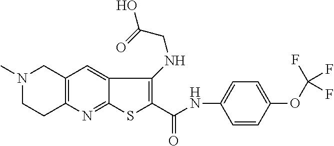 [SMILES: CN1CCc2nc3sc(C(=O)Nc4ccc(OC(F)(F)F)cc4)c(NCC(=O)O)c3cc2C1] | C21 H19 F3 N4 O4 S | 1H NMR in CD3OD: δ 8.44 (s, 1H), 7.73 (d, 2H), 7.48 (dd, 1H), 7.25 (d, 2H), 6.41 (d, 1H), 5.61 (d, 1H), 3.81 (s, 2H), 3.12 (s, 2H), 2.32 (s, 3H); Mass Spec: 481.1 (M + H)+ | 2-[[6-methyl-2-[[4- (trifluoromethoxy)phe- nyl]carbamoyl]-7,8- dihydro-5H- thieno[2,3- b][1,6]naphthyridin-3- yl]amino]acetic acid |
| 304 | 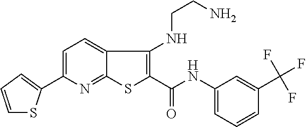 [SMILES: NCCNc1c(C(=O)Nc2cccc(C(F)(F)F)c2)sc2nc(-c3cccs3)ccc12] | C21 H17 F3 N4 O S2 | Mass Spec: 463.1 (M + H)+ | 3-(2- aminoethylamino)-6- (2-thienyl)-N-[3- (trifluoromethyl)phenyl]- thieno[2,3- b]pyridine-2-carboxamide |
| 305 | 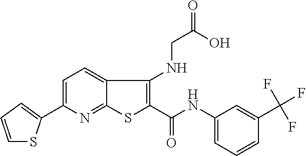 [SMILES: O=C(O)CNc1c(C(=O)Nc2cccc(C(F)(F)F)c2)sc2nc(-c3cccs3)ccc12] | C21 H14 F3 N3 O3 S2 | 1H NMR in DMSO-d6: δ 12.80 (s, 1H), 8.23-8.42 (m, 2H), 7.95-8.07 (m, 3H), 7.74 (d, 1H), 7.55 (t, 1H), 7.38 (d, 1H), 7.21 (s, 1H), 6.89 (s, 1H), 3.88 (s, 2H); Mass Spec: 478.1 (M + H)+ | 2-[[6-(2-thienyl)-2-[[3- (trifluoromethyl)phenyl]- carbamoyl]thieno[2,3- b]pyridin-3- yl]amino]acetic acid |
| 307 | 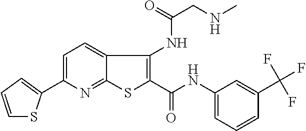 [SMILES: CNCC(=O)Nc1c(C(=O)Nc2cccc(C(F)(F)F)c2)sc2nc(-c3cccs3)ccc12] | C22 H17 F3 N4 O2 S2 | 1H NMR in DMSO-d6: δ 11.59 (s, 1H), 10.89 (s, 1H), 9.12 (s, 2H), 8.45 (d, 1H), 8.31 (s, 1H), 8.14 (d, 1H), 8.03-8.07 (m, 2H), 7.78 (d, 1H), 7.62 (t, 1H), 7.50 (d, 1H), 7.23-7.26 (m, 1H), 4.13 (s, 2H), 2.59 (s, 3H); Mass Spec: 491.1 (M + H)+ | 3-[[2- (methylamino)acetyl]- amino]-6-(2-thienyl)-N- [3-(trifluoromethyl)phenyl]- thieno[2,3- b]pyridine-2- carboxamide |
| 308 | 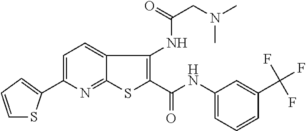 [SMILES: CN(C)CC(=O)Nc1c(C(=O)Nc2cccc(C(F)(F)F)c2)sc2nc(-c3cccs3)ccc12] | C23 H19 F3 N4 O2 S2 | 1H NMR in DMSO-d6: δ 11.44 (s, 1H), 10.85 (s, 1H), 9.95 (s, 1H), 8.44 (d, 1H), 8.26 (s, 1H), 8.16 (d, 1H), 7.97-8.03 (m, 2H), 7.78 (d, 1H), 7.62 (t, 1H), 7.49 (d, 1H), 7.25 (t, 1H), 4.32 (d, 2H), 2.83 (d, 6H); Mass Spec: 505.1 (M + H)+ | 3-[[2- (dimethylamino)acetyl]- amino]-6-(2-thienyl)- N-[3- (trifluoromethyl)phenyl]- thieno[2,3- b]pyridine-2- carboxamide |
| 309 | 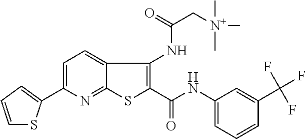 [SMILES: C[N+](C)(C)CC(=O)Nc1c(C(=O)Nc2cccc(C(F)(F)F)c2)sc2nc(-c3cccs3)ccc12] | C24 H22 F3 N4 O2 S2 | 1H NMR in DMSO-d6: δ 11.12 (s, 1H), 10.79 (s, 1H), 8.39 (d, 1H), 8.16-8.22 (m, 2H), 8.02 (d, 1H), 7.93 (d, 1H), 7.79 (d, 1H), 7.63 (t, 1H), 7.51 (d, 1H), 7.25 (t, 1H), 4.49 (s, 2H), 3.28 (s, 9H); Mass Spec: 519.1 (M + H)+ | N,N,N-trimethyl-2- oxo-2-((6-(thiophen-2- yl)-2-((3- (trifluoromethyl)phenyl)- carbamoyl)thieno[2,3- b]pyridin-3- yl)amino)ethanaminium |
| 310 | 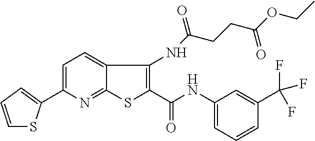 [SMILES: CCOC(=O)CCC(=O)Nc1c(C(=O)Nc2cccc(C(F)(F)F)c2)sc2nc(-c3cccs3)ccc12] | C25 H20 F3 N3 O4 S2 | 1H NMR in DMSO-d6: δ 10.67 (s, 1H), 10.56 (s, 1H), 8.25 (d, 1H), 8.21 (s, 1H), 8.11 (d, 1H), 7.97-8.01 (m, 2H), 7.77 (d, 1H), 7.62 (t, 1H), 7.49 (d, 1H), 7.22-7.25 (m, 1H), 3.96-4.03 (m, 2H), 2.74-2.78 (m, 2H), 2.59-2.63 (m, 2H), 1.14 (t, 3H); Mass Spec: 548.1 (M + H)+ | ethyl 4-oxo-4-[[6-(2- thienyl)-2-[[3- (trifluoromethyl)phenyl]- carbamoyl]thieno[2,3- b]pyridin-3- yl]amino]butanoate |
| 311 | 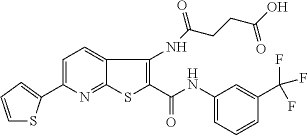 [SMILES: O=C(O)CCC(=O)Nc1c(C(=O)Nc2cccc(C(F)(F)F)c2)sc2nc(-c3cccs3)ccc12] | C23 H16 F3 N3 O4 S2 | 1H NMR in CD3OD: δ 8.32 (s, 1H), 8.11 (d, 1H), 7.77-7.79 (m, 2H), 7.69 (d, 1H), 7.53-7.57 (m, 2H), 7.36 (d, 1H), 7.16 (t, 1H), 2.82-2.87 (m, 2H), 2.71-2.76 (m, 2H); Mass Spec: 520.0 (M + H)+ | 4-oxo-4-[[6-(2- thienyl)-2-[[3- (trifluoromethyl)phenyl]- carbamoyl]thieno[2,3- b]pyridin-3- yl]amino]butanoic acid |
| 312 | 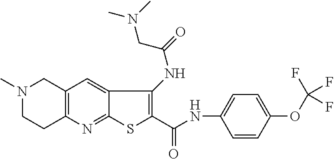 [SMILES: CN(C)CC(=O)Nc1c(C(=O)Nc2ccc(OC(F)(F)F)cc2)sc2nc3c(cc12)CN(C)CC3] | C23 H24 F3 N5 O3 S | 1H NMR in CD3OD: δ 8.04 (s, 1H), 7.76 (d, 2H), 7.28 (d, 2H), 3.79 (s, 2H), 3.25 (s, 2H), 3.19 (s, 2H), 2.92 (s, 2H), 2.52 (s, 3H), 2.41 (s, 6H); Mass Spec: 508.2 (M + H)+ | 3-[[2- (dimethylamino)acetyl]- amino]-6-methyl-N-[4- (trifluoromethoxy)phe- nyl]-7,8-dihydro-5H- thieno[2,3- b][1,6]naphthyridine- 2-carboxamide |
| 313 | 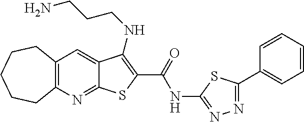 [SMILES: NCCCNc1c(C(=O)Nc2nnc(-c3ccccc3)s2)sc2nc3c(cc12)CCCCC3] | C24 H26 N6 O S2 | 1H NMR in DMSO-d6: δ 8.36 (s, 1H), 8.05 (s, 3H), 7.89-7.91 (m, 2H), 7.58-7.60 (m, 3H), 4.48-4.59 (m, 2H), 3.13 (s, 2H), 2.94-2.99 (m, 2H), 2.87-2.92 (m, 2H), 2.21-2.30 (m, 2H), 1.86 (s, 2H), 1.68 (s, 4H); | 3-((3- aminopropyl)amino)- N-(5-phenyl-1,3,4- thiadiazol-2-yl)- 6,7,8,9-tetrahydro-5H- cyclohepta[b]thieno[3, 2-e]pyridine-2- carboxamide |
| 314 | 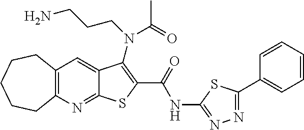 [SMILES: CC(=O)N(CCCN)c1c(C(=O)Nc2nnc(-c3ccccc3)s2)sc2nc3c(cc12)CCCCC3] | C25 H26 N6 O2 S2 | 1H NMR in DMSO-d6: δ 10.34 (s, 1H), 8.30 (s, 3H), 7.96 (d, 2H), 7.92 (s, 1H), 7.60-7.62 (m, 3H), 4.80 (t, 2H), 3.48-3.55 (m, 2H), 3.13 (d, 2H), 2.91 (d, 2H), 2.25 (s, 3H), 1.86 (s, 2H), 1.67 (s, 4H); | 3-(N-(2- aminoethyl)acetamido)- N-(5-phenyl-1,3,4- thiadiazol-2-yl)- 6,7,8,9-tetrahydro-5H- cyclohepta[b]thieno[3, 2-e]pyridine-2- carboxamide |
| 315 | 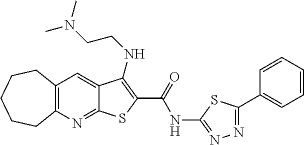 [SMILES: CN(C)CCNc1c(C(=O)Nc2nnc(-c3ccccc3)s2)sc2nc3c(cc12)CCCCC3] | C25 H28 N6 O S2 | 1H NMR in CDCl3: δ 7.84-7.86 (m, 2H), 7.60 (s, 1H), 7.48-7.49 (s, 3H), 4.60 (t, 2H), 3.14-3.16 (m, 2H), 2.88-2.92 (m, 4H), 2.39 (s, 6H), 1.88-1.93 (m, 2H), 1.69- 1.19 (m, 4H); | 3-((2- (dimethylamino)ethyl)- 1,3,4-thiadiazol-2-yl)- amino)-N-(5-phenyl- 6,7,8,9-tetrahydro-5H- cyclohepta[b]thieno[3, 2-e]pyridine-2- carboxamide |
| 321 | 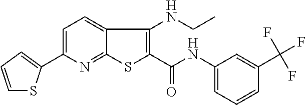 [SMILES: CCNc1c(C(=O)Nc2cccc(C(F)(F)F)c2)sc2nc(-c3cccs3)ccc12] | C21 H16 F3 N3 O S2 | 1H NMR in DMSO-d6: δ 9.93 (s, 1H), 8.57 (d, 1H), 8.17 (s, 1H), 7.95-8.08 (m, 4H), 7.77 (d, 1H), 7.58 (t, 1H), 7.44 (d, 1H), 7.24 (t, 1H), 3.65-3.71 (m, 2H), 1.28 (t, 3H); Mass Spec: 448.0 (M + H)+ | 3-(ethylamino)-6-(2- thienyl)-N-[3- (trifluoromethyl)phenyl]- thieno[2,3- b]pyridine-2- carboxamide |
| 358 | 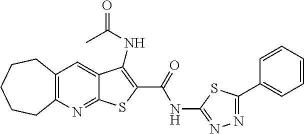 [SMILES: CC(=O)Nc1c(C(=O)Nc2nnc(-c3ccccc3)s2)sc2nc3c(cc12)CCCCC3] | C23 H21 N5 O2 S2 | 1H NMR in CDCl3: δ 7.83 (d, 2H), 7.46- 7.53 (m, 4H), 6.88 (s, 2H), 3.13 (d, 2H), 2.82-2.88 (m, 5H), 1.90 (s, 2H), 1.73 (s, 4H); Mass Spec: 464.1 (M + H)+ | 3-acetamido-N-(5- phenyl-1,3,4- thiadiazol-2-yl)- 6,7,8,9-tetrahydro-5H- cyclohepta[b]thieno[3, 2-e]pyridine-2- carboxamide |
| 359 | 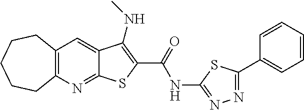 [SMILES: CNc1c(C(=O)Nc2nnc(-c3ccccc3)s2)sc2nc3c(cc12)CCCCC3] | C22 H21 N5 O S2 | 1H NMR in DMSO-d6: δ 8.16 (s, 1H), 7.85-7.88 (m, 2H), 7.55-7.57 (m, 3H), 7.32 (s, 2H), 4.01 (s, 3H), 3.06 (d, 2H), 2.86 (d, 2H), 1.84 (s, 2H), 1.66 (s, 4H); Mass Spec: 436.2 (M + H)+ | 3-(methylamino)-N- (5-phenyl-1,3,4- thiadiazol-2-yl)- 6,7,8,9-tetrahydro-5H- cyclohepta[b]thieno[3, 2-e]pyridine-2- carboxamide |
| 360 | 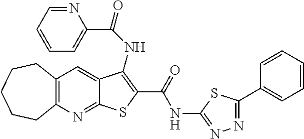 [SMILES: O=C(Nc1c(C(=O)Nc2nnc(-c3ccccc3)s2)sc2nc3c(cc12)CCCCC3)c1ccccn1] | C27 H22 N6 O2 S2 | 1H NMR in DMSO-d6: δ 12.04 (s, 1H), 8.89-8.92 (m, 1H), 8.25-8.28 (m, 2H), 8.14-8.18 (m, 1H), 7.93-7.95 (m, 2H), 7.77-7.81 (m, 1H), 7.56-7.59 (m, 3H), 3.15 (s, 2H), 2.92 (s, 2H), 1.87 (s, 2H), 1.69 (s, 4H); Mass Spec: 527.1 (M + H)+ | N-(5-phenyl-1,3,4- thiadiazol-2-yl)-3- (picolinamido)- 6,7,8,9-tetrahydro-5H- cyclohepta[b]thieno[3, 2-e]pyridine-2- carboxamide |
| 361 | 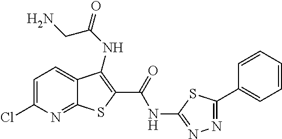 [SMILES: NCC(=O)Nc1c(C(=O)Nc2nnc(-c3ccccc3)s2)sc2nc(Cl)ccc12] | C18 H13 Cl N6 O2 S2 | 1H NMR in DMSO-d6: δ 11.08 (s, 1H), 8.32 (s, 3H), 7.93 (s, 2H), 7.59-7.70 (m, 4H), 4.05 (s, 2H); Mass Spec: 445.1 (M + H)+ | 3-[(2- aminoacetyl)amino]-6- chloro-N-(5-phenyl- 1,3,4-thiadiazol-2- yl)thieno[2,3- b]pyridine-2- carboxamide |
| 362 | 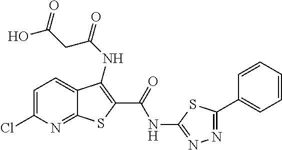 [SMILES: O=C(O)CC(=O)Nc1c(C(=O)Nc2nnc(-c3ccccc3)s2)sc2nc(Cl)ccc12] | C19 H12 Cl N5 O4 S2 | 1H NMR in DMSO-d6: δ 8.35 (s, 1H), 7.90 (s, 2H), 7.50 (s, 4H), 3.24 (s, 2H); Mass Spec: 474.0 (M + H)+ | 3-[[6-chloro-2-[(5- phenyl-1,3,4- thiadiazol-2- yl)carbamoyl]thieno[2, 3-b]pyridin-3- yl]amino]-3-oxo- propanoic acid |
| 363 | 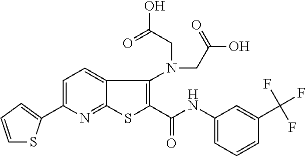 [SMILES: O=C(O)CN(CC(=O)O)c1c(C(=O)Nc2cccc(C(F)(F)F)c2)sc2nc(-c3cccs3)ccc12] | C23 H16 F3 N3 O5 S2 | 1H NMR in D2O: δ 8.50 (d, 1H), 8.22 (s, 1H), 7.96 (d, 1H), 7.83 (d, 1H), 7.76 (d, 1H), 7.54-7.68 (m, 3H), 7.09 (t, 1H), 3.99 (s, 4H); Mass Spec: 536.0 (M + H)+ | 2-[carboxymethyl-[6- (2-thienyl)-2-[[3- (trifluoromethyl)phenyl]- carbamoyl]thieno[2,3- b]pyridin-3- yl]amino]acetic acid |

TABLE 3
Novel Compounds of Formula III activity against
Dengue Virus in Vero cells.

Activity (EC₅₀ in μM)
A: EC₅₀ ≦ 5 μM; B: 5 < EC₅₀ ≦ 25 μM;
C: EC₅₀ > 25 μM; n.d.: not determined

| Cmpd | DENV-1 | DENV-2 | DENV-3 | DENV-4 |
|---|---|---|---|---|
| 285 | A | A | A | A |
| 289 | A | A | A | A |
| 293 | A | A | A | A |
| 294 | A | A | A | A |
| 295 | A | A | A | A |
| 296 | A | A | A | A |
| 297 | A | A | A | A |
| 298 | A | A | A | A |
| 299 | B | B | n.d. | B |
| 300 | A | A | A | A |
| 302 | A | A | B | A |
| 303 | B | A | B | A |
| 304 | A | A | A | A |
| 305 | A | A | B | A |
| 307 | A | A | A | A |
| 308 | n.d. | A | n.d. | n.d. |
| 309 | A | A | A | A |
| 310 | A | A | A | A |
| 311 | A | A | A | A |
| 312 | A | A | A | A |
| 313 | n.d. | A | n.d. | n.d. |
| 314 | n.d. | A | n.d. | n.d. |
| 315 | n.d. | A | n.d. | n.d. |
| 321 | A | A | A | A |
| 358 | A | A | B | C |
| 359 | A | A | C | B |
| 360 | C | A | C | A |
| 361 | A | A | A | C |
| 362 | B | B | C | C |
| 363 | B | A | C | C |

TABLE 4
Novel compounds of the present invention outside the
scope of Formula III.

| | | Molecular | | |
|---|---|---|---|---|
| Cmpd | Chemical Structure | Formula | Analytical Data | Chemical Name |
| 281 | 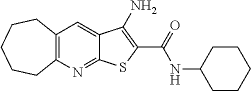 [SMILES: Nc1c(C(=O)NC2CCCCC2)sc2nc3c(cc12)CCCCC3] | C19 H25 N3 O S | 1H NMR in DMSO-d6: δ 8.11 (s, 1H), 7.32 (d, 1H), 7.05 (s, 2H), 3.72-3.74 (m, 1H), 3.06 (dd, 2H), 2.86 (dd, 2H), 1.64- 1.84 (m, 11H), 1.20-1.41 (m, 3H), 1.03- 1.15 (m, 2H); Mass Spec: 344.2 (M + H)⁺ | 3-amino-N- cyclohexyl-6,7,8,9- tetrahydro-5H- cyclohepta[b]thieno[3, 2-e]pyridine-2- carboxamide |
| 282 | 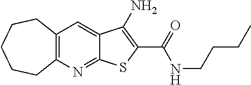 [SMILES: CCCCNC(=O)c1sc2nc3c(cc2c1N)CCCCC3] | C17 H23 N3 O S | 1H NMR in DMSO-d6: δ 8.08 (s, 1H), 7.58 (t, 1H), 7.02 (s, 2H), 3.14-3.20 (m, 2H), 3.02 (d, 2H), 2.81 (s, 2H), 1.80 (s, 2H), 1.60 (s, 4H), 1.41-1.48 (m, 2H), 1.24- 1.31 (m, 2H), 0.87 (t, 3H); Mass Spec: 318.1 (M + H)+ | 3-amino-N-butyl- 6,7,8,9-tetrahydro-5H- cyclohepta[b]thieno[3, 2-e]pyridine-2- carboxamide |
| 283 | 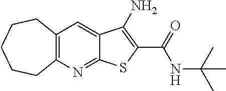 [SMILES: CC(C)(C)NC(=O)c1sc2nc3c(cc2c1N)CCCCC3] | C17 H23 N3 O S | 1H NMR in DMSO-d6: δ 8.09 (s, 1H), 6.95 (s, 2H), 6.55 (s, 1H), 3.03 (d, 2H), 2.83 (d, 2H), 1.81 (s, 2H), 1.63 (s, 4H), 1.36 (s, 9H); Mass Spec: 318.2 (M + H)+ | 3-amino-N-(tert- butyl)-6,7,8,9- tetrahydro-5H- cyclohepta[b]thieno[3, 2-e]pyridine-2- carboxamide |
| 284 | 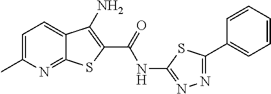 [SMILES: Cc1ccc2c(N)c(C(=O)Nc3nnc(-c4ccccc4)s3)sc2n1] | C17 H13 N5 O S2 | 1H NMR in DMSO-d6: δ 8.18 (d, 1H), 7.85 (d, 2H), 7.37-7.49 (m, 4H), 7.23 (d, 1H), 7.10 (s, 2H), 2.57 (s, 3H); Mass Spec: 368.1 (M + H)+ | 3-amino-6-methyl-N- (5-phenyl-1,3,4- thiadiazol-2- yl)thieno[2,3- b]pyridine-2- carboxamide |
| 286 | 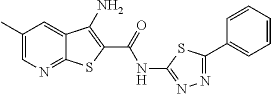 [SMILES: Cc1cnc2sc(C(=O)Nc3nnc(-c4ccccc4)s3)c(N)c2c1] | C17 H13 N5 O S2 | 1H NMR in DMSO-d6: δ 8.39 (s, 1H), 8.10 (s, 1H), 7.83-7.85 (m, 2H), 7.33-7.47 (m, 3H), 7.05 (s, 2H), 2.41 (s, 3H); Mass Spec: 368.1 (M + H)+ | 3-amino-5-methyl-N- (5-phenyl-1,3,4- thiadiazol-2- yl)thieno[2,3- b]pyridine-2- carboxamide |
| 287 | 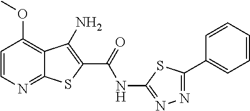 [SMILES: COc1ccnc2sc(C(=O)Nc3nnc(-c4ccccc4)s3)c(N)c12] | C17 H13 N5 O2 S2 | 1H NMR in DMSO-d6: δ 8.37 (d, 1H), 7.85 (d, 2H), 7.34-7.48 (m, 3H), 6.90 (s, 3H), 4.00 (s, 3H); Mass Spec: 384.1 (M + H)+ | 3-amino-4-methoxy- N-(5-phenyl-1,3,4- thiadiazol-2- yl)thieno[2,3- b]pyridine-2- carboxamide |
| 288 | 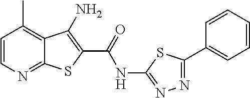 [SMILES: Cc1ccnc2sc(C(=O)Nc3nnc(-c4ccccc4)s3)c(N)c12] | C17 H13 N5 O S2 | 1H NMR in DMSO-d6: δ 8.33 (d, 1H), 7.85 (d, 2H), 7.36-7.48 (m, 3H), 7.06 (d, 1H), 6.84 (s, 2H), 2.79 (s, 3H); Mass Spec: 368.1 (M + H)+ | 3-amino-4-methyl-N- (5-phenyl-1,3,4- thiadiazol-2- yl)thieno[2,3- b]pyridine-2- carboxamide |
| 290 | 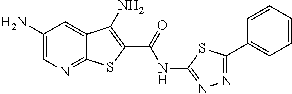 [SMILES: Nc1cnc2sc(C(=O)Nc3nnc(-c4ccccc4)s3)c(N)c2c1] | C16 H12 N6 O S2 | 1H NMR in DMSO-d6: δ 8.02 (d, 1H), 7.83 (d, 2H), 7.32-7.47 (m, 4H), 6.89 (s, 2H), 5.28 (s, 2H); Mass Spec: 369.1 (M + H)+ | 3,5-diamino-N-(5- phenyl-1,3,4- thiadiazol-2- yl)thieno[2,3- b]pyridine-2- carboxamide |
| 291 | 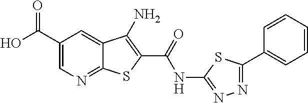 [SMILES: Nc1c(C(=O)Nc2nnc(-c3ccccc3)s2)sc2ncc(C(=O)O)cc12] | C17 H11 N5 O3 S2 | 1H NMR in DMSO-d6: δ 12.81 (s, 1H), 8.13 (d, 2H), 7.91 (s, 3H), 7.49-7.56 (m, 5H); Mass Spec: 398.0 (M + H)+ | 3-amino-2-((5-phenyl- 1,3,4-thiadiazol-2- yl)carbamoyl)thieno[2, 3-b]pyridine-5- carboxylic acid |
| 292 | 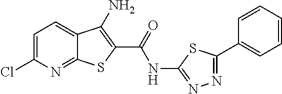 [SMILES: Nc1c(C(=O)Nc2nnc(-c3ccccc3)s2)sc2nc(Cl)ccc12] | C16 H10 Cl N5 O S2 | 1H NMR in DMSO-d6: δ 8.57 (s, 1H), 7.91 (s, 2H), 7.56 (s, 5H); Mass Spec: 388.0 (M + H)+ | 3-amino-6-chloro-N- (5-phenyl-1,3,4- thiadiazol-2- yl)thieno[2,3- b]pyridine-2- carboxamide |
| 301 | 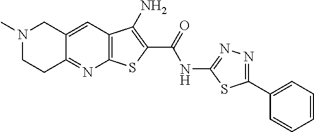 [SMILES: CN1CCc2nc3sc(C(=O)Nc4nnc(-c5ccccc5)s4)c(N)c3cc2C1] | C20 H18 N6 O S2 | 1H NMR in DMSO-d6: δ 7.99 (s, 1H), 7.83-7.85 (m, 2H), 7.33-7.47 (m, 3H), 7.09 (s, 2H), 3.63 (s, 2H), 3.01 (s, 2H), 2.74 (s, 2H), 2.40 (s, 3H); Mass Spec: 423.2 (M + H)+ | 3-amino-6-methyl-N- (5-phenyl-1,3,4- thiadiazol-2-yl)-7,8- dihydro-5H- thieno[2,3- b][1,6]naphthyridine- 2-carboxamide |
| 306 | 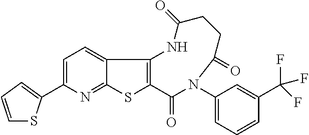 [SMILES: O=C1CCC(=O)N(c2cccc(C(F)(F)F)c2)C(=O)c2sc3nc(-c4cccs4)ccc3c2N1] | C23 H14 F3 N3 O3 S2 | 1H NMR in DMSO-d6: δ 10.81 (s, 1H), 8.28 (d, 1H), 8.16 (d, 1H), 8.10 (s, 1H), 8.04 (d, 1H), 7.91 (d, 1H), 7.80 (d, 1H), 7.62 (t, 1H), 7.52 (d, 1H), 7.25 (t, 1H), 2.81-3.03 (m, 4H); Mass Spec: 502.0 (M + H)+ | 2-(thiophen-2-yl)-10- (3-trifluoromethyl)- phenyl)- 7,8-dihydro-5H- pyrido[3′,2′:4,5]thieno- [3,2-b][1,5]diazonine- 6,9,11(10H)-trione |
| 316 | 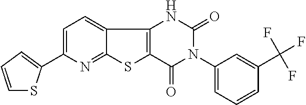 [SMILES: O=c1[nH]c2c(sc3nc(-c4cccs4)ccc32)c(=O)n1-c1cccc(C(F)(F)F)c1] | C20 H10 F3 N3 O2 S2 | 1H NMR in DMSO-d6: δ 8.35 (d, 1H), 8.01 (d, 1H), 7.95 (d, 1H), 7.74 (d, 1H), 7.63-7.68 (m, 2H), 7.50-7.53 (m, 2H), 7.23 (t, 1H); Mass Spec: 446.0 (M + H)+ | 7-(thiophen-2-yl)-3-(3- (trifluoromethyl)- phenyl)- pyrido[3′,2′:4,5]thieno- [3,2-d]pyrimidine- 2,4(1H,3H)-dione |
| 317 | 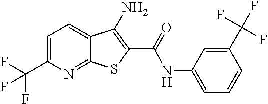 [SMILES: Nc1c(C(=O)Nc2cccc(C(F)(F)F)c2)sc2nc(C(F)(F)F)ccc12] | C16 H9 F6 N3 O S | 1H NMR in DMSO-d6: δ 9.97 (s, 1H), 8.83 (d, 1H), 8.22 (s, 1H), 7.98-8.02 (m, 2H), 7.55-7.63 (m, 3H), 7.43 (d, 1H); Mass Spec: 406.0 (M + H)+ | 3-amino-6- (trifluoromethyl)-N- [3-(trifluoromethyl)- phenyl] thieno[2,3- b]pyridine-2- carboxamide |
| 318 | 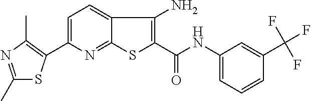 [SMILES: Cc1nc(C)c(-c2ccc3c(N)c(C(=O)Nc4cccc(C(F)(F)F)c4)sc3n2)s1] | C20 H15 F3 N4 O S2 | 1H NMR in DMSO-d6: δ 9.76 (s, 1H), 8.59 (d, 1H), 8.23 (s, 1H), 8.00 (d, 1H), 7.75 (d, 1H), 7.55-7.60 (m, 3H), 7.72 (d, 1H), 2.65-2.66 (m, 6H); Mass Spec: 449.1 (M + H)+ | 3-amino-6-(2,4- dimethylthiazol-5-yl)- N-[3- (trifluoromethyl)- phenyl]- thieno[2,3- b]pyridine-2- carboxamide |
| 319 | 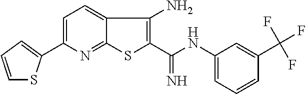 [SMILES: N=C(Nc1cccc(C(F)(F)F)c1)c1sc2nc(-c3cccs3)ccc2c1N] | C19 H13 F3 N4 S2 | 1H NMR in DMSO-d6: δ 8.44 (d, 1H), 8.01 (d, 1H), 7.93 (dd, 1H), 7.72 (d, 1H), 7.54 (t, 1H), 7.39 (s, 2H), 7.32 (d, 1H), 7.20-7.23 (m, 3H), 6.20 (s, 2H); Mass Spec: 419.0 (M + H)+ | 3-amino-6-(2-thienyl)- N-[3- (trifluoromethyl)- phenyl]- thieno[2,3- b]pyridine-2- carboxamidine |
| 320 | 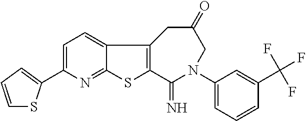 [SMILES: N=C1c2sc3nc(-c4cccs4)ccc3c2CC(=O)CN1c1cccc(C(F)(F)F)c1] | C21 H12 F3 N3 O2 S2 | 1H NMR in DMSO-d6: δ 8.62 (d, 1H), 8.17 (d, 1H), 8.03 (dd, 1H), 7.95 (s, 1H), 7.79-7.82 (m, 2H), 7.71-7.78 (m, 2H), 7.23-7.26 (m, 1H), 4.56 (s, 2H); Mass Spec: 460.0 (M + H)+ | 8-(thiophen-2-yl)-4-(3- (trifluoromethyl)- phenyl)- 3,4-dihydro-1H- pyrido[3′,2′: 4,5]thieno[3,2- e][1,4]diazepine- 2,5-dione |
| 322 | 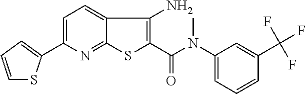 [SMILES: CN(C(=O)c1sc2nc(-c3cccs3)ccc2c1N)c1cccc(C(F)(F)F)c1] | C20 H14 F3 N3 O S2 | 1H NMR in DMSO-d6: δ 8.50 (d, 1H), 7.95 (d, 1H), 7.90 (d, 1H), 7.85 (s, 1H), 7.78-7.81 (m, 1H), 7.69-7.70 (m, 3H), 7.54 (s, 2H), 7.16-7.19 (m, 1H), 3.35 (s, 3H); Mass Spec: 434.0 (M + H)+ | 3-amino-N-methyl-6- (2-thienyl)-N-[3- (trifluoromethyl)- phenyl]- thieno[2,3- b]pyridine-2- carboxamide |
| 323 | 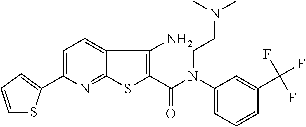 [SMILES: CN(C)CCN(C(=O)c1sc2nc(-c3cccs3)ccc2c1N)c1cccc(C(F)(F)F)c1] | C23 H21 F3 N4 O S2 | 1H NMR in DMSO-d6: δ 8.53 (d, 1H), 7.69-8.01 (m, 7H), 7.18 (t, 1H), 6.69 (bs, 2H), 4.18 (t, 2H), 3.29 (q, 2H), 2.85-2.86 (m, 6H); Mass Spec: 491.1 (M + H)+ | 3-amino-N-(2- dimethylaminoethyl)- 6-(2-thienyl)-N-[3- (trifluoromethyl)- phenyl]- thieno[2,3- b]pyridine-2- carboxamide |
| 324 | 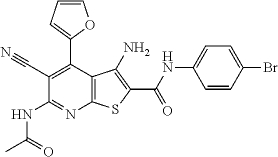 [SMILES: CC(=O)Nc1nc2sc(C(=O)Nc3ccc(Br)cc3)c(N)c2c(-c2ccco2)c1C#N] | C21 H14 Br N5 O3 S | 1H NMR in DMSO-d6: δ 11.09 (s, 1H), 10.37 (s, 1H), 8.23 (d, 1H), 7.49-7.57 (m, 5H), 6.91-6.92 (m, 1H), 4.28 (s, 2H), 2.17 (s, 3H); Mass Spec: 497.0 (M + H)+ | 6-acetamido-3-amino- N-(4-bromophenyl)-5- cyano-4-(2- furyl)thieno[2,3- b]pyridine-2- carboxamide |
| 325 | 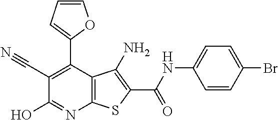 [SMILES: N#Cc1c(O)nc2sc(C(=O)Nc3ccc(Br)cc3)c(N)c2c1-c1ccco1] | C19 H11 Br N4 O3 S | 1H NMR in DMSO-d6: δ 9.50 (s, 1H), 8.09 (t, 1H), 7.47-7.65 (m, 5H), 7.13 (d, 1H), 6.85 (d, 1H), 6.35 (s, 2H); Mass Spec: 456.0 (M + 2H)+ | 3-amino-N-(4- bromophenyl)-5- cyano-4-(2-furyl)-6- hydroxy-thieno[2,3- b]pyridine-2- carboxamide |
| 326 | 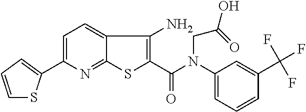 [SMILES: Nc1c(C(=O)N(CC(=O)O)c2cccc(C(F)(F)F)c2)sc2nc(-c3cccs3)ccc12] | C21 H14 F3 N3 O3 S2 | 1H NMR in DMSO-d6: δ 8.26 (d, 1H), 7.87-7.90 (m, 2H), 7.68-7.70 (m, 2H), 7.44-7.53 (m, 3H), 7.31 (s, 2H), 7.16-7.19 (m, 1H), 4.07 (s, 2H); Mass Spec: 478.0 (M + H)+ | 2-[N-[3-amino-6-(2- thienyl)thieno[2,3- b]pyridine-2- carbonyl]-3- (trifluoromethyl)- anilino]- acetic acid |
| 327 | 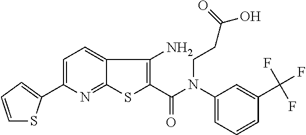 [SMILES: Nc1c(C(=O)N(CCC(=O)O)c2cccc(C(F)(F)F)c2)sc2nc(-c3cccs3)ccc12] | C22 H16 F3 N3 O3 S2 | 1H NMR in DMSO-d6: δ 8.44 (d, 1H), 7.88-7.94 (m, 2H), 7.82 (s, 1H), 7.76-7.77 (m, 1H), 7.66-7.69 (m, 3H), 7.52 (s, 2H), 7.15-7.18 (m, 1H), 3.90 (t, 2H), 2.17 (t, 2H); Mass Spec: 492.1 (M + H)+ | 3-[N-[3-amino-6-(2- thienyl)thieno[2,3- b]pyridine-2- carbonyl]-3- (trifluoromethyl)- anilino]- propanoic acid |
| 328 | 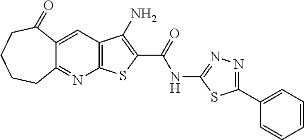 [SMILES: Nc1c(C(=O)Nc2nnc(-c3ccccc3)s2)sc2nc3c(cc12)C(=O)CCCC3] | C21 H17 N5 O2 S2 | 1H NMR in DMSO-d6: δ 8.58 (s, 1H), 7.83-7.86 (m, 2H), 7.43-7.48 (m, 2H), 7.34-7.39 (m, 1H), 7.29 (s, 2H), 3.22 (t, 2H), 2.82 (t, 2H), 1.91 (t, 2H), 1.74-1.82 (m, 2H); Mass Spec: 436.1 (M + H)+ | 3-amino-5-oxo-N-(5- phenyl-1,3,4- thiadiazol-2-yl)- 6,7,8,9-tetrahydro-5H- cyclohepta[b]thieno[3, 2-e]pyridine-2- carboxamide |
| 329 | 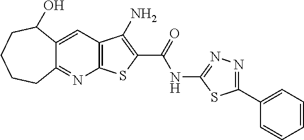 [SMILES: Nc1c(C(=O)Nc2nnc(-c3ccccc3)s2)sc2nc3c(cc12)C(O)CCCC3] | C21 H19 N5 O2 S2 | 1H NMR in DMSO-d6: δ 8.53 (s, 1H), 7.91-7.93 (m, 2H), 7.55-7.57 (m, 3H), 5.62 (d, 1H), 4.88-4.90 (m, 1H), 2.96-3.11 (m, 2H), 1.81-2.02 (m, 4H), 1.35-1.58 (m, 2H); Mass Spec: 438.1 (M + H)+ | 3-amino-5-hydroxy-N- (5-phenyl-1,3,4- thiadiazol-2-yl)- 6,7,8,9-tetrahydro-5H- cyclohepta[b]thieno[3, 2-e]pyridine-2- carboxamide |
| 330 | 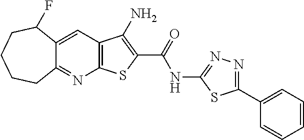 [SMILES: Nc1c(C(=O)Nc2nnc(-c3ccccc3)s2)sc2nc3c(cc12)C(F)CCCC3] | C21 H18 F N5 O S2 | Mass Spec: 440.0 (M + H)+ | 3-amino-5-fluoro-N- (5-phenyl-1,3,4- thiadiazol-2-yl)- 6,7,8,9-tetrahydro-5H- cyclohepta[b]thieno[3, 2-e]pyridine-2- carboxamide |
| 331 | 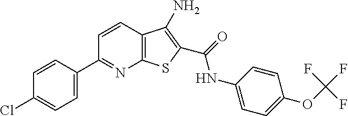 [SMILES: Nc1c(C(=O)Nc2ccc(OC(F)(F)F)cc2)sc2nc(-c3ccc(Cl)cc3)ccc12] | C21 H13 Cl F3 N3 O2 S | 1H NMR in DMSO-d6: δ 9.69 (s, 1H), 8.61 (d, 1H), 8.24 (d, 2H), 8.12 (d, 1H), 7.83 (d, 2H), 7.61 (d, 2H), 7.48 (s, 2H), 7.35 (d, 2H); Mass Spec: 463.8 (M + H)+ | 3-amino-6-(4- chlorophenyl)-N-[4- (trifluoromethoxy)- phenyl]- thieno[2,3- b]pyridine-2- carboxamide |
| 332 | 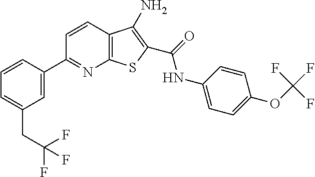 [SMILES: Nc1c(C(=O)Nc2ccc(OC(F)(F)F)cc2)sc2nc(-c3cccc(CC(F)(F)F)c3)ccc12] | C22 H13 F6 N3 O3 S | 1H NMR in DMSO-d6: δ 9.70 (s, 1H), 8.64 (d, 2H), 8.17-8.27 (m, 3H), 7.83 (d, 2H), 7.69 (t, 1H), 7.49-7.53 (m, 3H), 7.35 (d, 2H); Mass Spec: 513.8 (M + H)+ | 3-amino-6-[3- (trifluoromethoxy)- phenyl]- N-[4- (trifluoromethoxy)- phenyl]- thieno[2,3- b]pyridine-2- carboxamide |
| 333 | 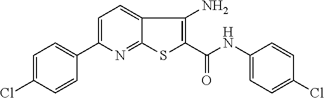 [SMILES: Nc1c(C(=O)Nc2ccc(Cl)cc2)sc2nc(-c3ccc(Cl)cc3)ccc12] | C20 H13 Cl2 N3 O S | 1H NMR in DMSO-d6: δ 9.62 (s, 1H), 8.61 (d, 1H), 8.23 (d, 2H), 8.12 (d, 1H), 7.76 (d, 2H), 7.60 (d, 2H), 7.47 (s, 2H), 7.39 (d, 2H); Mass Spec: 413.8 (M + H)+ | 3-amino-N,6-bis(4- chlorophenyl)thieno[2, 3-b]pyridine-2- carboxamide |
| 334 | 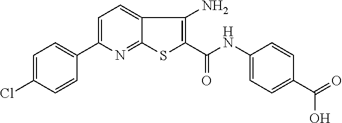 [SMILES: Nc1c(C(=O)Nc2ccc(C(=O)O)cc2)sc2nc(-c3ccc(Cl)cc3)ccc12] | C21 H14 Cl N3 O3 S | 1H NMR in DMSO-d6: δ 9.77 (s, 1H), 8.63 (d, 1H), 8.24 (d, 2H), 8.12 (d, 1H), 7.86-7.94 (m, 4H), 7.55-7.62 (m, 4H); Mass Spec: 423.9 (M + H)+ | 4-[[3-amino-6-(4- chlorophenyl)thieno[2, 3-b]pyridine-2- carbonyl]amino]benzo- ic acid |
| 335 | 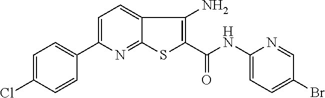 [SMILES: Nc1c(C(=O)Nc2ccc(Br)cn2)sc2nc(-c3ccc(Cl)cc3)ccc12] | C19 H12 Br Cl N4 O S | 1H NMR in DMSO-d6: δ 9.99 (s, 1H), 8.62 (d, 1H), 8.48 (d, 1H), 8.23 (d, 2H), 8.11 (d, 1H), 8.01-8.06 (m, 2H), 7.54-7.61 (m, 4H); Mass Spec: 460.8 (M + H)+ | 3-amino-N-(5-bromo- 2-pyridyl)-6-(4- chlorophenyl)thieno[2, 3-b]pyridine-2- carboxamide |
| 336 | 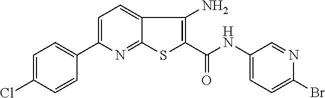 [SMILES: Nc1c(C(=O)Nc2ccc(Br)nc2)sc2nc(-c3ccc(Cl)cc3)ccc12] | C19 H12 Br Cl N4 O S | 1H NMR in DMSO-d6: δ 9.82 (s, 1H), 8.77 (d, 1H), 8.63 (d, 1H), 8.24 (d, 2H), 8.09-8.14 (m, 2H), 7.56-7.63 (m, 5H); Mass Spec: 460.8 (M + H)+ | 3-amino-N-(6-bromo- 3-pyridyl)-6-(4- chlorophenyl)thieno[2, 3-b]pyridine-2- carboxamide |
| 337 | 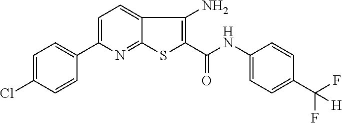 [SMILES: Nc1c(C(=O)Nc2ccc(C(F)F)cc2)sc2nc(-c3ccc(Cl)cc3)ccc12] | C21 H14 Cl F2 N3 O S | 1H NMR in DMSO-d6: δ 9.69 (s, 1H), 8.61 (d, 1H) 8.23 (d, 2H), 8.11 (d, 1H) 7.87 (d, 2H), 7.52-7.61 (m, 6H), 6.99 (t, 1H); Mass Spec: 429.9 (M + H)+ | 3-amino-6-(4- chlorophenyl)-N-[4- (difluoromethyl)- phenyl]- thieno[2,3- b]pyridine-2- carboxamide |
| 338 | 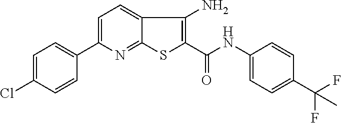 [SMILES: CC(F)(F)c1ccc(NC(=O)c2sc3nc(-c4ccc(Cl)cc4)ccc3c2N)cc1] | C22 H16 Cl F2 N3 O S | 1H NMR in DMSO-d6: δ 9.67 (s, 1H), 8.62 (d, 1H), 8.24 (d, 2H), 8.12 (d, 1H), 7.85 (d, 2H), 7.50-7.62 (m, 6H), 1.98 (t, 3H); Mass Spec: 443.9 (M + H)+ | 3-amino-6-(4- chlorophenyl)-N-[4- (1,1- difluoroethyl)phenyl]- thieno[2,3-b]pyridine- 2-carboxamide |
| 339 | 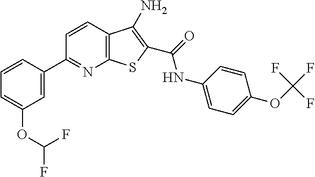 [SMILES: Nc1c(C(=O)Nc2ccc(OC(F)(F)F)cc2)sc2nc(-c3cccc(OC(F)F)c3)ccc12] | C22 H14 F5 N3 O3 S | 1H NMR in DMSO-d6: δ 9.69 (s, 1H), 8.63 (d, 1H), 8.08-8.17 (m, 2H), 7.99 (s, 1H), 7.83 (d, 2H), 7.62 (d, 1H), 7.48 (s, 2H), 7.34-7.39 (m, 4H); Mass Spec: 495.9 (M + H)+ | 3-amino-6-[3- (difluoromethoxy)- phenyl]- N-[4- (trifluoromethoxy)- phenyl]- thieno[2,3- b]pyridine-2- carboxamide |
| 340 | 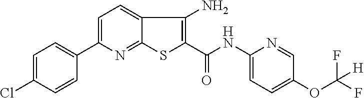 [SMILES: Nc1c(C(=O)Nc2ccc(OC(F)F)cn2)sc2nc(-c3ccc(Cl)cc3)ccc12] | C21 H14 Cl F2 N3 O2 S | 1H NMR in DMSO-d6: δ 9.59 (s, 1H), 8.60 (d, 1H), 8.24 (d, 2H), 8.12 (d, 1H), 7.75 (d, 2H), 7.61 (d, 2H), 6.94-7.45 (m, 5H); Mass Spec: 445.8 (M + H)+ | 3-amino-6-(4- chlorophenyl)-N-[4- (difluoromethoxy)- phenyl]- thieno[2,3- b]pyridine-2- carboxamide |
| 341 | 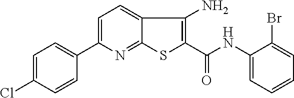 [SMILES: Nc1c(C(=O)Nc2ccccc2Br)sc2nc(-c3ccc(Cl)cc3)ccc12] | C20 H13 Br Cl N3 O S | 1H NMR in DMSO-d6: δ 9.19 (s, 1H), 8.60 (d, 1H), 8.23 (d, 2H), 8.12 (d, 1H), 7.59-7.72 (m, 4H), 7.38-7.46 (m, 3H), 7.16-7.22 (m, 1H); Mass Spec: 457.7 (M + H)+ | 3-amino-N-(2- bromophenyl)-6-(4- chlorophenyl)thieno[2, 3-b]pyridine-2- carboxamide |
| 342 | 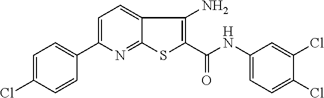 [SMILES: Nc1c(C(=O)Nc2ccc(Cl)c(Cl)c2)sc2nc(-c3ccc(Cl)cc3)ccc12] | C20 H12 Cl3 N3 O S | 1H NMR in DMSO-d6: δ 9.74 (s, 1H), 8.62 (d, 1H), 8.24 (d, 2H), 8.11-8.14 (m, 2H), 7.73 (dd, 1H), 7.55-7.62 (m, 5H); Mass Spec: 447.8 (M + H)+ | 3-amino-6-(4- chlorophenyl)-N-(3,4- dichlorophenyl)- thieno[2,3- b]pyridine-2- carboxamide |
| 343 | 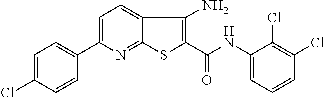 [SMILES: Nc1c(C(=O)Nc2cccc(Cl)c2Cl)sc2nc(-c3ccc(Cl)cc3)ccc12] | C20 H12 Cl3 N3 O S | 1H NMR in DMSO-d6: δ 9.46 (s, 1H), 8.61 (d, 1H), 8.24 (d, 2H), 8.13 (d, 1H), 7.53-7.62 (m, 4H), 7.37-7.46 (m, 3H); Mass Spec: 447.8 (M + H)+ | 3-amino-6-(4- chlorophenyl)-N-(2,3- dichlorophenyl)- thieno[2,3- b]pyridine-2- carboxamide |
| 344 | 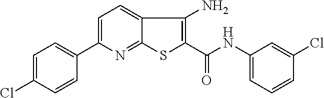 [SMILES: Nc1c(C(=O)Nc2cccc(Cl)c2)sc2nc(-c3ccc(Cl)cc3)ccc12] | C20 H13 Cl2 N3 O S | 1H NMR in DMSO-d6: δ 9.63 (s, 1H), 8.60 (d, 1H), 8.22 (d, 2H), 8.10 (d, 1H), 7.92 (s, 1H), 7.49-7.66 (m, 5H), 7.34 (t, 1H), 7.12 (d, 1H); Mass Spec: 413.8 (M + H)+ | 3-amino-N-(3- chlorophenyl)-6-(4- chlorophenyl)thieno[2, 3-b]pyridine-2- carboxamide |
| 345 | 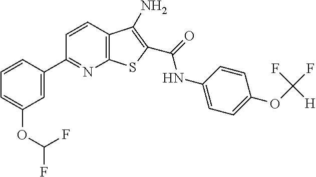 [SMILES: Nc1c(C(=O)Nc2ccc(OC(F)F)cc2)sc2nc(-c3cccc(OC(F)F)c3)ccc12] | C22 H15 F4 N3 O3 S | 1H NMR in DMSO-d6: δ 9.59 (s, 1H), 8.62 (d, 1H), 8.15 (d, 1H), 8.09 (d, 1H), 7.99 (s, 1H), 7.75 (d, 2H), 6.94-7.64 (m, 8H); Mass Spec: 477.9 (M + H)+ | 3-amino-6-[3- (difluoromethoxy)- phenyl]- N-[4- (difluoromethoxy)- phenyl]- thieno[2,3- b]pyridine-2- carboxamide |
| 346 | 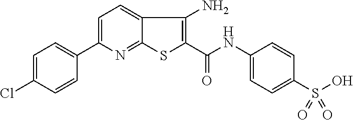 [SMILES: Nc1c(C(=O)Nc2ccc(S(=O)(=O)O)cc2)sc2nc(-c3ccc(Cl)cc3)ccc12] | C20 H14 Cl N3 O4 S2 | 1H NMR in DMSO-d6: δ 9.54 (s, 1H), 8.59 (d, 1H), 8.22 (d, 2H), 8.09 (d, 1H), 7.53-7.66 (m, 6H); Mass Spec: 459.8 (M + H)+ | 4-[[3-amino-6-(4- chlorophenyl)thieno[2, 3-b]pyridine-2- carbonyl]amino]- benzene- sulfonic acid |
| 347 | 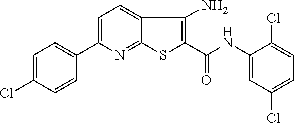 [SMILES: Nc1c(C(=O)Nc2cc(Cl)ccc2Cl)sc2nc(-c3ccc(Cl)cc3)ccc12] | C20 H12 Cl3 N3 O S | 1H NMR in DMSO-d6: δ 9.27 (s, 1H), 8.61 (d, 1H), 8.23 (d, 2H), 8.13 (d, 1H), 7.84 (s, 1H), 7.58-7.62 (m, 3H), 7.44 (s, 2H), 7.34 (dd, 1H); Mass Spec: 447.8 (M + H)+ | 3-amino-6-(4- chlorophenyl)-N-(2,5- dichlorophenyl)- thieno[2,3- b]pyridine-2- carboxamide |
| 348 | 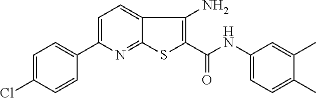 [SMILES: Cc1ccc(NC(=O)c2sc3nc(-c4ccc(Cl)cc4)ccc3c2N)cc1C] | C22 H18 Cl N3 O S | 1H NMR in DMSO-d6: δ 9.32 (s, 1H), 8.57 (d, 1H), 8.21 (d, 2H), 8.09 (d, 1H), 7.58 (d, 2H), 7.48 (s, 1H), 7.38-7.40 (m, 3H), 7.06 (d, 1H), 2.20 (s, 3H), 2.17 (s, H); Mass Spec: 407.9 (M + H)+ | 3-amino-6-(4- chlorophenyl)-N-(3,4- dimethylphenyl)thieno- [2,3-b]pyridine-2- carboxamide |
| 349 | 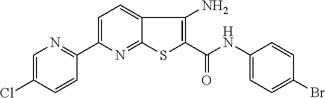 [SMILES: Nc1c(C(=O)Nc2ccc(Br)cc2)sc2nc(-c3ccc(Cl)cn3)ccc12] | C19 H12 Br Cl 5 N4 O S | 1H NMR in DMSO-d6: δ 9.64 (s, 1H), 8.78 (s, 1H), 8.65 (d, 1H), 8.40-8.48 (m, 2H), 8.11 (d, 1H), 7.70 (d, 2H), 7.48-7.52 (m, 4H); Mass Spec: 458.8 (M + H)+ | 3-amino-N-(4- bromophenyl)-6-(5- chloro-2- pyridyl)thieno[2,3- b]pyridine-2- carboxamide |
| 350 | 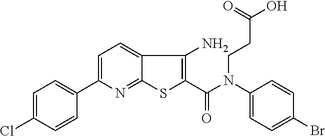 [SMILES: Nc1c(C(=O)N(CCC(=O)O)c2ccc(Br)cc2)sc2nc(-c3ccc(Cl)cc3)ccc12] | C23 H17 Br Cl N3 O3 S | 1H NMR in DMSO-d6: δ 8.51 (d, 1H), 8.13 (d, 2H), 8.00 (d, 1H), 7.66 (d, 2H), 7.51 (d, 2H), 7.35 (d, 2H), 3.92 (t, 2H), 2.53 (t, 2H); Mass Spec: 529.8 (M + H)+ | 3-(N-[3-amino-6-(4- chlorophenyl)- thieno[2, 3-b]pyridine-2- carbonyl]-4-bromo- anilino)propanoic acid |
| 351 | 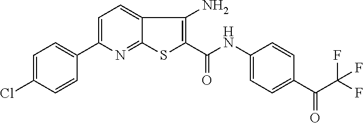 [SMILES: Nc1c(C(=O)Nc2ccc(C(=O)C(F)(F)F)cc2)sc2nc(-c3ccc(Cl)cc3)ccc12] | C22 H13 Cl F3 N3 O2 S | 1H NMR in DMSO-d6: δ 10.05 (s, 1H), 8.64 (d, 1H), 8.23 (d, 2H), 8.06-8.13 (m, 5H), 7.59-7.65 (m, 4H); Mass Spec: 475.8 (M + H)+ | 3-amino-6-(4- chlorophenyl)-N-[4- (2,2,2- trifluoroacetyl)phenyl]- thieno[2,3-b]pyridine- 2-carboxamide |
| 352 | 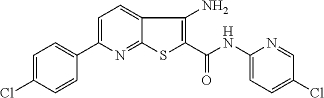 [SMILES: Nc1c(C(=O)Nc2ccc(Cl)cn2)sc2nc(-c3ccc(Cl)cc3)ccc12] | C19 H12 Cl2 N4 O S | 1H NMR in DMSO-d6: δ 10.00 (s, 1H), 8.61 (d, 1H), 8.41 (s, 1H), 8.23 (d, 2H), 8.11 (d, 2H), 7.93 (d, 1H), 7.54-7.60 (m, 4H); Mass Spec: 414.9 (M + H)+ | 3-amino-6-(4- chlorophenyl)-N-(5- chloro-2- pyridyl)thieno[2,3- b]pyridine-2- carboxamide |
| 353 | 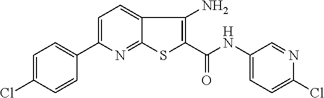 [SMILES: Nc1c(C(=O)Nc2ccc(Cl)nc2)sc2nc(-c3ccc(Cl)cc3)ccc12] | C19 H12 Cl2 N4 O S | 1H NMR in DMSO-d6: δ 9.83 (s, 1H), 8.77 (s, 1H), 8.62 (d, 1H), 8.10-8.24 (m, 4H), 7.48-7.61 (m, 5H); Mass Spec: 414.8 (M + H)+ | 3-amino-6-(4- chlorophenyl)-N-(6- chloro-3- pyridyl)thieno[2,3- b]pyridine-2- carboxamide |
| 354 | 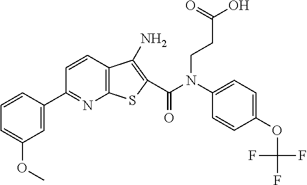 [SMILES: COc1cccc(-c2ccc3c(N)c(C(=O)N(CCC(=O)O)c4ccc(OC(F)(F)F)cc4)sc3n2)c1] | C25 H20 F3 N3 O5 S | 1H NMR in CD3OD: δ 8.28 (d, 1H), 7.78 (d, 1H), 7.48-7.54 (m, 3H), 7.33-7.29 (m, 4H), 6.98 (d, 1H), 4.09 (t, 2H), 3.85 (s, 3H), 2.67 (t, 2H); Mass Spec: 531.9 (M + H)+ | 3-[N-[3-amino-6-(3- methoxyphenyl)thieno- [2,3-b]pyridine-2- carbonyl]-4- (trifluoromethoxy)- anilino]- propanoic acid |
| 355 | 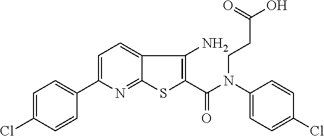 [SMILES: Nc1c(C(=O)N(CCC(=O)O)c2ccc(Cl)cc2)sc2nc(-c3ccc(Cl)cc3)ccc12] | C23 H17 Cl2 N3 O3 | 1H NMR in DMSO-d6: δ 8.50 (d, 1H), 8.12 (d, 2H), 8.00 (d, 1H), 7.49-7.54 (m, 6H), 7.42 (d, 2H), 3.92 (t, 2H), 2.52 (t, 2H); Mass Spec: 485.8 (M + H)+ | 3-(N-[3-amino-6-(4- chlorophenyl)thieno[2, 3-b]pyridine-2- carbonyl]-4-chloro- anilino)propanoic acid |
| 356 | 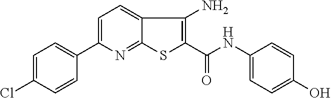 [SMILES: Nc1c(C(=O)Nc2ccc(O)cc2)sc2nc(-c3ccc(Cl)cc3)ccc12] | C20 H14 Cl N3 O2 S | 1H NMR in DMSO-d6: δ 9.27 (d, 2H), 8.57 (d, 1H), 8.23 (d, 2H), 8.10 (d, 1H), 7.60 (d, 2H), 7.42 (d, 2H), 7.34 (s, 2H), 6.72 (d, 2H); Mass Spec: 395.9 (M + H)+ | 3-amino-6-(4- chlorophenyl)-N-(4- hydroxyphenyl)- thieno[2,3- b]pyridine-2- carboxamide |
| 357 | 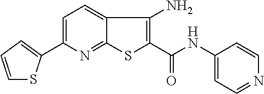 [SMILES: Nc1c(C(=O)Nc2ccncc2)sc2nc(-c3cccs3)ccc12] | C17 H12 N4 O S2 | 1H NMR in CDCl3: δ 8.54 (d, 2H), 7.92 (d, 1H), 7.69-7.73 (m, 2H), 7.57 (d, 2H), 7.48 (d, 1H), 7.24 (s, 1H), 7.15 (t, 1H), 6.25 (s, 2H); | 3-amino-N-(4- pyridyl)-6-(2- thienyl)thieno[2,3- b]pyridine-2- carboxamide |

TABLE 5
Activity against Dengue virus of novel compounds of
the present invention outside the scope of Formula III.

Activity (EC₅₀ in μM)
A: EC₅₀ ≦ 5 μM; B: 5 < EC₅₀ ≦ 25 μM;
C: EC₅₀ > 25 μM; n.d.: not determined

| Cmpd | DENV-1 | DENV-2 | DENV-3 | DENV-4 |
|---|---|---|---|---|
| 281 | n.d. | B | n.d. | n.d. |
| 282 | n.d. | B | n.d. | n.d. |
| 283 | n.d. | A | n.d. | n.d. |
| 284 | A | A | B | C |
| 286 | n.d. | A | n.d. | n.d. |
| 287 | n.d. | B | n.d. | n.d. |
| 288 | A | A | B | A |
| 290 | n.d. | A | n.d. | n.d. |
| 291 | n.d. | B | n.d. | n.d. |
| 292 | A | A | A | A |
| 301 | A | A | B | A |
| 306 | A | A | A | A |
| 316 | n.d. | A | n.d. | n.d. |
| 317 | n.d. | A | n.d. | n.d. |
| 318 | n.d. | A | n.d. | n.d. |
| 319 | n.d. | A | n.d. | n.d. |
| 320 | n.d. | A | n.d. | n.d. |
| 322 | A | A | A | A |
| 323 | n.d. | A | n.d. | n.d. |
| 324 | n.d. | A | n.d. | n.d. |
| 325 | A | A | A | A |
| 326 | n.d. | A | n.d. | n.d. |
| 327 | A | A | A | A |
| 328 | A | A | B | A |
| 329 | A | A | B | A |
| 330 | B | A | B | B |
| 331 | A | A | A | B |
| 332 | A | A | A | A |
| 333 | A | A | A | A |
| 334 | n.d. | A | n.d. | n.d. |
| 335 | A | A | A | A |
| 336 | A | A | A | A |
| 337 | A | A | A | A |
| 338 | A | A | A | A |
| 339 | A | A | A | A |
| 340 | A | A | A | A |
| 341 | A | A | A | A |
| 342 | A | A | A | A |
| 343 | A | A | A | A |
| 344 | A | A | A | A |
| 345 | A | A | A | A |
| 346 | n.d. | A | n.d. | n.d. |
| 347 | n.d. | A | n.d. | n.d. |
| 348 | n.d. | A | n.d. | n.d. |
| 349 | A | A | A | A |
| 350 | A | A | A | A |
| 351 | n.d. | A | n.d. | n.d. |
| 352 | A | A | A | A |
| 353 | A | A | A | A |
| 354 | n.d. | B | n.d. | n.d. |
| 355 | n.d. | A | n.d. | n.d. |
| 356 | n.d. | B | n.d. | n.d. |
| 357 | n.d. | A | n.d. | n.d. |

TABLE 6
Compounds of the present invention.

| | | Molecular | |
|---|---|---|---|
| Cmpd | Chemical Structure | Formula | Chemical Name |
| 364 | 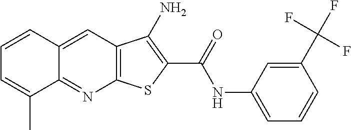 [SMILES: Cc1cccc2cc3c(N)c(C(=O)Nc4cccc(C(F)(F)F)c4)sc3nc12] | C20 H14 F3 N3 O S | 3-amino-8-methyl-N-(3- (trifluoromethyl)- phenyl)thieno[2,3- b]quinoline-2-carboxamide |
| 365 | 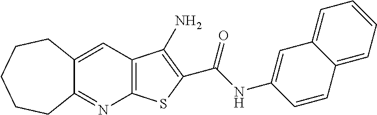 [SMILES: Nc1c(C(=O)Nc2ccc3ccccc3c2)sc2nc3c(cc12)CCCCC3] | C23 H21 N3 O S | 3-amino-N-(naphthalen-2-yl)- 6,7,8,9-tetrahydro- 5H-cyclohepta[b]thieno[3,2- e]pyridine-2- carboxamide |
| 366 | 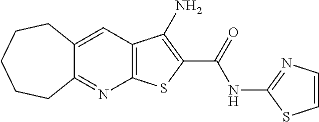 [SMILES: Nc1c(C(=O)Nc2nccs2)sc2nc3c(cc12)CCCCC3] | C16 H16 N4 O S2 | 3-amino-N-(thiazol-2- yl)-6,7,8,9-tetrahydro-5H- cyclohepta[b]thieno[3,2- e]pyridine-2-carboxamide |
| 367 | 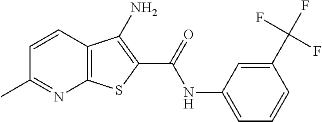 [SMILES: Cc1ccc2c(N)c(C(=O)Nc3cccc(C(F)(F)F)c3)sc2n1] | C16 H12 F3 N3 O S | 3-amino-6-methyl-N-(3- (trifluoromethyl)- phenyl)thieno[2,3- b]pyridine-2- carboxamide |
| 368 | 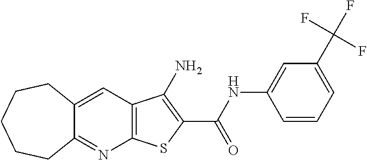 [SMILES: Nc1c(C(=O)Nc2cccc(C(F)(F)F)c2)sc2nc3c(cc12)CCCCC3] | C20 H18 F3 N3 O S | 3-amino-N-(3- (trifluoromethyl)phenyl)- 6,7,8,9- tetrahydro-5H- cyclohepta[b]thieno[3,2- e]pyridine- 2-carboxamide |
| 369 | 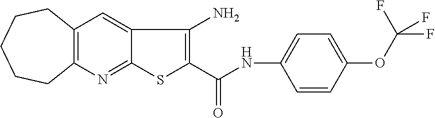 [SMILES: Nc1c(C(=O)Nc2ccc(OC(F)(F)F)cc2)sc2nc3c(cc12)CCCCC3] | C20 H18 F3 N3 O2 S | 3-amino-N-(4- (trifluoromethoxy)phenyl)- 6,7,8,9- tetrahydro-5H- cyclohepta[b]thieno[3,2- e]pyridine- 2-carboxamide |
| 370 | 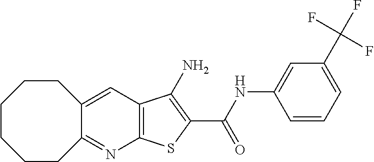 [SMILES: Nc1c(C(=O)Nc2cccc(C(F)(F)F)c2)sc2nc3c(cc12)CCCCCC3] | C21 H2O F3 N3 O S | 3-amino-N-(3- (trifluoromethyl)phenyl)- 5,6,7,8,9,10- hexahydrocycloocta- [b]thieno[3,2- e]pyridine-2-carboxamide |
| 371 | 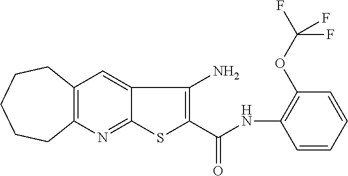 [SMILES: Nc1c(C(=O)Nc2ccccc2OC(F)(F)F)sc2nc3c(cc12)CCCCC3] | C20 H18 F3 N3 O2 S | 3-amino-N-(2- (trifluoromethoxy)phenyl)- 6,7,8,9- tetrahydro-5H- cyclohepta[b]thieno[3,2- e]pyridine- 2-carboxamide |
| 372 | 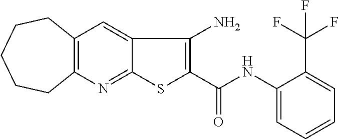 [SMILES: Nc1c(C(=O)Nc2ccccc2C(F)(F)F)sc2nc3c(cc12)CCCCC3] | C20 H18 F3 N3 O S | 3-amino-N-(2- (trifluoromethyl)phenyl)- 6,7,8,9- tetrahydro-5H- cyclohepta[b]thieno[3,2- e]pyridine- 2-carboxamide |
| 373 | 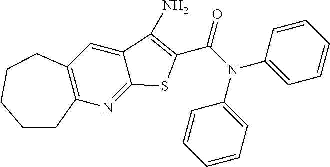 [SMILES: Nc1c(C(=O)N(c2ccccc2)c2ccccc2)sc2nc3c(cc12)CCCCC3] | C25 H23 N3 O S | 3-amino-N,N-diphenyl- 6,7,8,9-tetrahydro-5H- cyclohepta[b]thieno[3,2- e]pyridine-2-carboxamide |
| 374 | 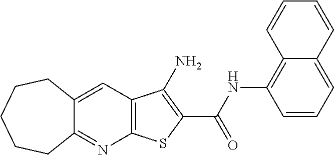 [SMILES: Nc1c(C(=O)Nc2cccc3ccccc23)sc2nc3c(cc12)CCCCC3] | C23 H21 N3 O S | 3-amino-N-(naphthalen-1- yl)-6,7,8,9-tetrahydro- 5H-cyclohepta[b]thieno[3,2- e]pyridine-2- carboxamide |
| 375 | 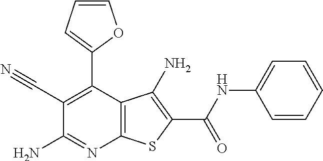 [SMILES: N#Cc1c(N)nc2sc(C(=O)Nc3ccccc3)c(N)c2c1-c1ccco1] | C19 H13 N5 O2 S | 3,6-diamino-5-cyano-4- (2-furyl)-N-phenyl- thieno[2,3-b]pyridine- 2-carboxamide |
| 376 | 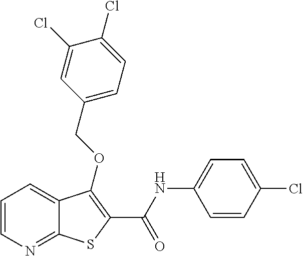 [SMILES: O=C(Nc1ccc(Cl)cc1)c1sc2ncccc2c1OCc1ccc(Cl)c(Cl)c1] | C21 H13 Cl3 N2 O2 S | N-(4-chlorophenyl)-3-[(3,4- dichlorophenyl)methoxy]- thieno[2,3-b]pyridine-2- carboxamide |
| 377 | 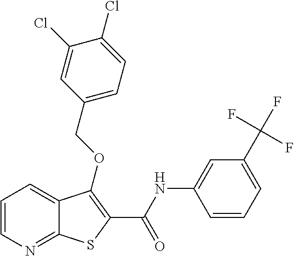 [SMILES: O=C(Nc1cccc(C(F)(F)F)c1)c1sc2ncccc2c1OCc1ccc(Cl)c(Cl)c1] | C22 H13 Cl2 F3 N2 O2 S | 3-[(3,4-dichloro- phenyl)methoxy]- N-[3- (trifluoromethyl)phenyl]- thieno[2,3-b]pyridine-2- carboxamide |
| 378 | 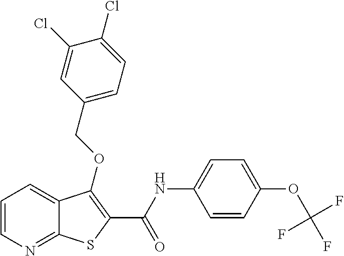 [SMILES: O=C(Nc1ccc(OC(F)(F)F)cc1)c1sc2ncccc2c1OCc1ccc(Cl)c(Cl)c1] | C22 H13 Cl2 F3 N2 O3 S | 3-[(3,4-dichlorophenyl)- methoxy]-N-[4- (trifluoromethoxy)phenyl]- thieno[2,3-b]pyridine-2- carboxamide |
| 379 | 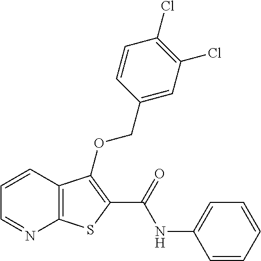 [SMILES: O=C(Nc1ccccc1)c1sc2ncccc2c1OCc1ccc(Cl)c(Cl)c1] | C21 H14 Cl2 N2 O2 S | 3-[(3,4-dichlorophenyl)- methoxy]-N-phenyl- thieno[2,3-b]pyridine- 2-carboxamide |
| 380 | 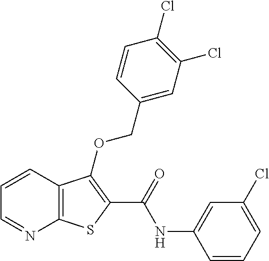 [SMILES: O=C(Nc1cccc(Cl)c1)c1sc2ncccc2c1OCc1ccc(Cl)c(Cl)c1] | C21 H13 Cl3 N2 O2 S | N-(3-chlorophenyl)-3-[(3,4- dichlorophenyl)methoxy]- thieno[2,3-b]pyridine-2- carboxamide |
| 381 | 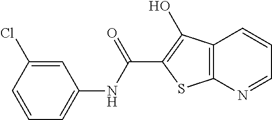 [SMILES: O=C(Nc1cccc(Cl)c1)c1sc2ncccc2c1O] | C14 H9 Cl N2 O2 S | N-(3-chlorophenyl)-3- hydroxy-thieno[2,3- b]pyridine-2-carboxamide |
| 382 | 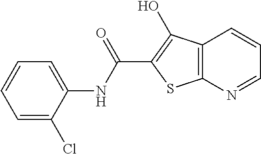 [SMILES: O=C(Nc1ccccc1Cl)c1sc2ncccc2c1O] | C14 H9 Cl N2 O2 S | N-(2-chlorophenyl)-3- hydroxy-thieno[2,3- b]pyridine-2-carboxamide |
| 383 | 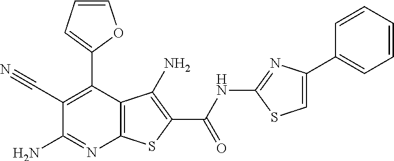 [SMILES: N#Cc1c(N)nc2sc(C(=O)Nc3nc(-c4ccccc4)cs3)c(N)c2c1-c1ccco1] | C22 H14 N6 O2 S2 | 3,6-diamino-5-cyano-4- (2-furyl)-N-(4- phenylthiazol-2- yl)thieno[2,3-b]pyridine-2- carboxamide |
| 384 | 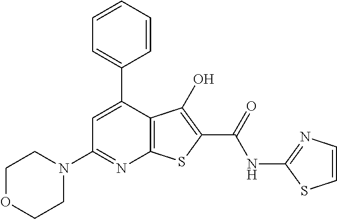 [SMILES: O=C(Nc1nccs1)c1sc2nc(N3CCOCC3)cc(-c3ccccc3)c2c1O] | C21 H18 N4 O3 S2 | 3-hydroxy-6-morpholino-4- phenyl-N-thiazol-2-yl- thieno[2,3- b]pyridine-2-carboxamide |
| 385 | 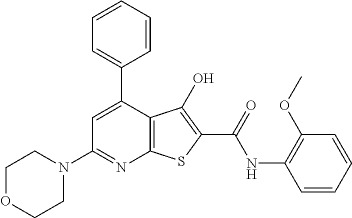 [SMILES: COc1ccccc1NC(=O)c1sc2nc(N3CCOCC3)cc(-c3ccccc3)c2c1O] | C25 H23 N3 O4 S | 3-hydroxy-N-(2- methoxyphenyl)- 6-morpholino-4- phenyl-thieno[2,3- b]pyridine-2-carboxamide |
| 386 | 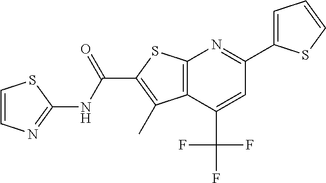 [SMILES: Cc1c(C(=O)Nc2nccs2)sc2nc(-c3cccs3)cc(C(F)(F)F)c12] | C17 H10 F3 N3 O S3 | 3-methyl-N-thiazol-2- yl-6-(2-thienyl)-4- (trifluoromethyl)thieno[2,3- b]pyridine-2- carboxamide |
| 387 | 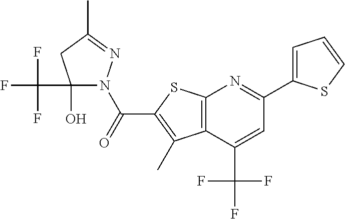 [SMILES: CC1=NN(C(=O)c2sc3nc(-c4cccs4)cc(C(F)(F)F)c3c2C)C(O)(C(F)(F)F)C1] | C19 H13 F6 N3 O2 S2 | [5-hydroxy-3-methyl- 5-(trifluoromethyl)-4H- pyrazol-1-yl]-[3- methyl-6-(2-thienyl)-4- (trifluoromethyl)thieno[2,3- b]pyridin-2- yl]methanone |
| 388 | 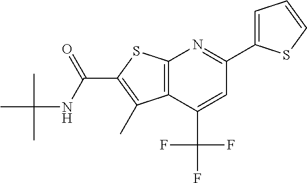 [SMILES: Cc1c(C(=O)NC(C)(C)C)sc2nc(-c3cccs3)cc(C(F)(F)F)c12] | C18 H17 F3 N2 O S2 | N-tert-butyl-3-methyl- 6-(2-thienyl)-4- (trifluoromethyl)thieno[2,3- b]pyridine-2- carboxamide |
| 389 | 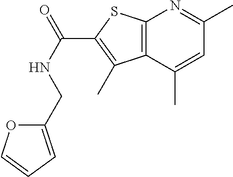 [SMILES: Cc1cc(C)c2c(C)c(C(=O)NCc3ccco3)sc2n1] | C16 H16 N2 O2 S | N-(2-furylmethyl)-3,4,6- trimethyl-thieno[2,3- b]pyridine-2-carboxamide |
| 390 | 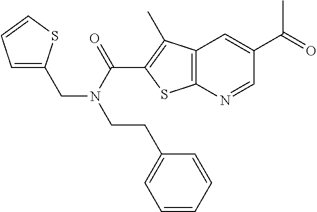 [SMILES: CC(=O)c1cnc2sc(C(=O)N(CCc3ccccc3)Cc3cccs3)c(C)c2c1] | C24 H22 N2 O2 S2 | 5-acetyl-3-methyl-N- phenethyl-N-(2- thienylmethyl)thieno[2,3- b]pyridine-2- carboxamide |
| 391 | 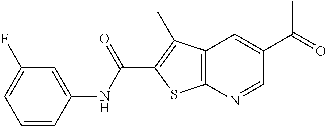 [SMILES: CC(=O)c1cnc2sc(C(=O)Nc3cccc(F)c3)c(C)c2c1] | C17 H13 F N2 O2 S | 5-acetyl-N-(3-fluorophenyl)-3- methyl-thieno[2,3- b]pyridine-2-carboxamide |
| 392 | 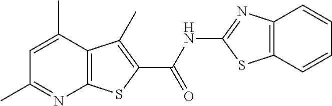 [SMILES: Cc1cc(C)c2c(C)c(C(=O)Nc3nc4ccccc4s3)sc2n1] | C18 H15 N3 O S2 | N-(1,3-benzothiazol-2- yl)-3,4,6-trimethyl- thieno[2,3- b]pyridine-2-carboxamide |
| 393 | 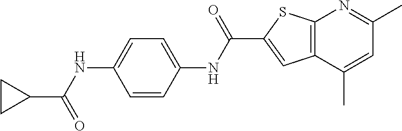 [SMILES: Cc1cc(C)c2cc(C(=O)Nc3ccc(NC(=O)C4CC4)cc3)sc2n1] | C21 H21 N3 O2 S | N-[4-(cyclopropane- carbonylamino)phenyl]-3,4,6- trimethyl-thieno[2,3- b]pyridine-2-carboxamide |
| 394 | 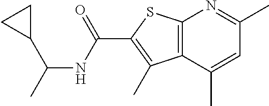 [SMILES: Cc1cc(C)c2c(C)c(C(=O)NC(C)C3CC3)sc2n1] | C16 H20 N2 O S | N-(1-cyclopropylethyl)-3,4,6- trimethyl-thieno[2,3- b]pyridine-2-carboxamide |
| 395 | 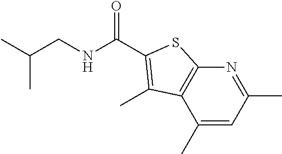 [SMILES: Cc1cc(C)c2c(C)c(C(=O)NCC(C)C)sc2n1] | C15 H20 N2 O S | N-isobutyl-3,4,6-trimethyl- thieno[2,3-b]pyridine- 2-carboxamide |
| 396 | 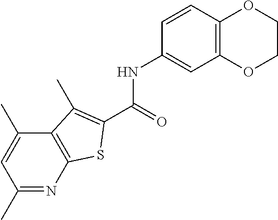 [SMILES: Cc1cc(C)c2c(C)c(C(=O)Nc3ccc4c(c3)OCCO4)sc2n1] | C19 H18 N2 O3 S | N-(2,3-dihydro-1,4- benzodioxin-6-yl)-3,4,6- trimethyl-thieno[2,3- b]pyridine-2-carboxamide |
| 397 | 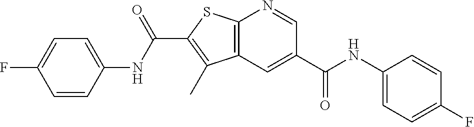 [SMILES: Cc1c(C(=O)Nc2ccc(F)cc2)sc2ncc(C(=O)Nc3ccc(F)cc3)cc12] | C22 H15 F2 N3 O2 S | N2,N5-bis(4-fluorophenyl)- 3-methyl-thieno[2,3- b]pyridine-2,5-dicarboxamide |
| 398 | 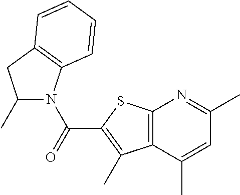 | C20 H20 H20 N2 O S | (2-methylindolin-1-yl)- (3,4,6-trimethylthieno[2,3- b]pyridin-2-yl)methanone |
| 399 | 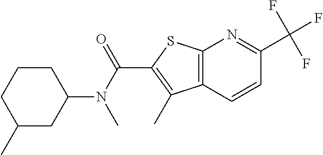 [SMILES: Cc1c(C(=O)N(C)C2CCCC(C)C2)sc2nc(C(F)(F)F)ccc12] | C18 H21 F3 N2 O S | N,3-dimethyl-N-(3- methylcyclohexyl)-6- (trifluoromethyl)thieno- [2,3-b]pyridine-2- carboxamide |
| 400 | 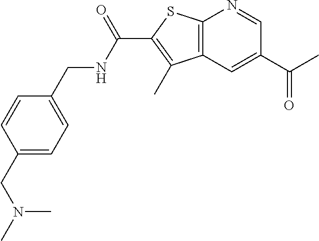 [SMILES: CC(=O)c1cnc2sc(C(=O)NCc3ccc(CN(C)C)cc3)c(C)c2c1] | C21 H23 N3 O2 S | 5-acetyl-N-[[4- (dimethylaminomethyl)- phenyl]methyl]-3-methyl- thieno[2,3-b]pyridine- 2-carboxamide |
| 401 | 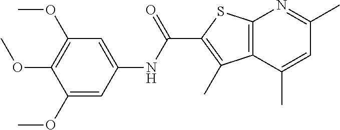 | C20 H22 N2 O4 S | 3,4,6-trimethyl-N-(3,4,5- trimethoxyphenyl)thieno[2,3- b]pyridine-2- carboxamide |
| 402 | 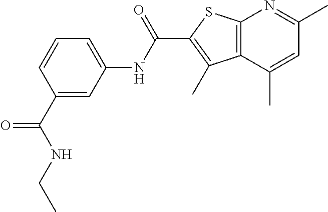 [SMILES: CCNC(=O)c1cccc(NC(=O)c2sc3nc(C)cc(C)c3c2C)c1] | C20 H21 N3 O2 S | N-[3-(ethylcarbamoyl)phenyl]- 3,4,6-trimethyl- thieno[2,3-b]pyridine- 2-carboxamide |
| 403 | 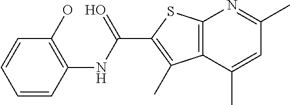 [SMILES: Cc1cc(C)c2c(C)c(C(=O)Nc3ccccc3O)sc2n1] | C17 H16 N2 O2 S | N-(2-hydroxyphenyl)-3,4,6- trimethyl-thieno[2,3- b]pyridine-2-carboxamide |
| 404 | 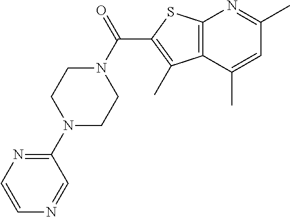 [SMILES: Cc1cc(C)c2c(C)c(C(=O)N3CCN(c4cnccn4)CC3)sc2n1] | C19 H21 N5 O S | (4-pyrazin-2- ylpiperazin-1-yl)-(3,4,6- trimethylthieno[2,3- b]pyridin-2-yl)methanone |
| 405 | 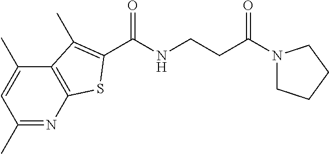 [SMILES: Cc1cc(C)c2c(C)c(C(=O)NCCC(=O)N3CCCC3)sc2n1] | C18 H23 N3 O2 S | 3,4,6-trimethyl-N- (3-oxo-3-pyrrolidin-1-yl- propyl)thieno[2,3- b]pyridine-2-carboxamide |
| 406 | 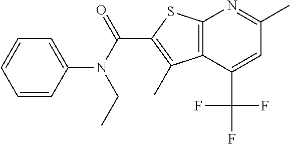 [SMILES: CCN(C(=O)c1sc2nc(C)cc(C(F)(F)F)c2c1C)c1ccccc1] | C19 H17 F3 N2 O S | N-ethyl-3,6-dimethyl-N- phenyl-4- (trifluoromethyl)thieno[2,3- b]pyridine-2- carboxamide |
| 407 | 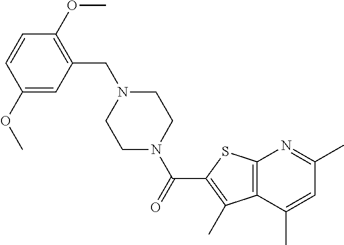 [SMILES: COc1ccc(OC)c(CN2CCN(C(=O)c3sc4nc(C)cc(C)c4c3C)CC2)c1] | C24 H29 N3 O3 S | [4-[(2,5-dimethoxyphenyl)- methyl]piperazin-1-yl]- (3,4,6-trimethylthieno[2,3- b]pyridin-2- yl)methanone |
| 408 | 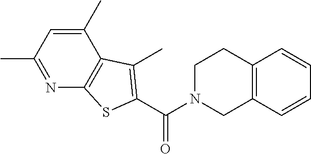 [SMILES: Cc1cc(C)c2c(C)c(C(=O)N3CCc4ccccc4C3)sc2n1] | C20 H20 N2 O S | 3,4-dihydro-1H-isoquinolin- 2-yl-(3,4,6- trimethylthieno[2,3- b]pyridin-2-yl)methanone |
| 409 | 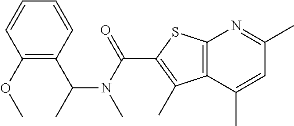 [SMILES: COc1ccccc1C(C)N(C)C(=O)c1sc2nc(C)cc(C)c2c1C] | C21 H24 N2 O2 S | N-[1-(2-methoxy- phenyl)ethyl]-N,3,4,6- tetramethyl-thieno[2,3- b]pyridine-2-carboxamide |
| 410 | 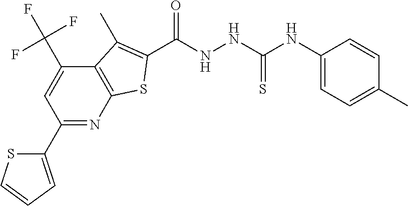 [SMILES: Cc1ccc(NC(=S)NNC(=O)c2sc3nc(-c4cccs4)cc(C(F)(F)F)c3c2C)cc1] | C22 H17 F3 N4 O S3 | 1-[[3-methyl-6-(2-thienyl)-4- (trifluoromethyl)thieno[2,3- b]pyridine-2- carbonyl]amino]-3- (p-tolyl)thiourea |
| 411 | 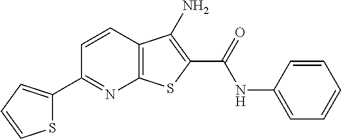 [SMILES: Nc1c(C(=O)Nc2ccccc2)sc2nc(-c3cccs3)ccc12] | C18 H13 N3 O S2 | 3-amino-N-pheny-6- (2-thienyl)thieno[2,3- b]pyridine-2-carboxamide |
| 412 | 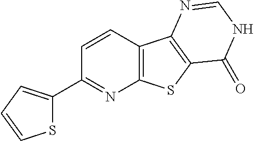 [SMILES: O=c1[nH]cnc2c1sc1nc(-c3cccs3)ccc12] | C13 H7 N3 O S2 | 7-(thiophen-2-yl)pyrido- [3′,2′:4,5]thieno[3,2- d]pyrimidin-4(3H)-one |
| 413 | 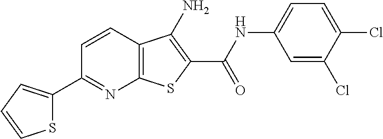 [SMILES: Nc1c(C(=O)Nc2ccc(Cl)c(Cl)c2)sc2nc(-c3cccs3)ccc12] | C18 H11 Cl2 N3 O S2 | 3-amino-N-(3,4- dichlorophenyl)-6-(2- thienyl)thieno[2,3- b]pyridine-2-carboxamide |
| 414 | 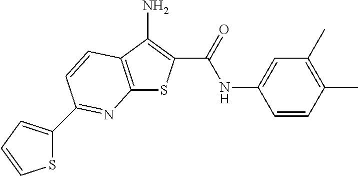 [SMILES: Cc1ccc(NC(=O)c2sc3nc(-c4cccs4)ccc3c2N)cc1C] | C20 H17 N3 O S2 | 3-amino-N-(3,4- dimethylphenyl)-6-(2- thienyl)thieno[2,3- b]pyridine-2-carboxamide |
| 415 | 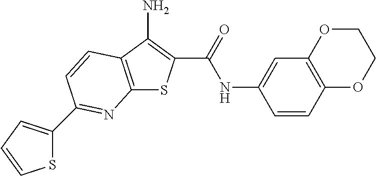 [SMILES: Nc1c(C(=O)Nc2ccc3c(c2)OCCO3)sc2nc(-c3cccs3)ccc12] | C20 H15 N3 O3 S2 | 3-amino-N-(2,3-dihydro- 1,4-benzodioxin-6-yl)-6- (2-thienyl)thieno[2,3-b]- pyridine-2-carboxamide |
| 416 | 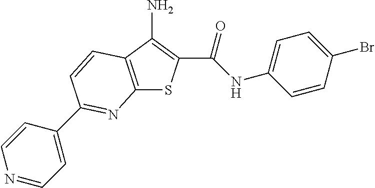 [SMILES: Nc1c(C(=O)Nc2ccc(Br)cc2)sc2nc(-c3ccncc3)ccc12] | C19 H13 Br N4 O S | 3-amino-N-(4- bromophenyl)-6-(4- pyridyl)thieno[2,3-b]- pyridine-2-carboxamide |
| 417 | 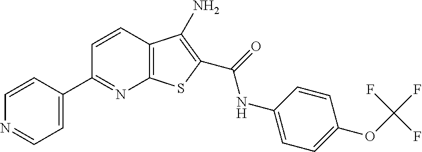 [SMILES: Nc1c(C(=O)Nc2ccc(OC(F)(F)F)cc2)sc2nc(-c3ccncc3)ccc12] | C20 H13 F3 N4 O2 S | 3-amino-6-(4-pyridyl)-N-[4- (trifluoromethoxy)phenyl]- thieno[2,3-b]pyridine-2- carboxamide |
| 418 | 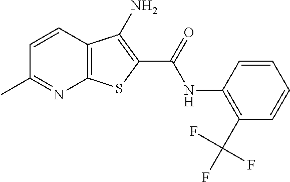 [SMILES: Cc1ccc2c(N)c(C(=O)Nc3ccccc3C(F)(F)F)sc2n1] | C16 H12 F3 N3 O S | 3-amino-6-methyl-N-[2- (trifluoromethyl)phenyl]- thieno[2,3-b]pyridine-2- carboxamide |
| 419 | 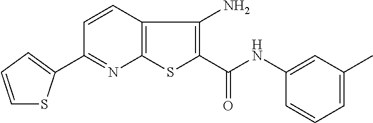 [SMILES: Cc1cccc(NC(=O)c2sc3nc(-c4cccs4)ccc3c2N)c1] | C19 H15 N3 O S2 | 3-amino-N-(m-tolyl)-6-(2- thienyl)thieno[2,3- b]pyridine-2-carboxamide |
| 420 | 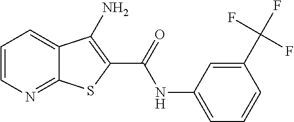 [SMILES: Nc1c(C(=O)Nc2cccc(C(F)(F)F)c2)sc2ncccc12] | C15 H10 F3 N3 O S | 3-amino-N-[3- (trifluoromethyl)- phenyl]thieno[2,3- b]pyridine-2-carboxamide |
| 421 | 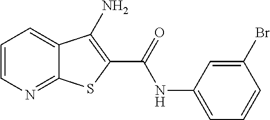 [SMILES: Nc1c(C(=O)Nc2cccc(Br)c2)sc2ncccc12] | C14 H10 Br N3 O S | 3-amino-N-(3-bromophenyl)- thieno[2,3-b]pyridine- 2-carboxamide |
| 422 | 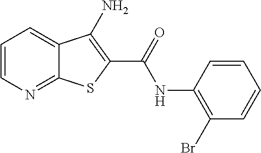 [SMILES: Nc1c(C(=O)Nc2ccccc2Br)sc2ncccc12] | C14 H10 Br N3 O S | 3-amino-N-(2-bromophenyl)- thieno[2,3-b]pyridine- 2-carboxamide |
| 423 | 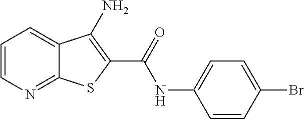 [SMILES: Nc1c(C(=O)Nc2ccc(Br)cc2)sc2ncccc12] | C14 H10 Br N3 O S | 3-amino-N-(4-bromophenyl)- thieno[2,3-b]pyridine- 2-carboxamide |
| 424 | 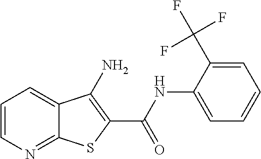 [SMILES: Nc1c(C(=O)Nc2ccccc2C(F)(F)F)sc2ncccc12] | C15 H10 F3 N3 O S | 3-amino-N-[2-(trifluoromethyl)- phenyl]thieno[2,3- b]pyridine-2-carboxamide |
| 425 | 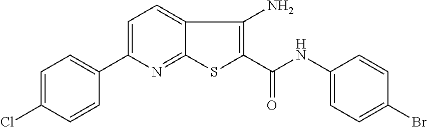 [SMILES: Nc1c(C(=O)Nc2ccc(Br)cc2)sc2nc(-c3ccc(Cl)cc3)ccc12] | C20 H13 Br Cl N3 O S | 3-amino-N-(4- bromophenyl)-6-(4- chlorophenyl)thieno[2,3- b]pyridine-2- carboxamide |
| 426 | 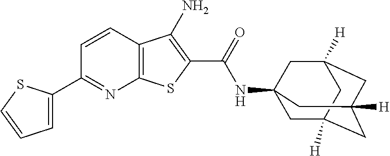 [SMILES: Nc1c(C(=O)N[C@]23C[C@H]4C[C@H](C[C@H](C4)C2)C3)sc2nc(-c3cccs3)ccc12] | C22 H23 N3 O S2 | N-(1-adamantyl)-3-amino- 6-(2-thienyl)thieno[2,3- b]pyridine-2-carboxamide |
| 427 | 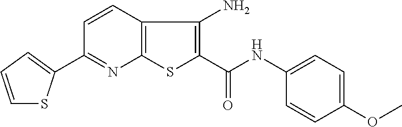 [SMILES: COc1ccc(NC(=O)c2sc3nc(-c4cccs4)ccc3c2N)cc1] | C19 H15 N3 O2 S2 | 3-amino-N-(4- methoxyphenyl)-6-(2- thienyl)thieno[2,3- b]pyridine-2-carboxamide |
| 428 | 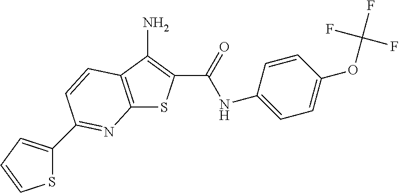 [SMILES: Nc1c(C(=O)Nc2ccc(OC(F)(F)F)cc2)sc2nc(-c3cccs3)ccc12] | C19 H12 F3 N3 O2 S2 | 3-amino-6-(2-thienyl)-N-[4- (trifluoromethoxy)phenyl]- thieno[2,3-b]pyridine-2- carboxamide |
| 429 | 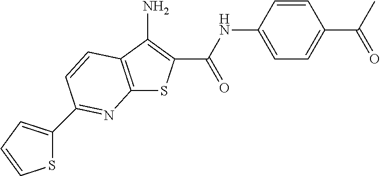 [SMILES: CC(=O)c1ccc(NC(=O)c2sc3nc(-c4cccs4)ccc3c2N)cc1] | C20 H15 N3 O2 S2 | N-(4-acetylphenyl)- 3-amino-6-(2- thienyl)thieno[2,3- b]pyridine-2-carboxamide |
| 430 | 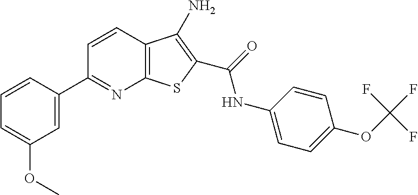 [SMILES: COc1cccc(-c2ccc3c(N)c(C(=O)Nc4ccc(OC(F)(F)F)cc4)sc3n2)c1] | C22 H16 F3 N3 O3 S | 3-amino-6-(3- methoxyphenyl)-N-[4- (trifluoromethoxy)phenyl]- thieno[2,3-b]pyridine-2- carboxamide |
| 431 | 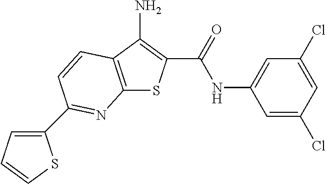 [SMILES: Nc1c(C(=O)Nc2cc(Cl)cc(Cl)c2)sc2nc(-c3cccs3)ccc12] | C18 H11 Cl2 N3 O S2 | 3-amino-N-(3,5- dichlorophenyl)-6-(2- thienyl)thieno[2,3- b]pyridine-2-carboxamide |
| 432 | 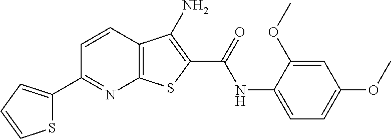 [SMILES: COc1ccc(NC(=O)c2sc3nc(-c4cccs4)ccc3c2N)c(OC)c1] | C20 H17 N3 O3 S2 | 3-amino-N-(2,4- dimethoxyphenyl)-6-(2- thienyl)thieno[2,3- b]pyridine-2-carboxamide |
| 433 | 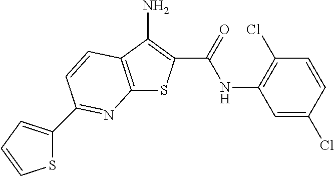 [SMILES: Nc1c(C(=O)Nc2cc(Cl)ccc2Cl)sc2nc(-c3cccs3)ccc12] | C18 H11 Cl2 N3 O S2 | 3-amino-N-(2,5- dichlorophenyl)-6-(2- thienyl)thieno[2,3- b]pyridine-2-carboxamide |
| 434 | 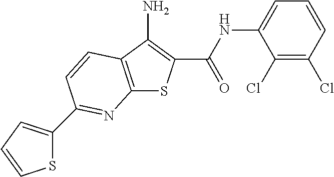 [SMILES: Nc1c(C(=O)Nc2cccc(Cl)c2Cl)sc2nc(-c3cccs3)ccc12] | C18 H11 Cl2 N3 O S2 | 3-amino-N-(2,3- dichlorophenyl)-6-(2- thienyl)thieno[2,3- b]pyridine-2-carboxamide |
| 435 | 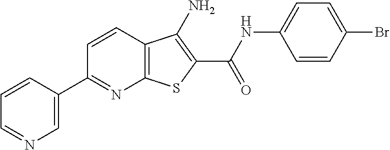 [SMILES: Nc1c(C(=O)Nc2ccc(Br)cc2)sc2nc(-c3cccnc3)ccc12] | C19 H13 Br N4 O S | 3-amino-N-(4- bromophenyl)-6-(3- pyridyl)thieno[2,3- b]pyridine-2-carboxamide |
| 436 | 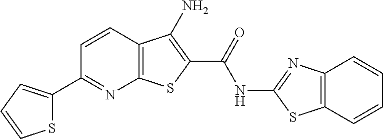 [SMILES: Nc1c(C(=O)Nc2nc3ccccc3s2)sc2nc(-c3cccs3)ccc12] | C19 H12 N4 O S3 | 3-amino-N-(1,3-benzothiazol- 2-yl)-6-(2- thienyl)thieno[2,3- b]pyridine-2-carboxamide |
| 437 | 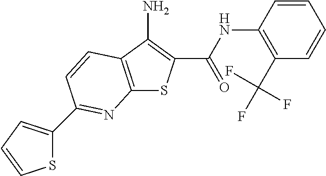 [SMILES: Nc1c(C(=O)Nc2ccccc2C(F)(F)F)sc2nc(-c3cccs3)ccc12] | C19 H12 F3 N3 O S2 | 3-amino-6-(2-thienyl)-N-[2- (trifluoromethyl)phenyl]- thieno[2,3-b]pyridine-2- carboxamide |
| 438 | 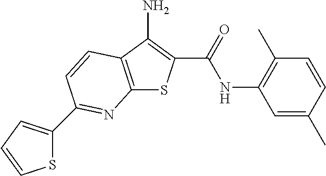 [SMILES: Cc1ccc(C)c(NC(=O)c2sc3nc(-c4cccs4)ccc3c2N)c1] | C20 H17 N3 O S2 | 3-amino-N-(2,5- dimethylphenyl)-6-(2- thienyl)thieno[2,3- b]pyridine-2-carboxamide |
| 439 | 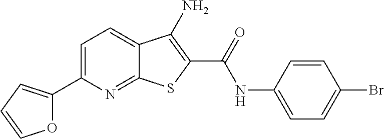 [SMILES: Nc1c(C(=O)Nc2ccc(Br)cc2)sc2nc(-c3ccco3)ccc12] | C18 H12 Br N3 O2 S | 3-amino-N-(4- bromophenyl)-6-(2- furyl)thieno[2,3- b]pyridine-2-carboxamide |
| 440 | 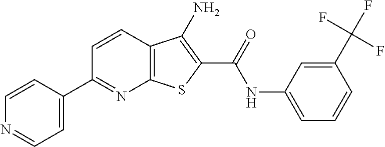 [SMILES: Nc1c(C(=O)Nc2cccc(C(F)(F)F)c2)sc2nc(-c3ccncc3)ccc12] | C20 H13 F3 N4 O S | 3-amino-6-(4-pyridyl)-N-[3- (trifluoromethyl)phenyl]- thieno[2,3-b]pyridine-2- carboxamide |
| 441 | 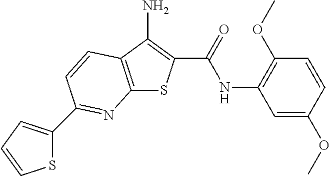 [SMILES: COc1ccc(OC)c(NC(=O)c2sc3nc(-c4cccs4)ccc3c2N)c1] | C20 H17 N3 O3 S2 | 3-amino-N-(2,5- dimethoxyphenyl)-6-(2- thienyl)thieno[2,3- b]pyridine-2-carboxamide |
| 442 | 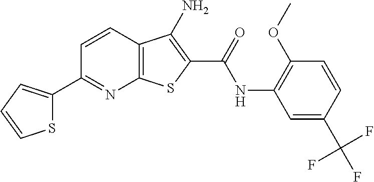 [SMILES: COc1ccc(C(F)(F)F)cc1NC(=O)c1sc2nc(-c3cccs3)ccc2c1N] | C20 H14 F3 N3 O2 S2 | 3-amino-N-[2-methoxy-5- (trifluoromethyl)phenyl]-6- (2-thienyl)thieno[2,3- b]pyridine-2-carboxamide |
| 443 | 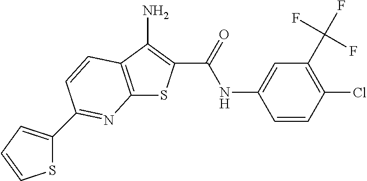 [SMILES: Nc1c(C(=O)Nc2ccc(Cl)c(C(F)(F)F)c2)sc2nc(-c3cccs3)ccc12] | C19 H11 Cl F3 N3 O S2 | 3-amino-N-[4-chloro- 3-(trifluoromethyl)phenyl- 6-(2-thienyl)thieno[2,3- b]pyridine-2-carboxamide |
| 444 | 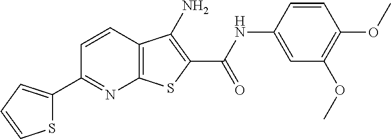 [SMILES: COc1ccc(NC(=O)c2sc3nc(-c4cccs4)ccc3c2N)cc1OC] | C20 H17 N3 O3 S2 | 3-amino-N-(3,4- dimethoxyphenyl)-6-(2- thienyl)thieno[2,3- b]pyridine-2-carboxamide |
| 445 | 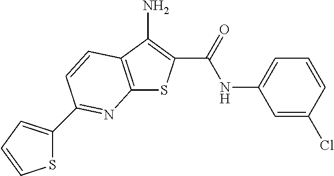 [SMILES: Nc1c(C(=O)Nc2cccc(Cl)c2)sc2nc(-c3cccs3)ccc12] | C18 H12 Cl N3 O S2 | 3-amino-N-(3- chlorophenyl)-6-(2- thienyl)thieno[2,3- b]pyridine-2-carboxamide |
| 446 | 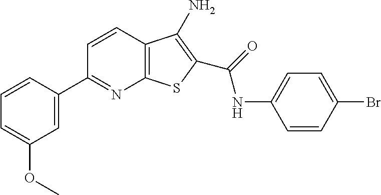 [SMILES: COc1cccc(-c2ccc3c(N)c(C(=O)Nc4ccc(Br)cc4)sc3n2)c1] | C21 H16 Br N3 O2 S | 3-amino-N-(4- bromophenyl)-6-(3- methoxyphenyl)- thieno[2,3-b]pyridine-2- carboxamide |
| 447 | 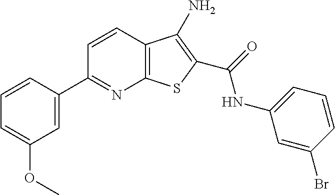 [SMILES: COc1cccc(-c2ccc3c(N)c(C(=O)Nc4cccc(Br)c4)sc3n2)c1] | C21 H16 Br N3 O2 S | 3-amino-N-(3- bromophenyl)-6-(3- methoxyphenyl)thieno[2,3- b]pyridine-2- carboxamide |
| 448 | 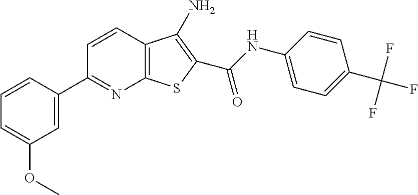 [SMILES: COc1cccc(-c2ccc3c(N)c(C(=O)Nc4ccc(C(F)(F)F)cc4)sc3n2)c1] | C22 H16 F3 N3 | 3-amino-6-(3-methoxyphenyl)- N-[4-(trifluoromethyl)phenyl]- thieno[2,3-b]pyridine-2- carboxamide |
| 449 | 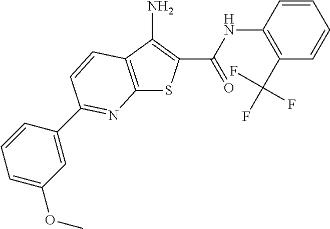 [SMILES: COc1cccc(-c2ccc3c(N)c(C(=O)Nc4ccccc4C(F)(F)F)sc3n2)c1] | C22 H16 F3 N3 O2 S | 3-amino-6-(3- methoxyphenyl)-N-[2- (trifluoromethyl)phenyl]- thieno[2,3-b]pyridine-2- carboxamide |
| 450 | 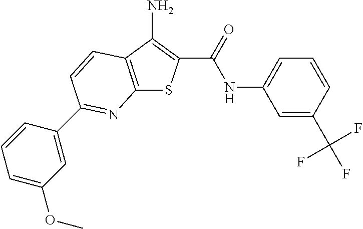 [SMILES: COc1cccc(-c2ccc3c(N)c(C(=O)Nc4cccc(C(F)(F)F)c4)sc3n2)c1] | C22 H16 F3 N3 O2 S | 3-amino-6-(3- methoxyphenyl)-N-[3- (trifluoromethyl)phenyl]- thieno[2,3-b]pyridine-2- carboxamide |
| 451 | 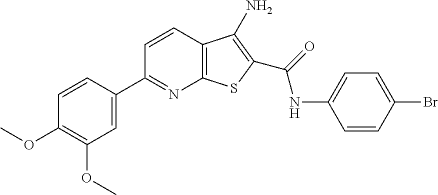 [SMILES: COc1ccc(-c2ccc3c(N)c(C(=O)Nc4ccc(Br)cc4)sc3n2)cc1OC] | C22 H18 Br N3 O3 S | 3-amino-N-(4- bromophenyl)-6-(3,4- dimethoxyphenyl)- thieno[2,3-b]pyridine-2- carboxamide |
| 452 | 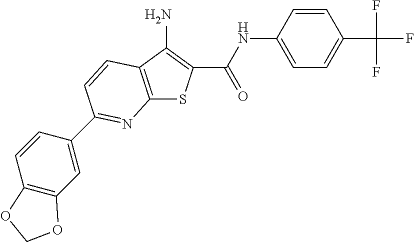 [SMILES: Nc1c(C(=O)Nc2ccc(C(F)(F)F)cc2)sc2nc(-c3ccc4c(c3)OCO4)ccc12] | C22 H14 F3 N3 O3 S | 3-amino-6-(1,3- benzodioxol-5-yl)-N-[4- (trifluoromethyl)phenyl]- thieno[2,3-b]pyridine-2- carboxamide |
| 453 | 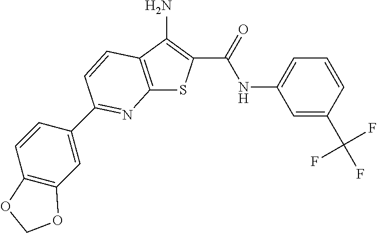 [SMILES: Nc1c(C(=O)Nc2cccc(C(F)(F)F)c2)sc2nc(-c3ccc4c(c3)OCO4)ccc12] | C22 H14 F3 N3 O3 S | 3-amino-6-(1,3- benzodioxol-5-yl)- N-[3-(trifluoromethyl)phenyl]- thieno[2,3-b]pyridine-2- carboxamide |
| 454 | 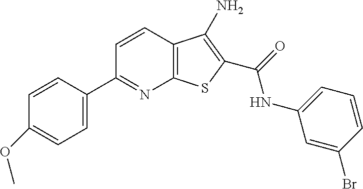 [SMILES: COc1ccc(-c2ccc3c(N)c(C(=O)Nc4cccc(Br)c4)sc3n2)cc1] | C21 H16 Br N3 O2 S | 3-amino-N-(3-bromophenyl)- 6-(4-methoxyphenyl)- thieno[2,3-b]pyridine-2- carboxamide |
| 455 | 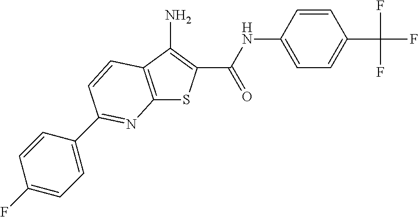 [SMILES: Nc1c(C(=O)Nc2ccc(C(F)(F)F)cc2)sc2nc(-c3ccc(F)cc3)ccc12] | C21 H13 F4 N3 O S | 3-amino-6-(4- fluorophenyl)-N-[4- (trifluoromethyl)phenyl]- thieno[2,3-b]pyridine-2- carboxamide |
| 456 | 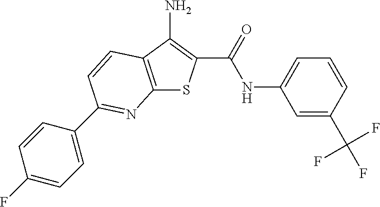 [SMILES: Nc1c(C(=O)Nc2cccc(C(F)(F)F)c2)sc2nc(-c3ccc(F)cc3)ccc12] | C21 H13 F4 N3 O S | 3-amino-6-(4- fluorophenyl)-N-[3- (trifluoromethyl)phenyl]- thieno[2,3-b]pyridine-2- carboxamide |
| 457 | 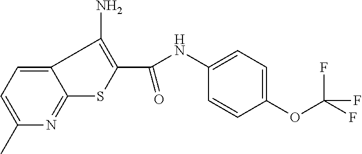 [SMILES: Cc1ccc2c(N)c(C(=O)Nc3ccc(OC(F)(F)F)cc3)sc2n1] | C16 H12 F3 N3 O2 S | 3-amino-6-methyl-N-[4- (trifluoromethoxy)phenyl]- thieno[2,3-b]pyridine-2- carboxamide |
| 458 | 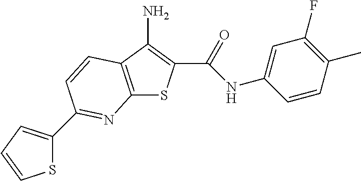 [SMILES: Cc1ccc(NC(=O)c2sc3nc(-c4cccs4)ccc3c2N)cc1F] | C19 H14 F N3 O S2 | 3-amino-N-(3-fluoro- 4-methyl-phenyl)-6-(2- thienyl)thieno[2,3- b]pyridine-2-carboxamide |
| 459 | 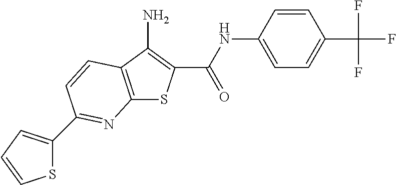 [SMILES: Nc1c(C(=O)Nc2ccc(C(F)(F)F)cc2)sc2nc(-c3cccs3)ccc12] | C19 H12 F3 N3 O S2 | 3-amino-6-(2-thienyl)-N-[4- (trifluoromethyl)phenyl]- thieno[2,3-b]pyridine-2- carboxamide |
| 460 | 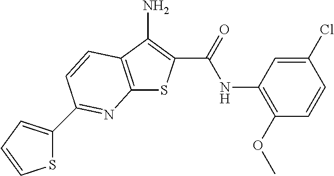 [SMILES: COc1ccc(Cl)cc1NC(=O)c1sc2nc(-c3cccs3)ccc2c1N] | C19 H14 Cl N3 O2 S2 | 3-amino-N-(5-chloro- 2-methoxy-phenyl)-6-(2- thienyl)thieno[2,3- b]pyridine-2-carboxamide |
| 461 | 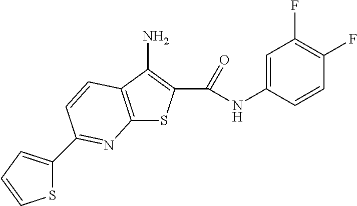 [SMILES: Nc1c(C(=O)Nc2ccc(F)c(F)c2)sc2nc(-c3cccs3)ccc12] | C18 H11 F2 N3 O S2 | 3-amino-N-(3,4- difluorophenyl)-6-(2- thienyl)thieno[2,3- b]pyridine-2-carboxamide |
| 462 | 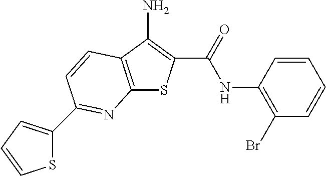 [SMILES: Nc1c(C(=O)Nc2ccccc2Br)sc2nc(-c3cccs3)ccc12] | C18 H12 Br N3 O S2 | 3-amino-N-(2-bromophenyl)- 6-(2-thienyl)thieno[2,3- b]pyridine-2-carboxamide |
| 463 | 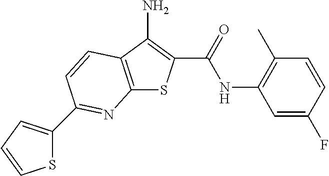 [SMILES: Cc1ccc(F)cc1NC(=O)c1sc2nc(-c3cccs3)ccc2c1N] | C19 H14 F N3 O S2 | 3-amino-N-(5-fluoro- 2-methyl-phenyl)-6-(2- thienyl)thieno[2,3- b]pyridine-2-carboxamide |
| 464 | 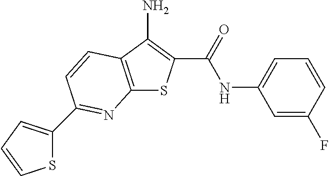 [SMILES: Nc1c(C(=O)Nc2cccc(F)c2)sc2nc(-c3cccs3)ccc12] | C18 H12 F N3 O S2 | 3-amino-N-(3- fluorophenyl)-6-(2- thienyl)thieno[2,3- b]pyridine-2-carboxamide |
| 465 | 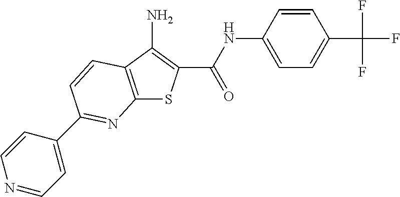 [SMILES: Nc1c(C(=O)Nc2ccc(C(F)(F)F)cc2)sc2nc(-c3ccncc3)ccc12] | C20 H13 F3 N4 O S | 3-amino-6-(4-pyridyl)-N-[4- (trifluoromethyl)phenyl]- thieno[2,3-b]pyridine-2- carboxamide |
| 466 | 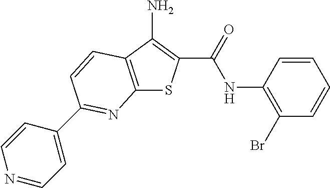 [SMILES: Nc1c(C(=O)Nc2ccccc2Br)sc2nc(-c3ccncc3)ccc12] | C19 H13 Br N4 O S | 3-amino-N-(2- bromophenyl)-6-(4- pyridyl)thieno[2,3- b]pyridine-2-carboxamide |
| 467 | 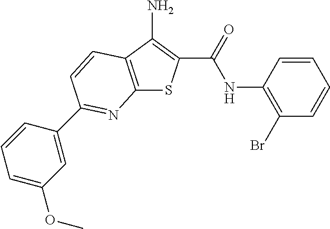 [SMILES: COc1cccc(-c2ccc3c(N)c(C(=O)Nc4ccccc4Br)sc3n2)c1] | C21 H16 Br N3 O2 S | 3-amino-N-(2-bromophenyl)- 6-(3-methoxyphenyl)- thieno[2,3-b]pyridine-2- carboxamide |
| 468 | 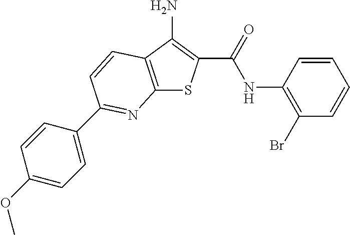 [SMILES: COc1ccc(-c2ccc3c(N)c(C(=O)Nc4ccccc4Br)sc3n2)cc1] | C21 H16 Br N3 O2 S | 3-amino-N-(2- bromophenyl)-6-(4- methoxyphenyl)thieno[2,3- b]pyridine-2- carboxamide |
| 469 | 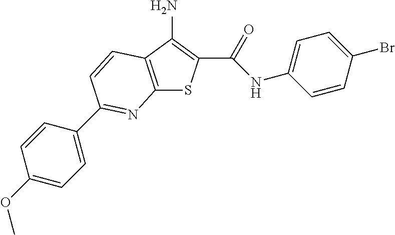 [SMILES: COc1ccc(-c2ccc3c(N)c(C(=O)Nc4ccc(Br)cc4)sc3n2)cc1] | C21 H16 Br N3 O2 S | 3-amino-N-(4- bromophenyl)-6-(4- methoxyphenyl)thieno[2,3- b]pyridine-2- carboxamide |
| 470 | 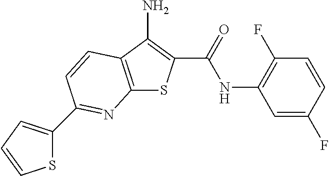 [SMILES: Nc1c(C(=O)Nc2cc(F)ccc2F)sc2nc(-c3cccs3)ccc12] | C18 H11 F2 N3 O S2 | 3-amino-N-(2,5- difluorophenyl)- 6-(2-thienyl)thieno[2,3- b]pyridine-2-carboxamide |
| 471 | 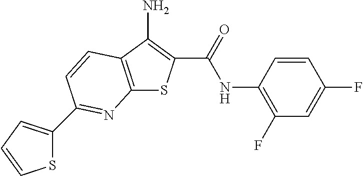 [SMILES: Nc1c(C(=O)Nc2ccc(F)cc2F)sc2nc(-c3cccs3)ccc12] | C18 H11 F2 N3 O S2 | 3-amino-N-(2,4- difluorophenyl)-6-(2- thienyl)thieno[2,3- b]pyridine-2-carboxamide |
| 472 | 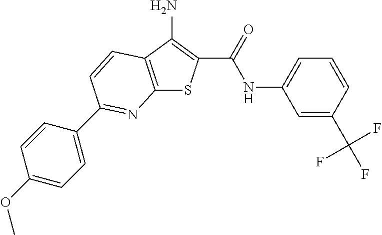 [SMILES: COc1ccc(-c2ccc3c(N)c(C(=O)Nc4cccc(C(F)(F)F)c4)sc3n2)cc1] | C22 H16 F3 N3 O2 S | 3-amino-6-(4- methoxyphenyl)-N-[3- (trifluoromethyl)phenyl]- thieno[2,3-b]pyridine-2- carboxamide |
| 473 | 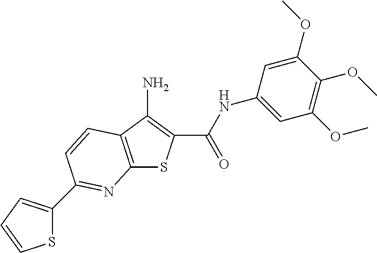 [SMILES: COc1cc(NC(=O)c2sc3nc(-c4cccs4)ccc3c2N)cc(OC)c1OC] | C21 H19 N3 O4 S2 | 3-amino-6-(2-thienyl)- N-(3,4,5- trimethoxyphenyl)- thieno[2,3-b]pyridine-2- carboxamide |
| 474 | 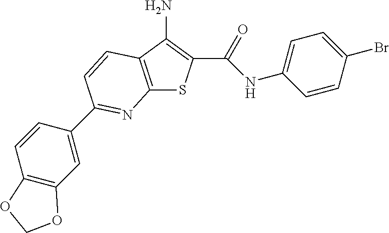 [SMILES: Nc1c(C(=O)Nc2ccc(Br)cc2)sc2nc(-c3ccc4c(c3)OCO4)ccc12] | C21 H14 Br N3 O3 S | 3-amino-6-(1,3- benzodioxol-5-yl)-N-(4- bromophenyl)thieno- [2,3-b]pyridine-2- carboxamide |
| 475 | 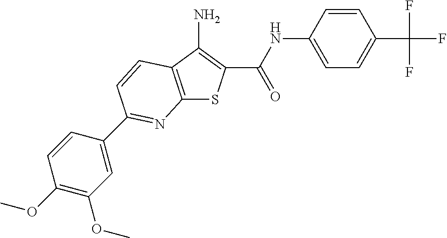 [SMILES: COc1ccc(-c2ccc3c(N)c(C(=O)Nc4ccc(C(F)(F)F)cc4)sc3n2)cc1OC] | C23 H18 F3 N3 O3 S | 3-amino-6-(3,4- dimethoxyphenyl)-N-[4- (trifluoromethyl)phenyl]- thieno[2,3-b]pyridine-2- carboxamide |
| 476 | 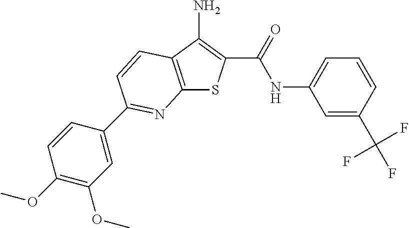 [SMILES: COc1ccc(-c2ccc3c(N)c(C(=O)Nc4cccc(C(F)(F)F)c4)sc3n2)cc1OC] | C23 H18 F3 N3 O3 S | 3-amino-6-(3,4- dimethoxyphenyl)- N-[3-(trifluoromethyl)phenyl]- thieno[2,3-b]pyridine-2- carboxamide |
| 477 | 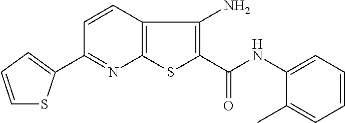 [SMILES: Cc1ccccc1NC(=O)c1sc2nc(-c3cccs3)ccc2c1N] | C19 H15 N3 O S2 | 3-amino-N-(o-tolyl)-6-(2- thienyl)thieno[2,3- b]pyridine-2-carboxamide |
| 478 | 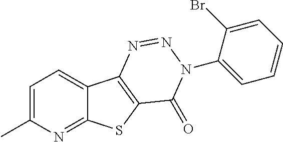 [SMILES: Cc1ccc2c(n1)sc1c(=O)n(-c3ccccc3Br)nnc12] | C15 H9 Br N4 O S | 3-(2-bromophenyl)-7- methylpyrido[3′,2′:4,5]- thieno[3,2-d][1,2,3]triazin- 4(3H)-one |
| 479 | 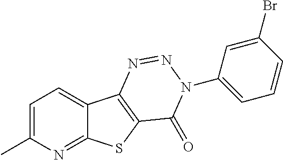 [SMILES: Cc1ccc2c(n1)sc1c(=O)n(-c3cccc(Br)c3)nnc12] | C15 H9 Br N4 O S | 3-(3-bromophenyl)-7- methylpyrido[3′,2′:4,5]- thieno[3,2-d][1,2,3]triazin- 4(3H)-one |
| 480 | 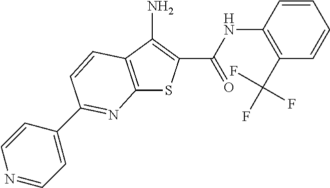 [SMILES: Nc1c(C(=O)Nc2ccccc2C(F)(F)F)sc2nc(-c3ccncc3)ccc12] | C20 H13 F3 N4 O S | 3-amino-6-(4-pyridyl)-N-[2- (trifluoromethyl)phenyl]- thieno[2,3-b]pyridine-2- carboxamide |
| 481 | 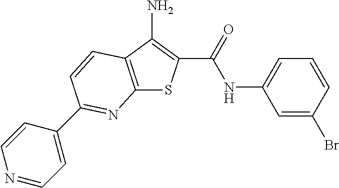 [SMILES: Nc1c(C(=O)Nc2cccc(Br)c2)sc2nc(-c3ccncc3)ccc12] | C19 H13 Br N4 O S | 3-amino-N-(3- bromophenyl)-6-(4- pyridyl)thieno[2,3- b]pyridine-2-carboxamide |
| 482 | 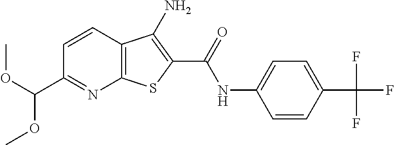 [SMILES: COC(OC)c1ccc2c(N)c(C(=O)Nc3ccc(C(F)(F)F)cc3)sc2n1] | C18 H16 F3 N3 O3 S | 3-amino-6- (dimethoxymethyl)-N-[4- (trifluoromethyl)phenyl]- thieno[2,3-b]pyridine-2- carboxamide |
| 483 | 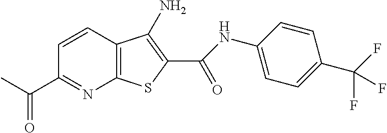 [SMILES: CC(=O)c1ccc2c(N)c(C(=O)Nc3ccc(C(F)(F)F)cc3)sc2n1] | C17 H12 F3 N3 O2 S | 6-acetyl-3-amino-N-[4- (trifluoromethyl)phenyl]- thieno[2,3-b]pyridine-2- carboxamide |
| 484 | 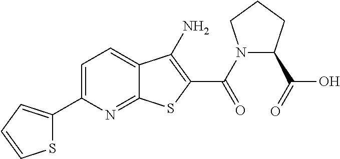 [SMILES: Nc1c(C(=O)N2CCC[C@H]2C(=O)O)sc2nc(-c3cccs3)ccc12] | C17 H15 N3 O3 S2 | 1-[3-amino-6-(2- thienyl)thieno[2,3-b]pyridine-2- carbonyl]pyrrolidine- 2-carboxylic acid |
| 485 | 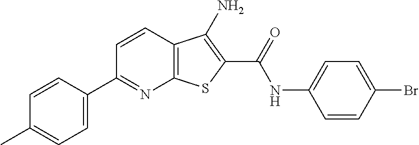 [SMILES: Cc1ccc(-c2ccc3c(N)c(C(=O)Nc4ccc(Br)cc4)sc3n2)cc1] | C21 H16 Br N3 O S | 3-amino-N-(4-bromophenyl)-6- (p-tolyl)thieno[2,3- b]pyridine-2-carboxamide |
| 486 | 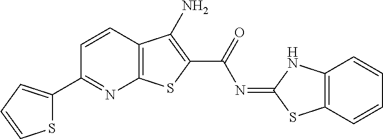 [SMILES: Nc1c(C(=O)/N=c2\[nH]c3ccccc3s2)sc2nc(-c3cccs3)ccc12] | C19 H12 N4 O S3 | (NE)-3-amino-N-(3H-1,3- benzothiazol-2-ylidene)- 6-(2-thienyl)thieno[2,3- b]pyridine-2-carboxamide |
| 487 | 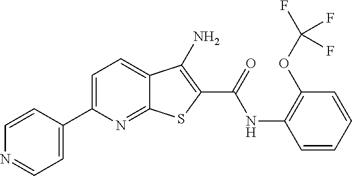 [SMILES: Nc1c(C(=O)Nc2ccccc2OC(F)(F)F)sc2nc(-c3ccncc3)ccc12] | C20 H13 F3 N4 O2 S | 3-amino-6-(4-pyridyl)-N-[2- (trifluoromethoxy)phenyl]- thieno[2,3-b]pyridine-2- carboxamide |
| 488 | 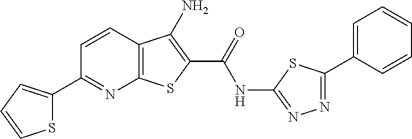 [SMILES: Nc1c(C(=O)Nc2nnc(-c3ccccc3)s2)sc2nc(-c3cccs3)ccc12] | C20 H13 N5 O S3 | 3-amino-N-(5-phenyl-1,3,4- thiadiazol-2-yl)-6-(2- thienyl)thieno[2,3- b]pyridine-2-carboxamide |
| 489 | 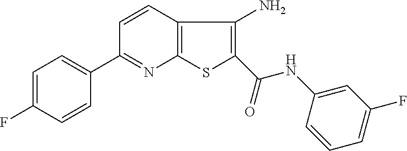 [SMILES: Nc1c(C(=O)Nc2cccc(F)c2)sc2nc(-c3ccc(F)cc3)ccc12] | C20 H13 F2 N3 O S | 3-amino-N-(3- fluorophenyl)-6-(4- fluorophenyl)thieno[2,3- b]pyridine-2-carboxamide |
| 490 | 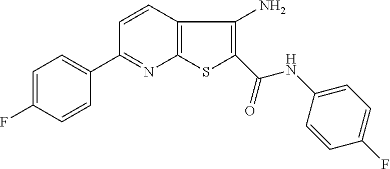 [SMILES: Nc1c(C(=O)Nc2ccc(F)cc2)sc2nc(-c3ccc(F)cc3)ccc12] | C20 H13 F2 N3 O S | 3-amino-N,6-bis(4- fluorophenyl)thieno[2,3- b]pyridine-2-carboxamide |
| 491 | 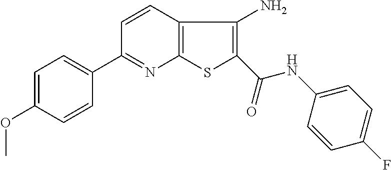 [SMILES: COc1ccc(-c2ccc3c(N)c(C(=O)Nc4ccc(F)cc4)sc3n2)cc1] | C21 H16 F N3 O2 S | 3-amino-N-(4- fluorophenyl)-6-(4- methoxyphenyl)thieno[2,3- b]pyridine-2- carboxamide |
| 492 | 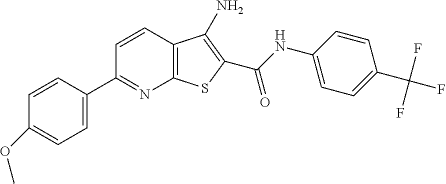 [SMILES: COc1ccc(-c2ccc3c(N)c(C(=O)Nc4ccc(C(F)(F)F)cc4)sc3n2)cc1] | C22 H16 F3 N3 O2 S | 3-amino-6-(4-methoxyphenyl)- N-[4- (trifluoromethyl)phenyl]- thieno[2,3-b]pyridine-2- carboxamide |
| 493 | 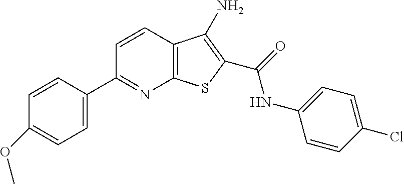 [SMILES: COc1ccc(-c2ccc3c(N)c(C(=O)Nc4ccc(Cl)cc4)sc3n2)cc1] | C21 H16 Cl N3 O2 S | 3-amino-N-(4- chlorophenyl)-6-(4- methoxyphenyl)thieno[2,3- b]pyridine-2- carboxamide |
| 494 | 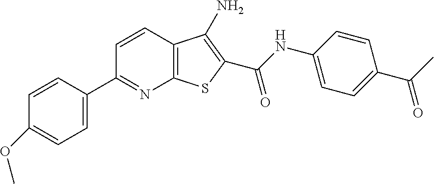 [SMILES: COc1ccc(-c2ccc3c(N)c(C(=O)Nc4ccc(C(C)=O)cc4)sc3n2)cc1] | C23 H19 N3 O3 S | N-(4-acetylphenyl)-3- amino-6-(4- methoxyphenyl)thieno[2,3- b]pyridine-2- carboxamide |
| 495 | 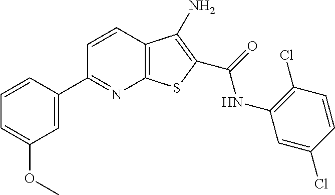 [SMILES: COc1cccc(-c2ccc3c(N)c(C(=O)Nc4cc(Cl)ccc4Cl)sc3n2)c1] | C21 H15 Cl2 N3 O2 S | 3-amino-N-(2,5- dichlorophenyl)-6-(3- methoxyphenyl)thieno[2,3- b]pyridine-2- carboxamide |
| 496 | 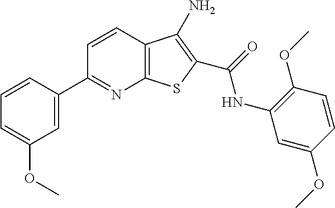 [SMILES: COc1cccc(-c2ccc3c(N)c(C(=O)Nc4cc(OC)ccc4OC)sc3n2)c1] | C23 H21 N3 O4 S | 3-amino-N-(2,5- dimethoxyphenyl)-6-(3- methoxyphenyl)thieno[2,3- b]pyridine-2- carboxamide |
| 497 | 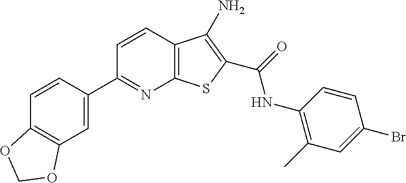 [SMILES: Cc1cc(Br)ccc1NC(=O)c1sc2nc(-c3ccc4c(c3)OCO4)ccc2c1N] | C22 H16 Br N3 O3 S | 3-amino-6-(1,3-benzodioxol-5- yl)-N-(4-bromo-2- methyl-phenyl)thieno[2,3- b]pyridine-2- carboxamide |
| 498 | 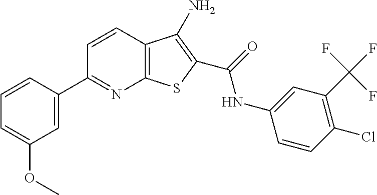 [SMILES: COc1cccc(-c2ccc3c(N)c(C(=O)Nc4ccc(Cl)c(C(F)(F)F)c4)sc3n2)c1] | C22 H15 Cl F3 N3 O2 S | 3-amino-N-[4-chloro-3- (trifluoromethyl)phenyl]- 6-(3-methoxyphenyl)thieno[2,3- b]pyridine-2- carboxamide |
| 499 | 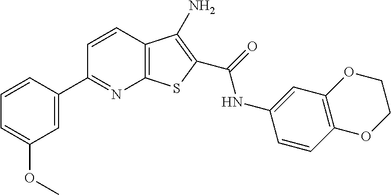 [SMILES: COc1cccc(-c2ccc3c(N)c(C(=O)Nc4ccc5c(c4)OCCO5)sc3n2)c1] | C23 H19 N3 O4 S | 3-amino-N-(2,3-dihydro-1,4- benzodioxin-6-yl)-6- (3-methoxyphenyl)thieno[2,3- b]pyridine-2- carboxamide |
| 500 | 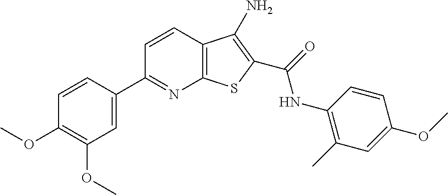 | C24 H23 N3 O4 S | 3-amino-6-(3,4- dimethoxyphenyl)- N-(4-methoxy- 2-methyl-phenyl)thieno[2,3- b]pyridine-2- carboxamide |
| 501 | 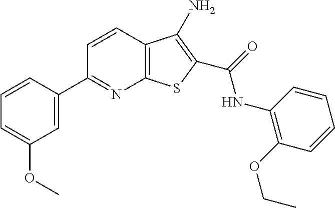 [SMILES: CCOc1ccccc1NC(=O)c1sc2nc(-c3cccc(OC)c3)ccc2c1N] | C23 H21 N3 O3 S | 3-amino-N-(2-ethoxyphenyl)- 6-(3-methoxyphenyl)thieno[2,3- b]pyridine-2- carboxamide |
| 502 | 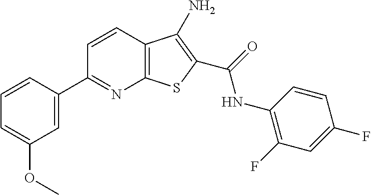 [SMILES: COc1cccc(-c2ccc3c(N)c(C(=O)Nc4ccc(F)cc4F)sc3n2)c1] | C21 H15 F2 N3 O2 S | 3-amino-N-(2,4- difluorophenyl)-6-(3- methoxyphenyl)thieno[2,3- b]pyridine-2- carboxamide |
| 503 | 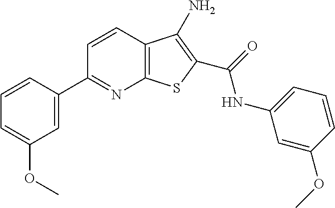 [SMILES: COc1cccc(NC(=O)c2sc3nc(-c4cccc(OC)c4)ccc3c2N)c1] | C22 H19 N3 O3 S | 3-amino-N,6-bis(3- methoxyphenyl)thieno[2,3- b]pyridine-2-carboxamide |
| 504 | 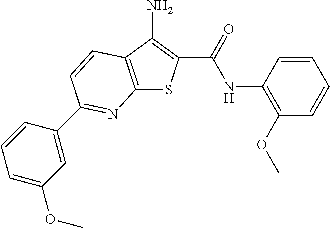 [SMILES: COc1cccc(-c2ccc3c(N)c(C(=O)Nc4ccccc4OC)sc3n2)c1] | C22 H19 N3 O3 S | 3-amino-N-(2- methoxyphenyl)-6-(3- methoxyphenyl)thieno[2,3- b]pyridine-2- carboxamide |
| 505 | 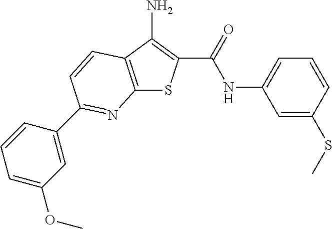 [SMILES: COc1cccc(-c2ccc3c(N)c(C(=O)Nc4cccc(SC)c4)sc3n2)c1] | C22 H19 N3 O2 S2 | 3-amino-6-(3- methoxyphenyl)-N-(3- methylsulfanylphenyl)- thieno[2,3-b]pyridine-2- carboxamide |
| 506 | 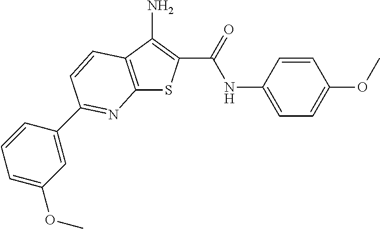 [SMILES: COc1ccc(NC(=O)c2sc3nc(-c4cccc(OC)c4)ccc3c2N)cc1] | C22 H19 N3 O3 S | 3-amino-6-(3- methoxyphenyl)-N-(4- methoxyphenyl)thieno[2,3- b]pyridine-2- carboxamide |
| 507 | 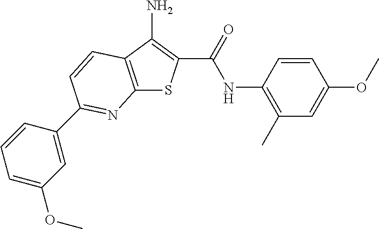 [SMILES: COc1cccc(-c2ccc3c(N)c(C(=O)Nc4ccc(OC)cc4C)sc3n2)c1] | C23 H21 N3 O3 S | 3-amino-N-(4-methoxy-2- methyl-phenyl)-6-(3- methoxyphenyl)thieno[2,3- b]pyridine-2- carboxamide |
| 508 | 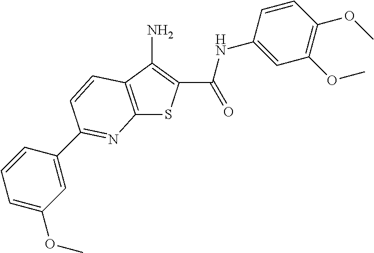 [SMILES: COc1cccc(-c2ccc3c(N)c(C(=O)Nc4ccc(OC)c(OC)c4)sc3n2)c1] | C23 H21 N3 O4 S | 3-amino-N-(3,4- dimethoxyphenyl)-6-(3- methoxyphenyl)thieno[2,3- b]pyridine-2- carboxamide |
| 509 | 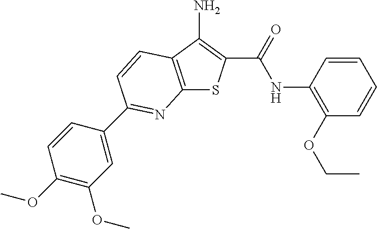 [SMILES: CCOc1ccccc1NC(=O)c1sc2nc(-c3ccc(OC)c(OC)c3)ccc2c1N] | C24 H23 N3 O4 S | 3-amino-6-(3,4- dimethoxyphenyl)-N-(2- ethoxyphenyl)thieno[2,3- b]pyridine-2- carboxamide |
| 510 | 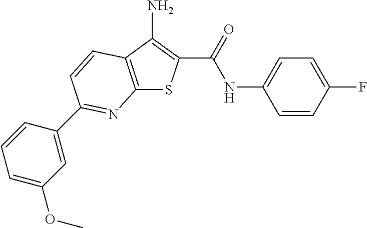 [SMILES: COc1cccc(-c2ccc3c(N)c(C(=O)Nc4ccc(F)cc4)sc3n2)c1] | C21 H16 F N3 O2 S | 3-amino-N-(4- fluorophenyl)-6-(3- methoxyphenyl)thieno[2,3- b]pyridine-2- carboxamide |
| 511 | 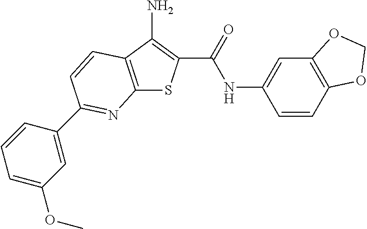 [SMILES: COc1cccc(-c2ccc3c(N)c(C(=O)Nc4ccc5c(c4)OCO5)sc3n2)c1] | C22 H17 N3 O4 S | 3-amino-N-(1,3-benzodioxol- 5-yl)-6-(3- methoxyphenyl)thieno[2,3- b]pyridine-2- carboxamide |
| 512 | 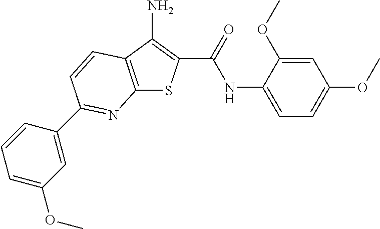 [SMILES: COc1cccc(-c2ccc3c(N)c(C(=O)Nc4ccc(OC)cc4OC)sc3n2)c1] | C23 H21 N3 O4 S | 3-amino-N-(2,4- dimethoxyphenyl)-6-(3- methoxyphenyl)thieno[2,3- b]pyridine-2- carboxamide |
| 513 | 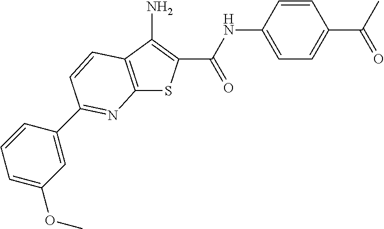 [SMILES: COc1cccc(-c2ccc3c(N)c(C(=O)Nc4ccc(C(C)=O)cc4)sc3n2)c1] | C23 H19 N3 O3 S | N-(4-acetylphenyl)-3- amino-6-(3- methoxyphenyl)thieno[2,3- b]pyridine-2- carboxamide |
| 514 | 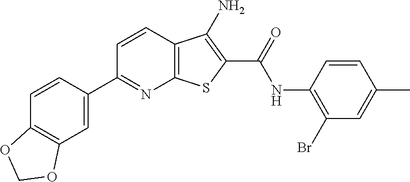 [SMILES: Cc1ccc(NC(=O)c2sc3nc(-c4ccc5c(c4)OCO5)ccc3c2N)c(Br)c1] | C22 H16 Br N3 O3 S | 3-amino-6-(1,3-benzodioxol- 5-yl)-N-(2-bromo-4- methyl-phenyl)thieno[2,3- b]pyridine-2- carboxamide |
| 515 | 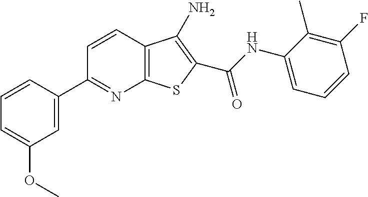 [SMILES: COc1cccc(-c2ccc3c(N)c(C(=O)Nc4cccc(F)c4C)sc3n2)c1] | C22 H18 F N3 O2 S | 3-amino-N-(3-fluoro- 2-methyl-phenyl)-6-(3- methoxyphenyl)thieno[2,3- b]pyridine-2- carboxamide |
| 516 | 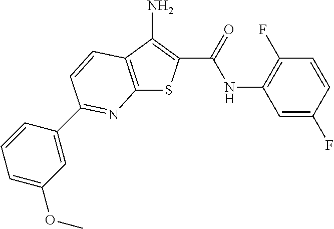 [SMILES: COc1cccc(-c2ccc3c(N)c(C(=O)Nc4cc(F)ccc4F)sc3n2)c1] | C21 H15 F2 N3 O2 S | 3-amino-N-(2,5- difluorophenyl)-6-(3- methoxyphenyl)thieno[2,3- b]pyridine-2- carboxamide |
| 517 | 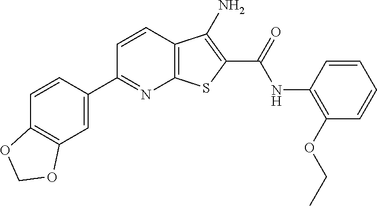 [SMILES: CCOc1ccccc1NC(=O)c1sc2nc(-c3ccc4c(c3)OCO4)ccc2c1N] | C23 H19 N3 O4 S | 3-amino-6-(1,3-benzodioxol- 5-yl)-N-(2- ethoxyphenyl)thieno[2,3- b]pyridine-2- carboxamide |
| 518 | 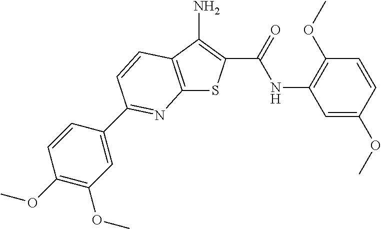 [SMILES: COc1ccc(OC)c(NC(=O)c2sc3nc(-c4ccc(OC)c(OC)c4)ccc3c2N)c1] | C24 H23 N3 O5 S | 3-amino-N-(2,5- dimethoxyphenyl)-6-(3,4- dimethoxyphenyl)thieno[2,3- b]pyridine-2- carboxamide |
| 519 | 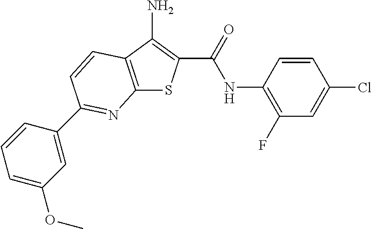 [SMILES: COc1cccc(-c2ccc3c(N)c(C(=O)Nc4ccc(Cl)cc4F)sc3n2)c1] | C21 H15 Cl F N3 O2 S | 3-amino-N-(4-chloro-2- fluoro-phenyl)-6-(3- methoxyphenyl)thieno[2,3- b]pyridine-2- carboxamide |
| 520 | 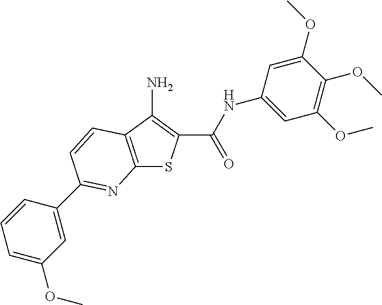 [SMILES: COc1cccc(-c2ccc3c(N)c(C(=O)Nc4cc(OC)c(OC)c(OC)c4)sc3n2)c1] | C24 H23 N3 O5 S | 3-amino-6-(3- methoxyphenyl)-N-(3,4,5- trimethoxyphenyl)- thieno[2,3-b]pyridine-2- carboxamide |
| 521 | 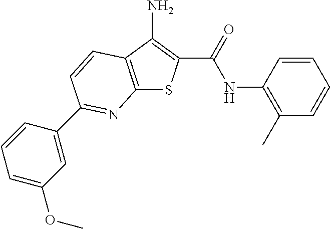 [SMILES: COc1cccc(-c2ccc3c(N)c(C(=O)Nc4ccccc4C)sc3n2)c1] | C22 H19 N3 O2 S3 | amino-6-(3- methoxyphenyl)-N-(o- tolyl)thieno[2,3- b]pyridine-2-carboxamide |
| 522 | 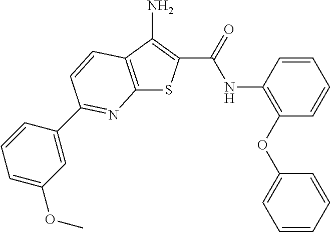 [SMILES: COc1cccc(-c2ccc3c(N)c(C(=O)Nc4ccccc4Oc4ccccc4)sc3n2)c1] | C27 H21 N3 O3 S | 3-amino-6-(3- methoxyphenyl)-N-(2- phenoxyphenyl)thieno[2,3- b]pyridine-2- carboxamide |
| 523 | 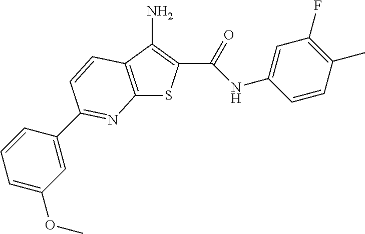 [SMILES: COc1cccc(-c2ccc3c(N)c(C(=O)Nc4ccc(C)c(F)c4)sc3n2)c1] | C22 H18 F N3 O2 S | 3-amino-N-(3-fluoro-4- methyl-phenyl)-6-(3- methoxyphenyl)thieno[2,3- b]pyridine-2- carboxamide |
| 524 | 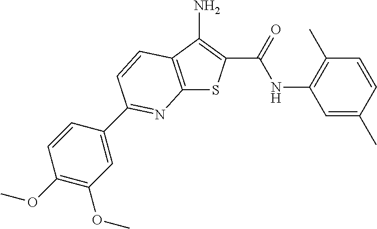 | C24 H23 N3 O3 S | 3-amino-6-(3,4- dimethoxyphenyl)-N-(2,5- dimethylphenyl)thieno[2,3- b]pyridine-2- carboxamide |
| 525 | 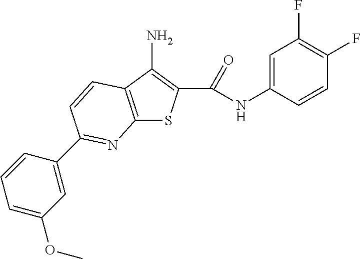 [SMILES: COc1cccc(-c2ccc3c(N)c(C(=O)Nc4ccc(F)c(F)c4)sc3n2)c1] | C21 H15 F2 N3 O2 S | 3-amino-N-(3,4- difluorophenyl)-6-(3- methoxyphenyl)thieno[2,3- b]pyridine-2- carboxamide |
| 526 | 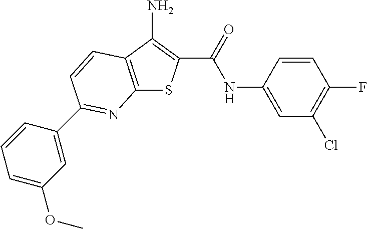 [SMILES: COc1cccc(-c2ccc3c(N)c(C(=O)Nc4ccc(F)c(Cl)c4)sc3n2)c1] | C21 H15 Cl F N3 O2 S | 3-amino-N-(3-chloro-4- fluoro-phenyl)-6-(3- methoxyphenyl)thieno[2,3- b]pyridine-2- carboxamide |
| 527 | 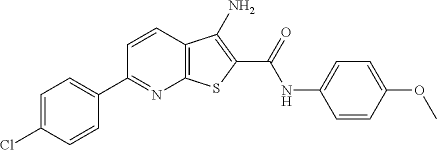 [SMILES: COc1ccc(NC(=O)c2sc3nc(-c4ccc(Cl)cc4)ccc3c2N)cc1] | C21 H16 Cl N3 O2 S | 3-amino-6-(4- chlorophenyl)-N-(4- methoxyphenyl)thieno[2,3- b]pyridine-2- carboxamide |
| 528 | 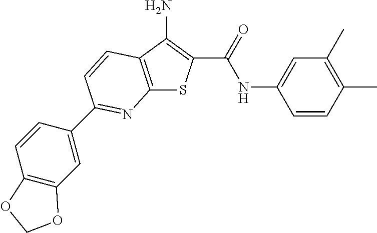 [SMILES: Cc1ccc(NC(=O)c2sc3nc(-c4ccc5c(c4)OCO5)ccc3c2N)cc1C] | C23 H19 N3 O3 S | 3-amino-6-(1,3- benzodioxol-5-yl)-N-(3,4- dimethylphenyl)thieno[2,3- b]pyridine-2- carboxamide |
| 529 | 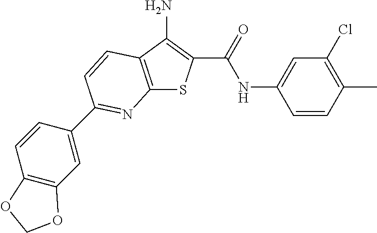 [SMILES: Cc1ccc(NC(=O)c2sc3nc(-c4ccc5c(c4)OCO5)ccc3c2N)cc1Cl] | C22 H16 Cl N3 O3 S | 3-amino-6-(1,3-benzodioxol- 5-yl)-N-(3-chloro-4- methyl-phenyl)thieno[2,3- b]pyridine-2- carboxamide |
| 530 | 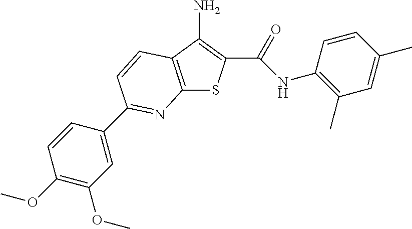 | C24 H23 N3 O3 S | 3-amino-6-(3,4- dimethoxyphenyl)-N-(2,4- dimethylphenyl)thieno[2,3- b]pyridine-2- carboxamide |
| 531 | 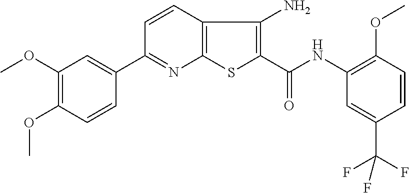 [SMILES: COc1ccc(C(F)(F)F)cc1NC(=O)c1sc2nc(-c3ccc(OC)c(OC)c3)ccc2c1N] | C24 H20 F3 N3 O4 S | 3-amino-6-(3,4- dimethoxyphenyl)-N- [2-methoxy- 5-(trifluoromethyl)phenyl]- thieno[2,3-b]pyridine-2- carboxamide |
| 532 | 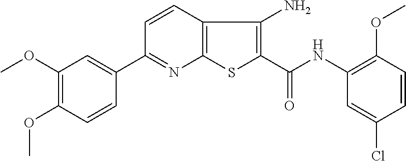 | C23 H20 Cl N3 O4 S | 3-amino-N-(5-chloro-2- methoxy-phenyl)-6-(3,4- dimethoxyphenyl)thieno[2,3- b]pyridine-2- carboxamide |
| 533 | 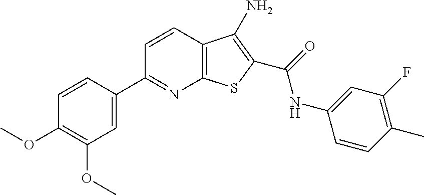 | C23 H20 F N3 O3 S | 3-amino-6-(3,4- dimethoxyphenyl)- N-(3-fluoro-4- methyl-phenyl)thieno[2,3- b]pyridine-2- carboxamide |
| 534 | 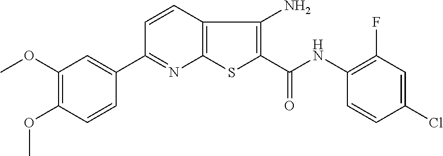 [SMILES: COc1ccc(-c2ccc3c(N)c(C(=O)Nc4ccc(Cl)cc4F)sc3n2)cc1OC] | C22 H17 Cl F N3 O3 S | 3-amino-N-(4-chloro-2-fluoro- phenyl)-6-(3,4- dimethoxyphenyl)thieno[2,3- b]pyridine-2- carboxamide |
| 535 | 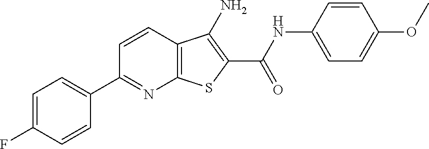 [SMILES: COc1ccc(NC(=O)c2sc3nc(-c4ccc(F)cc4)ccc3c2N)cc1] | C21 H16 F N3 O2 S | 3-amino-6-(4-fluorophenyl)- N-(4- methoxyphenyl)thieno[2,3- b]pyridine-2- carboxamide |
| 536 | 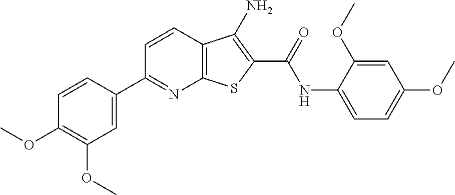 | C24 H23 N3 O5 S | 3-amino-N-(2,4- dimethoxyphenyl)-6-(3,4- dimethoxyphenyl)thieno[2,3- b]pyridine-2- carboxamide |
| 537 | 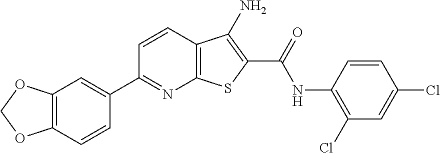 [SMILES: Nc1c(C(=O)Nc2ccc(Cl)cc2Cl)sc2nc(-c3ccc4c(c3)OCO4)ccc12] | C21 H13 Cl2 N3 O3 S | 3-amino-6-(1,3-benzodioxol- 5-yl)-N-(2,4- dichlorophenyl)thieno[2,3- b]pyridine-2- carboxamide |
| 538 | 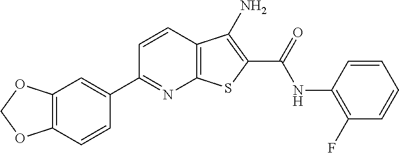 [SMILES: Nc1c(C(=O)Nc2ccccc2F)sc2nc(-c3ccc4c(c3)OCO4)ccc12] | C21 H14 F N3 O3 S | 3-amino-6-(1,3-benzodioxol-5- yl)-N-(2- fluorophenyl)thieno[2,3- b]pyridine-2-carboxamide |
| 539 | 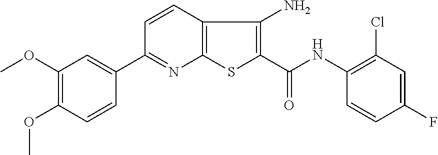 [SMILES: COc1ccc(-c2ccc3c(N)c(C(=O)Nc4ccc(F)cc4Cl)sc3n2)cc1OC] | C22 H17 Cl F N3 O3 S | 3-amino-N-(2-chloro-4-fluoro- phenyl)-6-(3,4- dimethoxyphenyl)thieno[2,3- b]pyridine-2- carboxamide |
| 540 | 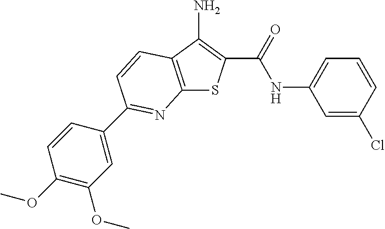 [SMILES: COc1ccc(-c2ccc3c(N)c(C(=O)Nc4cccc(Cl)c4)sc3n2)cc1OC] | C22 H18 Cl N3 O3 S | 3-amino-N-(3-chlorophenyl)- 6-(3,4- dimethoxyphenyl)thieno[2,3- b]pyridine-2- carboxamide |
| 541 | 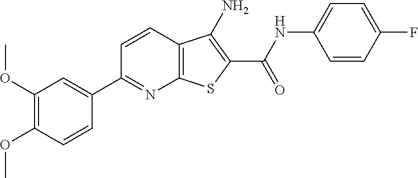 [SMILES: COc1ccc(-c2ccc3c(N)c(C(=O)Nc4ccc(F)cc4)sc3n2)cc1OC] | C22 H18 F N3 O3 S | 3-amino-6-(3,4- dimethoxyphenyl)-N-(4- fluorophenyl)thieno[2,3- b]pyridine-2-carboxamide |
| 542 | 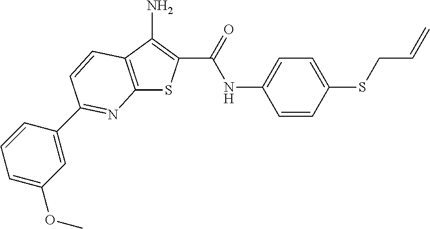 [SMILES: C=CCSc1ccc(NC(=O)c2sc3nc(-c4cccc(OC)c4)ccc3c2N)cc1] | C24 H21 N3 O2 S2 | N-(4-allylsulfanylphenyl)- 3-amino-6-(3- methoxyphenyl)thieno[2,3- b]pyridine-2- carboxamide |
| 543 | 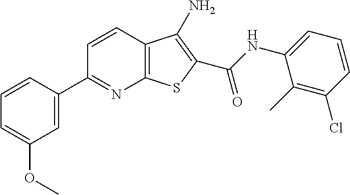 [SMILES: COc1cccc(-c2ccc3c(N)c(C(=O)Nc4cccc(Cl)c4C)sc3n2)c1] | C22 H18 Cl N3 O2 S | 3-amino-N-(3-chloro-2-methyl- phenyl)-6-(3- methoxyphenyl)thieno[2,3- b]pyridine-2- carboxamide |
| 544 | 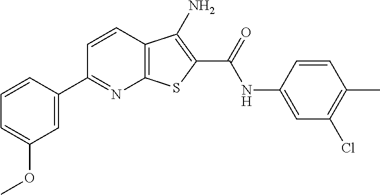 [SMILES: COc1cccc(-c2ccc3c(N)c(C(=O)Nc4ccc(C)c(Cl)c4)sc3n2)c1] | C22 H18 Cl N3 O2 S | 3-amino-N-(3-chloro-4-methyl- phenyl)-6-(3- methoxyphenyl)thieno[2,3- b]pyridine-2- carboxamide |
| 545 | 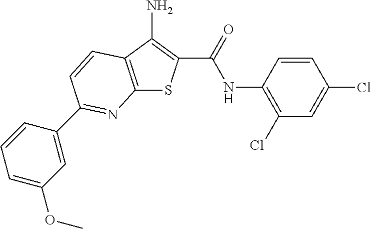 [SMILES: COc1cccc(-c2ccc3c(N)c(C(=O)Nc4ccc(Cl)cc4Cl)sc3n2)c1] | C21 H15 Cl2 N3 O2 S | 3-amino-N-(2,4- dichlorophenyl)-6-(3- methoxyphenyl)thieno[2,3- b]pyridine-2- carboxamide |
| 546 | 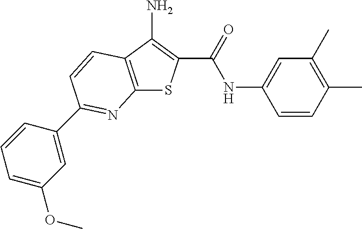 [SMILES: COc1cccc(-c2ccc3c(N)c(C(=O)Nc4ccc(C)c(C)c4)sc3n2)c1] | C23 H21 N3 O2 S | 3-amino-N-(3,4- dimethylphenyl)-6-(3- methoxyphenyl)thieno[2,3- b]pyridine-2- carboxamide |
| 547 | 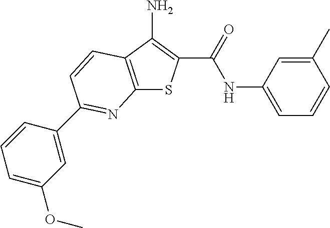 [SMILES: COc1cccc(-c2ccc3c(N)c(C(=O)Nc4cccc(C)c4)sc3n2)c1] | C22 H19 N3 O2 S | 3-amino-6-(3- methoxyphenyl)-N-(m- tolyl)thieno[2,3- b]pyridine-2-carboxamide |
| 548 | 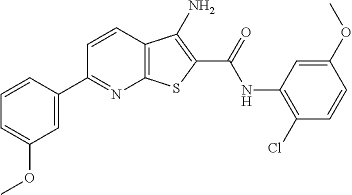 [SMILES: COc1cccc(-c2ccc3c(N)c(C(=O)Nc4cc(OC)ccc4Cl)sc3n2)c1] | C22 H18 Cl N3 O3 S | 3-amino-N-(2-chloro-5- methoxy-phenyl)-6-(3- methoxyphenyl)thieno[2,3- b]pyridine-2- carboxamide |
| 549 | 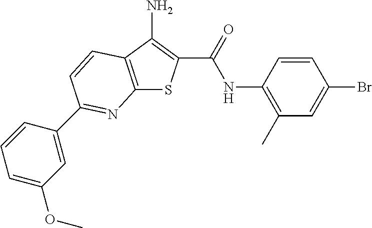 [SMILES: COc1cccc(-c2ccc3c(N)c(C(=O)Nc4ccc(Br)cc4C)sc3n2)c1] | C22 H18 Br N3 O2 S | 3-amino-N-(4-bromo-2-methyl- phenyl)-6-(3- methoxyphenyl)thieno[2,3- b]pyridine-2- carboxamide |
| 550 | 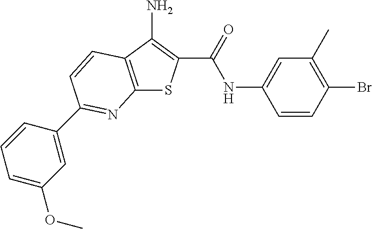 [SMILES: COc1cccc(-c2ccc3c(N)c(C(=O)Nc4ccc(Br)c(C)c4)sc3n2)c1] | C22 H18 Br N3 O2 S | 3-amino-N-(4-bromo-3-methyl- phenyl)-6-(3- methoxyphenyl)thieno[2,3- b]pyridine-2- carboxamide |
| 551 | 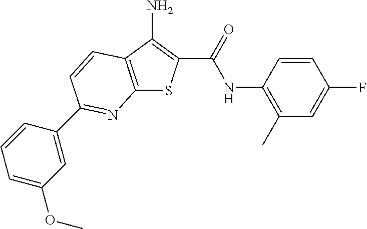 [SMILES: COc1cccc(-c2ccc3c(N)c(C(=O)Nc4ccc(F)cc4C)sc3n2)c1] | C22 H18 F N3 O2 S | 3-amino-N-(4-fluoro-2-methyl- phenyl)-6-(3- methoxyphenyl)thieno[2,3- b]pyridine-2-carboxamide |
| 552 | 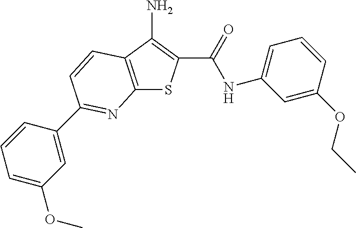 [SMILES: CCOc1cccc(NC(=O)c2sc3nc(-c4cccc(OC)c4)ccc3c2N)c1] | C23 H21 N3 O3 S | 3-amino-N-(3-ethoxyphenyl)-6- (3-methoxyphenyl)thieno[2,3- b]pyridine-2-carboxamide |
| 553 | 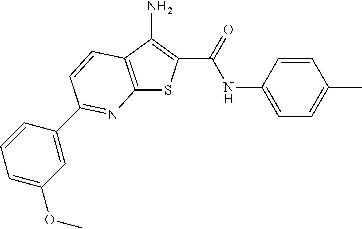 [SMILES: COc1cccc(-c2ccc3c(N)c(C(=O)Nc4ccc(C)cc4)sc3n2)c1] | C22 H19 N3 O2 S | 3-amino-6-(3-methoxyphenyl)- N-(p-tolyl)thieno[2,3-b]pyridine- 2-carboxamide |
| 554 | 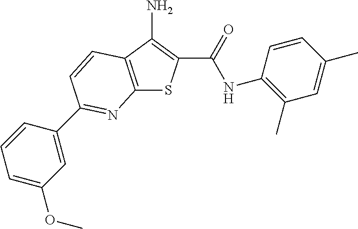 [SMILES: COc1cccc(-c2ccc3c(N)c(C(=O)Nc4ccc(C)cc4C)sc3n2)c1] | C23 H21 N3 O2 S | 3-amino-N-(2,4- dimethylphenyl)-6-(3- methoxyphenyl)thieno- [2,3-b]pyridine-2- carboxamide |
| 555 | 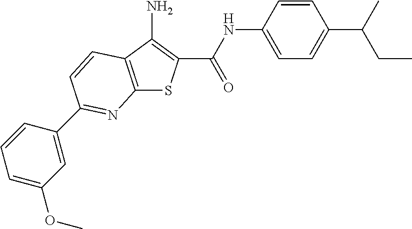 [SMILES: CCC(C)c1ccc(NC(=O)c2sc3nc(-c4cccc(OC)c4)ccc3c2N)cc1] | C25 H25 N3 O2 S | 3-amino-6-(3-methoxyphenyl)- N-(4-sec- butylphenyl)thieno[2,3- b]pyridine-2-carboxamide |
| 556 | 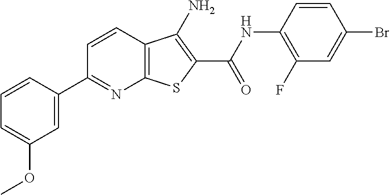 [SMILES: COc1cccc(-c2ccc3c(N)c(C(=O)Nc4ccc(Br)cc4F)sc3n2)c1] | C21 H15 Br F N3 O2 S | 3-amino-N-(4-bromo-2- fluoro-phenyl)-6-(3- methoxyphenyl)thieno- [2,3-b]pyridine-2- carboxamide |
| 557 | 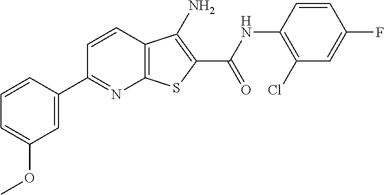 [SMILES: COc1cccc(-c2ccc3c(N)c(C(=O)Nc4ccc(F)cc4Cl)sc3n2)c1] | C21 H15 Cl F N3 O2 S | 3-amino-N-(2-chloro-4-fluoro- phenyl)-6-(3- methoxyphenyl)thieno[2,3- b]pyridine-2- carboxamide |
| 558 | 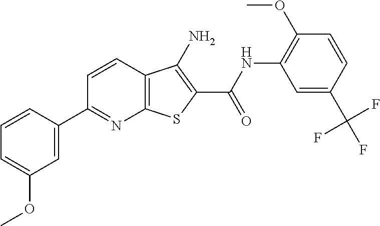 [SMILES: COc1cccc(-c2ccc3c(N)c(C(=O)Nc4cc(C(F)(F)F)ccc4OC)sc3n2)c1] | C23 H18 F3 N3 O3 S | 3-amino-6-(3-methoxyphenyl)- N-[2-methoxy-5- (trifluoromethyl)phenyl]- thieno[2,3-b]pyridine-2- carboxamide |
| 559 | 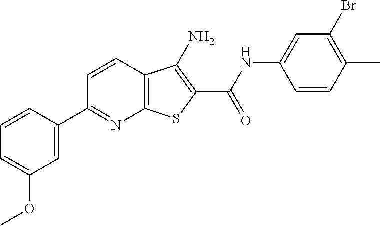 [SMILES: COc1cccc(-c2ccc3c(N)c(C(=O)Nc4ccc(C)c(Br)c4)sc3n2)c1] | C22 H18 Br N3 O2 S | 3-amino-N-(3-bromo- 4-methyl- phenyl)-6-(3- methoxyphenyl)thieno[2,3- b]pyridine-2- carboxamide |
| 560 | 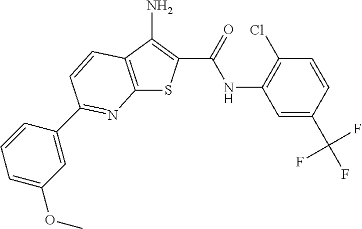 [SMILES: COc1cccc(-c2ccc3c(N)c(C(=O)Nc4cc(C(F)(F)F)ccc4Cl)sc3n2)c1] | C22 H15 O2 Cl F3 N3 O2 S | 3-amino-N-[2-chloro-5- (trifluoromethyl)phenyl]- 6-(3-methoxyphenyl)- thieno[2,3- b]pyridine-2- carboxamide |
| 561 | 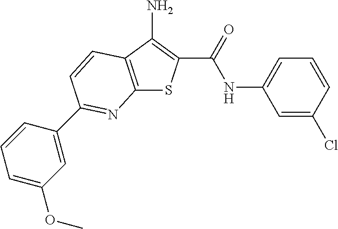 [SMILES: COc1cccc(-c2ccc3c(N)c(C(=O)Nc4cccc(Cl)c4)sc3n2)c1] | C21 H16 Cl N3 O2 S | 3-amino-N-(3-chlorophenyl)- 6-(3-methoxy- phenyl)thieno[2,3- b]pyridine-2- carboxamide |
| 562 | 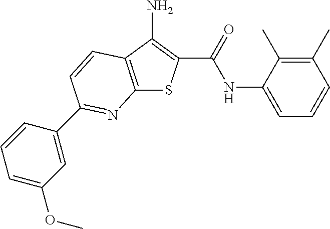 [SMILES: COc1cccc(-c2ccc3c(N)c(C(=O)Nc4cccc(C)c4C)sc3n2)c1] | C23 H21 N3 O2 S | 3-amino-N-(2,3- dimethylphenyl)-6-(3- methoxyphenyl)thieno[2,3- b]pyridine-2-carboxamide |
| 563 | 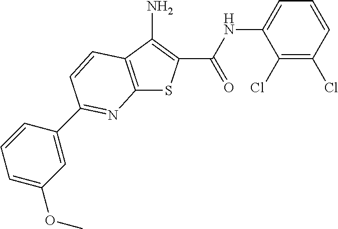 [SMILES: COc1cccc(-c2ccc3c(N)c(C(=O)Nc4cccc(Cl)c4Cl)sc3n2)c1] | C21 H15 Cl2 N3 O2 S | 3-amino-N-(2,3- dichlorophenyl)- 6-(3-methoxy- phenyl)thieno[2,3- b]pyridine-2- carboxamide |
| 564 | 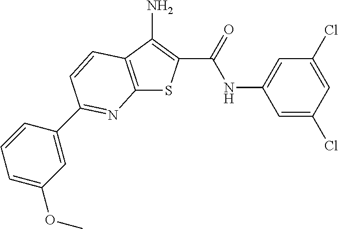 [SMILES: COc1cccc(-c2ccc3c(N)c(C(=O)Nc4cc(Cl)cc(Cl)c4)sc3n2)c1] | C21 H15 Cl2 N3 O2 S | 3-amino-N-(3,5- dichlorophenyl)- 6-(3-methoxy- phenyl)thieno[2,3- b]pyridine-2- carboxamide |
| 565 | 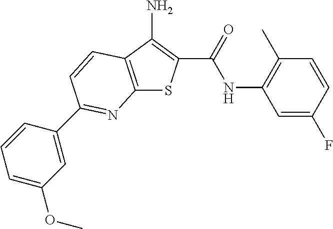 [SMILES: COc1cccc(-c2ccc3c(N)c(C(=O)Nc4cc(F)ccc4C)sc3n2)c1] | C22 H18 F N3 O2 S | 3-amino-N-(5-fluoro-2-methyl- phenyl)-6-(3- methoxyphenyl)thieno[2,3- b]pyridine-2- carboxamide |
| 566 | 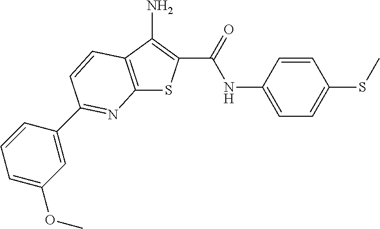 [SMILES: COc1cccc(-c2ccc3c(N)c(C(=O)Nc4ccc(SC)cc4)sc3n2)c1] | C22 H19 N3 O2 S2 | 3-amino-6-(3- methoxyphenyl)-N-(4- methylsulfanylphenyl)thieno- [2,3-b]pyridine-2- carboxamide |
| 567 | 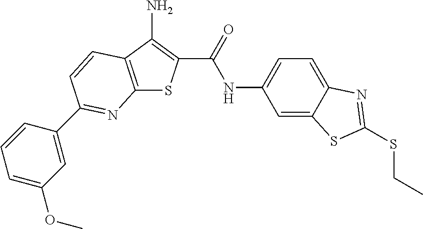 [SMILES: CCSc1nc2ccc(NC(=O)c3sc4nc(-c5cccc(OC)c5)ccc4c3N)cc2s1] | C24 H20 N4 O2 S3 | 3-amino-N-(2-ethylsulfanyl- 1,3-benzothiazol-6- yl)-6-(3-methoxyphenyl)- thieno[2,3-b]pyridine-2- carboxamide |
| 568 | 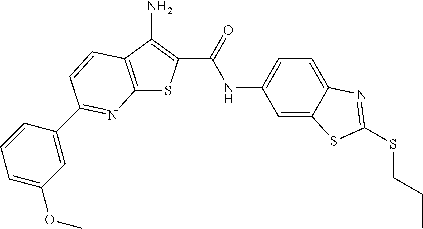 [SMILES: CCCSc1nc2ccc(NC(=O)c3sc4nc(-c5cccc(OC)c5)ccc4c3N)cc2s1] | C25 H22 N4 O2 S3 | 3-amino-6-(3-methoxyphenyl)- N-(2-propylsulfanyl-1,3- benzothiazol-6-yl)thieno[2,3- b]pyridine-2-carboxamide |
| 569 | 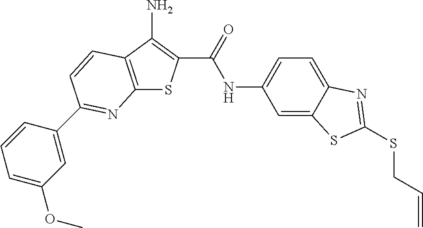 [SMILES: C=CCSc1nc2ccc(NC(=O)c3sc4nc(-c5cccc(OC)c5)ccc4c3N)cc2s1] | C25 H20 S3 N4 O2 | N-(2-allylsulfanyl-1,3- benzothiazol-6-yl)-3- amino-6-(3- methoxyphenyl)thieno[2,3- b]pyridine- 2-carboxamide |
| 570 | 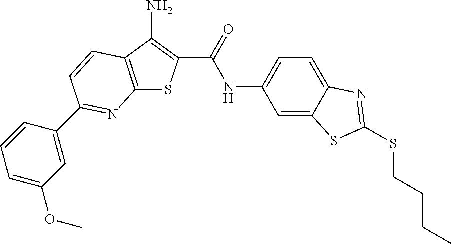 [SMILES: CCCCSc1nc2ccc(NC(=O)c3sc4nc(-c5cccc(OC)c5)ccc4c3N)cc2s1] | C26 H24 N4 O2 S3 | 3-amino-N-(2-butylsulfanyl- 1,3-benzothiazol-6- yl)-6-(3-methoxyphenyl)- thieno[2,3-b]pyridine-2- carboxamide |
| 571 | 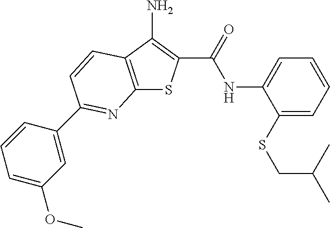 [SMILES: COc1cccc(-c2ccc3c(N)c(C(=O)Nc4ccccc4SCC(C)C)sc3n2)c1] | C25 H25 N3 O2 S2 | 3-amino-N-(2- isobutylsulfanylphenyl)-6-(3- methoxyphenyl)thieno[2,3- b]pyridine-2- carboxamide |
| 572 | 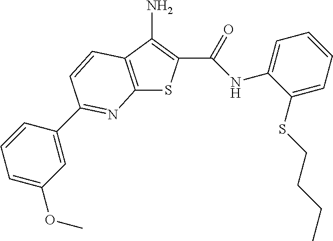 [SMILES: CCCCSc1ccccc1NC(=O)c1sc2nc(-c3cccc(OC)c3)ccc2c1N] | C25 H25 N3 O2 S2 | 3-amino-N-(2- butylsulfanylphenyl)-6-(3- methoxyphenyl)thieno[2,3- b]pyridine-2- carboxamide |
| 573 | 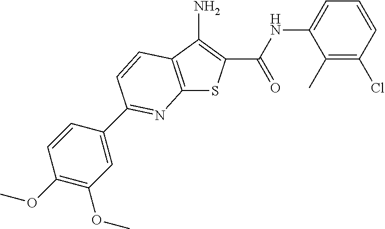 [SMILES: COc1ccc(-c2ccc3c(N)c(C(=O)Nc4cccc(Cl)c4C)sc3n2)cc1OC] | C23 H20 Cl N3 O3 S | 3-amino-N-(3-chloro- 2-methyl- phenyl)-6-(3,4- dimethoxyphenyl)thieno[2,3- b]pyridine-2- carboxamide |
| 574 | 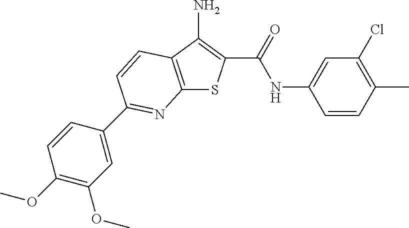 | C23 H20 Cl N3 O3 S | 3-amino-N-(3-chloro-4- methyl-phenyl)-6-(3,4- dimethoxyphenyl)thieno[2,3- b]pyridine-2- carboxamide |
| 575 | 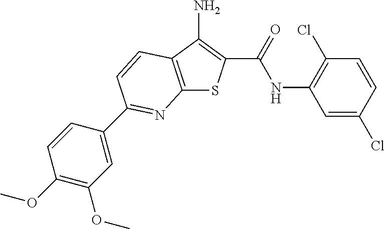 [SMILES: COc1ccc(-c2ccc3c(N)c(C(=O)Nc4cc(Cl)ccc4Cl)sc3n2)cc1OC] | C22 H17 Cl2 N3 O3 S | 3-amino-N-(2,5- dichlorophenyl)-6-(3,4- dimethoxyphenyl)thieno[2,3- b]pyridine-2- carboxamide |
| 576 | 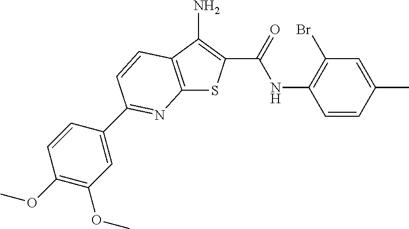 | C23 H20 Br N3 O3 S | 3-amino-N-(2-bromo- 4-methyl- phenyl)-6-(3,4- dimethoxyphenyl)thieno[2,3- b]pyridine-2- carboxamide |
| 577 | 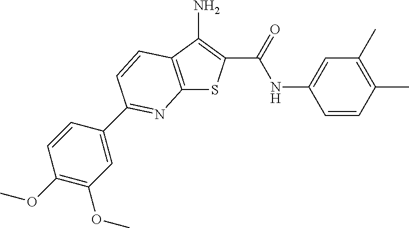 | C24 H23 N3 O3 S | 3-amino-6-(3,4- dimethoxyphenyl)- N-(3,4- dimethylphenyl)thieno[2,3- b]pyridine-2- carboxamide |
| 578 | 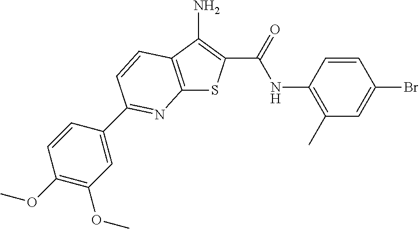 [SMILES: COc1ccc(-c2ccc3c(N)c(C(=O)Nc4ccc(Br)cc4C)sc3n2)cc1OC] | C23 H20 Br N3 O3 S | 3-amino-N-(4-bromo-2- methyl-phenyl)-6-(3,4- dimethoxyphenyl)thieno[2,3- b]pyridine-2- carboxamide |
| 579 | 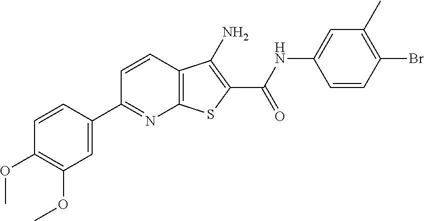 [SMILES: COc1ccc(-c2ccc3c(N)c(C(=O)Nc4ccc(Br)c(C)c4)sc3n2)cc1OC] | C23 H20 Br N3 O3 S | 3-amino-N-(4-bromo-3- methyl-phenyl)-6-(3,4- dimethoxyphenyl)thieno[2,3- b]pyridine-2- carboxamide |
| 580 | 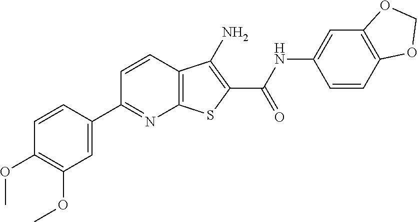 [SMILES: COc1ccc(-c2ccc3c(N)c(C(=O)Nc4ccc5c(c4)OCO5)sc3n2)cc1OC] | C23 H19 N3 O5 S | 3-amino-N-(1,3-benzodioxol- 5-yl)-6-(3,4- dimethoxyphenyl)thieno[2,3- b]pyridine-2- carboxamide |
| 581 | 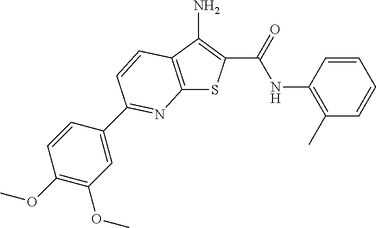 [SMILES: COc1ccc(-c2ccc3c(N)c(C(=O)Nc4ccccc4C)sc3n2)cc1OC] | C23 H21 N3 O3 S | 3-amino-6-(3,4- dimethoxyphenyl)-N-(o- tolyl)thieno[2,3- b]pyridine-2-carboxamide |
| 582 | 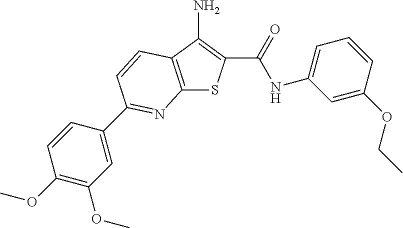 [SMILES: CCOc1cccc(NC(=O)c2sc3nc(-c4ccc(OC)c(OC)c4)ccc3c2N)c1] | C24 H23 N3 O4 S | 3-amino-6-(3,4- dimethoxyphenyl)-N-(3- ethoxyphenyl)thieno[2,3- b]pyridine-2- carboxamide |
| 583 | 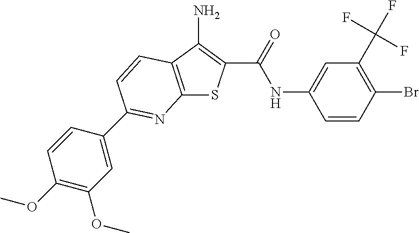 [SMILES: COc1ccc(-c2ccc3c(N)c(C(=O)Nc4ccc(Br)c(C(F)(F)F)c4)sc3n2)cc1OC] | C23 H17 Br F3 N3 O3 S | 3-amino-N-[4-bromo-3- (trifluoromethyl)phenyl]- 6-(3,4-dimethoxyphenyl)- thieno[2,3-b]pyridine-2- carboxamide |
| 584 | 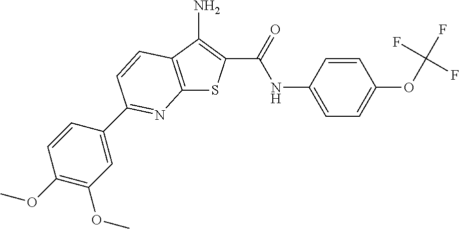 [SMILES: COc1ccc(-c2ccc3c(N)c(C(=O)Nc4ccc(OC(F)(F)F)cc4)sc3n2)cc1OC] | C23 H18 F3 N3 O4 S | 3-amino-6-(3,4- dimethoxyphenyl)- N-[4- (trifluoromethoxy)phenyl]- thieno[2,3-b]pyridine-2- carboxamide |
| 585 | 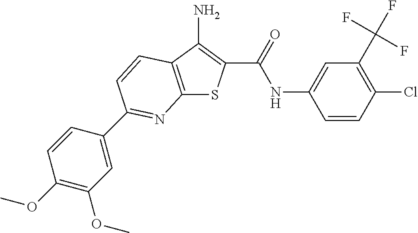 [SMILES: COc1ccc(-c2ccc3c(N)c(C(=O)Nc4ccc(Cl)c(C(F)(F)F)c4)sc3n2)cc1OC] | C23 H17 Cl F3 N3 O3 S | 3-amino-N-[4-chloro-3- (trifluoromethyl)phenyl]- 6-(3,4-dimethoxyphenyl)- thieno[2,3- b]pyridine-2- carboxamide |
| 586 | 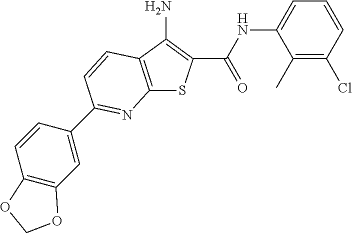 [SMILES: Cc1c(Cl)cccc1NC(=O)c1sc2nc(-c3ccc4c(c3)OCO4)ccc2c1N] | C22 H16 Cl N3 O3 S | 3-amino-6-(1,3- benzodioxol-5- yl)-N-(3-chloro-2- methyl-phenyl)thieno[2,3- b]pyridine-2- carboxamide |
| 587 | 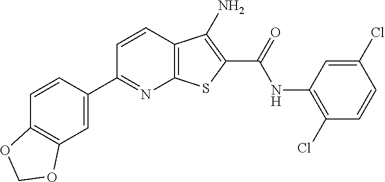 [SMILES: Nc1c(C(=O)Nc2cc(Cl)ccc2Cl)sc2nc(-c3ccc4c(c3)OCO4)ccc12] | C21 H13 Cl2 N3 O3 S | 3-amino-6-(1,3-benzodioxol- 5-yl)-N-(2,5- dichlorophenyl)thieno[2,3- b]pyridine-2- carboxamide |
| 588 | 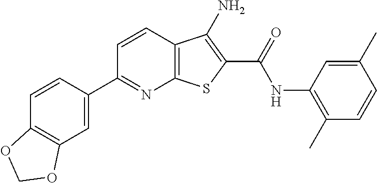 [SMILES: Cc1ccc(C)c(NC(=O)c2sc3nc(-c4ccc5c(c4)OCO5)ccc3c2N)c1] | C23 H19 N3 O3 S | 3-amino-6-(1,3-benzodioxol- 5-yl)-N-(2,5- dimethylphenyl)thieno[2,3- b]pyridine-2- carboxamide |
| 589 | 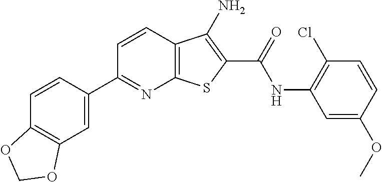 [SMILES: COc1ccc(Cl)c(NC(=O)c2sc3nc(-c4ccc5c(c4)OCO5)ccc3c2N)c1] | C22 H16 Cl N3 O4 S | 3-amino-6-(1,3-benzodioxol- 5-yl)-N-(2-chloro-5- methoxy-phenyl)thieno[2,3- b]pyridine-2- carboxamide |
| 590 | 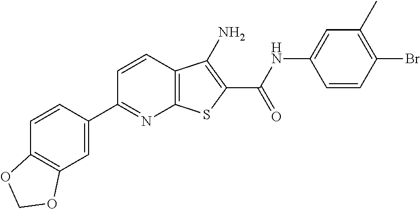 [SMILES: Cc1cc(NC(=O)c2sc3nc(-c4ccc5c(c4)OCO5)ccc3c2N)ccc1Br] | C22 H16 Br N3 O3 S | 3-amino-6-(1,3-benzodioxol- 5-yl)-N-(4-bromo-3- methyl- phenyl)thieno[2,3- b]pyridine-2- carboxamide |
| 591 | 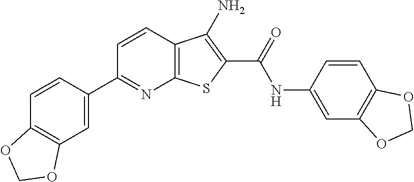 [SMILES: Nc1c(C(=O)Nc2ccc3c(c2)OCO3)sc2nc(-c3ccc4c(c3)OCO4)ccc12] | C22 H15 N3 O5 S | 3-amino-N,6-bis(1,3- benzodioxol-5-yl)thieno[2,3- b]pyridine-2-carboxamide |
| 592 | 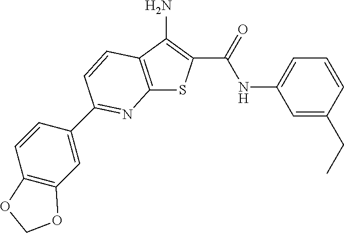 [SMILES: CCc1cccc(NC(=O)c2sc3nc(-c4ccc5c(c4)OCO5)ccc3c2N)c1] | C23 H19 N3 O3 S | 3-amino-6-(1,3-benzodioxol- 5-yl)-N-(3- ethylphenyl)thieno[2,3- b]pyridine-2-carboxamide |
| 593 | 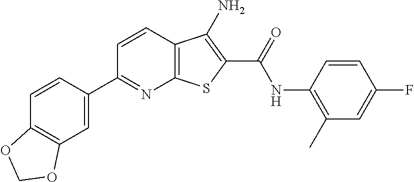 [SMILES: Cc1cc(F)ccc1NC(=O)c1sc2nc(-c3ccc4c(c3)OCO4)ccc2c1N] | C22 H16 F N3 O3 S | 3-amino-6-(1,3-benzodioxol- 5-yl)-N-(4-fluoro-2- methyl-phenyl)thieno[2,3- b]pyridine-2- carboxamide |
| 594 | 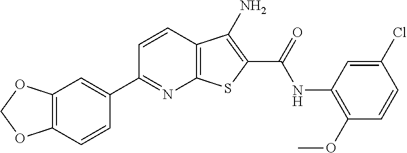 [SMILES: COc1ccc(Cl)cc1NC(=O)c1sc2nc(-c3ccc4c(c3)OCO4)ccc2c1N] | C22 H16 Cl N3 O4 S | 3-amino-6-(1,3- benzodioxol-5- yl)-N-(5-chloro-2- methoxy-phenyl)thieno[2,3- b]pyridine-2- carboxamide |
| 595 | 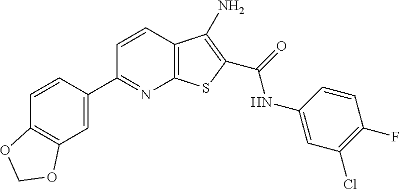 [SMILES: Nc1c(C(=O)Nc2ccc(F)c(Cl)c2)sc2nc(-c3ccc4c(c3)OCO4)ccc12] | C21 H13 Cl F N3 O3 S | 3-amino-6-(1,3- benzodioxol-5- yl)-N-(3-chloro-4- fluoro-phenyl)thieno[2,3- b]pyridine-2- carboxamide |
| 596 | 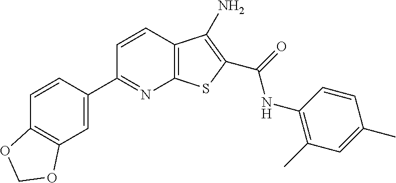 [SMILES: Cc1ccc(NC(=O)c2sc3nc(-c4ccc5c(c4)OCO5)ccc3c2N)c(C)c1] | C23 H19 N3 O3 S | 3-amino-6-(1,3-benzodioxol- 5-yl)-N-(2,4- dimethylphenyl)thieno[2,3- b]pyridine-2- carboxamide |
| 597 | 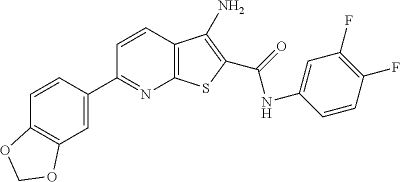 [SMILES: Nc1c(C(=O)Nc2ccc(F)c(F)c2)sc2nc(-c3ccc4c(c3)OCO4)ccc12] | C21 H13 F2 N3 O3 S | 3-amino-6-(1,3- benzodioxol-5-yl)-N-(3,4- difluorophenyl)thieno[2,3- b]pyridine-2- carboxamide |
| 598 | 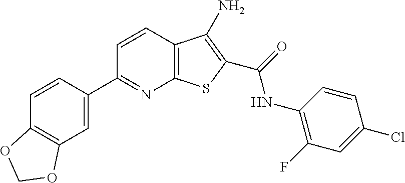 [SMILES: Nc1c(C(=O)Nc2ccc(Cl)cc2F)sc2nc(-c3ccc4c(c3)OCO4)ccc12] | C21 H13 Cl F N3 O3 S | 3-amino-6-(1,3-benzodioxol-5- yl)-N-(4-chloro-2- fluoro-phenyl)thieno[2,3- b]pyridine-2- carboxamide |
| 599 | 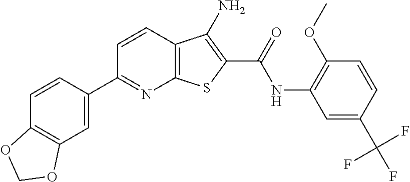 [SMILES: COc1ccc(C(F)(F)F)cc1NC(=O)c1sc2nc(-c3ccc4c(c3)OCO4)ccc2c1N] | C23 H16 F3 N3 O4 S | 3-amino-6-(1,3- benzodioxol-5-yl)-N- [2-methoxy- 5-(trifluoromethyl)- phenyl]thieno[2,3- b]pyridine-2- carboxamide |
| 600 | 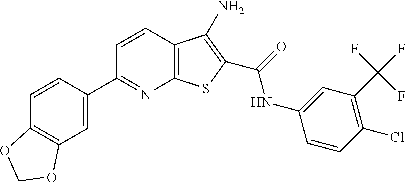 [SMILES: Nc1c(C(=O)Nc2ccc(Cl)c(C(F)(F)F)c2)sc2nc(-c3ccc4c(c3)OCO4)ccc12] | C22 H13 Cl F3 N3 O3 S | 3-amino-6-(1,3- benzodioxol-5-yl)-N- [4-chloro-3- (trifluoromethyl)- phenyl]thieno[2,3- b]pyridine-2- carboxamide |
| 601 | 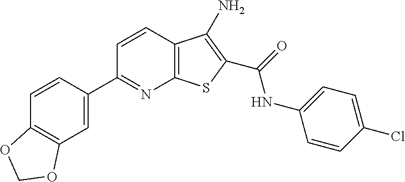 [SMILES: Nc1c(C(=O)Nc2ccc(Cl)cc2)sc2nc(-c3ccc4c(c3)OCO4)ccc12] | C21 H14 Cl N3 O3 S | 3-amino-6-(1,3- benzodioxol-5-yl)- N-(4-chlorophenyl)thieno[2,3- b]pyridine-2- carboxamide |
| 602 | 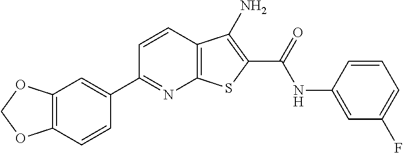 [SMILES: Nc1c(C(=O)Nc2cccc(F)c2)sc2nc(-c3ccc4c(c3)OCO4)ccc12] | C21 H14 F N3 O3 S | 3-amino-6-(1,3- benzodioxol-5-yl)-N-(3- fluorophenyl)thieno[2,3- b]pyridine-2-carboxamide |
| 603 | 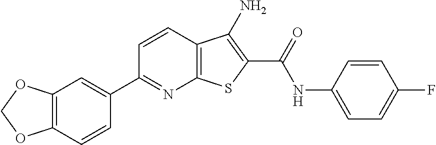 [SMILES: Nc1c(C(=O)Nc2ccc(F)cc2)sc2nc(-c3ccc4c(c3)OCO4)ccc12] | C21 H14 F N3 O3 S | 3-amino-6-(1,3- benzodioxol-5-yl)-N-(4- fluorophenyl)thieno[2,3- b]pyridine-2-carboxamide |
| 604 | 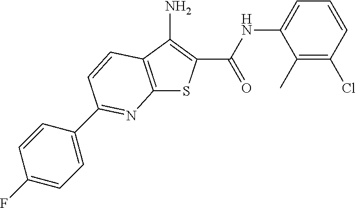 [SMILES: Cc1c(Cl)cccc1NC(=O)c1sc2nc(-c3ccc(F)cc3)ccc2c1N] | C21 H15 Cl F N3 O S | 3-amino-N-(3-chloro-2-methyl- phenyl)-6-(4- fluorophenyl)thieno[2,3- b]pyridine-2-carboxamide |
| 605 | 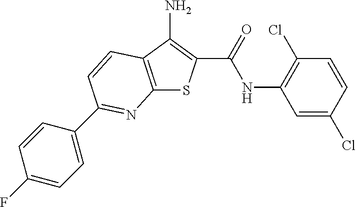 [SMILES: Nc1c(C(=O)Nc2cc(Cl)ccc2Cl)sc2nc(-c3ccc(F)cc3)ccc12] | C20 H12 Cl2 F N3 O S | 3-amino-N-(2,5- dichlorophenyl)-6-(4- fluorophenyl)thieno[2,3- b]pyridine-2-carboxamide |
| 606 | 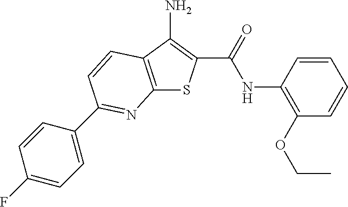 [SMILES: CCOc1ccccc1NC(=O)c1sc2nc(-c3ccc(F)cc3)ccc2c1N] | C22 H18 F N3 O2 S | 3-amino-N-(2- ethoxyphenyl)-6-(4- fluorophenyl)thieno[2,3- b]pyridine-2-carboxamide |
| 607 | 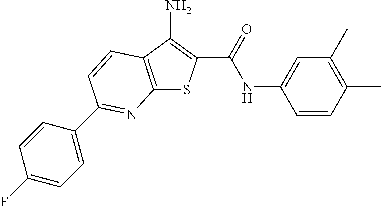 [SMILES: Cc1ccc(NC(=O)c2sc3nc(-c4ccc(F)cc4)ccc3c2N)cc1C] | C22 H18 F N3 O S | 3-amino-N-(3,4- dimethylphenyl)-6-(4- fluorophenyl)thieno[2,3- b]pyridine-2-carboxamide |
| 608 | 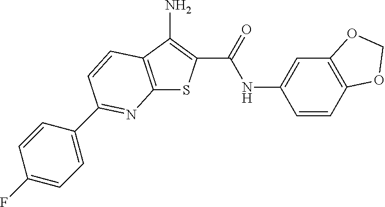 [SMILES: Nc1c(C(=O)Nc2ccc3c(c2)OCO3)sc2nc(-c3ccc(F)cc3)ccc12] | C21 H14 F N3 O3 S | 3-amino-N-(1,3- benzodioxol-5-yl)-6-(4- fluorophenyl)thieno[2,3- b]pyridine-2-carboxamide |
| 609 | 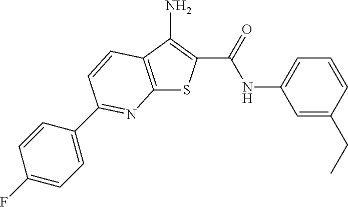 [SMILES: CCc1cccc(NC(=O)c2sc3nc(-c4ccc(F)cc4)ccc3c2N)c1] | C22 H18 F N3 O S | 3-amino-N-(3- ethylphenyl)-6-(4- fluorophenyl)thieno[2,3- b]pyridine-2-carboxamide |
| 610 | 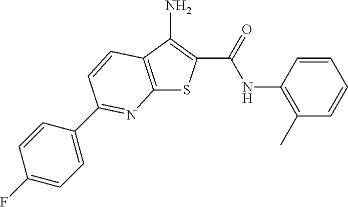 [SMILES: Cc1ccccc1NC(=O)c1sc2nc(-c3ccc(F)cc3)ccc2c1N] | C21 H16 F N3 O S | 3-amino-6-(4-fluorophenyl)-N- (o-tolyl)thieno[2,3- b]pyridine-2-carboxamide |
| 611 | 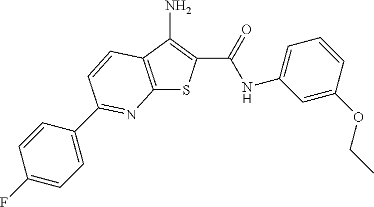 [SMILES: CCOc1cccc(NC(=O)c2sc3nc(-c4ccc(F)cc4)ccc3c2N)c1] | C22 H18 F N3 O2 S | 3-amino-N-(3- ethoxyphenyl)-6-(4- fluorophenyl)thieno[2,3- b]pyridine-2-carboxamide |
| 612 | 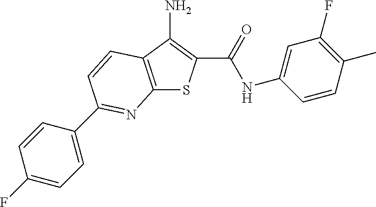 [SMILES: Cc1ccc(NC(=O)c2sc3nc(-c4ccc(F)cc4)ccc3c2N)cc1F] | C21 H15 F2 N3 O S | 3-amino-N-(3-fluoro-4-methyl- phenyl)-6-(4- fluorophenyl)thieno[2,3- b]pyridine-2-carboxamide |
| 613 | 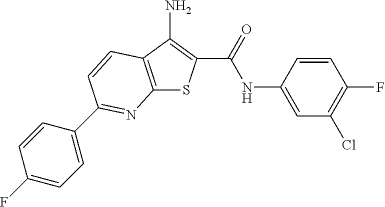 [SMILES: Nc1c(C(=O)Nc2ccc(F)c(Cl)c2)sc2nc(-c3ccc(F)cc3)ccc12] | C20 H12 Cl F2 N3 O S | 3-amino-N-(3-chloro-4- fluoro-phenyl)-6-(4- fluorophenyl)thieno[2,3- b]pyridine-2-carboxamide |
| 614 | 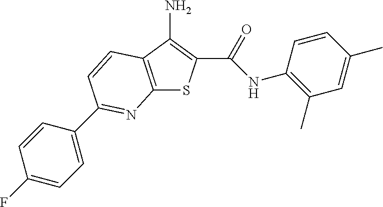 [SMILES: Cc1ccc(NC(=O)c2sc3nc(-c4ccc(F)cc4)ccc3c2N)c(C)c1] | C22 H18 F N3 O S | 3-amino-N-(2,4- dimethylphenyl)-6-(4- fluorophenyl)thieno[2,3- b]pyridine-2-carboxamide |
| 615 | 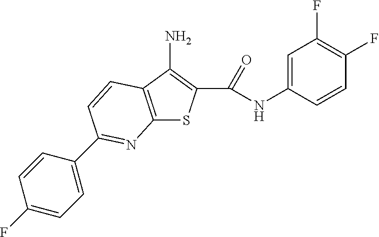 [SMILES: Nc1c(C(=O)Nc2ccc(F)c(F)c2)sc2nc(-c3ccc(F)cc3)ccc12] | C20 H12 F3 N3 O S | 3-amino-N-(3,4- difluorophenyl)-6-(4- fluorophenyl)thieno[2,3- b]pyridine-2-carboxamide |
| 616 | 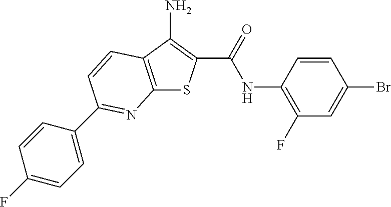 [SMILES: Nc1c(C(=O)Nc2ccc(Br)cc2F)sc2nc(-c3ccc(F)cc3)ccc12] | C20 H12 Br F2 N3 O S | 3-amino-N-(4-bromo-2-fluoro- phenyl)-6-(4- fluorophenyl)thieno[2,3- b]pyridine-2-carboxamide |
| 617 | 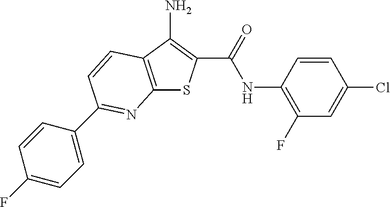 [SMILES: Nc1c(C(=O)Nc2ccc(Cl)cc2F)sc2nc(-c3ccc(F)cc3)ccc12] | C20 H12 Cl F2 N3 O S | 3-amino-N-(4-chloro-2-fluoro- phenyl)-6-(4- fluorophenyl)thieno[2,3- b]pyridine-2-carboxamide |
| 618 | 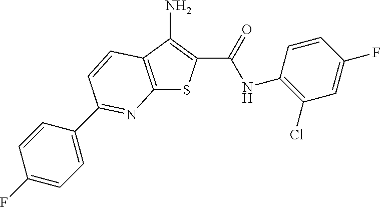 [SMILES: Nc1c(C(=O)Nc2ccc(F)cc2Cl)sc2nc(-c3ccc(F)cc3)ccc12] | C20 H12 Cl F2 N3 O S | 3-amino-N-(2-chloro-4-fluoro- phenyl)-6-(4- fluorophenyl)thieno[2,3- b]pyridine-2-carboxamide |
| 619 | 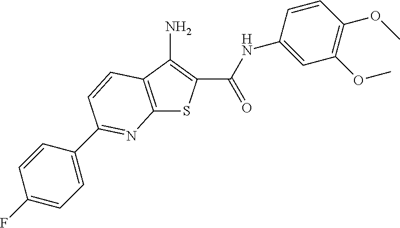 [SMILES: COc1ccc(NC(=O)c2sc3nc(-c4ccc(F)cc4)ccc3c2N)cc1OC] | C22 H18 F N3 O3 S | 3-amino-N-(3,4- dimethoxyphenyl)-6-(4- fluorophenyl)thieno[2,3- b]pyridine-2-carboxamide |
| 620 | 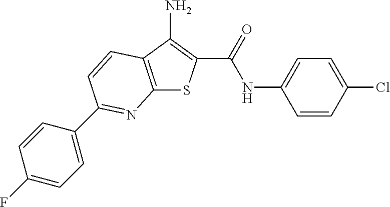 [SMILES: Nc1c(C(=O)Nc2ccc(Cl)cc2)sc2nc(-c3ccc(F)cc3)ccc12] | C20 H13 Cl F N3 O S | 3-amino-N-(4- chlorophenyl)-6-(4- fluorophenyl)thieno[2,3- b]pyridine-2-carboxamide |
| 621 | 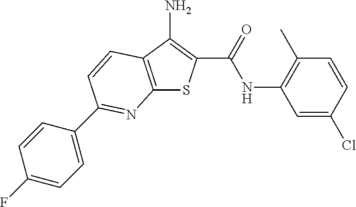 [SMILES: Cc1ccc(Cl)cc1NC(=O)c1sc2nc(-c3ccc(F)cc3)ccc2c1N] | C21 H15 Cl F N3 O S | 3-amino-N-(5-chloro-2-methyl- phenyl)-6-(4- fluorophenyl)thieno[2,3- b]pyridine-2-carboxamide |
| 622 | 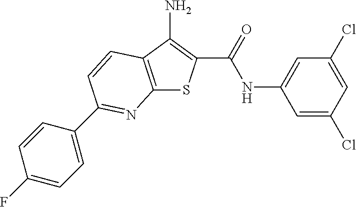 [SMILES: Nc1c(C(=O)Nc2cc(Cl)cc(Cl)c2)sc2nc(-c3ccc(F)cc3)ccc12] | C20 H12 Cl2 F N3 O S | 3-amino-N-(3,5- dichlorophenyl)-6-(4- fluorophenyl)thieno[2,3- b]pyridine-2-carboxamide |
| 623 | 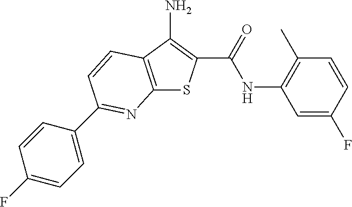 [SMILES: Cc1ccc(F)cc1NC(=O)c1sc2nc(-c3ccc(F)cc3)ccc2c1N] | C21 H15 F2 N3 O S | 3-amino-N-(5-fluoro- 2-methyl- phenyl)-6-(4- fluorophenyl)thieno[2,3- b]pyridine- 2-carboxamide |
| 624 | 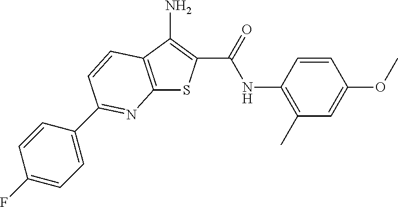 [SMILES: COc1ccc(NC(=O)c2sc3nc(-c4ccc(F)cc4)ccc3c2N)c(C)c1] | C22 H18 F N3 O2 S | 3-amino-6-(4-fluorophenyl)- N-(4-methoxy-2- methyl-phenyl)thieno[2,3- b]pyridine-2-carboxamide |
| 625 | 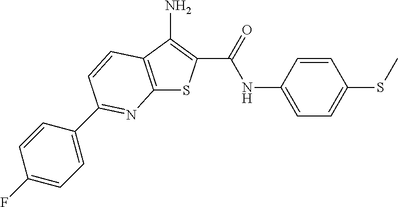 [SMILES: CSc1ccc(NC(=O)c2sc3nc(-c4ccc(F)cc4)ccc3c2N)cc1] | C21 H16 F N3 O S2 | 3-amino 6-(4- fluorophenyl)-N-(4- methylsulfanyl- phenypthieno[2,3- b]pyridine-2-carboxamide |
| 626 | 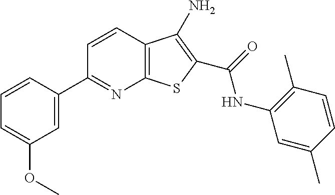 [SMILES: COc1cccc(-c2ccc3c(N)c(C(=O)Nc4cc(C)ccc4C)sc3n2)c1] | C23 H21 N3 O2 S | 3-amino-N-(2,5- dimethylphenyl)-6-(3- methoxyphenyl)thieno[2,3- b]pyridine-2- carboxamide |
| 627 | 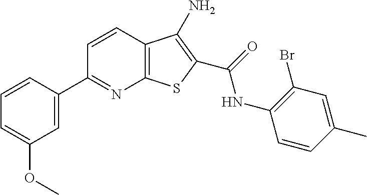 [SMILES: COc1cccc(-c2ccc3c(N)c(C(=O)Nc4ccc(C)cc4Br)sc3n2)c1] | C22 H18 Br N3 O2 S | 3-amino-N-(2- bromo-4-methyl- phenyl)-6-(3- methoxyphenyl)thieno[2,3- b]pyridine-2- carboxamide |
| 628 | 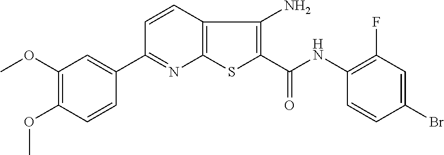 [SMILES: COc1ccc(-c2ccc3c(N)c(C(=O)Nc4ccc(Br)cc4F)sc3n2)cc1OC] | C22 H17 Br F N3 O3 S | 3-amino-N-(4-bromo-2-fluoro- phenyl)-6-(3,4- dimethoxyphenyl)thieno[2,3-b]- pyridine-2- carboxamide |
| 629 | 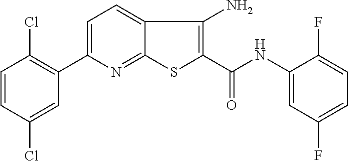 [SMILES: Nc1c(C(=O)Nc2cc(F)ccc2F)sc2nc(-c3cc(Cl)ccc3Cl)ccc12] | C20 H11 Cl2 F2 N3 O S | 3-amino-6-(2,5- dichlorophenyl)- N-(2,5- difluorophenyl)thieno[2,3- b]pyridine-2- carboxamide |
| 630 | 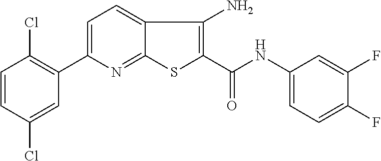 [SMILES: Nc1c(C(=O)Nc2ccc(F)c(F)c2)sc2nc(-c3cc(Cl)ccc3Cl)ccc12] | C20 H11 Cl2 F2 N3 O S | 3-amino-6-(2,5- dichlorophenyl)-N-(3,4- difluorophenyl)thieno[2,3- b]pyridine-2- carboxamide |
| 631 | 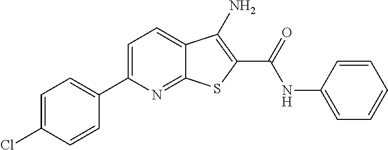 [SMILES: Nc1c(C(=O)Nc2ccccc2)sc2nc(-c3ccc(Cl)cc3)ccc12] | C20 H14 Cl N3 O S | 3-amino-6-(4- chlorophenyl)-N- phenyl-thieno[2,3- b]pyridine-2-carboxamide |
| 632 | 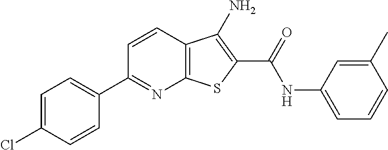 [SMILES: Cc1cccc(NC(=O)c2sc3nc(-c4ccc(Cl)cc4)ccc3c2N)c1] | C21 H16 Cl N3 O S | 3-amino-6-(4- chlorophenyl)-N-(m- tolyl)thieno[2,3- b]pyridine-2-carboxamide |
| 633 | 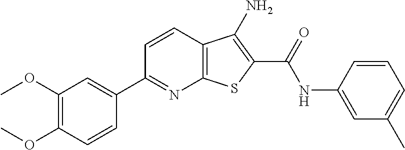 | C23 H21 N3 O3 S | 3-amino-6-(3,4- dimethoxyphenyl)-N-(m- tolyl)thieno[2,3- b]pyridine-2-carboxamide |
| 634 | 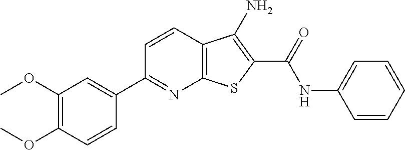 [SMILES: COc1ccc(-c2ccc3c(N)c(C(=O)Nc4ccccc4)sc3n2)cc1OC] | C22 H19 N3 O3 S | 3-amino-6-(3,4- dimethoxyphenyl)-N-phenyl- thieno[2,3-b]pyridine-2- carboxamide |
| 635 | 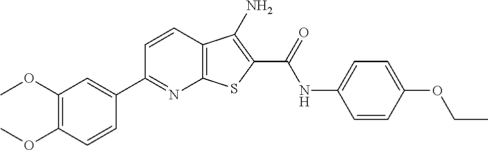 [SMILES: CCOc1ccc(NC(=O)c2sc3nc(-c4ccc(OC)c(OC)c4)ccc3c2N)cc1] | C24 H23 N3 O4 S | 3-amino-6-(3,4- dimethoxyphenyl)-N-(4- ethoxyphenyl)thieno[2,3- b]pyridine-2- carboxamide |
| 636 | 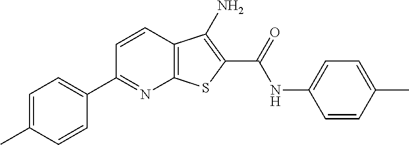 | C22 H19 N3 O S | 3-amino-N,6-bis(p- tolyl)thieno[2,3- b]pyridine-2- carboxamide |
| 637 | 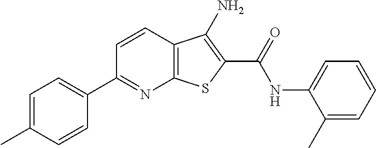 [SMILES: Cc1ccc(-c2ccc3c(N)c(C(=O)Nc4ccccc4C)sc3n2)cc1] | C22 H19 N3 O S | 3-amino-N-(o-tolyl)-6-(p- tolyl)thieno[2,3- b]pyridine-2-carboxamide |
| 638 | 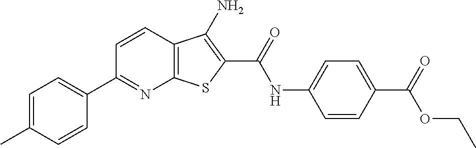 [SMILES: CCOC(=O)c1ccc(NC(=O)c2sc3nc(-c4ccc(C)cc4)ccc3c2N)cc1] | C24 H21 N3 O3 S | ethyl 4-[[3-amino-6-(p- tolyl)thieno[2,3-b]pyridine- 2-carbonyl]amino]benzoate |
| 639 | 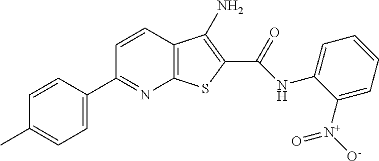 [SMILES: Cc1ccc(-c2ccc3c(N)c(C(=O)Nc4ccccc4[N+](=O)[O-])sc3n2)cc1] | C21 H16 N4 O3 S | 3-amino-N-(2- nitrophenyl)-6-(p- tolyl)thieno[2,3- b]pyridine-2-carboxamide |
| 640 | 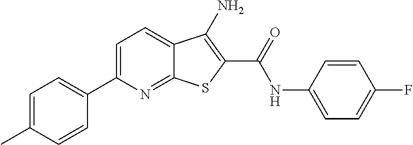 [SMILES: Cc1ccc(-c2ccc3c(N)c(C(=O)Nc4ccc(F)cc4)sc3n2)cc1] | C21 H16 F N3 O S | 3-amino-N-(4- fluorophenyl)-6-(p- tolyl)thieno[2,3- b]pyridine-2-carboxamide |
| 641 | 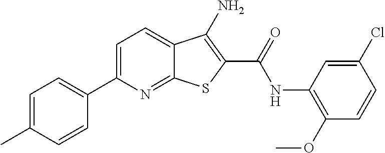 [SMILES: COc1ccc(Cl)cc1NC(=O)c1sc2nc(-c3ccc(C)cc3)ccc2c1N] | C22 H18 Cl N3 O2 S | 3-amino-N-(5-chloro-2- methoxy-phenyl)-6-(p- tolyl)thieno[2,3- b]pyridine-2-carboxamide |
| 642 | 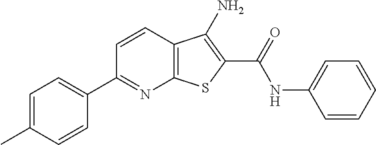 [SMILES: Cc1ccc(-c2ccc3c(N)c(C(=O)Nc4ccccc4)sc3n2)cc1] | C21 H17 N3 O S | 3-amino-N-phenyl-6- (p-tolyl)thieno[2,3- b]pyridine-2-carboxamide |
| 643 | 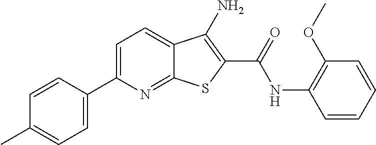 [SMILES: COc1ccccc1NC(=O)c1sc2nc(-c3ccc(C)cc3)ccc2c1N] | C22 H19 N3 O2 S | 3-amino-N-(2- methoxyphenyl)-6-(p- tolyl)thieno[2,3- b]pyridine-2-carboxamide |
| 644 | 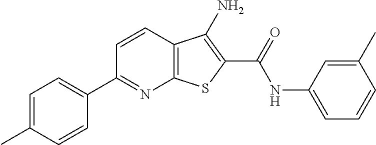 | C22 H19 N3 O S | 3-amino-N-(m-tolyl)-6- (p-tolyl)thieno[2,3- b]pyridine-2-carboxamide |
| 645 | 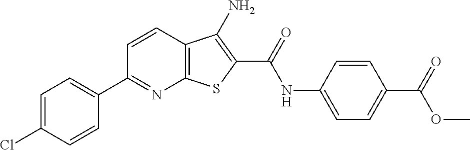 [SMILES: COC(=O)c1ccc(NC(=O)c2sc3nc(-c4ccc(Cl)cc4)ccc3c2N)cc1] | C22 H16 Cl N3 O3 S | methyl 4-[[3-amino-6-(4- chlorophenyl)thieno[2,3- b]pyridine-2- carbonyl]amino]benzoate |
| 646 | 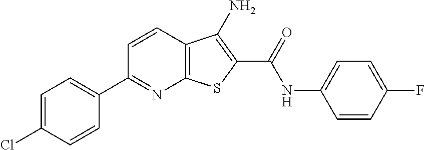 [SMILES: Nc1c(C(=O)Nc2ccc(F)cc2)sc2nc(-c3ccc(Cl)cc3)ccc12] | C20 H13 Cl F N3 O S | 3-amino-6-(4- chlorophenyl)-N-(4- fluorophenyl)thieno[2,3- b]pyridine-2- carboxamide |
| 647 | 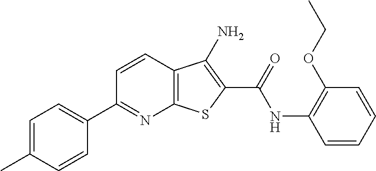 [SMILES: CCOc1ccccc1NC(=O)c1sc2nc(-c3ccc(C)cc3)ccc2c1N] | C23 H21 N3 O2 S | 3-amino-N-(2- ethoxyphenyl)-6-(p- tolyl)thieno[2,3- b]pyridine-2-carboxamide |
| 648 | 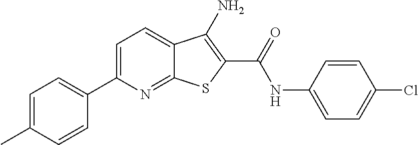 [SMILES: Cc1ccc(-c2ccc3c(N)c(C(=O)Nc4ccc(Cl)cc4)sc3n2)cc1] | C21 H16 Cl N3 O S | 3-amino-N-(4- chlorophenyl)-6- (p-tolyl)thieno[2,3- b]pyridine-2-carboxamide |
| 649 | 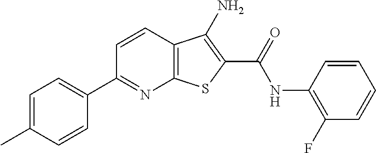 [SMILES: Cc1ccc(-c2ccc3c(N)c(C(=O)Nc4ccccc4F)sc3n2)cc1] | C21 H16 F N3 O S | 3-amino-N-(2- fluorophenyl)-6- (p-tolyl)thieno[2,3- b]pyridine-2-carboxamide |
| 650 | 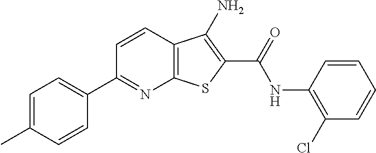 [SMILES: Cc1ccc(-c2ccc3c(N)c(C(=O)Nc4ccccc4Cl)sc3n2)cc1] | C21 H16 Cl N3 O S | 3-amino-N-(2- chlorophenyl)-6-(p- tolyl)thieno[2,3- b]pyridine-2-carboxamide |
| 651 | 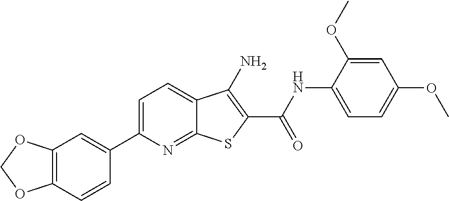 [SMILES: COc1ccc(NC(=O)c2sc3nc(-c4ccc5c(c4)OCO5)ccc3c2N)c(OC)c1] | C23 H19 N3 O5 S | 3-amino-6-(1,3-benzodioxol- 5-yl)-N-(2,4- dimethoxyphenyl)thieno[2,3- b]pyridine-2- carboxamide |
| 652 | 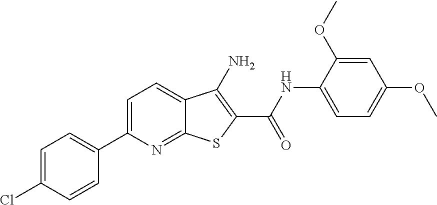 [SMILES: COc1ccc(NC(=O)c2sc3nc(-c4ccc(Cl)cc4)ccc3c2N)c(OC)c1] | C22 H18 Cl N3 O3 S | 3-amino-6-(4- chlorophenyl)-N-(2,4- dimethoxyphenyl)thieno[2,3- b]pyridine-2- carboxamide |
| 653 | 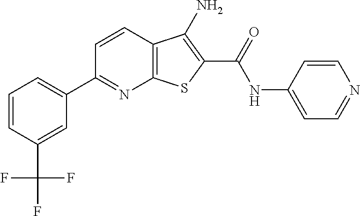 [SMILES: Nc1c(C(=O)Nc2ccncc2)sc2nc(-c3cccc(C(F)(F)F)c3)ccc12] | C20 H13 F3 N4 O S | 3-amino-N-(4-pyridyl)-6-[3- (trifluoromethyl)- phenyl]thieno[2,3-b]- pyridine-2-carboxamide |
| 654 | 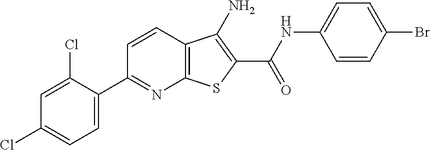 [SMILES: Nc1c(C(=O)Nc2ccc(Br)cc2)sc2nc(-c3ccc(Cl)cc3Cl)ccc12] | C20 H12 Br Cl2 N3 O S | 3-amino-N-(4- bromophenyl)-6-(2,4- dichlorophenyl)thieno[2,3- b]pyridine-2- carboxamide |
| 655 | 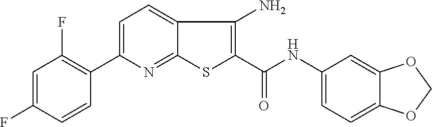 [SMILES: Nc1c(C(=O)Nc2ccc3c(c2)OCO3)sc2nc(-c3ccc(F)cc3F)ccc12] | C21 H13 F2 N3 O3 S | 3-amino-N-(1,3- benzodioxol-5-yl)-6- (2,4-difluorophenyl)thieno[2,3- b]pyridine-2- carboxamide |

TABLE 7
Activity against Dengue virus of compounds of the present invention.

Activity (EC₅₀ in μM)
A: EC₅₀ ≦ 5 μM; B: 5 < EC₅₀ ≦ 25 μM;
C: EC₅₀ > 25 μM; n.d.: not determined

| Cmpd | DENV-1 | DENV-2 | DENV-3 | DENV-4 |
|---|---|---|---|---|
| 364 | A | A | A | A |
| 365 | B | A | B | C |
| 366 | n.d. | A | n.d. | n.d. |
| 367 | n.d. | A | n.d. | n.d. |
| 368 | A | A | A | A |
| 369 | B | A | A | A |
| 370 | n.d. | A | n.d. | n.d. |
| 371 | n.d. | A | n.d. | n.d. |
| 372 | n.d. | A | n.d. | n.d. |
| 373 | A | A | A | A |
| 374 | n.d. | A | n.d. | n.d. |
| 375 | n.d. | B | n.d. | n.d. |
| 376 | n.d. | B | n.d. | n.d. |
| 377 | n.d. | A | n.d. | n.d. |
| 378 | n.d. | A | n.d. | n.d. |
| 379 | n.d. | A | n.d. | n.d. |
| 380 | n.d. | A | n.d. | n.d. |
| 381 | n.d. | B | n.d. | n.d. |
| 382 | n.d. | B | n.d. | n.d. |
| 383 | n.d. | A | n.d. | n.d. |
| 384 | n.d. | B | n.d. | n.d. |
| 385 | n.d. | B | n.d. | n.d. |
| 386 | n.d. | A | n.d. | n.d. |
| 387 | n.d. | B | n.d. | n.d. |
| 388 | n.d. | A | n.d. | n.d. |
| 389 | n.d. | B | n.d. | n.d. |
| 390 | n.d. | A | n.d. | n.d. |
| 391 | n.d. | A | n.d. | n.d. |
| 392 | n.d. | B | n.d. | n.d. |
| 393 | n.d. | B | n.d. | n.d. |
| 394 | n.d. | A | n.d. | n.d. |
| 395 | n.d. | B | n.d. | n.d. |
| 396 | n.d. | B | n.d. | n.d. |
| 397 | n.d. | A | n.d. | n.d. |
| 398 | n.d. | B | n.d. | n.d. |
| 399 | n.d. | A | n.d. | n.d. |
| 400 | n.d. | A | n.d. | n.d. |
| 401 | n.d. | C | n.d. | n.d. |
| 402 | n.d. | C | n.d. | n.d. |
| 403 | n.d. | A | n.d. | n.d. |
| 404 | n.d. | A | n.d. | n.d. |
| 405 | n.d. | A | n.d. | n.d. |
| 406 | n.d. | A | n.d. | n.d. |
| 407 | n.d. | C | n.d. | n.d. |
| 408 | n.d. | C | n.d. | n.d. |
| 409 | n.d. | C | n.d. | n.d. |
| 410 | n.d. | A | n.d. | n.d. |
| 411 | A | A | A | A |
| 412 | n.d. | B | n.d. | n.d. |
| 413 | A | A | A | A |
| 414 | A | A | A | A |
| 415 | A | A | A | A |
| 416 | n.d. | A | n.d. | n.d. |
| 417 | A | A | A | A |
| 418 | n.d. | B | n.d. | n.d. |
| 419 | n.d. | A | n.d. | n.d. |
| 420 | n.d. | A | n.d. | n.d. |
| 421 | n.d. | B | n.d. | n.d. |
| 422 | n.d. | A | n.d. | n.d. |
| 423 | n.d. | A | n.d. | n.d. |
| 424 | n.d. | B | n.d. | n.d. |
| 425 | A | A | A | A |
| 426 | n.d. | A | n.d. | n.d. |
| 427 | A | A | A | A |
| 428 | A | A | A | A |
| 429 | A | A | A | A |
| 430 | A | A | A | A |
| 431 | A | A | A | A |
| 432 | n.d. | B | n.d. | n.d. |
| 433 | A | A | A | A |
| 434 | A | A | A | A |
| 435 | n.d. | A | n.d. | n.d. |
| 436 | n.d. | A | n.d. | n.d. |
| 437 | A | A | A | A |
| 438 | A | A | A | A |
| 439 | A | A | A | A |
| 440 | n.d. | B | n.d. | n.d. |
| 441 | A | A | A | A |
| 442 | n.d. | A | n.d. | n.d. |
| 443 | n.d. | A | n.d. | n.d. |
| 444 | n.d. | A | n.d. | n.d. |
| 445 | A | A | A | A |
| 446 | A | A | A | A |
| 447 | A | A | A | A |
| 448 | A | A | A | A |
| 449 | A | A | A | A |
| 450 | A | A | A | A |
| 451 | n.d. | A | n.d. | n.d. |
| 452 | A | A | A | A |
| 453 | A | A | A | A |
| 454 | A | A | A | A |
| 455 | A | A | A | B |
| 456 | n.d. | A | n.d. | n.d. |
| 457 | n.d. | B | n.d. | n.d. |
| 458 | A | A | A | A |
| 459 | A | A | A | A |
| 460 | n.d. | A | n.d. | n.d. |
| 461 | A | A | A | A |
| 462 | A | A | A | A |
| 463 | n.d. | A | n.d. | n.d. |
| 464 | A | A | A | A |
| 465 | A | A | A | A |
| 466 | n.d. | B | n.d. | n.d. |
| 467 | n.d. | A | n.d. | n.d. |
| 468 | A | A | A | A |
| 469 | A | A | A | A |
| 470 | A | A | A | A |
| 471 | A | A | A | A |
| 472 | A | A | A | A |
| 473 | A | A | A | A |
| 474 | n.d. | A | n.d. | n.d. |
| 475 | A | A | A | A |
| 476 | A | A | A | A |
| 477 | n.d. | A | n.d. | n.d. |
| 478 | n.d. | B | n.d. | n.d. |
| 479 | n.d. | A | n.d. | n.d. |
| 480 | n.d. | A | n.d. | n.d. |
| 481 | n.d. | B | n.d. | n.d. |
| 482 | A | A | A | A |
| 483 | A | A | A | A |
| 484 | n.d. | A | n.d. | n.d. |
| 485 | A | A | A | A |
| 486 | A | A | A | A |
| 487 | n.d. | A | n.d. | n.d. |
| 488 | A | A | A | A |
| 489 | A | A | A | A |
| 490 | A | A | B | A |
| 491 | C | A | B | A |
| 492 | A | A | A | A |
| 493 | A | A | A | A |
| 494 | A | A | B | A |
| 495 | A | A | A | A |
| 496 | n.d. | A | n.d. | n.d. |
| 497 | A | A | A | A |
| 498 | A | A | A | A |
| 499 | n.d. | A | n.d. | n.d. |
| 500 | n.d. | A | n.d. | n.d. |
| 501 | n.d. | A | n.d. | n.d. |
| 502 | n.d. | A | n.d. | n.d. |
| 503 | n.d. | A | n.d. | n.d. |
| 504 | n.d. | A | n.d. | n.d. |
| 505 | n.d. | A | n.d. | n.d. |
| 506 | A | A | A | A |
| 507 | A | A | A | A |
| 508 | n.d. | A | n.d. | n.d. |
| 509 | n.d. | A | n.d. | n.d. |
| 510 | A | A | A | A |
| 511 | n.d. | A | n.d. | n.d. |
| 512 | A | A | A | A |
| 513 | n.d. | A | n.d. | n.d. |
| 514 | A | A | A | A |
| 515 | n.d. | A | n.d. | n.d. |
| 516 | n.d. | A | n.d. | n.d. |
| 517 | n.d. | A | n.d. | n.d. |
| 518 | n.d. | A | n.d. | n.d. |
| 519 | n.d. | A | n.d. | n.d. |
| 520 | n.d. | A | n.d. | n.d. |
| 521 | n.d. | A | n.d. | n.d. |
| 522 | A | A | A | A |
| 523 | n.d. | A | n.d. | n.d. |
| 524 | n.d. | A | n.d. | n.d. |
| 525 | n.d. | A | n.d. | n.d. |
| 526 | n.d. | A | n.d. | n.d. |
| 527 | n.d. | A | n.d. | n.d. |
| 528 | n.d. | A | n.d. | n.d. |
| 529 | A | A | A | A |
| 530 | A | A | A | A |
| 531 | n.d. | A | n.d. | n.d. |
| 532 | A | A | A | A |
| 533 | A | A | A | A |
| 534 | A | A | A | A |
| 535 | A | A | A | A |
| 536 | n.d. | A | n.d. | n.d. |
| 537 | n.d. | A | n.d. | n.d. |
| 538 | n.d. | A | n.d. | n.d. |
| 539 | n.d. | A | n.d. | n.d. |
| 540 | n.d. | A | n.d. | n.d. |
| 541 | n.d. | A | n.d. | n.d. |
| 542 | A | A | A | A |
| 543 | A | A | A | A |
| 544 | n.d. | A | n.d. | n.d. |
| 545 | n.d. | A | n.d. | n.d. |
| 546 | A | A | A | A |
| 547 | A | A | A | A |
| 548 | n.d. | A | n.d. | n.d. |
| 549 | n.d. | A | n.d. | n.d. |
| 550 | A | A | A | A |
| 551 | n.d. | A | n.d. | n.d. |
| 552 | n.d. | A | n.d. | n.d. |
| 553 | n.d. | A | n.d. | n.d. |
| 554 | n.d. | A | n.d. | n.d. |
| 555 | A | A | A | A |
| 556 | n.d. | A | n.d. | n.d. |
| 557 | n.d. | A | n.d. | n.d. |
| 558 | n.d. | A | n.d. | n.d. |
| 559 | n.d. | A | A | A |
| 560 | n.d. | A | n.d. | n.d. |
| 561 | A | A | A | A |
| 562 | n.d. | A | n.d. | n.d. |
| 563 | n.d. | A | n.d. | n.d. |
| 564 | n.d. | A | n.d. | n.d. |
| 565 | n.d. | A | n.d. | n.d. |
| 566 | A | A | A | A |
| 567 | n.d. | A | n.d. | n.d. |
| 568 | n.d. | A | n.d. | n.d. |
| 569 | A | A | B | A |
| 570 | A | A | A | A |
| 571 | A | A | A | A |
| 572 | A | A | A | A |
| 573 | n.d. | A | n.d. | n.d. |
| 574 | A | A | A | A |
| 575 | A | A | A | A |
| 576 | A | A | A | A |
| 577 | A | A | A | A |
| 578 | n.d. | A | n.d. | n.d. |
| 579 | n.d. | A | n.d. | n.d. |
| 580 | n.d. | A | n.d. | n.d. |
| 581 | n.d. | A | n.d. | n.d. |
| 582 | n.d. | A | n.d. | n.d. |
| 583 | A | A | A | A |
| 584 | n.d. | A | n.d. | A |
| 585 | n.d. | A | n.d. | n.d. |
| 586 | n.d. | A | n.d. | n.d. |
| 587 | n.d. | A | n.d. | n.d. |
| 588 | n.d. | A | n.d. | n.d. |
| 589 | n.d. | A | n.d. | n.d. |
| 590 | n.d. | A | n.d. | n.d. |
| 591 | A | A | A | A |
| 592 | n.d. | A | n.d. | n.d. |
| 593 | n.d. | A | n.d. | n.d. |
| 594 | n.d. | A | n.d. | n.d. |
| 595 | n.d. | A | n.d. | n.d. |
| 596 | A | A | A | A |
| 597 | A | A | A | A |
| 598 | A | A | A | A |
| 599 | A | A | A | A |
| 600 | A | A | A | A |
| 601 | n.d. | A | n.d. | n.d. |
| 602 | A | A | A | B |
| 603 | n.d. | A | n.d. | A |
| 604 | n.d. | A | n.d. | n.d. |
| 605 | n.d. | A | n.d. | n.d. |
| 606 | n.d. | A | n.d. | n.d. |
| 607 | n.d. | A | n.d. | n.d. |
| 608 | n.d. | A | n.d. | n.d. |
| 609 | A | A | B | B |
| 610 | n.d. | A | n.d. | n.d. |
| 611 | n.d. | A | n.d. | n.d. |
| 612 | A | A | A | A |
| 613 | n.d. | A | n.d. | n.d. |
| 614 | n.d. | A | n.d. | n.d. |
| 615 | A | A | A | A |
| 616 | A | A | A | A |
| 617 | A | A | A | A |
| 618 | A | A | n.d. | n.d. |
| 619 | n.d. | A | n.d. | n.d. |
| 620 | A | A | A | A |
| 621 | n.d. | A | n.d. | n.d. |
| 622 | n.d. | A | n.d. | n.d. |
| 623 | n.d. | A | n.d. | n.d. |
| 624 | n.d. | A | n.d. | n.d. |
| 625 | A | A | A | C |
| 626 | n.d. | A | n.d. | n.d. |
| 627 | n.d. | A | n.d. | n.d. |
| 628 | A | A | A | A |
| 629 | n.d. | A | n.d. | n.d. |
| 630 | A | A | A | A |
| 631 | A | A | A | A |
| 632 | n.d. | A | n.d. | n.d. |
| 633 | n.d. | A | n.d. | n.d. |
| 634 | n.d. | A | n.d. | n.d. |
| 635 | A | A | C | A |
| 636 | A | A | A | A |
| 637 | n.d. | A | n.d. | n.d. |
| 638 | A | A | A | A |
| 639 | n.d. | A | n.d. | n.d. |
| 640 | n.d. | A | n.d. | n.d. |
| 641 | n.d. | A | n.d. | n.d. |
| 642 | n.d. | A | n.d. | n.d. |
| 643 | n.d. | A | n.d. | n.d. |
| 644 | n.d. | A | n.d. | n.d. |
| 645 | A | A | A | A |
| 646 | A | A | A | A |
| 647 | n.d. | A | n.d. | n.d. |
| 648 | A | A | A | A |
| 649 | n.d. | A | n.d. | n.d. |
| 650 | A | A | A | A |
| 651 | A | A | A | A |
| 652 | A | A | A | A |
| 653 | n.d. | A | n.d. | n.d. |
| 654 | n.d. | A | n.d. | n.d. |
| 655 | n.d. | A | n.d. | n.d. |

### Example 14

### Synthesis of 3-Amino-6,7,8,9-tetrahydro-5H-1-thia-7,10-diaza-cyclohepta[f]indene-2-carboxylic acid (5-phenyl-[1,3,4]thiadiazol-2-yl)-amide hydrochloride (C12 or Compound 115 in Table 1)

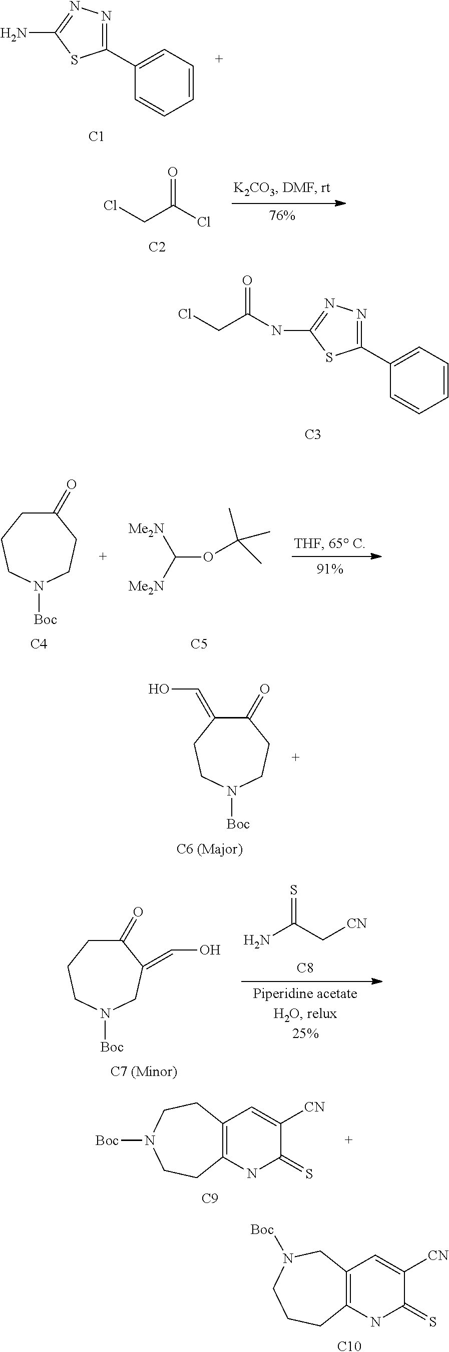

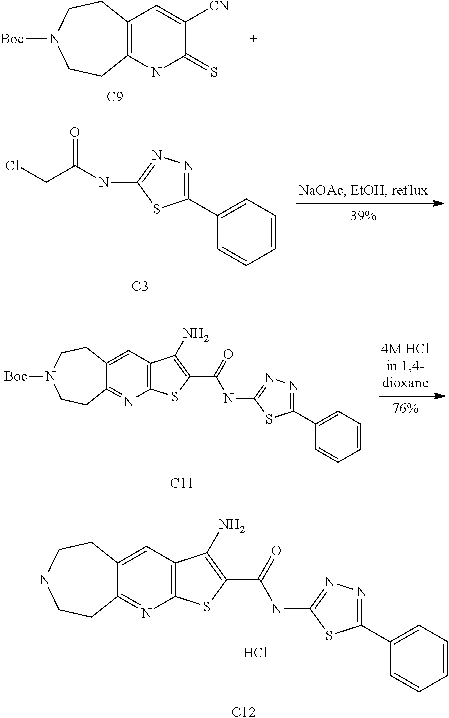

### Step A—Synthesis of 2-chloro-N-(5-phenyl-1,3,4-thiadiazol-2-yl)acetamide (C3)

To a mixture of 5-phenyl-1,3,4-thiadiazol-2-amine (C1, 1.06 g, 6 mmol) and K₂CO₃ (0.83 g, 6 mmol) in anhydrous DMF (20 mL), was added chloroacetyl chloride (C2, 0.48 mL, 6 mmol). The mixture was stirred at room temperature for 4 h. The reaction mixture was then poured into ice-water (100 mL), stirred, and then filtered. The resulting solid was washed with water, and then dried in the oven under vacuum to afford compound C3 (1.15 g, 76%) as a white solid.

### Step B—Synthesis of tert-butyl (4E)-4-(hydroxymethylene)-5-oxoazepane-1-carboxylate (C6) and tert-butyl (3E)-3-(hydroxymethylene)-4-oxoazepane-1-carboxylate (C7)

A solution of tert-butyl 4-oxoazepane-1-carboxylate (C4, 2.56 g, 12.0 mmol) and N-[tert-butoxy(dimethylamino)methyl]-N,N-dimethylamine (C5, 2.97 mL, 14.4 mmol) in THF (30 mL) was refluxed for 8 h. After cooling, the reaction mixture was treated with water (20 mL), stirred at room temperature for 15 min, and then extracted with EtOAc. The organic layer was dried over Na₂SO₄, and concentrated under reduced pressure to give C6 (major) and C7 (minor) as a colorless oil (2.63 g, 91%), which was used as a mixture in the next step reaction directly.

### Step C—Synthesis of tert-butyl 3-cyano-2-thioxo-1,2,5,6,8,9-hexahydro-7H-pyrido[2,3-d]azepine-7-carboxylate (C9) and tert-butyl 3-cyano-2-thioxo-1,2,5,7,8,9-hexahydro-6H-pyrido[3,2-c]azepine-6-carboxylate (C10)

A solution of a mixture of C6 and C7 (2.36 g, 9.8 mmol), 2-cyanoethanethioamide (C8, 0.98 g, 9.8 mmol) and piperidine acetate (10 mL) [prepared from glacial acetic acid (4.2 mL), water (10 mL) and piperidine (7.2 mL)] in H₂O (50 mL) was refluxed for 2 h. After cooling, the reaction mixture was extracted with EtOAc. The combined organic layer was dried over Na₂SO₄, and concentrated under reduced pressure. The given residue was purified through silica gel chromatography (EtOAc/Hexane 60:40) to afford the desired compound C9, a yellow solid (0.75 g, 25%) as the major product. MS: MH⁺=306 and C10 (0.188 g, 6.3%) as the minor product. MS: MH⁺=306.

### Step D—Synthesis of 3-amino-7-tert-butyloxycarbonyl-6,7,8,9-tetrahydro-5H-1-thia-7,10-diaza-cyclohepta[f]indene-2-carboxylic acid (5-phenyl-[1,3,4]thiadiazol-2-yl)-amide (C11)

A mixture of C9 (750 mg, 2.46 mmol), C3 (623 mg, 2.46 mmol) and sodium acetate (302 mg, 3.68 mmol) in EtOH (20 mL) was refluxed for 4 h. After cooling, the reaction mixture was poured into water (100 mL), stirred, and then filtered. The given solid was dried in the oven under vacuum, and then recrystallized in EtOAc to afford compound C11 (500 mg, 39%) as a yellow solid. MS: MNa⁺=545.

### Step E—Synthesis of 3-Amino-6,7,8,9-tetrahydro-5H-1-thia-7,10-diaza-cyclohepta[f]indene-2-carboxylic acid (5-phenyl-[1,3,4]thiadiazol-2-yl)-amide hydrochloride (C12, Compound 115 in the Table)

The Boc-protected amine C11 (150 mg, 0.29 mmol) was stirred in a solution of 4 M HCl in 1,4-dioxane (5 mL) at room temperature for 2 h. Then the mixture was concentrated under reduced pressure and the product was precipitate out in hexane. The given solid was further purified by recrystallization from MeOH/CH₂Cl₂ to afford the target compound C12 (100 mg, 76%) as a red solid. HPLC: purity >97%. MS: MH⁺=423. ¹H NMR (DMSO-d₆+D₂O): δ 8.02 (s, 1H), 7.60 (d, 2H), 7.42 (m, 3H), 4.26 (s, 2H), 3.45 (s, 2H), 3.12 (m, 2H), 1.96 (s, 2H).

### Example 15

### Synthesis of 3-Amino-6,7,8,9-tetrahydro-5H-1-thia-6,10-diaza-cyclohepta[f]indene-2-carboxylic acid (5-phenyl-[1,3,4]thiadiazol-2-yl)-amide hydrochloride (C14 or Compound 52 in Table 1)

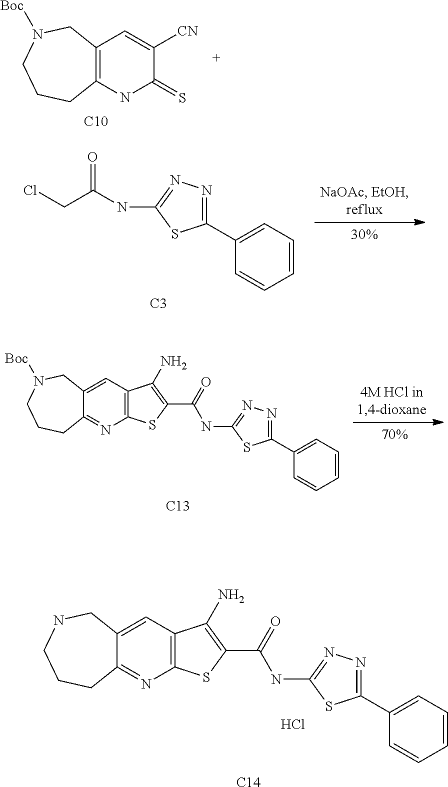

The compound C14 was synthesized in a manner similar to Compound 115 (C12) by utilizing isolated tert-butyl 3-cyano-2-thioxo-1,2,5,7,8,9-hexahydro-6H-pyrido[3,2-c]azepine-6-carboxylate (C10). The compound 3-amino-6-tert-butyloxycarbonyl-6,7,8,9-tetrahydro-5H-1-thia-6,10-diaza-cyclohepta[f]indene-2-carboxylic acid (5-phenyl-[1,3,4]thiadiazol-2-yl)-amide (C13) was confirmed with mass spectroscopy. C14 was obtained as a yellow solid. MS: MH⁺=423. ¹H NMR (DMSO-d₆+D₂O): δ 8.24 (s, 1H), 7.86 (s, 2H), 7.53 (s, 3H), 3.36 (s, 2H), 3.28 (s, 4H), 3.17 (s, 2H).

### Example 16

### Synthesis of Compounds 281, 282 and 283

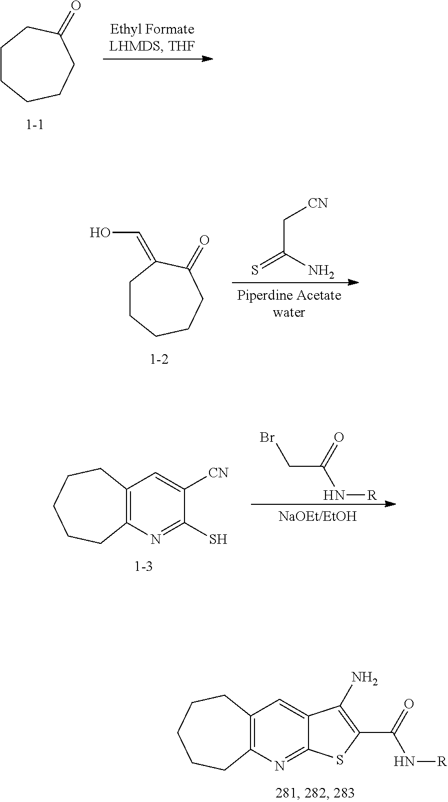

### Synthesis of 2-(hydroxymethylene)cycloheptanone (1-2)

A solution of 1-1 (19.04 g, 169.7 mmol) in anhydrous THF (50 mL) was cooled to 0° C. A solution of LHMDS (1.0 M in THF, 190 mL, 190 mmol) was added dropwise, followed by ethyl formate (13.8 g, 186.3 mmol). The resulting mixture was stirred for 3 h at 0° C. under N₂ and quenched by slow addition of water (300 mL) and hexanes (200 mL). The layers were separated, the aqueous layer was neutralized with 5% citric acid (350 mL), followed by extraction with ethyl acetate (300 mL×2). Organic layers were combined, washed with water (300 mL), brine (300 mL) and dried (Na₂SO₄). The solvent was removed under reduced pressure and 1-2 was obtained as an oil (20.0 g, 84% yield). This was used in the next step without further purification.

### Synthesis of 2-sulfanyl-6,7,8,9-tetrahydro-5H-cyclohepta[b]pyridine-3-carbonitrile (1-3)

A mixture of 1-2 (18.0 g, 128.6 mmol), 2-cyanothioacetamide (12.9 g, 128.6 mmol) and a piperidine solution (122 mL, prepared from piperidine (90 mL) and AcOH (53 mL) in water (125 mL)) in water (643 mL) was heated to reflux for 15 minutes. Additional AcOH (193 mL) was added and the reaction mixture was allowed to cool to room temperature slowly, when compound 1-3 precipitated out as a red solid. The reaction mixture was filtered and the cake was washed with water (100 mL) and dried under vacuum (18.5 g, 70% yield).

### General Procedure for the Preparation of 2-Bromoacetoamide

To a solution of the corresponding primary amine (25 mmol) in anhydrous DCM (100 mL) was added a mixture of 2-bromoacetyl bromide (25 mmol) and triethylamine (30 mmol) in anhydrous DCM (20 mL) at −30° C. under N₂. After the addition, the reaction mixture was stirred at room temperature for 1.5 h and then concentrated. The residue was re-dissolved in acetone (50 mL), precipitated triethylamine hydrobromide was removed by filtration, and the filtrate was evaporated to yield the product. The product was further purified by trituration with diethyl ether.

### General Procedure for the Preparation of Final Products

To a slurry of compound 1-3 (1 mmol, 204 mg) in anhydrous EtOH (5 mL) was added the corresponding 2-bromoacetamide (1 mmol), followed by a solution of sodium ethoxide in EtOH (2.6 M solution, 1.5 mmol, 0.58 mL) at room temperature under N₂. The reaction was heated to reflux for 2 hours and during that time, the desired product precipitated out. The mixture was cooled to room temperature and filtered. The solid was washed by EtOH (2 mL), diethyl ether (5 mL) and dried under vacuum to yield the final products.

### Example 17

### Synthesis of Compounds 284, 286, 287 and 288

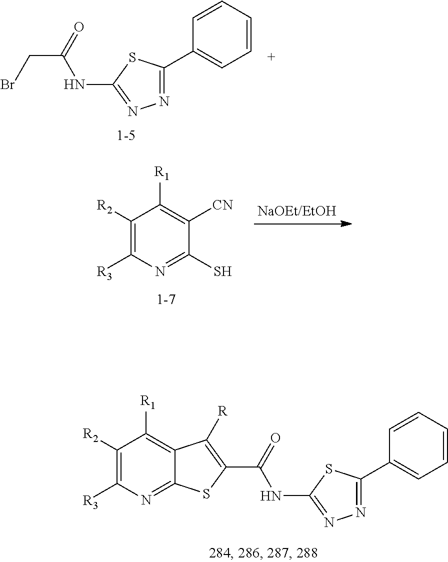

To a slurry of 1-5 (100 mg, 0.333 mmol) in anhydrous EtOH (2.5 mL) was added the corresponding sulfanylpyridine carbonitrile (1-7) followed by a solution of sodium ethoxide in EtOH (2.6 M solution, 0.2 mL, 0.56 mmol) at room temperature under N₂. The reaction was heated to reflux for 2 hours and during that time, the desired product precipitated out. The mixture was cooled to room temperature and filtered. The solid was washed with EtOH (2 mL) and ether (5 mL), and dried under vacuum to give the final compounds.

### Example 18

### Synthesis of Compounds 285, 289, 293 and 294, 295, 296, 297, 298, 358, 359 and 360

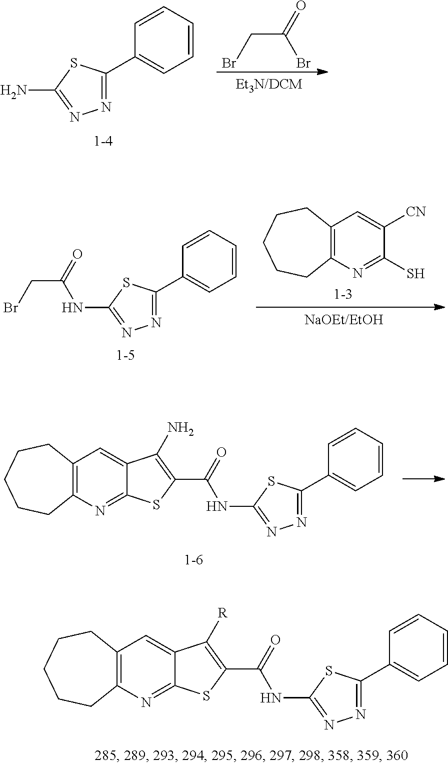

### Synthesis of 2-bromo-N-(5-phenyl-1,3,4-thiadiazol-2-yl)acetamide (1-5)

A slurry of 1-4 (4.0 g, 22.57 mmol) and TEA (4.55 g, 45.14 mmol) in anhydrous DCM (400 mL) was cooled to 10° C. followed by the dropwise addition of 2-bromoacetyl bromide (9.12 g, 45.14 mmol). After the addition was complete, the mixture was stirred at room temperature overnight under N₂ and then filtered. The cake was washed with DCM (100 mL), aqueous saturated NaHCO₃ (100 mL), diethyl ether (100 mL) and dried under vacuum to give 1-5 (4.85 g, yield 72%).

### Synthesis of 3-amino-N-(5-phenyl-1,3,4-thiadiazol-2-yl)-6,7,8,9-tetrahydro-5H-cyclohepta[b]thieno[3,2-e]pyridine-2-carboxamide (1-6)

To a slurry of 1-3 (2.04 g, 10 mmol) in anhydrous EtOH (100 mL) was added 1-5 (2.99 g, 10 mmol), followed by a solution of sodium ethoxide in EtOH (2.6 M solution, 5.8 mL, 15 mmol) at room temperature under N₂. The reaction was heated to reflux for 2 hours and during that time, the desired product precipitated out. The mixture was cooled to room temperature and filtered. The solid was washed with EtOH (20 mL), diethyl ether (50 mL), and dried under vacuum to give 1-6 (3.30 g, yield 78%).

### Synthesis of 3-benzamido-N-(5-phenyl-1,3,4-thiadiazol-2-yl)-6,7,8,9-tetrahydro-5H-cyclohepta[b]thieno[3,2-e]pyridine-2-carboxamide (285)

To a solution of 1-6 (500 mg, 1.18 mmol) in anhydrous DMF (5 mL) was added pyridine (0.15 mL) at room temperature under N₂, followed by benzoic anhydride (401 mg, 1.77 mmol). Then the mixture was stirred at 50° C. overnight. HPLC revealed about 60% conversion. More benzoic anhydride (267 mg) and pyridine (0.15 mL) were added and the mixture was stirred at 50° C. for another 5 hours. DCM (100 mL) was added and the mixture was washed with water (10 mL), aqueous saturated NaHCO₃ (10 mL), brine (10 mL) and dried (Na₂SO₄). The solvent was removed under reduced pressure and the residue was purified by silica gel column chromatography to give 285 (35 mg, yield 7%).

### Synthesis of 3-(butylamino)-N-(5-phenyl-1,3,4-thiadiazol-2-yl)-6,7,8,9-tetrahydro-5H-cyclohepta[b]thieno[3,2-e]pyridine-2-carboxamide (289)

To a solution of 1-6 (200 mg, 0.475 mmol) in anhydrous NMP (2 mL) was added n-BuI (131 mg, 0.713 mmol) and the mixture was stirred at room temperature for 1 h under N₂. Then, DCM (100 mL) was added and the mixture was washed with water (10 mL), aqueous saturated NaHCO₃ (10 mL), brine (10 mL) and dried (Na₂SO₄). Most of the solvent was removed under reduced pressure and the precipitated solid was filtered. The cake was washed with diethyl ether (10 mL) and dried under vacuum to yield 289 (70 mg, 31% yield).

### Synthesis of 2-((2-((5-phenyl-1,3,4-thiadiazol-2-yl)carbamoyl)-6,7,8,9-tetrahydro-5H-cyclohepta[b]thieno[3,2-e]pyridin-3-yl)amino)acetic acid (293)

To a mixture of intermediate 1-6 (0.63 g, 1.5 mmol) and TEA (0.9 mL, 6.0 mmol, 4.0 eq) in anhydrous THF (20 mL) was slowly added ethyl bromoacetate (0.4 mL, 3.0 mmol, 2.0 eq) and the contents were stirred overnight at room temperature. The volatiles were removed under vacuum and the residue was purified by flash chromatography on silica gel eluting 0-5% MeOH/DCM affording the desired intermediate. This material was treated with aqueous 1M LiOH (4 mL) in THF—H₂O (3:1, 20 mL) at room temperature overnight. Most of the THF was removed under vacuum and the aqueous layer was washed with MTBE:EtOAc (1:1, 10 mL) and acidified to pH=3-5 using acetic acid. The free acid obtained was stirred with sodium methoxide (1 eq) in MTBE to give the desired sodium salt of 293 (0.12 g, 9% overall yield) as a solid.

### Synthesis of 3-((2-aminoethyl)amino)-N-(5-phenyl-1,3,4-thiadiazol-2-yl)-6,7,8,9-tetrahydro-5H-cyclohepta[b]thieno[3,2-e]pyridine-2-carboxamide (294)

To a solution of intermediate 1-6 (0.42 g, 1 mmol) and triethylamine (2 mL) in N-methylpyrrolidinone (20 mL) was added N(Boc)-2-bromoethylamine (1.8 g, 8.0 mmol) and the contents were heated at 100° C. for 16 h. The reaction mixture was cooled to room temperature and poured into ice-cold water. The solid obtained was filtered and air-dried to give the free base (0.23 g). Treatment of the free base with 2M HCl in diethyl ether (10 mL) at room temperature overnight followed by filtration afforded 294 in the HCl salt form (0.19 g, 38% overall yield).

### Synthesis of 3-oxo-3-((2-((5-phenyl-1,3,4-thiadiazol-2-yl)carbamoyl)-6,7,8,9-tetrahydro-5H-cyclohepta[b]thieno[3,2-e]pyridin-3-yl)amino)propanoic acid (295)

To a solution of intermediate 1-6 (0.63 g, 1.5 mmol) and TEA (1 mL) in anhydrous DCM (30 mL) at 0° C. was added methylmalonyl chloride (0.4 g, 3.0 mmol, 2.0 eq) dropwise and the contents were slowly warmed to room temperature and stirred for 24 h. The organic portion was washed with 1M NaOH, brine, dried (Na₂SO₄), filtered and concentrated. The crude methyl ester was stirred with 1M LiOH (4 mL) in THF (12 mL) and water (4 mL) at room temperature overnight. Most of the THF was removed under vacuum and the solid obtained was filtered, dried and treated with sodium methoxide (1.0 eq) in MTBE at room temperature overnight. The solid obtained was filtered and dried under vacuum to give the sodium salt of 295 (0.3 g, 38% overall yield) as a brown solid.

### Synthesis of 3-(2-aminoacetamido)-N-(5-phenyl-1,3,4-thiadiazol-2-yl)-6,7,8,9-tetrahydro-5H-cyclohepta[b]thieno[3,2-e]pyridine-2-carboxamide (296)

To a solution of intermediate 1-6 (1.26 g, 3.0 mmol) and Boc-glycine (1.05 g, 6.0 mmol, 2.0 eq) in anhydrous DMF (30 mL) at room temperature was sequentially added HBTU (2.27 g, 6.0 mmol, 2.0 eq) and DIEA (2.6 mL, 15 mmol, 5.0e q). The contents were stirred at room temperature for 36 h. The reaction mixture was poured into ice-cold water and the solid obtained was filtered, and dried under vacuum. The solid was dissolved in TFA (10 mL) and DCM (20 mL) and stirred overnight. The volatiles were removed under vacuum. The residue obtained was stirred in 2M HCl in diethyl ether (20 mL) at room temperature overnight and the solid was filtered, dried under vacuum to yield 296 as the HCl salt (0.6 g, 39% overall yield).

### Synthesis of 3-acetamido-N-(5-phenyl-1,3,4-thiadiazol-2-yl)-6,7,8,9-tetrahydro-5H-cyclohepta[b]thieno[3,2-e]pyridine-2-carboxamide (358)

To a solution of 1-6 (200 mg, 0.475 mmol) in anhydrous DMF (2 mL) was added pyridine (0.05 mL) followed by acetic anhydride (60 mg, 0.57 mmol). The reaction mixture was stirred at room temperature overnight and then DCM (100 mL) was added. The mixture was washed with water (10 mL), aqueous saturated NaHCO₃ (10 mL), brine (10 mL) and dried (Na₂SO₄). The solvent was removed under reduced pressure and the residue was purified by silica gel column chromatography to give 358 (40 mg, yield 19%).

### Synthesis of 3-(methylamino)-N-(5-phenyl-1,3,4-thiadiazol-2-yl)-6,7,8,9-tetrahydro-5H-cyclohepta[b]thieno[3,2-e]pyridine-2-carboxamide (359)

To a solution of 1-6 (200 mg, 0.475 mmol) in anhydrous NMP (2 mL) was added CH₃I (102 mg, 0.712 mmol) and stirred for 1 hour at room temperature under N₂. Then, DCM (100 mL) was added and the mixture was washed with water (10 mL), saturated aqueous NaHCO₃ (10 mL), brine (10 mL) and dried (Na₂SO₄). Most of the solvent was removed under reduced pressure and the precipitated solid was filtered. The cake was washed with diethyl ether (10 mL) and dried under vacuum to give 359 (95 mg, 48% yield).

### General Procedure for Compounds 297, 298 and 360

To a solution of intermediate 1-6 (0.84 g, 2.0 mmol) and the corresponding pyridine carboxylic acid (0.49 g, 4.0 mmol, 2.0 eq) in anhydrous DMF (25 mL) at room temperature was sequentially added HBTU (1.52 g, 4.0 mmol, 2.0 eq) and DIEA (3.5 mL, 20 mmol, 10 eq) and the contents were stirred at room temperature overnight. The reaction mixture was poured into ice-cold water and the solid obtained was filtered and dried under vacuum. The free base obtained above was stirred in 2M HCl in diethyl ether (10 mL), filtered and dried to give the appropriate HCl salt form of the final compounds.

### Example 19

### Synthesis of Compound 290

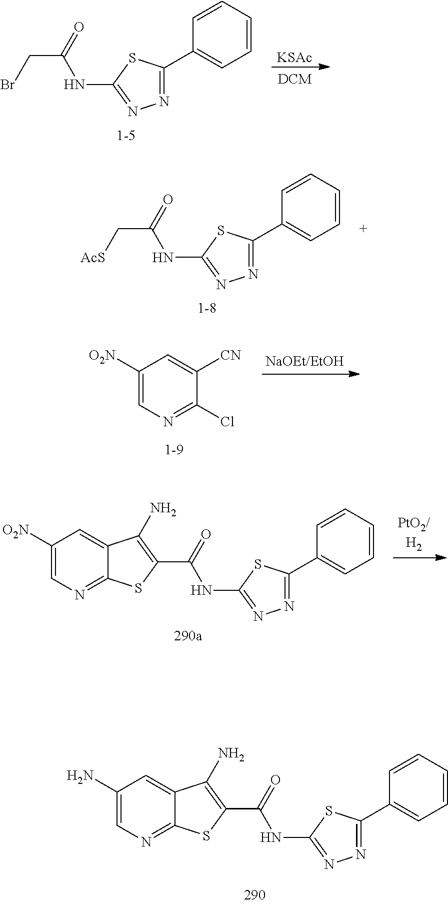

### Synthesis of S-[2-oxo-2-[(5-phenyl-1,3,4-thiadiazol-2-yl)amino]ethyl]ethanethioate (1-8)

To a slurry of 1-5 (300 mg, 1 mmol) in anhydrous DCM (30 mL) was added potassium thioacetate (171 mg, 1.5 mmol) and the mixture was stirred at room temperature overnight. The precipitate was filtered, the filter cake was washed with diethyl ether (30 mL), and dried under vacuum to give intermediate 1-8 (287 mg, yield 95%).

### Synthesis of 3-amino-5-nitro-N-(5-phenyl-1,3,4-thiadiazol-2-yl)thieno[2,3-b]pyridine-2-carboxamide (290a)

To a slurry of 1-8 (100 mg, 0.34 mmol) in anhydrous EtOH (5 mL) was added a solution of NaOEt in EtOH (2.6 M solution, 0.2 mL, 0.52 mmol) at room temperature under N₂ for 1 h. Then, 1-9 (62 mg, 0.34 mmol) was added to the mixture and the reaction was heated to reflux for 2 hours. During that time, the desired product precipitated out. The mixture was cooled to room temperature and filtered. The solid was washed with EtOH (10 mL) and diethyl ether (15 mL), and dried under vacuum to give 290a (53 mg, 39% overall yield).

### Synthesis of 3,5-diamino-N-(5-phenyl-1,3,4-thiadiazol-2-yl)thieno[2,3-b]pyridine-2-carboxamide (290)

To a slurry of 17 (280 mg, 0.704 mmol) in anhydrous EtOH (60 mL) was added PtO₂ (28 mg), and the mixture was hydrogenated at 30 psi for 3 days. The mixture was filtered through Celite, the filtrate was concentrated and the resulting residue was recrystallized with MeOH/diethyl ether (1:4, 5 mL) to give 290 (45 mg, 18% yield).

### Example 20

### Synthesis of Compound 291

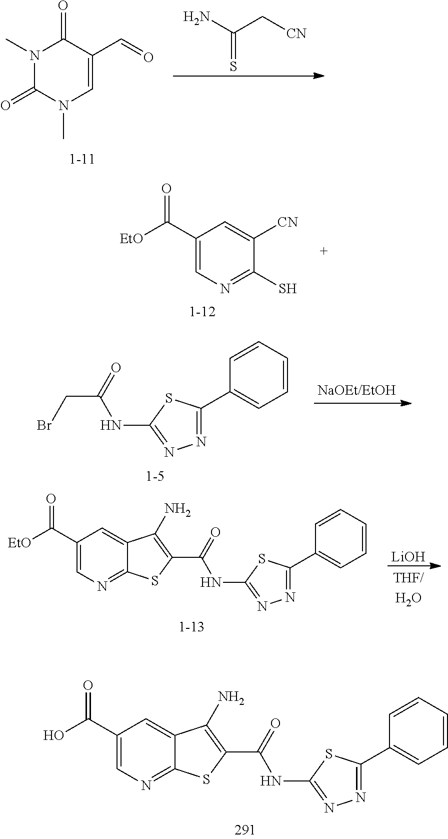

### Synthesis of Ethyl 5-cyano-6-sulfanyl-pyridine-3-carboxylate (1-12)

To a solution of 1-11 (500 mg, 3.00 mmol) and 2-cyanothioacetamide (1.0 g, 10.0 mmol) in anhydrous EtOH (36 mL) was added a solution of NaOEt in EtOH (2.6 M solution, 4.0 mL, 1.04 mmol) at room temperature and then the mixture was heated to reflux for 1 hour. The mixture was cooled to room temperature, concentrated and the residue was dissolved in water (20 mL). Concentrated HCl was added dropwise to adjust the pH to 8-9 when a solid precipitated out. The precipitate was collected by filtration and filter cake was washed with water and dried under vacuum to yield 1-12 (212 mg, 34% yield).

### Synthesis of Ethyl 3-amino-2-[(5-phenyl-1,3,4-thiadiazol-2-yl)carbamoyl]thieno[2,3-b]pyridine-5-carboxylate (1-13)

To a slurry of compound 1-12 (150 mg, 0.721 mmol) in anhydrous EtOH (5 mL) was added 1-5 (216 mg, 0.721 mmol), followed by a solution of NaOEt in EtOH (2.6 M solution, 0.5 mL, 1.3 mmol) at room temperature under N₂. The reaction was heated to reflux for 2 hours and during that time, the desired product precipitated out. The mixture was cooled to room temperature and filtered. The solid was washed with EtOH (2 mL), diethyl ether (5 mL), and dried under vacuum to give 1-13 (230 mg, 75% yield).

### Synthesis of 3-amino-2-[(5-phenyl-1,3,4-thiadiazol-2-yl)carbamoyl]thieno[2,3-b]pyridine-5-carboxylic acid (291)

To a slurry of compound 1-13 (230 mg, 0.54 mmol) in THF (5 mL) was added a solution of LiOH in water (1 M solution, 1.35 mL, 1.35 mmol). The reaction was stirred at room temperature for 2 hours and during that time the desired product precipitated out. After filtration, the solid was washed with EtOH (2 mL) and diethyl ether (5 mL), and dried under vacuum to give 291 (48 mg, 22% yield).

### Example 21

### Synthesis of Compound 292

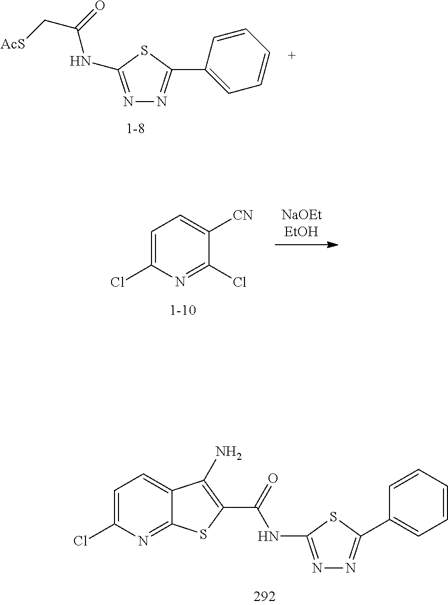

To a slurry of 1-8 (200 mg, 0.669 mmol) in anhydrous EtOH (10 mL) was added a solution of NaOEt in EtOH (2.6 M solution, 0.4 mL, 1.04 mmol) at room temperature under nitrogen for one hour. Then, 1-10 (116 mg, 0.669 mmol) was added to the mixture and the reaction was heated to reflux for 2 hours. During that time, the desired product precipitated out. The mixture was cooled to room temperature and filtered. The solid was washed with EtOH (10 mL), diethyl ether (15 mL), and dried under vacuum to yield 292 (35 mg, 15% overall yield).

### Example 22

### Synthesis of Compounds 299, 300, 361 and 362

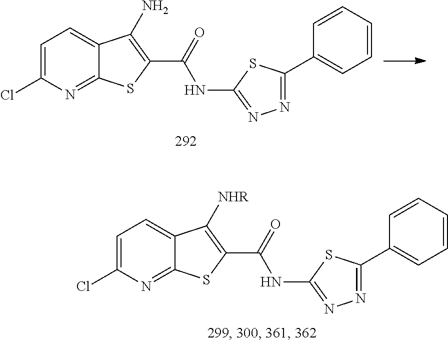

### Synthesis of 2-[[6-chloro-2-[(5-phenyl-1,3,4-thiadiazol-2-yl)carbamoyl]thieno[2,3-b]pyridin-3-yl]amino]acetic acid (299)

A solution of 292 (200 mg, 1 eq), TEA (0.32 mL, 6 eq) in DMF (3 ml) with ethyl bromoacetate (172 mg, 2 eq) was stirred at room temperature for 2 h. The reaction was poured into ice water (10 mL), filtered, and dried to afford the ethyl ester intermediate. This material was dissolved in 3:1 THF/H₂O (10 mL) and 1M NaOH (1.5 mL, 3 eq) and stirred at room temperature for 2 h. Following removal of THF, the resulting solid was collected by filtration and dried under vacuum to afford product 299 as the sodium salt (105 mg, 43% overall yield).

### Synthesis of 3-(2-aminoethylamino)-6-chloro-N-(5-phenyl-1,3,4-thiadiazol-2-yl)thieno[2,3-b]pyridine-2-carboxamide (300)

A solution of 292 (350 mg, 1 eq), TEA (2 ml), and N-(Boc)-2-bromoethylamine (1 g, 5 eq) in NMP (20 mL) was heated at 100° C. for 16 h. The reaction mixture was cooled to room temperature, poured into ice water (60 mL), and the solid was filtered and dried to give the Boc-protected intermediate. This solid dissolved in 10% HCl in MeOH (20 mL) and stirred at room temperature for 3 h. The volume of the reaction mixture was reduced to 3 mL, the solid was collected by filtration and washed by diethyl ether (3×3 mL) to afford product 300 (85 mg, 20% yield) as a light-yellow powder.

### Synthesis of 3-[(2-aminoacetyl)amino]-6-chloro-N-(5-phenyl-1,3,4-thiadiazol-2-yl)thieno[2,3-b]pyridine-2-carboxamide (361)

A solution of 292 (200 mg, 1 eq), Boc-glycine (180 mg, 2 eq), HBTU (390 mg, 2 eq) and DIPEA (0.447 mL, 5 eq) in DMF (5 mL) were stirred at room temperature for 3 days. The reaction was poured into ice water (20 mL), filtered, and dried to isolate the Boc-protected intermediate. This material was dissolved in 10% HCl in MeOH (10 mL) and the reaction was stirred at room temperature for 2 h. After removing solvents, the resulting solid was washed with EtOH (3×10 mL) and DCM (3×10 mL) to afford 361 as the HCl salt (30 mg, 12% overall yield).

### Synthesis of 3-[[6-chloro-2-[(5-phenyl-1,3,4-thiadiazol-2-yl)carbamoyl]thieno[2,3-b]pyridin-3-yl]amino]-3-oxo-propanoic acid (362)

A mixture of 292 (1 g, 1 eq) and TEA (3.33 ml) in anhydrous DCM (100 mL) was stirred at 0° C., then methyl malonyl chloride (0.833 mL, 3 eq) was added slowly. After stirring at room temperature for 18 h, DMF (5 mL) was added and the reaction was stirred for an additional 6 h in attempt to drive to completion. The mixture was concentrated to dryness, triturated in water (500 mL) for 1 h, filtered, and the solid was washed by MTBE (3×30 mL). This crude ester intermediate was purified by silica gel column chromatography using 0-5% MeOH/DCM to give pure material (385 mg, 31% yield). The hydrolysis reaction was performed with the purified ester intermediate (386 mg, 1 eq) in 3:1 THF/H₂O (30 mL) and 1M NaOH (3.4 mL, 4.3 eq). The reaction was stirred at room temperature and then concentrated to dryness. The resulting solid was collected by filtration, washed by MTBE (3×50 mL), and dried to give 362 as a light-yellow solid (215 mg, 17% overall yield).

### Example 23

### Synthesis of Compound 301

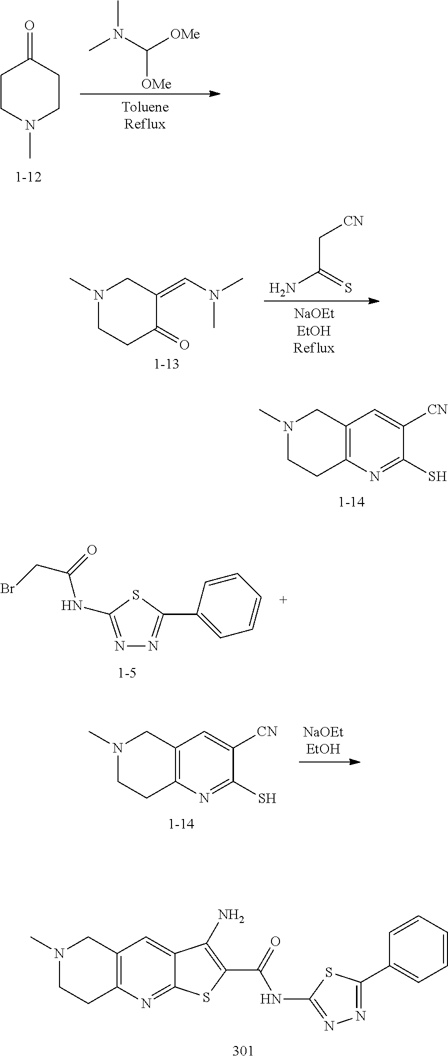

### Synthesis of 3-(dimethylaminomethylene)-1-methyl-piperidin-4-one (1-13)

A mixture of 1-12 (25 mL, 203 mmol, 1.0 eq), and N,N-dimethylformamide dimethylacetal (30 mL, 223.3 mmol, 1.1 eq) in toluene (200 mL) was heated to reflux for 12 h. Additional N,N-dimethylformamide dimethylacetal (30 mL, 223.3 mmol, 1.1 eq) was added and the heating was continued for another 24 h. Volatiles were removed under reduced pressure and N,N-dimethylformamide dimethylacetal (60 mL, 446.6 mmol, 2.2 eq) was added to the residue yet again and it was heated at 100° C. overnight. The reaction mixture was evaporated under reduced pressure, and twice azeotroped toluene twice to afford 48 g (˜70% purity by LC-MS) of crude 1-13 as a dark brown liquid.

### Synthesis of 6-methyl-2-sulfanyl-7,8-dihydro-5H-1,6-naphthyridine-3-carbonitrile (1-14)

To a mixture of crude compound 1-13 (15 g, 89 mmol, 1.3 eq) and 2-cyanothioacetamide (6.9 g, 68.5 mmol, 1 eq) in anhydrous EtOH (150 mL) at room temperature, was added NaOEt (21 wt % in EtOH, 55 mL, 144 mmol, 2.1 eq) and the reaction mixture was heated to reflux overnight. The reaction mixture was cooled to room temperature, poured into ice water and acidified with aqueous HCl (2N) to pH˜2. The mixture was filtered and the filtrate was evaporated under reduced pressure. The residue was triturated with MeOH, filtered and dried under vacuum to afford 12 g (66% yield, >85% purity by LC-MS) of crude compound 1-14 as a yellow solid.

### Synthesis of 3-amino-6-methyl-N-(5-phenyl-1,3,4-thiadiazol-2-yl)-7,8-dihydro-5H-thieno[2,3-b][1,6]naphthyridine-2-carboxamide (301)

See procedure used for the synthesis of 1-6.

### Example 24

### Synthesis of Compounds 302, 304-311, 321 and 363

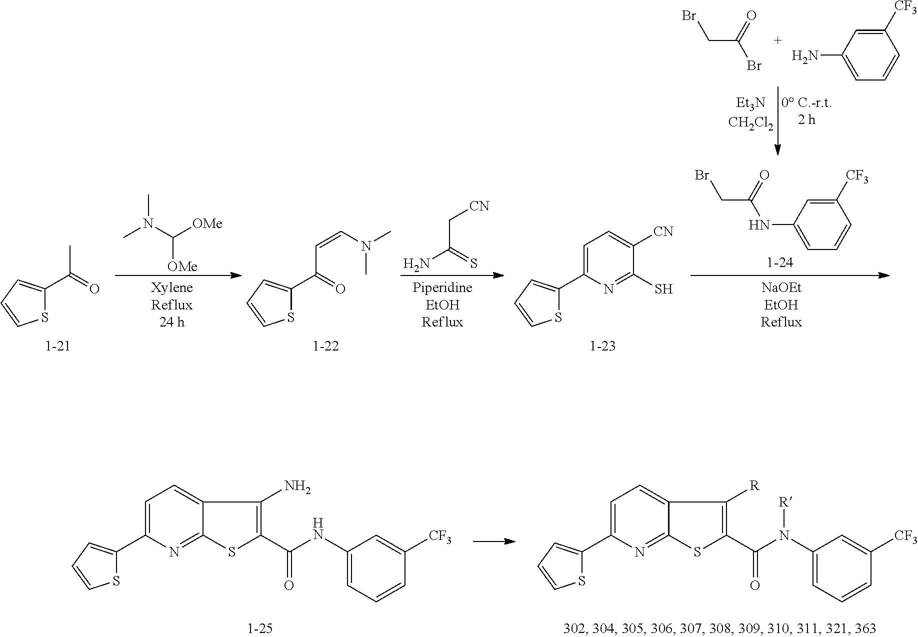

### Synthesis of 3-(dimethylamino)-1-(2-thienyl)prop-2-en-1-one (1-22)

See procedure used for the synthesis of 1-13.

### Synthesis of 2-sulfanyl-6-(2-thienyl)pyridine-3-carbonitrile (1-23)

See procedure used for the synthesis of 1-14.

### Synthesis of 2-bromo-N-[3-(trifluoromethyl)phenyl]acetamide (1-24)

See procedure used for the synthesis of 1-5.

### Synthesis of 3-amino-6-(2-thienyl)-N-[3-(trifluoromethyl)phenyl]thieno[2,3-b]pyridine-2-carboxamide (1-25)

See procedure used for the synthesis of 1-6.

### Synthesis of 3-oxo-3-[[6-(2-thienyl)-2-[[3-(trifluoromethyl)phenyl]carbamoyl]thieno[2,3-b]pyridin-3-yl]amino]propanoic acid (302)

See procedure used for the synthesis of compound 295.

### Synthesis of 3-(2-aminoethylamino)-6-(2-thienyl)-N-[3-(trifluoromethyl)phenyl]thieno[2,3-b]pyridine-2-carboxamide (304)

See procedure used for the synthesis of compound 294.

### Synthesis of 2-[[6-(2-thienyl)-2-[[3-(trifluoromethyl)phenyl]carbamoyl]thieno[2,3-b]pyridin-3-yl]amino]acetic acid (305)

See procedure used for the synthesis of compound 299.

### Synthesis of 2-[carboxymethyl-[6-(2-thienyl)-2-[[3-(trifluoromethyl)phenyl]carbamoyl]thieno[2,3-b]pyridin-3-yl]amino]acetic acid (363)

By-product resulting from disubstitution of the glycine reagent during the synthesis of compound 305.

### Synthesis of 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione (306)

By-product resulting from intramolecular cyclization of the bromoacetyl intermediate used for the synthesis of compounds 307, 308, and 309.

### Synthesis of 3-[[2-(methylamino)acetyl]amino]-6-(2-thienyl)-N-[3-(trifluoromethyl)phenyl]thieno[2,3-b]pyridine-2-carboxamide (307)

A solution of 1-25 (500 mg) in 1,4-dioxane was reacted with bromoacetyl bromide and TEA. After stirring at room temperature for 20 minutes, the reaction mixture was poured into cold diethyl ether, stirred for 10 min, filtered, washed with diethyl ether and dried in vacuo to afford 760 mg (quantitative yield) of the bromoacetyl intermediate as the hydrobromide salt. On 200 mg scale, this bromoacetyl intermediate was reacted with a methylamine solution (33% wt. solution in EtOH) for 2 hours at room temperature. The reaction mixture was evaporated to dryness and triturated with DCM to afford pure compound. This material was treated with 1.25M HCl in MeOH and stirred for 2 hours. Following evaporation in vacuo and trituration with diethyl ether, 75 mg of compound 307 was isolated as the HCl salt (44% yield).

### Synthesis of 3-[[2-(dimethylamino)acetyl]amino]-6-(2-thienyl)-N-[3-(trifluoromethyl)phenyl]thieno[2,3-b]pyridine-2-carboxamide (308)

On 200 mg scale, the bromoacetyl intermediate used for the synthesis of compound 307 was reacted with a 2M dimethylamine solution in THF for 1 hour at room temperature. The reaction mixture was evaporated to dryness and treated with 2M HCl in diethyl ether and stirred for 1 hour. The reaction mixture was filtered and triturated with DCM to afford 135 mg of 308 as the HCl salt (79% yield).

### Synthesis of Trimethyl-[2-oxo-2-[[6-(2-thienyl)-2-[[3-(trifluoromethyl)phenyl]carbamoyl]thieno[2,3-b]pyridin-3-yl]amino]ethyl]ammonium (309)

On 150 mg scale, the bromoacetyl intermediate used for the synthesis of compound 307 was mixed with a 25% trimethylamine in MeOH solution for 1 hour at room temperature. The reaction mixture was evaporated to dryness and triturated with DCM to afford 100 mg of 309 (71% yield).

### Synthesis of Ethyl 4-oxo-4-[[6-(2-thienyl)-2-[[3-(trifluoromethyl)phenyl]carbamoyl]thieno[2,3-b]pyridin-3-yl]amino]butanoate (310)

A solution of compound 1-25 (0.71 g, 1.69 mmol, 1.0 equiv) in 1,4-dioxane (20 mL) was treated with succinyl chloride (5.0 mL, excess) at room temperature under N₂. The reaction mixture was stirred for 2 h. The reaction mixture was poured into cold diethyl ether and the resulting solid was filtered, washed with diethyl ether and dried to afford 0.9 g, (99% yield) of 310 as a pale yellow solid.

### Synthesis of 4-oxo-4-[[6-(2-thienyl)-2-[[3-(trifluoromethyl)phenyl]carbamoyl]thieno[2,3-b]pyridin-3-yl]amino]butanoic acid (311)

Compound 310 (0.548 g, 1.0 mmol, 1.0 equiv) was dissolved in THF/H₂O (3:1; 120 mL) and treated with sodium hydroxide (0.4 g, 10 mmol, 10 equiv) at room temperature for 2 h. The reaction mixture was evaporated to reduce the volume. The resulting precipitate was filtered and washed with DCM and hexanes. After drying, 0.44 g (81% yield) of the sodium salt of 311 was isolated as a yellow solid.

### Synthesis of 3-(ethylamino)-6-(2-thienyl)-N-[3-(trifluoromethyl)phenyl]thieno[2,3-b]pyridine-2-carboxamide (321)

To a solution of compound 1-25 (0.5 g, 1.2 mmol, 1 eq) in anhydrous 1,4-dioxane (30 mL) was added dropwise triethyloxonium tetrafluoroborate (0.29 g, 1.55 mmol, 1.3 eq) in DCM (5 mL) at 5° C. The reaction mixture was allowed to warm to room temperature and stir overnight. The reaction mixture was evaporated in vacuo, triturated with diethyl ether, filtered and washed with diethyl ether. This crude material was purified by trituration with MeOH to afford 70 mg of 321 (13% yield) as a bright yellow solid.

### Example 25

### Synthesis of Compounds 303 and 312

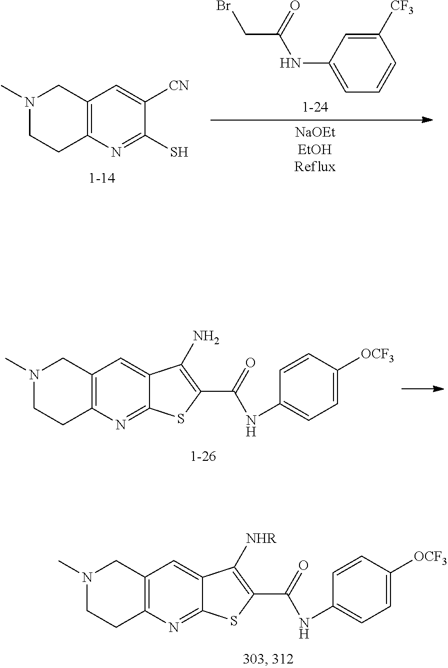

### Synthesis of 3-amino-6-methyl-N-[4-(trifluoromethoxy)phenyl]-7,8-dihydro-5H-thieno[2,3-b][1,6]naphthyridine-2-carboxamide (1-26)

See procedure used for the synthesis of 1-6.

### Synthesis of 2-[[6-methyl-2-[[4-(trifluoromethoxy)phenyl]carbamoyl]-7,8-dihydro-5H-thieno[2,3-b][1,6]naphthyridin-3-yl]amino]acetic acid (303)

See procedure used for the synthesis of compound 299.

### Synthesis of 3-[[2-(dimethylamino)acetyl]amino]-6-methyl-N-[4-(trifluoromethoxy)phenyl]-7,8-dihydro-5H-thieno[2,3-b][1,6]naphthyridine-2-carboxamide (312)

See procedure used for the synthesis of compound 308.

### Example 26

### Synthesis of Compound 316

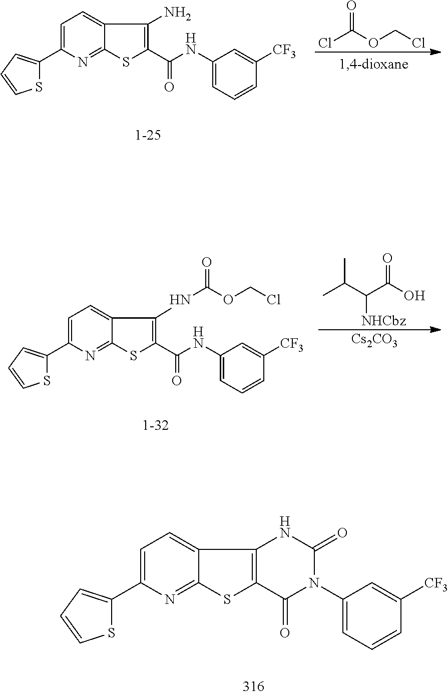

### Synthesis of Chloromethyl N-[6-(2-thienyl)-2-[[3-(trifluoromethyl)phenyl]carbamoyl]thieno[2,3-b]pyridin-3-yl]carbamate (1-32)

To a solution of intermediate 1-25 (1.26 g, 3 mmol) in anhydrous 1,4-dioxane (60 mL) at room temperature was added chloromethyl chloroformate (1 mL, 12 mmol) and the contents were stirred overnight. The solid obtained was filtered, triturated with MTBE (2×20 mL) and dried to afford the desired intermediate 1-32 (1 g) as the HCl salt.

### Synthesis of 7-(thiophen-2-yl)-3-(3-(trifluoromethyl)phenyl)pyrido[3′,2′:4,5]thieno[3,2-d]pyrimidine-2,4(1H,3H)-dione (316)

To a solution of (L)-Cbz-valine (2.5 g, 10 mmol) in anhydrous DMF (100 mL) at room temperature was added cesium carbonate (3.3 g, 10 mmol) and the mixture was stirred for 1 h. To the reaction flask was added the intermediate 1-32 (1 g) and the contents were stirred at room temperature overnight. The reaction mixture was added to ice-cold water and the precipitate obtained was filtered, washed with MTBE (2×30 mL) and dried to afford 316 as a yellow solid (0.5 g).

### Example 27

### Synthesis of Compound 317

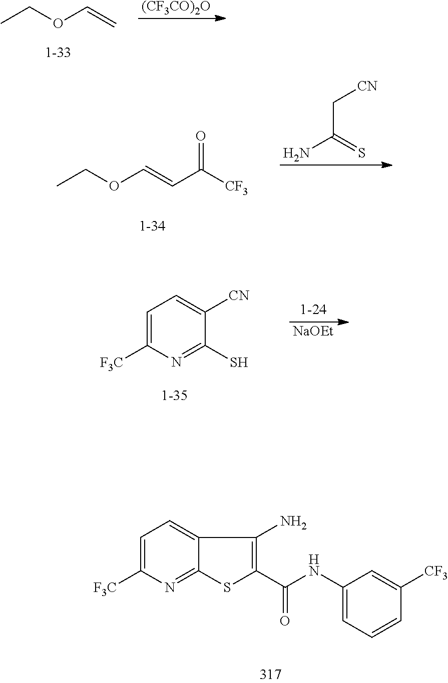

### Synthesis of 4-ethoxy-1,1,1-trifluoro-but-3-en-2-one (1-34)

To a solution of trifluoroacetic anhydride (8.6 mL, 61.9 mmol, 1.05 eq) and N,N-dimethylamino pyridine (0.43 g, 3.54 mmol, 0.06 eq) in DCM (90 mL) at −10° C. was added dropwise methyl vinyl ether (5.6 mL, 59 mmol, 1 eq). The reaction mixture was stirred at −10° C. and warmed to room temperature overnight. GC-MS analysis of the reaction mixture showed completion of reaction. The reaction mixture was poured into a cold saturated sodium bicarbonate solution and extracted with DCM. The combined organic layers were washed with brine, dried (Na₂SO₄), filtered and concentrated to afford 8.5 g (85% yield) of compound 1-34 as a dark brown liquid.

### Synthesis of 2-sulfanyl-6-(trifluoromethyl)pyridine-3-carbonitrile (1-35)

To a mixture of 1-34 (3 g, 17.8 mmol, 1 eq) and 2-cyanothioacetamide (2.7 g, 26.8 mmol, 1.5 eq) in ethanol (30 mL) was added N-methylmorpholine (2.5 mL) and refluxed for 24 h. The reaction mixture was evaporated in vacuo to afford 7 g of crude 1-35 which was used in the next step without purification.

### Synthesis of 3-amino-6-(trifluoromethyl)-N-[3-(trifluoromethyl)phenyl]thieno[2,3-b]pyridine-2-carboxamide (317)

See procedure used for the synthesis of 1-6.

### Example 28

### Synthesis of Compound 318

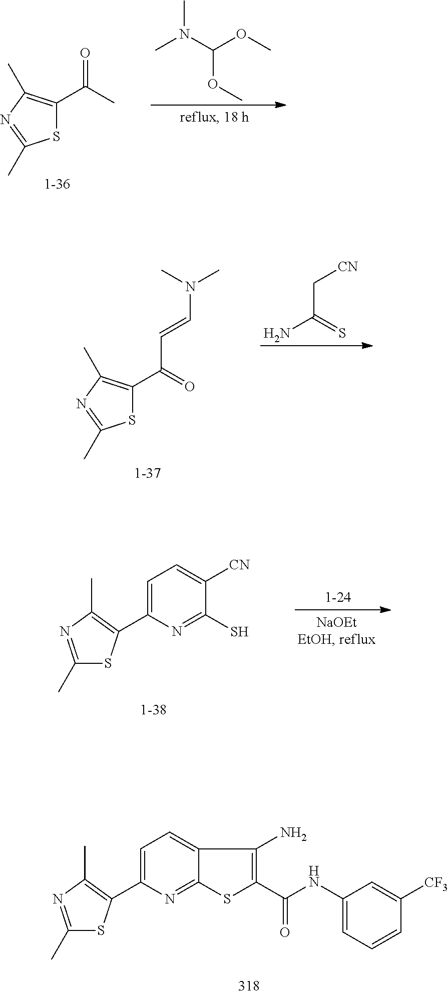

### Synthesis of 3-(dimethylamino)-1-(2,4-dimethylthiazol-5-yl)prop-2-en-1-one (1-37)

A solution of 1-acetyl-2,4-dimethyl-thiazole (10 g, 64 mmol) in N,N-dimethylformamide dimethylacetal (100 mL) was refluxed overnight. GC/MS analysis showed completion. The contents were cooled to room temperature and poured into ice-cold water. The solid 1-37 obtained (10 g, 80%) was dried and used in the next step as such.

### Synthesis of 6-(2,4-dimethylthiazol-5-yl)-2-sulfanyl-pyridine-3-carbonitrile (1-38)

To a mixture of 1-37 (10 g, 48 mmol) and 2-cyanothioacetamide (10 g, 100 mmol) in EtOH (200 mL) was added NMP (10 mL) and the contents were heated at 80° C. overnight. The volatiles were removed under vacuum and the residue was triturated with a 2:1 mixture of hexane/EtOAc affording the desired intermediate 1-38 (7.2 g, 60% yield) as an orange solid, which was used in the next step as such.

### Synthesis of 3-amino-6-(2,4-dimethylthiazol-5-yl)-N-[3-(trifluoromethyl)phenyl]thieno[2,3-b]pyridine-2-carboxamide (318)

See procedure used for the synthesis of 1-6.

### Example 29

### Synthesis of Compound 319

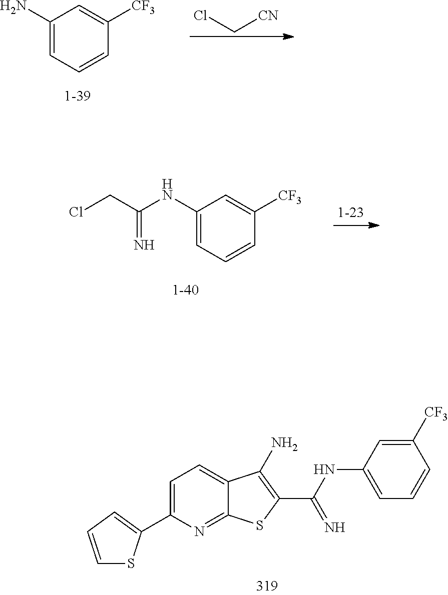

### Synthesis of 2-chloro-N-[3-(trifluoromethyl)phenyl]acetamidine (1-40)

Chloroacetonitrile (2.0 g, 26.7 mmol) and 3-(trifluoromethyl)benzenamine (4.20 g, 26.7 mmol) was treated with 4N HCl in 1,4-dioxane (50 mL). The reaction mixture was stirred at room temperature overnight. The reaction mixture was concentrated under vacuum and crude 1-40 was used for next step without further purification.

### Synthesis of 3-amino-6-(2-thienyl)-N-[3-(trifluoromethyl)phenyl]thieno[2,3-b]pyridine-2-carboxamidine (319)

See procedure used for the synthesis of 1-6.

### Example 30

### Synthesis of Compound 326

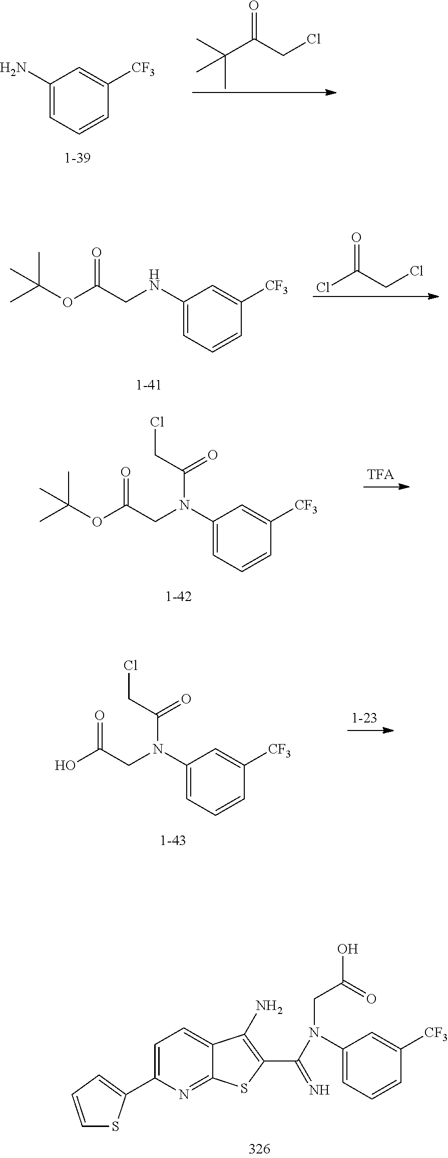

### Synthesis of tert-butyl 2-[3-(trifluoromethyl)anilino]acetate (1-41)

3-(trifluoromethyl)benzenamine (5.0 g, 31 mmol), tert-butyl 2-chloroacetate (33 g, 172 mmol) and K₂CO₃ (35 g, 253 mmol) in acetone (200 mL) was heated to 60° C. overnight and then the solid was removed by filtration. The filtrate was concentrated and the residue was purified by silica gel column chromatography eluting 5:1 hexane/MTBE to yield 10 g of 1-41 as a yellowish oil (quantitative yield).

### Synthesis of tert-butyl 2-[N-(2-chloroacetyl)-3-(trifluoromethyl)anilino]acetate (1-42)

To compound 1-41 (5 g, 18 mmol) and 2-chloroacetyl chloride (3.0 g, 27 mmol) in DCM (100 mL) was added a catalytic amount of tetrabutylammonium hydrosulfate followed by a solution of K₂CO₃ (5 g, 36 mmol) in water (100 mL). The reaction mixture was stirred at room temperature for 40 min and the organic portion was isolated and concentrated which was combined with another reaction product done on the same scale. The residue was purified via silica gel column chromatography eluting with 5:1 hexanes/MTBE to give 8 g of 1-42 as a yellowish oil (62% yield).

### Synthesis of 2-[N-(2-chloroacetyl)-3-(trifluoromethyl)anilino]acetic acid (1-43)

To a solution of compound 1-42 (1.0 g, 2.8 mmol) in DCM was added 10 mL of TFA. The resulting mixture was stirred at room temperature for 2 h and then the solvents were removed. The crude mixture was used for the next step directly.

### Synthesis of 2-[N-[3-amino-6-(2-thienyl)benzothiophene-2-carbonyl]-3-(trifluoromethyl)anilino]acetic acid (326)

To a crude mixture of compound 1-43, compound 1-23 (0.4 g, 1.8 mmol), K₂CO₃ (8 g, 58 mmol), was added DMF (20 mL). The reaction mixture was stirred at 50° C. for 1 h, then diluted with water (200 mL) and acidified with 2N HCl to pH 2. The solid was collected, triturated with of 1:1 THF/MTBE (40 mL) to give 120 mg of 326 as the potassium salt (14% yield).

### Synthesis of 8-(2-thienyl)-4-[3-(trifluoromethyl)phenyl]-1,3-dihydrobenzothiopheno[3,2-e][1,4]diazepine-2,5-dione (320)

This was a by-product formed resulting from intramolecular cyclization of the ethyl ester version of compound 326. After performing base catalyzed hydrolysis of the ester group of this intermediate, compound 320 was the major product isolated. Note: originally this was an alternate scheme to synthesize compound 326.

### Example 31

### Synthesis of Compound 322

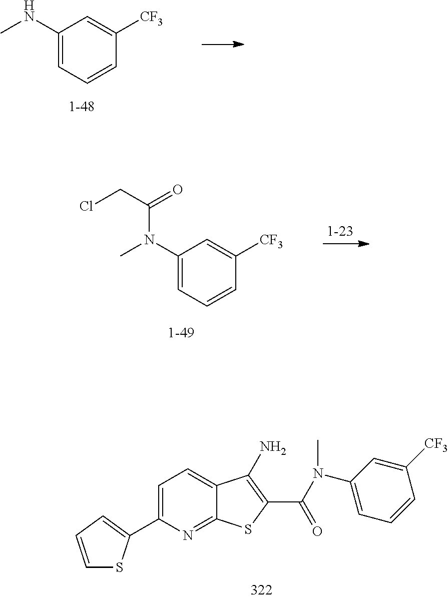 [SMILES: CNc1cccc(C(F)(F)F)c1 ; CN(C(=O)CCl)c1cccc(C(F)(F)F)c1 ; CN(C(=O)c1sc2nc(-c3cccs3)ccc2c1N)c1cccc(C(F)(F)F)c1]

### Synthesis of 2-chloro-N-methyl-N-[3-(trifluoromethyl)phenyl]acetamide (1-49)

3-(trifluoromethyl)-N-methylbenzenamine (3.0 g, 28 mmol) and 2-chloroacetyl chloride (12.6 g, 112 mmol) in 30 mL of DCM was added a catalytic amount of tetrabutylammonium hydrosulfate, followed by a solution of K₂CO₃ (15 g, 112 mmol) in 100 mL of water. The reaction mixture was stirred at room temperature for 40 min and the DCM layer was collected and combined with another same scale reaction. The residue was purified through a silica gel column eluting with 5:1 hexane/MTBE to give 2.7 g of 1-49 as a yellowish oil (38% yield).

### Synthesis of 3-amino-N-methyl-6-(2-thienyl)-N-[3-(trifluoromethyl)phenyl]thieno[2,3-b]pyridine-2-carboxamide (322)

To a mixture of compound 1-49 (2.7 g, 10.7 mmol) and 1-23 (1.5 g, 7.2 mmol) in 20 mL of EtOH was added 15 mL of 21% NaOEt in EtOH. The reaction mixture was heated for 2 h and then filtered. The solid was washed 20 mL of EtOH and dried to give 1.8 g of 322 (58% yield).

### Example 32

### Synthesis of Compound 323

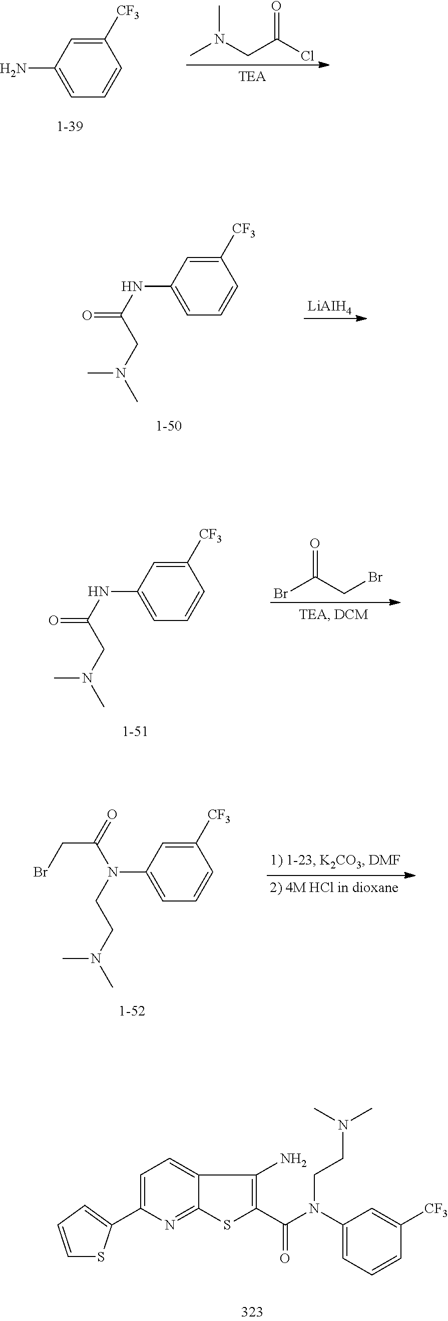

### Synthesis of 2-(dimethylamino)-N-[3-(trifluoromethyl)phenyl]acetamide (1-50)

To a solution of 2-(N,N-dimethylamino)-acetylchloride (25 g, 160 mmol) and TEA (14 mL, 100 mmol) in anhydrous DCM (100 mL) at 0° C. was added dropwise 3-(trifluoromethyl)-aniline (15 g, 93 mmol). The contents were slowly warmed to room temperature while stirring overnight. The reaction mixture was washed with water (2×20 mL), a saturated sodium bicarbonate solution, dried (Na₂SO₄), filtered and concentrated. Crude 1-50 (20 g) was obtained and used in the next step as such.

### Synthesis of N′,N′-dimethyl-N-[3-(trifluoromethyl)phenyl]ethane-1,2-diamine (1-51)

To a solution of crude 1-50 (20 g) in anhydrous THF (200 mL) at 0° C. was added dropwise a solution of LiAlH₄ (1M solution in THF, 186 mL, 186 mmol) and the contents were slowly warmed to 70° C. and refluxed overnight. The contents were cooled to 0° C., quenched with the addition of a saturated sodium potassium tartrate solution and filtered through a pad of Celite. The clear solution was concentrated and the residue was partitioned between EtOAc (500 mL) and water (100 mL). The layers were separated and the organic layer was washed with a saturated NaHCO₃ solution, dried (Na₂SO₄), filtered and concentrated. The residue obtained was left at high-vacuum overnight affording the desired intermediate 1-51 (8 g) as a brown oil.

### Synthesis of 2-bromo-N-(2-dimethylaminoethyl)-N-[3-(trifluoromethyl)phenyl]acetamide (1-52)

See procedure used for the synthesis of 1-5.

### Synthesis of 3-amino-N-(2-dimethylaminoethyl)-6-(2-thienyl)-N-[3-(trifluoromethyl)phenyl]thieno[2,3-b]pyridine-2-carboxamide (323)

To a mixture of 1-23 and 1-52 in anhydrous DMF (30 mL) at room temperature was added K₂CO₃ (13.8 g, 100 mmol) and the contents were stirred at 90° C. for 2 days. The contents were cooled to room temperature and poured into ice-cold water. The solid obtained was filtered, washed with MTBE (3×50 mL) and dried. The orange solid obtained (1.5 g) was treated with 4M HCl in dioxane (20 mL) at room temperature for 5 h and filtered. The orange solid was dried under high-vacuum affording 323 as the HCl salt (1.2 g).

### Example 33

### Synthesis of Compound 324

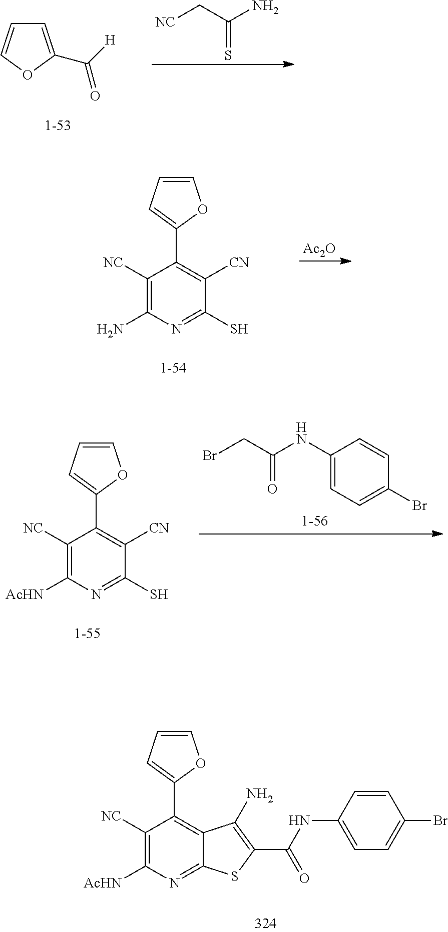

### Synthesis of 2-amino-4-(2-furyl)-6-sulfanyl-pyridine-3,5-dicarbonitrile (1-54)

Fufural (3.0 g, 31 mmol), 2-cyanoethanethioamide (6.0 g, 60 mmol) and 5 mL of 4-methylmorpholine in 50 mL of EtOH was heated at 80° C. for 6 h. The reaction mixture was added to water (200 mL) and acidified with 2N HCl to pH 2. The resulting solid was collected, washed with water (20 mL), and dried to afford 3.3 g of 1-54 (44% yield).

### Synthesis of N-[3,5-d]cyano-4-(2-furyl)-6-sulfanyl-2-pyridyl]acetamide (1-55)

To a suspension of compound 1-54 (3.3 g, 13 mmol) in 50 mL of DCM was added 5 mL of pyridine followed by 3 mL of acetic anhydride. The reaction mixture was stirred for 2 h and filtered. The solid was collected and triturated with EtOH (50 mL) at 60° C. for 30 minutes. The solid was collected and dried to give 2.5 g of 1-55 (67% yield).

### Synthesis of 6-acetamido-3-amino-N-(4-bromophenyl)-5-cyano-4-(2-furyl)thieno[2,3-b]pyridine-2-carboxamide (324)

To a solution of 2-bromo-N-(4-bromophenyl)acetamide (1 g, 3.52 mmol, 2 eq) and 1-55 (0.5 g, 1.76 mmol, 1 eq) in anhydrous DMF (20 mL), was added K₂CO₃ (0.36 g, 2.64 mmol, 1.5 eq) at room temp. The reaction mixture was heated at 80° C. for 2 h and then evaporated in vacuo. The residue was treated with ice water, stirred and the solid was collected by filtration. The solid was triturated with EtOAc to afford 95 mg of compound 324 (11% yield) as a light brown solid.

The intermediate 2-bromo-N-(4-bromophenyl)acetamide was prepared as follows: To a solution of 4-bromo aniline (20 g, 116.3 mmol, 1 eq) in anhydrous DCM (200 mL) and TEA (24.3 mL, 174.5 mmol, 1.5 eq) at 0° C., was added bromoacetyl bromide (11.1 mL, 127.9 mmol, 1.1 eq) dropwise over 30 min. The reaction mixture was stirred at room temperature for 2 h. Volatiles were removed under reduced pressure and the residue was partitioned between EtOAc and water. The layers were separated and the organic layer was washed with brine, dried (Na₂SO₄), filtered and concentrated to afford 24 g of 2-bromo-N-(4-bromophenyl)acetamide as a dark brown solid.

### Example 34

### Synthesis of Compound 325

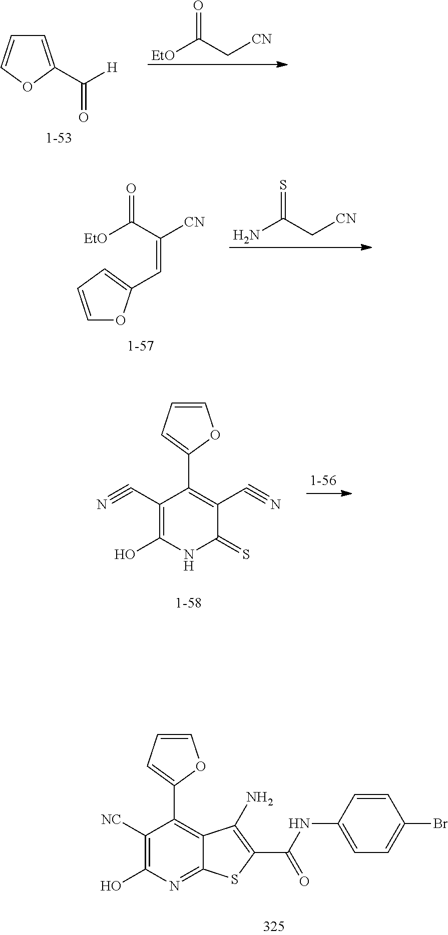 [SMILES: O=Cc1ccco1 ; CCOC(=O)CC#N ; CCOC(=O)/C(C#N)=C\c1ccco1 ; N#CCC(N)=S ; N#Cc1c(O)[nH]c(=S)c(C#N)c1-c1ccco1 ; N#Cc1c(O)nc2sc(C(=O)Nc3ccc(Br)cc3)c(N)c2c1-c1ccco1]

### Synthesis of Ethyl 2-cyano-3-(2-furyl)prop-2-enoate (1-57)

To a mixture of fufural (5 g, 52 mmol) and ethyl 2-cyanoacetate (5 g, 44 mmol) in EtOH (50 mL) was added TEA (0.5 mL). The reaction mixture was stirred for 30 minutes. The resulting white solid was collected and dried to give 6 g of 1-57 (71% yield).

### Synthesis of 4-(2-furyl)-2-hydroxy-6-thioxo-1H-pyridine-3,5-dicarbonitrile (1-58)

See procedure for 1-54.

### Synthesis of 3-amino-N-(4-bromophenyl)-5-cyano-4-(2-furyl)-6-hydroxy-thieno[2,3-b]pyridine-2-carboxamide (325)

To a mixture of 1-58 (750 mg, 3.0 mmol), 1-56 (1.0 g, 4.0 mmol), K₂CO₃ (2.1 g, 15 mmol) was added DMF (15 mL). The resulting mixture was stirred at 50° C. for 2 h, diluted with water (1000 mL) and acidified to a pH 2. The solid was collected and dried to give 250 mg of 325 as brown solid (18% yield).

### Example 35

### Synthesis of Compound 327

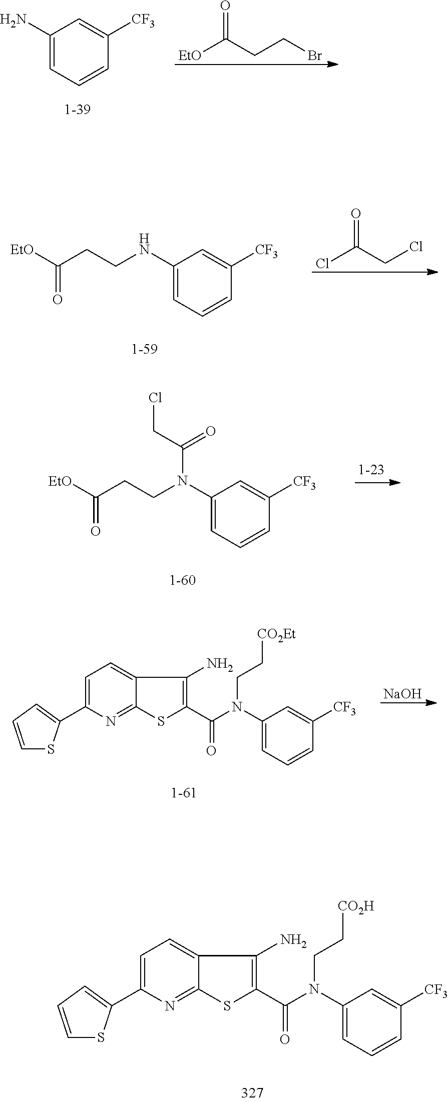

### Synthesis of Ethyl 3-[3-(trifluoromethyl)anilino]propanoate (1-59)

To a solution of ethyl 3-bromopropanoate (10 g, 60 mmol) and 3-(trifluoromethyl)benzenamine (5 g, 31 mmol) in DMF (100 mL) was added K₂CO₃ (10 g, 77 mmol). The resulting mixture was heated to 120° C. for 2 days. The solid was removed by filtration, washed with MTBE (200 mL), and the filtrate was diluted with water (1000 mL). The organic layer was collected, dried, filtered, and concentrated. The crude mixture was purified by silica gel column chromatography eluting 15:1 hexanes/MTBE to give 2 g of 1-59 as a yellow oil (25% yield).

### Synthesis of ethyl 3-[N-(2-chloroacetyl)-3-(trifluoromethyl)anilino]propanoate (1-60)

To a solution of 1-59 (2 g, 7.6 mmol), 2-chloroacetyl chloride (3.4 g, 30 mmol), a catalytic amount of tetrabutylammonium hydrosulfate in 40 mL of DCM was added a solution of K₂CO₃ (4.0 g, 30 mmol) in water (40 mL). The resulting mixture was stirred at room temperature for 40 min and then the organic layer was collected and concentrated. The crude mixture was purified through silica gel column chromatography eluting 4:1 hexanes/MTBE to give 2.8 g of 1-60 as a yellow oil in quantitative yield.

### Synthesis of Ethyl 3-[N-[3-amino-6-(2-thienyl)thieno[2,3-b]pyridine-2-carbonyl]-3-(trifluoromethyl)-anilino]propanoate (1-61)

To a mixture of 1-60 (2.8 g, 8.3 mmol), 1-23 (1.5 g, 6.9 mmol), and K₂CO₃ (11.5 g, 83 mmol) was added 25 mL of DMF. The resulting mixture was stirred at 50° C. for 2 h and then diluted with water (1000 mL). Following extraction with EtOAc (1000 mL), the combined organic layers were dried, filtered, and concentrated. The crude mixture was triturated with MTBE to give 2 g of 1-61 as a yellow solid (56% yield).

### Synthesis of 3-[N-[3-amino-6-(2-thienyl)thieno[2,3-b]pyridine-2-carbonyl]-3-(trifluoromethyl)-anilino]propanoic acid (327)

To solution of 1-61 (500 mg, 0.96 mmol) in THF was added 40 a 4N NaOH solution (40 mL). The resulting mixture was stirred at room temperature overnight. Solvents were removed and the solid was collected, washed with water (50 mL), THF (5 mL), and dried to give 400 mg of 327 as yellow solid (85% yield).

### Example 36

### Synthesis of Compounds 329 and 330

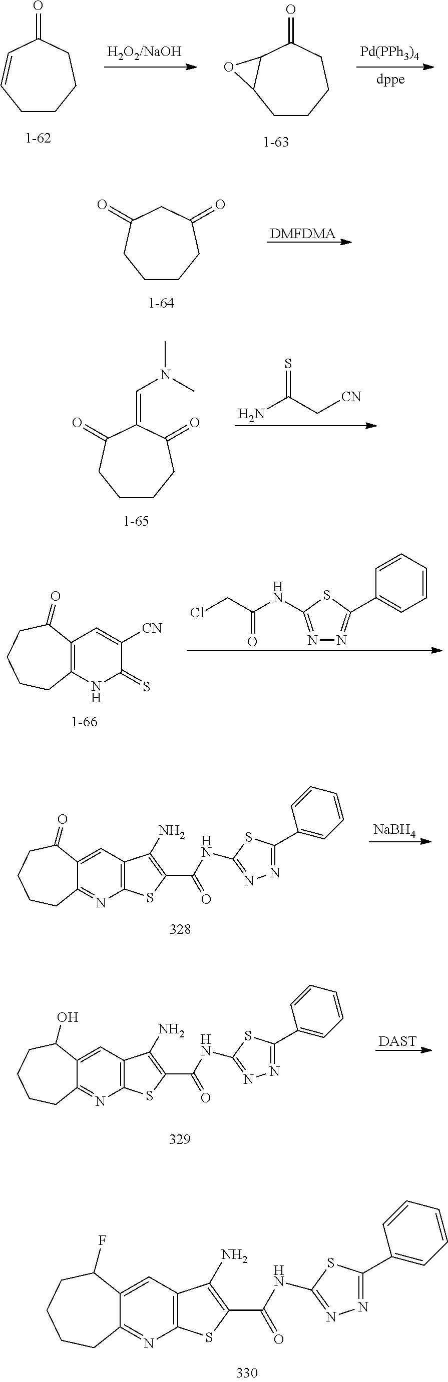

### Synthesis of 8-oxabicyclo[5.1.0]octan-6-one (1-63)

To a solution of cyclohept-2-enone (6.0 g, 45.5 mmol) in MeOH (40 mL) was added 13.6 ml of H₂O₂ at −4° C., followed by 7 mL of 10% NaOH solution. The resulting mixture was stirred at room temperature for 1 h, diluted with brine (1000 mL), and extracted with MTBE (2×200 mL). The combined organic layers were dried, filtered, concentrated and the crude material was purified by silica gel column chromatography eluting 15:1 hexanes/MTBE to give 5.5 g of 1-63 as a yellowish oil (96% yield).

### Synthesis of Cycloheptane-1,3-dione (1-64)

To a solution of 1-63 (6.0 g, 47 mmol) in toluene (18 mL) was added Pd(PPh₃)₄ (2.7 g, 2.35 mmol) and 1,2-bis(diphenylphosphino)ethane (1.0 g, 2.35 mmol). The reaction was bubbled with N₂ for 10 min, sealed in a 75 mL pressure tube and heated at 100° C. overnight. The reaction was cooled to room temperature and the solid was filtered off. The filtrate was collected, concentrated and purified by silica gel column chromatography eluting 1:10 hexanes/diethyl ether to give 5.0 g of crude product. This material was distilled to give 3.0 g of 1-64 as a yellowish oil which was used in the next step directly.

### Synthesis of 2-(dimethylaminomethylene)cycloheptane-1,3-dione (1-65)

A solution of 1-64 (3.0 g, 23.8 mmol) in N,N-dimethylformamide dimethyl acetal (30 mL) was stirred at room temperature overnight. The reaction mixture was concentrated in vacuo, the solid was collected and washed with 1:1 of hexane/diethyl ether (50 mL) to give 3.4 g of 1-65 as a yellowish solid (79% yield).

### Synthesis of 5-oxo-2-thioxo-6,7,8,9-tetrahydro-1H-cyclohepta[b]pyridine-3-carbonitrile (1-66)

See procedure used for the synthesis of 1-14.

### Synthesis of 3-[N-[3-amino-6-(2-thienyl)thieno[2,3-b]pyridine-2-carbonyl]-3-(trifluoromethyl)anilino]propanoic acid (328)

See procedure used for the synthesis of 1-6.

### Synthesis of 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide (329)

To a solution of 328 (100 mg, 0.23 mmol) in EtOH was added NaBH₄ (100 mg, 2.6 mmol) and the reaction mixture was stirred at room temperature for 40 min and then quenched with a saturated NH₄Cl solution (20 mL). The solid was collected, washed with water (20 mL), and dried to give 110 mg of 329 as a yellow solid in quantitative yield.

### Synthesis of 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide (330)

To a solution of 329 (640 mg, 1.47 mmol) in DCM (60 mL) was added XtalFluor-E (503 mg, 2.2 mmol). The resulting mixture was stirred at room temperature for 40 min and then concentrated. The crude material was purified by silica gel column chromatography eluting DCM/THF to give 30 mg of 330 as a yellow solid (5% yield).

### Example 37

### Synthesis of Compounds 331, 333-338, 340-344, 347-349, 351-353 and 356

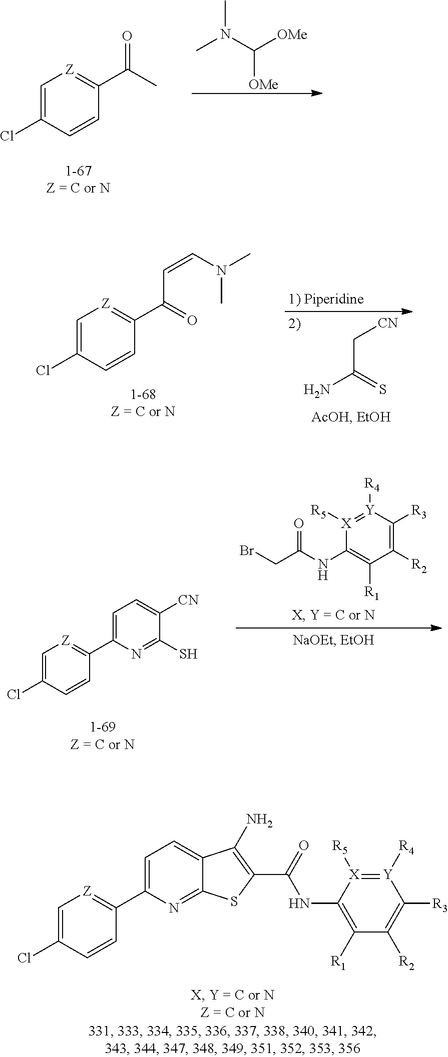

### Synthesis of 1-(4-chlorophenyl)-3-(dimethylamino)prop-2-en-1-one (1-68)

See procedure used for intermediate 1-37.

### Synthesis of 6-(4-chlorophenyl)-2-sulfanyl-pyridine-3-carbonitrile (1-69)

A solution of compound 1-68 (5 g, 23.84 mmol, 1.0 equiv.) in piperidine (18 mL) was refluxed for 2 h. The reaction mixture was cooled to ambient temp, concentrated under vacuum, and azeotroped with EtOH. To the crude intermediate was added EtOH (100 mL), 2-cyanothioacetamide (2.9 g, 28.6 mmol, 1.2 equiv.), and AcOH (1.7 mL). The mixture was refluxed for 16 h, cooled to room temperature, poured into an ice/water mixture (200 mL) and stirred for 15 minutes. Solids were removed by filtration, washed with water, and triturated with EtOH (50 mL) followed by 1:1 EtOAc/Hex mixture. The solids were dried under vacuum to give 4.3 g of compound 1-69 (73% overall yield).

### General Procedure for Compounds 331, 333, 334, 335, 336, 337, 338, 340, 341, 342, 343, 344, 347, 348, 349, 351, 352, 353, 356

For the synthesis of final compounds see the procedure used for intermediate 1-6. Compound 334 required an additional step involving hydrolysis of the ester following the cyclization reaction. Note: The bromoacetamide intermediate used in the final reaction was synthesized using the same procedure used for the synthesis of 1-24. Please note some compounds required reduction of the parent nitro moiety to the corresponding amine and was based upon commercial availability of the starting materials.

### Example 38

### Synthesis of Compounds 332, 339 and 345

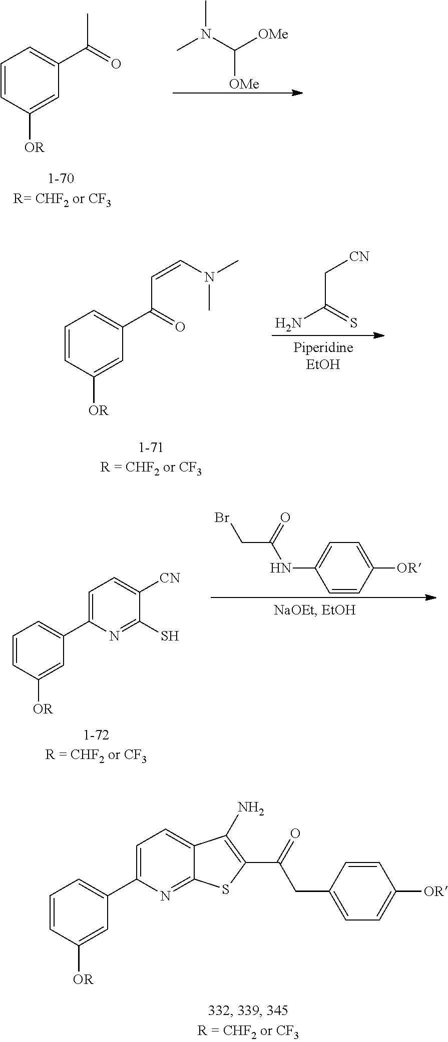

The same experimental procedures used for the compounds above (i.e., 331, 333, 334, etc.) were used for the synthesis of compounds 332, 339, and 345.

### Example 39

### Synthesis of Compound 346

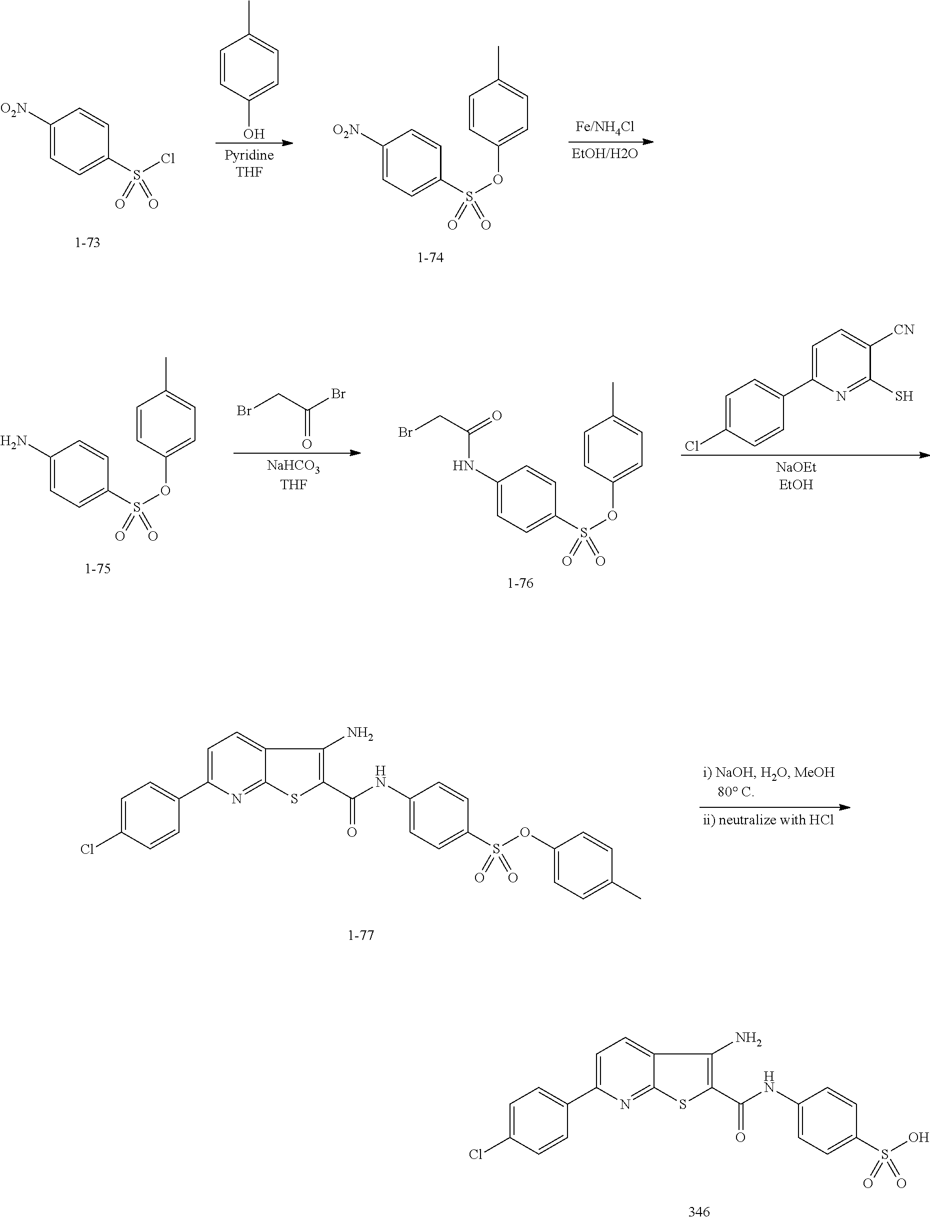

### Synthesis of p-tolyl 4-nitrobenzenesulfonate (1-74)

To a solution of compound 1-73 (4 g, 37 mmol), pyridine (4.5 mL) and THF (50 mL) was added a solution of p-cresol (9.8 g) in THF (25 mL) slowly over 10 min at 0° C. The reaction mixture was allowed to reach ambient temp and then heated to 65° C. for 48 h. The reaction was stopped by adding a saturated aqueous NH₄Cl solution and extracted with EtOAc. The organic layer was washed with brine, dried over Na₂SO₄, filtered and concentrated under vacuum to give a residue. The residue was purified by silica gel column chromatography eluting with 0-50% EtOAc/Hexanes to give 7.7 g of compound 1-74.

### Synthesis of p-tolyl 4-aminobenzenesulfonate (1-75)

To a mixture of compound 1-74 (2 g, 6.8 mmol, 1.0 equiv.) in EtOH (40 mL) was added a solution of NH₄Cl (1.5 g, 27 mmol, 4.0 equiv.) in 10 mL of water followed by iron (1.5 g, 27 mmol, 4.0 equiv.). The reaction mixture was heated to 80° C. for 20 min, cooled to ambient temp, filtered through a pad of Celite, and then washed with MeOH and DCM. The combined filtrates were concentrated under vacuum and extracted with DCM. The organic portion was washed with water, dried (Na₂SO₄), filtered and concentrated under vacuum to give crude material. The crude product was purified by silica gel column chromatography to give 1.1 g of compound 1-75 (61% yield).

### Synthesis of p-tolyl 4-[(2-bromoacetyl)amino]benzenesulfonate (1-76)

To a solution of compound 1-75 (1.1 g, 4.2 mmol, 1.0 equiv.) in THF (100 mL) was added NaHCO₃ (5.3 g, 6.3 mmol, 1.5 equiv.) and bromoacetyl bromide (0.44 mL, 5.02 mmol, 1.2 equiv.) at 0° C. The reaction mixture was warmed to ambient temp and stirred for 16 h. The reaction mixture was filtered through a pad of Celite, washed with DCM, and the combined filtrates were concentrated under vacuum to give crude compound 1-76. This material was carried to next step without further purification.

### Synthesis of p-tolyl 4-[[3-amino-6-(4-chlorophenyl)thieno[2,3-b]pyridine-2-carbonyl]amino]-benzenesulfonate (1-77)

See procedure used for the synthesis of 1-6.

### Synthesis of 4-[[3-amino-6-(4-chlorophenyl)thieno[2,3-b]pyridine-2-carbonyl]amino]benzenesulfonic acid (346)

A mixture of compound 1-77 (425 mg), 10 mL of 20% NaOH in water and MeOH (10 mL) was heated to 80° C. for 14 h. The mixture was cooled to ambient temperature and the solids were removed by filtration, washed with water, DCM, hexanes and dried under vacuum. The solids were suspended in water (5 mL) and acidified with 3N HCl to adjust the pH to 2-3 and stirred for 30 min. The solids were filtered, washed with water, DCM and hexanes. The solids were dried under vacuum at 35° C. for 14 h to give 210 mg of 346 (59% overall yield).

### Example 40

### Synthesis of Compounds 350, 354 and 355

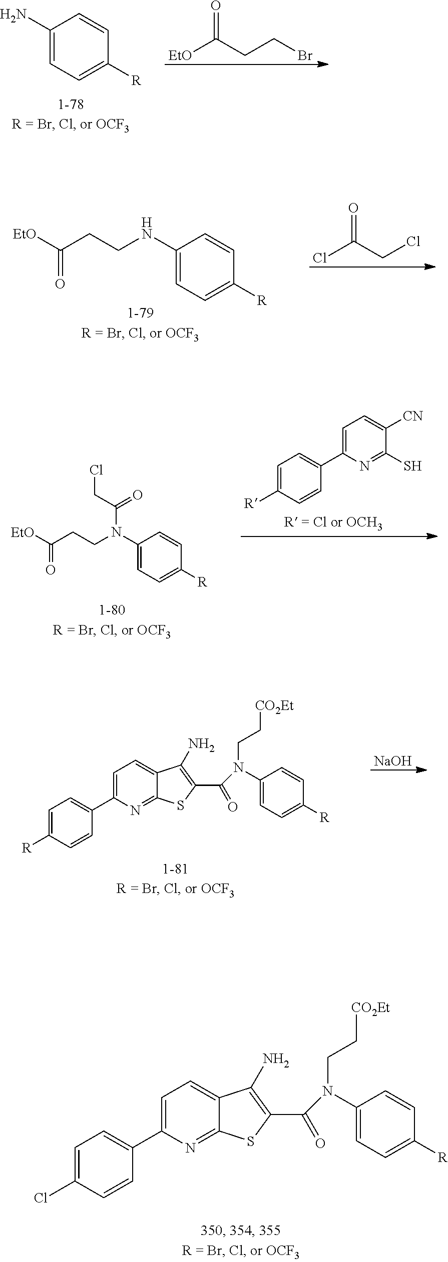

The same experimental procedures used for the compound 327 were used for the synthesis of compounds 350, 354, and 355.

### REFERENCES

1. Barth, O. M. 1999. Ultrastructural aspects of the dengue virus (flavivirus) particle morphogenesis. J Submicrosc Cytol Pathol 31:407-12.
2. Benarroch, D., M. P. Egloff, L. Mulard, C. Guerreiro, J. L. Romette, and B. Canard. 2004. A structural basis for the inhibition of the NS5 dengue virus mRNA 2′-O-methyltransferase domain by ribavirin 5′-triphosphate. J Biol Chem 279:35638-43.
3. Brinton, M. A., and J. H. Dispoto. 1988. Sequence and secondary structure analysis of the 5′-terminal region of flavivirus genome RNA. Virology 162:290-9.
4. CDC. 2005. Dengue Fever, http://www2.ncid.cdc.gov/travel/yb/utils/ybGet.asp?section=dis&obj=dengue.htm.
5. Edelman, R., S. S. Wasserman, S. A. Bodison, R. J. Putnak, K. H. Eckels, D. Tang, N. Kanesa-Thasan, D. W. Vaughn, B. L. Innis, and W. Sun. 2003. Phase I trial of 16 formulations of a tetravalent live-attenuated dengue vaccine. Am J Trop Med Hyg 69:48-60.
6. Falgout, B., M. Pethel, Y. M. Zhang, and C. J. Lai. 1991. Both nonstructural proteins NS2B and NS3 are required for the proteolytic processing of dengue virus nonstructural proteins. J Virol 65:2467-75.
7. Fink, J., F. Gu, and S. G. Vasudevan. 2006. Role of T cells, cytokines and antibody in dengue fever and dengue haemorrhagic fever. Rev Med Virol 16:263-75.
8. Halstead, S. B. 1988. Pathogenesis of dengue: challenges to molecular biology. Science 239:476-81.
9. Hase, T., P. L. Summers, K. H. Eckels, and W. B. Baze. 1987. An electron and immunoelectron microscopic study of dengue-2 virus infection of cultured mosquito cells: maturation events. Arch Virol 92:273-91.
10. Hillen, W., G. Klock, I. Kaffenberger, L. V. Wray, and W. S. Reznikoff. 1982. Purification of the TET repressor and TET operator from the transposon Tn10 and characterization of their interaction. J Biol Chem 257:6605-13.
11. Kanesa-thasan, N., W. Sun, G. Kim-Ahn, S. Van Albert, J. R. Putnak, A. King, B. Raengsakulsrach, H. Christ-Schmidt, K. Gilson, J. M. Zahradnik, D. W. Vaughn, B. L. Innis, J. F. Saluzzo, and C. H. Hoke, Jr. 2001. Safety and immunogenicity of attenuated dengue virus vaccines (Aventis Pasteur) in human volunteers. Vaccine 19:3179-88.
12. Kitchener, S., M. Nissen, P. Nasveld, R. Forrat, S. Yoksan, J. Lang, and J. F. Saluzzo. 2006. Immunogenicity and safety of two live-attenuated tetravalent dengue vaccine formulations in healthy Australian adults. Vaccine 24:1238-41.
13. Koff, W. C., J. L. Elm, Jr., and S. B. Halstead. 1982. Antiviral effects if ribavirin and 6-mercapto-9-tetrahydro-2-furylpurine against dengue viruses in vitro. Antiviral Res 2:69-79.
14. Koff, W. C., R. D. Pratt, J. L. Elm, Jr., C. N. Venkateshan, and S. B. Halstead. 1983. Treatment of intracranial dengue virus infections in mice with a lipophilic derivative of ribavirin. Antimicrob Agents Chemother 24:134-6.
15. Leitmeyer, K. C., D. W. Vaughn, D. M. Watts, R. Salas, I. Villalobos, C. de, C. Ramos, and R. Rico-Hesse. 1999. Dengue virus structural differences that correlate with pathogenesis. J Virol 73:4738-47.
16. Malinoski, F. J., S. E. Hasty, M. A. Ussery, and J. M. Dalrymple. 1990. Prophylactic ribavirin treatment of dengue type 1 infection in rhesus monkeys. Antiviral Res 13:139-49.
17. Markoff, L., A. Chang, and B. Falgout. 1994. Processing of flavivirus structural glycoproteins: stable membrane insertion of premembrane requires the envelope signal peptide. Virology 204:526-40.
18. Medin, C. L., K. A. Fitzgerald, and A. L. Rothman. 2005. Dengue virus nonstructural protein NS5 induces interleukin-8 transcription and secretion. J Virol 79:11053-61.
19. Modis, Y., S. Ogata, D. Clements, and S. C. Harrison. 2003. A ligand-binding pocket in the dengue virus envelope glycoprotein. Proc Natl Acad Sci USA 100:6986-91.
20. Monath, T. P. 1994. Dengue: the risk to developed and developing countries. Proc Natl Acad Sci USA 91:2395-400.
21. Mukhopadhyay, S., R. J. Kuhn, and M. G. Rossmann. 2005. A structural perspective of the flavivirus life cycle. Nat Rev Microbiol 3:13-22.
22. O'Brien, J., I. Wilson, T. Orton, and F. Pognan. 2000. Investigation of the Alamar Blue (resazurin) fluorescent dye for the assessment of mammalian cell cytotoxicity. Eur Biochem 267:5421-6.
23. PAHO. 2006. Dengue and dengue hemorrhagic fever http://www.paho.org/english/ad/dpc/cd/dengue.htm.
24. Raviprakash, K., M. Sinha, C. G. Hayes, and K. R. Porter. 1998. Conversion of dengue virus replicative form RNA (RF) to replicative intermediate (RI) by nonstructural proteins NS-5 and NS-3. Am J Trop Med Hyg 58:90-5.
25. Rothman, A. L., and F. A. Ennis. 1999. Immunopathogenesis of Dengue hemorrhagic fever. Virology 257:1-6.
26. Sabchareon, A., J. Lang, P. Chanthavanich, S. Yoksan, R. Forrat, P. Attanath, C. Sirivichayakul, K. Pengsaa, C. Pojjaroen-Anant, W. Chokejindachai, A. Jagsudee, J. F. Saluzzo, and N. Bhamarapravati. 2002. Safety and immunogenicity of tetravalent live-attenuated dengue vaccines in That adult volunteers: role of serotype concentration, ratio, and multiple doses. Am J Trop Med Hyg 66:264-72.
27. Schlesinger, S., and M. J. Schlesinger. 1990. Replication of togaviridae and flaviviridae, p. 697-710. In B. N. Fields, D. M. Knipe, R. M. Chanock, M. S. Hirsch, J. L. Melnick, T. P. Monath, and B. Roizrnan (ed.), Virology, 2 ed, vol. 1. Ravens Press, New York.
28. Takhampunya, R., S. Ubol, H. S. Houng, C. E. Cameron, and R. Padmanabhan. 2006. Inhibition of dengue virus replication by mycophenolic acid and ribavirin. J Gen Virol 87:1947-52.
29. Thein, S., M. M. Aung, T. N. Shwe, M. Aye, A. Zaw, K. Aye, K. M. Aye, and J. Aaskov. 1997. Risk factors in dengue shock syndrome. Am J Trop Med Hyg 56:566-72.
30. Uchil, P. D., and V. Satchidanandam. 2003. Architecture of the flaviviral replication complex. Protease, nuclease, and detergents reveal encasement within double-layered membrane compartments. J Biol Chem 278:24388-98.
31. Umareddy, I., A. Chao, A. Sampath, F. Gu, and S. G. Vasudevan. 2006. Dengue virus NS4B interacts with NS3 and dissociates it from single-stranded RNA. J Gen Virol 87:2605-14.
32. WHO. 2002. Dengue and dengue haemorrhagic fever, http://www.who.int/mediacentre/factsheets/fs117/en/.
33. WHO. 1997. Dengue haemorrhagic fever, http://www.who.int/csr/resources/publications/dengue/Dengue publication/en/index.html.

All references cited herein are herein incorporated by reference in their entirety for all purposes.

The invention has been described in terms of preferred embodiments thereof, but is more broadly applicable as will be understood by those skilled in the art. The scope of the invention is only limited by the following claims.

What is claimed is:

1. A compound having the following general Formula III or a pharmaceutically acceptable salt thereof: 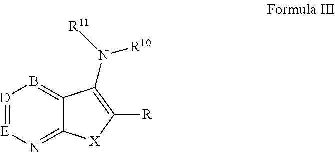
wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;
R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;
B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and
R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen.

2. The compound of claim 1, wherein X is S.

3. The compound of claim 1, wherein B is C—H.

4. The compound of claim 1, wherein D is a C—H.

5. The compound of claim 1, wherein E is C—R⁴ and R⁴ is a heteroaryl.

6. The compound of claim 1, wherein D is C—R³ and E is C—R⁴, and R³ and R⁴ form a ring.

7. The compound of claim 1, wherein R is a substituted aminocarbonyl.

8. The compound of claim 1 being 3-[N-[3-amino-6-(2-thienyl)thieno[2,3-b]pyridine-2-carbonyl]-3-(trifluoromethyl)anilino]propanoic acid.

9. A pharmaceutical composition comprising a pharmaceutically acceptable carrier and a compound having the following general Formula III or a pharmaceutically acceptable salt thereof: 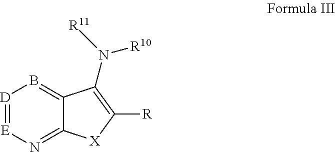
wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;
R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;
B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and
R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen,
wherein said composition is suitable for human or animal administration.

10. The composition of claim 9, wherein X is S.

11. The composition of claim 9, wherein B is C—H.

12. The composition of claim 9, wherein D is a C—H.

13. The composition of claim 9, wherein E is C—R⁴ and R⁴ is a heteroaryl.

14. The composition of claim 9, wherein D is C—R³ and E is C—R⁴, and R³ and R⁴ form a ring.

15. The composition of claim 9, wherein R is a substituted aminocarbonyl.

16. The composition of claim 9, wherein said compound is 3-[N-[3-amino-6-(2-thienyl)thieno[2,3-b]pyridine-2-carbonyl]-3-(trifluoromethyl)anilino]propanoic acid.

17. A method for the treatment of at least one type of a Dengue virus infection or disease associated therewith, comprising administering in a therapeutically effective amount to a mammal in need thereof, a compound of Formula III below or a pharmaceutically acceptable salt thereof: 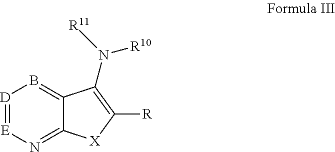
wherein X is selected from the groups consisting of: O, S and N—R′, wherein R′ is selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl;
R is selected from the group consisting of halogen, cyano, isocyano, nitro, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, hydroxysulfonyl, aminosulfonyl, substituted aminosulfonyl, acyl, arylacyl, heteroarylacyl, carboxy, alkoxycarbonyl, cycloalkyloxycarbonyl, aryloxycarbonyl, aminocarbonyl, and substituted aminocarbonyl;
B, D, and E are independently N or C—R², C—R³ and C—R⁴, respectively, wherein R², R³ and R⁴ are independently selected from the group consisting of hydrogen, substituted or unsubstituted alkyl, alkenyl, alkynyl, cycloalkyl, heterocycloalkyl, arylalkyl, aryl, heteroaryl, hydroxy, alkyloxy, aryloxy, heteroaryloxy, acyloxy, arylacyloxy, heteroarylacyloxy, alkylsulfonyloxy, arylsulfonyloxy, thio, alkylthio, arylthio, amino, alkylamino, dialkylamino, cycloalkylamino, heterocycloalkylamino, arylamino, heteroarylamino, acylamino, arylacylamino, heteroarylacylamino, alkylsulfonylamino, arylsulfonylamino, acyl, arylacyl, heteroarylacyl, alkylsulfinyl, arylsulfinyl, alkylsulfonyl, arylsulfonyl, aminosulfonyl, substituted aminosulfonyl, carboxy, alkoxycarbonyl, cycloalkyloxycarbonyl, aryloxycarbonyl, carbamoyl, substituted carbamoyl, halogen, cyano, isocyano and nitro; or R² and R³ or R³ and R⁴ together with the carbons they are attached to may form a substituted or unsubstituted ring, which may be aromatic or non-aromatic and may include one or more heteroatoms in the ring and may be fused with an aromatic or aliphatic ring; and
R¹⁰ and R¹¹ are independently selected from the groups consisting of hydrogen, alkyl, alkenyl, alkynyl, cycloalkyl, heterocycloalkyl, arylalkyl, aryl, heteroaryl, acyl, arylacyl, heteroarylacyl, sulfonyl, aminosulfonyl, substituted aminosulfonyl, alkoxycarbonyl, cycloalkyloxycarbonyl, aryloxycarbonyl, carbamoyl and substituted carbamoyl, provided that R¹⁰ and R¹¹ can't both be hydrogen.

18. The method of claim 17, wherein X is S.

19. The method of claim 17, wherein B is C—H.

20. The method of claim 17, wherein D is a C—H.

21. The method of claim 17, wherein E is C—R⁴ and R⁴ is a heteroaryl.

22. The method of claim 17, wherein D is C—R³ and E is C—R⁴, and R³ and R⁴ form a ring.

23. The method of claim 17, wherein R is a substituted aminocarbonyl.

24. The method of claim 17, wherein said compound is 3-[N-[3-amino-6-(2-thienyl)thieno[2,3-b]pyridine-2-carbonyl]-3-(trifluoromethyl)anilino]propanoic acid.

25. The method of claim 17, wherein the mammal is a human.

26. The method of claim 17, wherein said Dengue virus is selected from the group consisting of DEN-1, DEN-2, DEN-3, and DEN-4.

27. The method of claim 17, wherein said viral infection is associated with Dengue fever.

28. The method of claim 27, wherein said Dengue fever is selected from the group consisting of classical dengue fever and dengue hemorrhagic fever.

29. The method of claim 17, which further comprises co-administration of at least one agent selected from the group consisting of antiviral agent, vaccine, and interferon.

30. The method of claim 29, wherein said interferon is pegylated.

31. A compound selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide.

32. The compound of claim 31 being 3-amino-N,6-bis(4-chlorophenyl)thieno[2,3-b]pyridine-2-carboxamide.

33. The compound of claim 31 being 3-amino-6-[3-(difluoromethoxy)phenyl]-N-[4-(difluoromethoxy)phenyl]thieno[2,3-b]pyridine-2-carboxamide.

34. A pharmaceutical composition comprising a pharmaceutically acceptable carrier and a compound selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide, wherein said composition is suitable for human or animal administration.

35. The composition of claim 34, wherein said compound is 3-amino-N,6-bis(4-chlorophenyl)thieno[2,3-b]pyridine-2-carboxamide.

36. The composition of claim 34, wherein said compound is 3-amino-6-[3-(difluoromethoxy)phenyl]-N-[4-(difluoromethoxy)phenyl]thieno[2,3-b]pyridine-2-carboxamide.

37. A method for the treatment of at least one type of a Dengue virus infection or disease associated therewith, comprising administering in a therapeutically effective amount to a mammal in need thereof, a compound or a pharmaceutically acceptable salt thereof, wherein said compound is selected from the group consisting of: 3-amino-N-cyclohexyl-6,7,8,9-tetrahydro-5H-cyclohepta[b]thieno[3,2-e]pyridine-2-carboxamide; 3-amino-N-butyl-6,7,8,9-tetrahydro-5H-cyclohepta[b]thieno[3,2-e]pyridine-2-carboxamide; 3-amino-N-(tert-butyl)-6,7,8,9-tetrahydro-5H-cyclohepta[b]thieno[3,2-e]pyridine-2-carboxamide; 3-amino-6-methyl-N-(5-phenyl-1,3,4-thiadiazol-2-yl)thieno[2,3-b]pyridine-2-carboxamide; 3-amino-5-methyl-N-(5-phenyl-1,3,4-thiadiazol-2-yl)thieno[2,3-b]pyridine-2-carboxamide; 3-amino-4-methoxy-N-(5-phenyl-1,3,4-thiadiazol-2-yl)thieno[2,3-b]pyridine-2-carboxamide; 3-amino-4-methyl-N-(5-phenyl-1,3,4-thiadiazol-2-yl)thieno[2,3-b]pyridine-2-carboxamide; 3,5-diamino-N-(5-phenyl-1,3,4-thiadiazol-2-yl)thieno[2,3-b]pyridine-2-carboxamide; 3-amino-2-((5-phenyl-1,3,4-thiadiazol-2-yl)carbamoyl)thieno[2,3-b]pyridine-5-carboxylic acid; 3-amino-6-chloro-N-(5-phenyl-1,3,4-thiadiazol-2-yl)thieno[2,3-b]pyridine-2-carboxamide; 3-amino-6-methyl-N-(5-phenyl-1,3,4-thiadiazol-2-yl)-7,8-dihydro-5H-thieno[2,3-b][1,6]naphthyridine-2-carboxamide; 2-(thiophen-2-yl)-10-(3-(trifluoromethyl)phenyl)-7,8-dihydro-5H-pyrido[3′,2′:4,5]thieno[3,2-b][1,5]diazonine-6,9,11(10H)-trione; 7-(thiophen-2-yl)-3-(3-(trifluoromethyl)phenyl)pyrido[3′,2′:4,5]thieno[3,2-d]pyrimidine-2,4(1H,3H)-dione; 3-amino-6-(trifluoromethyl)-N-[3-(trifluoromethyl)phenyl]thieno[2,3-b]pyridine-2-carboxamide; 3-amino-6-(2,4-dimethylthiazol-5-yl)-N-[3-(trifluoromethyl)phenyl]thieno[2,3-b]pyridine-2-carboxamide; 3-amino-6-(2-thienyl)-N-[3-(trifluoromethyl)phenyl]thieno[2,3-b]pyridine-2-carboxamidine; 8-(thiophen-2-yl)-4-(3-(trifluoromethyl)phenyl)-3,4-dihydro-1H-pyrido[3′,2′:4,5]thieno[3,2-e][1,4]diazepine-2,5-dione; 3-amino-N-methyl-6-(2-thienyl)-N-[3-(trifluoromethyl)phenyl]thieno[2,3-b]pyridine-2-carboxamide; 3-amino-N-(2-dimethylaminoethyl)-6-(2-thienyl)-N-[3-(trifluoromethyl)phenyl]thieno[2,3-b]pyridine-2-carboxamide; 6-acetamido-3-amino-N-(4-bromophenyl)-5-cyano-4-(2-furyl)thieno[2,3-b]pyridine-2-carboxamide; 3-amino-N-(4-bromophenyl)-5-cyano-4-(2-furyl)-6-hydroxy-thieno[2,3-b]pyridine-2-carboxamide; 2-[N-[3-amino-6-(2-thienyl)thieno[2,3-b]pyridine-2-carbonyl]-3-(trifluoromethyl)anilino]acetic acid; 3-[N-[3-amino-6-(2-thienyl)thieno[2,3-b]pyridine-2-carbonyl]-3-(trifluoromethyl)anilino]propanoic acid; 3-amino-5-oxo-N-(5-phenyl-1,3,4-thiadiazol-2-yl)-6,7,8,9-tetrahydro-5H-cyclohepta[b]thieno[3,2-e]pyridine-2-carboxamide; 3-amino-5-hydroxy-N-(5-phenyl-1,3,4-thiadiazol-2-yl)-6,7,8,9-tetrahydro-5H-cyclohepta[b]thieno[3,2-e]pyridine-2-carboxamide; 3-amino-5-fluoro-N-(5-phenyl-1,3,4-thiadiazol-2-yl)-6,7,8,9-tetrahydro-5H-cyclohepta[b]thieno[3,2-e]pyridine-2-carboxamide; 3-amino-6-(4-chlorophenyl)-N-[4-(trifluoromethoxy)phenyl]thieno[2,3-b]pyridine-2-carboxamide; 3-amino-6-[3-(trifluoromethoxy)phenyl]-N-[4-(trifluoromethoxy)phenyl]thieno[2,3-b]pyridine-2-carboxamide; 3-amino-N,6-bis(4-chlorophenyl)thieno[2,3-b]pyridine-2-carboxamide; 4-[[3-amino-6-(4-chlorophenyl)thieno[2,3-b]pyridine-2-carbonyl]amino]benzoic acid; 3-amino-N-(5-bromo-2-pyridyl)-6-(4-chlorophenyl)thieno[2,3-b]pyridine-2-carboxamide; 3-amino-N-(6-bromo-3-pyridyl)-6-(4-chlorophenyl)thieno[2,3-b]pyridine-2-carboxamide; 3-amino-6-(4-chlorophenyl)-N-[4-(difluoromethyl)phenyl]thieno[2,3-b]pyridine-2-carboxamide; 3-amino-6-(4-chlorophenyl)-N-[4-(1,1-difluoroethyl)phenyl]thieno[2,3-b]pyridine-2-carboxamide; 3-amino-6-[3-(difluoromethoxy)phenyl]-N-[4-(trifluoromethoxy)phenyl]thieno[2,3-b]pyridine-2-carboxamide; 3-amino-6-(4-chlorophenyl)-N-[4-(difluoromethoxy)phenyl]thieno[2,3-b]pyridine-2-carboxamide; 3-amino-N-(2-bromophenyl)-6-(4-chlorophenyl)thieno[2,3-b]pyridine-2-carboxamide; 3-amino-6-(4-chlorophenyl)-N-(3,4-dichlorophenyl)thieno[2,3-b]pyridine-2-carboxamide; 3-amino-6-(4-chlorophenyl)-N-(2,3-dichlorophenyl)thieno[2,3-b]pyridine-2-carboxamide; 3-amino-N-(3-chlorophenyl)-6-(4-chlorophenyl)thieno[2,3-b]pyridine-2-carboxamide; 3-amino-6-[3-(difluoromethoxy)phenyl]-N-[4-(difluoromethoxy)phenyl]thieno[2,3-b]pyridine-2-carboxamide; 4-[[3-amino-6-(4-chlorophenyl)thieno[2,3-b]pyridine-2-carbonyl]amino]benzenesulfonic acid; 3-amino-6-(4-chlorophenyl)-N-(2,5-dichlorophenyl)thieno[2,3-b]pyridine-2-carboxamide; 3-amino-6-(4-chlorophenyl)-N-(3,4-dimethylphenyl)thieno[2,3-b]pyridine-2-carboxamide; 3-amino-N-(4-bromophenyl)-6-(5-chloro-2-pyridyl)thieno[2,3-b]pyridine-2-carboxamide; 3-(N-[3-amino-6-(4-chlorophenyl)thieno[2,3-b]pyridine-2-carbonyl]-4-bromo-anilino)propanoic acid; 3-amino-6-(4-chlorophenyl)-N-[4-(2,2,2-trifluoroacetyl)phenyl]thieno[2,3-b]pyridine-2-carboxamide; 3-amino-6-(4-chlorophenyl)-N-(5-chloro-2-pyridyl)thieno[2,3-b]pyridine-2-carboxamide; 3-amino-6-(4-chlorophenyl)-N-(6-chloro-3-pyridyl)thieno[2,3-b]pyridine-2-carboxamide; 3-[N-[3-amino-6-(3-methoxyphenyl)thieno[2,3-b]pyridine-2-carbonyl]-4-(trifluoromethoxy)anilino]propanoic acid; 3-(N-[3-amino-6-(4-chlorophenyl)thieno[2,3-b]pyridine-2-carbonyl]-4-chloro-anilino)propanoic acid; 3-amino-6-(4-chlorophenyl)-N-(4-hydroxyphenyl)thieno[2,3-b]pyridine-2-carboxamide; and 3-amino-N-(4-pyridyl)-6-(2-thienyl)thieno[2,3-b]pyridine-2-carboxamide.

38. The method of claim 37, wherein said compound is 3-amino-N,6-bis(4-chlorophenyl)thieno[2,3-b]pyridine-2-carboxamide.

39. The method of claim 37, wherein said compound is 3-amino-6-[3-(difluoromethoxy)phenyl]-N-[4-(difluoromethoxy)phenyl]thieno[2,3-b]pyridine-2-carboxamide.

40. The method of claim 37, wherein the mammal is a human.

41. The method of claim 37, wherein said Dengue virus is selected from the group consisting of DEN-1, DEN-2, DEN-3, and DEN-4.

42. The method of claim 37, wherein said viral infection is associated with Dengue fever.

43. The method of claim 42, wherein said Dengue fever is selected from the group consisting of classical dengue fever and dengue hemorrhagic fever.

44. The method of claim 37, which further comprises co-administration of at least one agent selected from the group consisting of antiviral agent, vaccine, and interferon.

45. The method of claim 44, wherein said interferon is pegylated.

46. A pharmaceutical composition comprising a pharmaceutically acceptable carrier and a compound or a pharmaceutically acceptable salt thereof, wherein said compound is selected from the group consisting of: 3-amino-N-(4-bromophenyl)-6-(4-chlorophenyl)thieno[2,3-b]pyridine-2-carboxamide; 3-amino-6-(3-methoxyphenyl)-N-[4-(trifluoromethoxy)phenyl]thieno[2,3-b]pyridine-2-carboxamide; 3-amino-N-(2,5-dichlorophenyl)-6-(2-thienyl)thieno[2,3-b]pyridine-2-carboxamide; 3-amino-N-(2,3-dichlorophenyl)-6-(2-thienyl)thieno[2,3-b]pyridine-2-carboxamide; 3-amino-N-(4-bromophenyl)-6-(3-methoxyphenyl)thieno[2,3-b]pyridine-2-carboxamide; 3-amino-6-(1,3-benzodioxol-5-yl)-N-(2-bromo-4-methyl-phenyl)thieno[2,3-b]pyridine-2-carboxamide; and 3-amino-6-(3-methoxyphenyl)-N-(2-phenoxyphenyl)thieno[2,3-b]pyridine-2-carboxamide, wherein said composition is suitable for human or animal administration.

47. The composition of claim 46, wherein said compound is 3-amino-N-(4-bromophenyl)-6-(4-chlorophenyl)thieno[2,3-b]pyridine-2-carboxamide.

48. The composition of claim 46, wherein said compound is 3-amino-6-(3-methoxyphenyl)-N-[4-(trifluoromethoxy)phenyl]thieno[2,3-b]pyridine-2-carboxamide.

49. A method for the treatment of at least one type of a Dengue virus infection or disease associated therewith, comprising administering in a therapeutically effective amount to a mammal in need thereof, a compound or a pharmaceutically acceptable salt thereof, wherein said compound is selected from the group consisting of: 3-amino-N-(4-bromophenyl)-6-(4-chlorophenyl)thieno[2,3-b]pyridine-2-carboxamide; 3-amino-6-(3-methoxyphenyl)-N-[4-(trifluoromethoxy)phenyl]thieno[2,3-b]pyridine-2-carboxamide; 3-amino-N-(2,5-dichlorophenyl)-6-(2-thienyl)thieno[2,3-b]pyridine-2-carboxamide; 3-amino-N-(2,3-dichlorophenyl)-6-(2-thienyl)thieno[2,3-b]pyridine-2-carboxamide; 3-amino-N-(4-bromophenyl)-6-(3-methoxyphenyl)thieno[2,3-b]pyridine-2-carboxamide; 3-amino-6-(1,3-benzodioxol-5-yl)-N-(2-bromo-4-methyl-phenyl)thieno[2,3-b]pyridine-2-carboxamide; and 3-amino-6-(3-methoxyphenyl)-N-(2-phenoxyphenyl)thieno[2,3-b]pyridine-2-carboxamide.

50. The method of claim 49, wherein said compound is 3-amino-N-(4-bromophenyl)-6-(4-chlorophenyl)thieno[2,3-b]pyridine-2-carboxamide.

51. The method of claim 49, wherein said compound is 3-amino-6-(3-methoxyphenyl)-N-[4-(trifluoromethoxy)phenyl]thieno[2,3-b]pyridine-2-carboxamide.

52. The method of claim 49, wherein the mammal is a human.

53. The method of claim 49, wherein said Dengue virus is selected from the group consisting of DEN-1, DEN-2, DEN-3, and DEN-4.

54. The method of claim 53, wherein said viral infection is associated with Dengue fever.

55. The method of claim 54, wherein said Dengue fever is selected from the group consisting of classical dengue fever and dengue hemorrhagic fever.

56. The method of claim 49, which further comprises co-administration of at least one agent selected from the group consisting of antiviral agent, vaccine, and interferon.

57. The method of claim 56, wherein said interferon is pegylated.
